(19) United States
(12) Patent Application Publication
(10) Pub. No.: US 2026/0124255 A1
(43) Pub. Date: May 7, 2026

(54) Salmonella vectored therapies for treatment of cancer

(71) Applicant: UNIVERSITY OF FLORIDA RESEARCH FOUNDATION, INCORPORATED, Gainesville, FL (US)

(72) Inventors: Roy CURTISS, III, Gainesville, FL (US); Shifeng WANG, Gainesville, FL (US)

(21) Appl. No.: 19/082,056

(22) Filed: Mar. 17, 2025

Related U.S. Application Data
(63) Continuation-in-part of application No. 18/870,052.
(60) Provisional application No. 63/348,492, filed on Jun. 3, 2022.

(51) Int. Cl. A61K 35/741; C07K 14/435; C07K 14/52; C07K 14/575; C07K 14/705; C07K 14/74; C07K 16/32; C12N 15/74

(52) U.S. Cl. CPC A61K 35/741; C07K 14/43595; C07K 14/521; C07K 14/575; C07K 14/70521; C07K 14/70539; C07K 16/32; C12N 15/74; C07K 2317/622; C07K 2319/00; C07K 2319/30

(57) ABSTRACT

A genetically modified Salmonella cell (GMSC) engineered to display pattern-associated and danger-associated molecular patterns to recruit and enhance innate immunity and exhibit specific targeting to cells and regulated delayed lysis in vivo, the GMSC comprising a first heterologous nucleic acid that encodes a first gene product that causes the GMSC to be selectively localized to and/or internalized by a target cell in vivo and a second heterologous nucleic acid that encodes a second gene product that facilitates killing of the target cells following internalization.

### REFERENCE TO ELECTRONIC SEQUENCE LISTING

The application contains a Sequence Listing which has been submitted electronically in .XML format and is hereby incorporated by reference in its entirety. Said .XML copy, created on Jan. 15, 2026, is named “10457-532US2 .xml” and is 338,511 bytes in size. The sequence listing contained in this .XML file is part of the specification and is hereby incorporated by reference herein in its entirety.

### BACKGROUND

Cancer represents a diversity of disease states characterized by unregulated proliferation of cells that are either freely multiplying in blood and/or lymph or organized into tumor masses. After cardiovascular disease, cancer ranks as the second most common cause of death in the US.

Bacteria have been used to target cancers since Coley's observation over 100 years ago that tumors regressed in cancer patients infected with Streptococcus pyogenes. Later, he used killed S. pyogenes, known as Coley's toxin, to treat cancer patients. Unfortunately, the trials of using bacteria as cancer therapy agents stopped for almost 70 years. After Malmgren demonstrated that Clostridium tetani could survive and replicate in necrotic tumors in 1955, studies using bacteria as cancer therapy recommenced and now are widespread in preclinical and clinical studies. Many bacteria have been investigated for their anti-cancer ability, including Bifidobacterium infantis, Escherichia coli, C. tetani, Listeria monocytogenes and Salmonella Typhimurium. While obligate anaerobes, such as Bifidobacterium and Clostridium, are highly effective at accumulating and replicating in necrotic tumors, they do not grow in viable tumor tissues, which limits their efficacy as anti-cancer agents. S. typhimurium is a facultative anaerobe, which can survive and grow in anoxic regions as well as in viable oxygenic regions of tumors. Salmonella also has the ability to identify and penetrate tumors by detecting small molecules such as serine and aspartate in tumors, and accumulates in tumors that contain free amino acids, purines and pyrimidines that facilitate Salmonella growth. As Salmonella are easily genetically manipulated, and attenuated Salmonella still retain their tumor-targeting and natural tumor-regressing capabilities, they became safe enough to evaluate in tumor-bearing mice and humans. Therefore, S. typhimurium is widely investigated as an anti-cancer agent. Currently, many researchers use S. typhimurium VNP20009 or its derivatives as the anti-cancer agent or as a vector to investigate the efficacy of anti-cancer activities. While VNP20009 carrying a purine auxotrophic mutation (purI) and lipid A mutation (msbB) and its derivatives demonstrated good anti-tumor efficacy in mice, anti-cancer efficacy in human trials was not achieved in phase I clinical trials in patients with metastatic melanoma and renal carcinoma. In VNP20009-immunized dogs with a variety of malignant tumors, bacterial colonization of tumors was observed but only 4 of 35 dogs tested were completely cured. Even intratumoral injection in humans with cancer only led to colonization in 2 out of 3 patients. The reasons for failure in human clinical trials may be that the parent of VNP20009 is not highly virulent and its genetic construction is not precise. VNP20009 is derived from ATCC 14028, which does not show high virulence and invasiveness compared to other S. typhimurium strains and we demonstrated that an attenuated aroA mutant of 14028 was not as immunogenic and did not induce as high protective immune levels as did an isogenic derivative of the S. typhimurium UK-1 strain and furthermore was not as effective as a UK-1-derived strain in ablating colorectal tumors in mice. In addition, the construction of VPN20009 is based on UV- and Tn 10 transposon-induced mutations, which may result in other mutations and over-attenuation. It has been shown that the design method caused strain VPN20009 to lose chemotactic ability. Also, the msbB mutation in VNP20009 is a bad choice because it leads to production of penta-acylated lipid A, which is a good pro-inflammatory stimulator in mice, but is an antagonist to inhibit stimulating human innate immunity. The second S. typhimurium strain widely used for cancer therapy is A1-R, which is also derived from ATCC 14028 and is a leu-arg auxotroph. Notable, the parent of A1-R, A1 is screened through nitrosoguanidine mutagenesis. A1-R exhibited good tumor-seeking features and has antitumor efficacy against major types of cancer in mice, but no clinical trials in humans or dogs have been performed. The 3^{rd} strain is VXM01, which is based on the S. typhi strain Ty21a vaccine carrying a eukaryotic expression plasmid for VEGFR2, could induce anti-angiogenic activity when delivered by the oral route in pancreatic cancer. But only 1 of 13 patients showed an improved clinical outcome. The 4^{th} strain tested was χ4550 delivering IL-2 to induce responses in dogs and humans, respectively. All these strains lack specific tumor targeting ability although VPN20009 and A1-R preferentially colonize tumors. Nevertheless, the results showed that their targeting ability is not enough for high efficacy.

Salmonella has ability to regress tumors because of its natural toxicity and can also be used as vectors to deliver other anti-cancer molecules including cytotoxic agents such as Cytolysin A (ClyA), FAS ligand (FasL) and TNF-related apoptosis-inducing ligand (TRAIL), cytokines such as IL-2, and tumor antigens such as 3urviving and other factors such as tyrosinase which enhance its anti-cancer effectiveness. FasL and TRAIL belong to the TNFα family. FasL specifically induces apoptosis in cells that possess the FAS receptor and TRAIL is cytotoxic to many cancer cells via death receptor pathways, which activate caspases 8 and 3. ClyA is a bacterial toxin inducing apoptosis and when delivered by S. typhimurium reduced tumor growth in mice. Cytokine IL-2 is widely investigated for its anti-cancer ability because IL-2 can activate the cytolytic function of NK cells and promotes lymphocyte proliferation. Cytotoxic agents and cytokines can induce apoptosis or stimulate immune cells to directly kill cancer cells, while tumor antigens such as survivin function to sensitize the immune system to fight against cancer cells. Survivin is a member of the inhibitor-of-apoptosis protein family involved in regulation of apoptosis and T-cell responses in anti-tumor immunity. It is over-expressed in many tumor cells. Blocking survivin function is thus a promising anti-tumor therapeutic method via induction of immune responses against.

In 1981, a patent application was filed on use of attenuated derivatives of pathogenic bacteria to deliver recombinant protective antigens from heterologous pathogens to induce protective immunity to the pathogens whose antigens were delivered by the vaccine construct. Salmonella was the chosen pathogen and it has been continuously improved and perfected as a means for using Salmonella as an antigen and DNA vaccine delivery vector. Traditionally, rendering live vaccines safe to be unable to cause adverse effects or disease symptoms has been accompanied with decreased immunogenicity because of the lessened abilities of the attenuated live vaccine to be invasive to colonize lymphoid tissues and/or with reduced abilities to multiply and/or persist to induce an adequate immune response unless administered in multiple doses. We have recently invented multiple means to eliminate all these problems that limit live vaccine efficacy by genetically programing the recombinant attenuated Salmonella vaccines (RASVs) to display the same as or even better infection proficiencies than wild-type Salmonella at the time of RASV administration. We thus invented means to increase invasiveness of RASVs and enhance their ability to better survive against host-defense barriers encountered during mucosal delivery. These modifications coupled with engineering strains with regulated delayed attenuation and regulated delayed antigen synthesis enable the vaccine constructed strains to colonize internal tissues almost to the same extent as wild-type virulent Salmonella but without causing any disease symptoms. RASVs are also designed to persist in these effector lymphoid tissues to serve as factories for the continuous synthesis and delivery of recombinant protective protein antigens. These protein antigens are encoded by pathogen genes to induce protective immunity against the pathogen. Alternatively, the recombinant protein might exert a physiological activity altering a host physiological or immunological activity. In either case, the protein is encoded by codon-optimized sequences to enhance mRNA stability and efficiency of transcription and translation in Salmonella. Since immune responses against recombinant proteins are improved by secretion of antigens rather than their retention in the RASV cytosol, we perfected use of type 3 and type 2 secretion systems (T3SS & T2SS) to export proteins out of the RASV or into the periplasmic space to enhance production of outer membrane vesicles that are highly immunogenic. In addition, we developed vaccine constructs with regulated delayed lysis in vivo to release in specified cell compartments a bolus of recombinant proteins or a DNA vaccine designed for maximal import to the nucleus for efficient high-level transcription and translation of encoded sequences. We observed in multiple recent studies that higher levels of induced protective immunity can be induced by vaccine strains displaying the regulated delayed lysis phenotype than by strains not undergoing lysis. We have engineered strains to eliminate or decrease synthesis of serotype-specific LPS O-antigen and other immune-dominant surface antigens to reduce inducing immune responses to Salmonella. Nevertheless, prior immunity including maternal immunity enhances success in immunizing neonates and individuals previously immunized with a different strain. We now term these much-improved vaccine vector strains as Protective Immunity Enhanced Salmonella Vaccine (PIESV) vector strains. Based on accumulated results demonstrating complete biological containment and safety of our self-destructing PIESV vectors encoding for delivery of protective antigens from various bacterial, viral and parasite pathogens in newborn, pregnant, malnourished and immune deficient SCID mice, in multiple studies with mice, chickens, pigs and in a human phase 1 trial with no adverse events, bacteremias or shedding in vaccinated human volunteers of viable recombinant vaccine cells in stools over a 12-day period at oral doses of 10¹⁰ CFU, the NIH Office of Science Policy and Recombinant Advisor Committee granted permission to us to evaluate our genetically modified vaccines at Biosafety level 1 containment and under settings simulating commercial rearing for farm animals and in out-patients for human trials. This reclassification was also approved by the University Florida Institutional Biosafety Committee.

We also discovered that these PIESV strains are superior adjuvants in recruiting innate immunity. We subsequently have been designing these adjuvant S. typhimurium UK-1 derived strains as Self-Destructing Attenuated Adjuvant Salmonella (SDAAS) strains to serve as adjuvants to recruit innate immune responses and enhance induction of immunity induced by subunit, killed, live attenuated and live vectored vaccines.

The innate immune system is the first line of host defense against pathogen invasion by sensing pathogen-associated molecular patterns (PAMPs) or danger-associated molecular patterns (DAMPs) with pattern-recognition receptors (PRRs). This system activates and maintains adaptive immune responses such as phagocytosis by macrophages and polymorphonuclear cells, and natural cytotoxicity of NK cells and can mediate antibody-dependent cellular phagocytosis or cell cytotoxicity through Fc receptors. Innate immunity involves various types of cells, including dendritic cells (DCs), mast cells, monocytes/macrophages, polymorphonuclear cells, natural killer (NK) cells, and innate lymphoid cells. These cells recognize tumor-derived DAMPs through recognition by PRRs to stimulate innate immune responses or tumor-associated antigens to prime and activate APC or T cells. The activation of innate immune cells promotes effector innate immune functions to lead to tumor cell destruction, which further generates more detection signals and propagates the response. In addition to their direct tumoricidal effect, tumor-activated innate immune cells participate in all steps of T-cell generation and activity against cancer cells, by participating in tumor-specific T-cell priming, expansion, and infiltration at the tumor site. Absence of innate immune cells increases tumor incidence (1, 2). It fully integrates the cancer-immunity cycle and is critical to the occurrence, progression and elimination of tumors (3-7). Thus, the innate immune response plays an important role in sensing tumor cells and recruitment, activation, and clonal expansion of tumor-specific T cells to help killing tumor cells in conjunction with conventional therapies or immunotherapies (8-12).

Salmonella is widely investigated as an anti-cancer bacterial strain (13-18). It can identify, penetrate, and accumulate in tumors that contain free amino acids, purines, and pyrimidines that facilitate Salmonella growth and survive in anoxic regions as well as in viable regions of tumors (19-23). It can be genetically manipulated to be safe while keeping its tumor-targeting and natural tumor-regressing abilities. Salmonella also have the adjuvant abilities through the multiple PAMPs, such as lipoproteins (TLR1/2/6/10), Lipid A (TLR4), flagellin (TLR5/NLRC4) (24), CpG-motif (TLR9), peptidoglycan (NODs), DNA and RNA (TLR7/TLR8) (25, 26).

Lipid A and flagellin have been widely used as adjuvant therapies by directly killing or activating downstream signals or cytokines to trigger the host innate and adapted immune responses to initiate anti-tumor responses (27-29). LPS can be recognized and activate caspase-11 (caspase-4/caspase-5 in humans) in mice to trigger cell pyroptosis by IFN-induced GTPase and canonical NLRP3/ASC inflammasome activation for IL-1β/18 release (30-33). Flagellin can activate NAIP5/NLRC4 (34-37) that could stimulate inflammasome assembly to activate caspase-1 (38) to induce tumor cell pyroptosis (39, 40) and apoptosis (41).

3-O-desacyl-4′-monophosphoryl lipid A (MPLA) is the only lipid A species that has been evaluated as a cancer vaccine adjuvant in human clinical trials (42-48). MPLA is at least 100-fold less toxic than other lipid A species (49). It has been proved safe in more than 300,000 human subjects in vaccine studies (50). MPLA induces IL-6 to activate effective cytotoxic T-cell differentiation to induce granzyme B production and reduce expression of inhibitory PD1 on the surface of the primed CD8 T cell (51). Combining MPLA with other immunostimulants has been shown to be advantageous in the therapy of colorectal, prostate, melanoma and breast cancer and in cancers with certain tumor-specific antigens (52) and be required to elicit the full complement of activities necessary to achieve effective immune responses and overcome the ability of tumors to evade attack by the immune system.

Flagellin has been proven to retard tumor growth and significantly prolong survival of tumor bearing mice by activating CD8 T cells secreting IFN-γ (53) that accelerates leukocyte infiltration and tumor necrosis (54). It can activate intratumoral macrophages with M1 phenotypes which promote Th1 function and a reciprocal reduction in M2-like suppressive activities which promoter Th2 functions (55, 56). Engineering Salmonella to deliver Vibrio flagellin FlaB was able to effectively suppress tumor growth and metastasis and prolonged survival in mouse models (55). Salmonella defective in synthesis of flagellum-specific ATPase FliHIJ or the inner membrane ring FliF displayed great immune stimulatory capacity and strong anti-tumor effects by increasing outer membrane vesicle production to show anti-tumor efficacy against the highly resistant cancer cell line RenCa (57). There is a syngeneic effect between TLR4 and TLR5. TLR4 is required for tumor-suppressive host reactions to recruit immune cells such as neutrophils and monocytes/macrophages, while TLR5 can augment this response by inducing infiltration of these cells (55).

However, to implement these principles and fully execute the advantages of SDAAS strains, they need to be improved for effective tumor and/or cancer cell targeting to ensure its activity in tumor/cancer and reduce the unneeded or unwanted activation in normal tissues.

### BRIEF DESCRIPTION OF DRAWINGS

FIG. 1. Plasmid maps of the cloning vector pYA3342, the suicide vector pRE112 and the regulated delayed lysis DNA vaccine vector pYA4545.

FIG. 2. Plasmid maps of the regulated delayed lysis cloning vectors pG8R110 (with T3SS) and pG8R114 (with T2SS) and the plasmid vectors pYA4090 encoding synthesis of GFP and pYA4685 encoding synthesis of EGFP.

FIG. 3. Plasmid map of pG8R314 encoding OmpA with PLZ4 insert (ompAΩplz4).

FIG. 4. Plasmid map of pG8R315 as suicide vector for insertion of sequence encoding ompA with PLZ4 insertion (ompAΩplz4) into the S. typhimurium chromosome.

FIG. 5. Plasmid maps of pG8R319 derived from pG8R314 by insertion of eukaryotic expression cassette and pG8R320 derived from pYA4545 by insertions of a prokaryotic expression cassette to express sequence encoding ompA with PLZ4.

FIG. 6. Salmonella with PLZ4 peptide exposed on the surface is attracted to and invades into bladder cancer cells.

FIG. 7. Plasmid maps of pG8R321, pG8R322, pG8R323 and pG8R324 all derived from pG8R319 by insertion of the nucleotide sequences encoding human CXCL11, Mouse CXCL11, KillerRed fused to the neuromodulin N-terminal sequence and KillerRed fused to mitochondrial targeting signals, respectively.

FIG. 8. Plasmid maps of pG8R325, pG8R326, pG8R327 and pG8R328 all derived from pG8R320 by insertion of the nucleotide sequences encoding human CXCL11, Mouse CXCL11, Killer Red fused to the neuromodulin N-terminal sequence and Killer Red fused to mitochondrial targeting signals, respectively.

FIG. 9. Plasmid maps of pG8R341 derived from pG8R314 by insertion of sequence encoding GFP3 as an operon fusion and pG8R342 derived from pG8R320 by insertion of sequence encoding EGFP.

FIG. 10. Plasmid maps of multicistronic pG8R343 and pG8R344 derived from pG8R320 by insertion of sequences encoding KillerRed fused to the neuromodulin N-terminal sequence and human CXCL11 or mouse CXCL11. P2A peptide is used to separate KillerRed and CXCL11

FIG. 11. Plasmid maps of pG8R345 derived from pG8R320 by insertion of sequences encoding HLAB leading and tail peptides and pG8R346 derived from pG8R345 by insertion of sequence encoding EGFP.

FIG. 12. Plasmid maps of pG8R347, pG8R348, pG8R349 and pG8R350. pG8R347 is derived from pG8R320 by insertion of sequences for 5′ HLA, HLA leading and Tail peptides and 3′ HLA. pG8R348, pG8R349 and pG8R350 are derived from pG8R347 by insertion of sequence encoding EGFP, neo-antigen BBM963 and MB49, respectively.

FIG. 13. Plasmid pG8R361 derived from pG8R320 by insertion of sequence from P_{EF1α} promoter.

FIG. 14. Plasmid maps of pG8R362, pG8R363, pG8R364 and pG8R365. pG8R362 and pG8R363 are derived from pG8R320 by insertion of sequence encoding HAC-PD1 fused with human CXCL11 and HAC-PD1 fused with mouse CXCL11, respectively. pG8R364 and pG8R365 are derived from pG8R361 by insertion of sequence encoding HAC-PD1 fused with human CXCL11, HAC-PD1 fused with mouse CXCL11, respectively.

FIG. 15. Plasmid maps of pG8R366 derived from pG8R320 by insertion of sequence encoding IL2 secretion signal (IL2 SS) and pG8R367 and pG8R368 derived from pG8R366 by insertion of sequence encoding HAC-PD1 and human CXCL11 and HAC-PD1 and mouse CXCL11, respectively.

FIG. 16. Plasmid maps of pG8R372, pG8R373, pG8R374 and pG8R375 derived from pG8R320 by insertion of sequence encoding IL2 SS fused with HAC-PD1. IL2 SS fused with HAC-PD1 and EGFP, human CXCL11 fused with EGFP, mouse CXCL11 fused with EGFP, respectively.

FIG. 17. Plasmid maps of pG8R380 derived from pG8R320 by insertion of sequence encoding operon fusion of OmpA with LHRH insertion and GFP and pG8R381 derived from pG8R380 by insertion of OmpA with LHRH insertion, respectively.

FIG. 18. Plasmid maps of pG8R382, pG8R383 and pG8R384 derived from pG8R320 by insertion of sequence encoding haPD1-IgG, haPD1-IgG and KillerRed fused with neuromodulin N terminal sequence, and KillerRed fused with neuromodulin N terminal sequence and haPD1-IgG, respectively.

FIG. 19. Plasmid maps of pG8R385 derived from pG8R320 by insertion of sequence encoding operon fusion of OmpA with Her2 scFv insertion and GFP and pG8R386 derived from pG8R385 by insertion of OmpA with Her2 scFv insertion, respectively.

FIG. 20. Plasmid maps of pG8R388 and pG8R389 derived from pG8R320 by insertion of sequence for P_{trc} promoter and P_{trc} promoter and optimized Bla secretion signal.

FIG. 21. Plasmid maps of pG8R390, pG8R391, and pG8R418. pG8R390 is derived from pG8R381 by insertion of sequence encoding KillerRed fused with neuromodulin N terminal sequence and pG8R391 is derived from pG8R386 by insertion of sequence encoding KillerRed fused with neuromodulin N terminal sequence. pG8R418 is derived from pG8R385 by insertion of sequence encoding KillerRed fused with neuromodulin N terminal sequence.

FIG. 22. The attachment and invasion of bladder cancer cells with strains derived from 112614 with O-antigen mutations. (A) Genotypic characterization of strains using primers specific for waaL, waaG, waaC, ompA and ompAΩplz4. Lane 1, χ3761; lane 2, χ12614; lane 3, χ12812; lane 4, χ12813; lane 5, χ12814. (B) LPS gel profile of strains. All strains are grown in LB media. (C) The percentage of attachment and invasion of strains in human bladder cancer cell line 5637 and mouse bladder cancer cell line MB49. All strains carry plasmid pG8R314 with multiple copies of ompAΩplz4. ****, P<0.0001, compared with other strains.

FIG. 23. The attachment and invasion of bladder cancer cells with strains derived from χ12619 with O-antigen mutations. All strains have only one copy of ompAΩplz4 in their chromosome. (A) Genotypic characterization of strains using primers specific for asdA, pagP, pagL, lpxR, waaL, waaG, waaC, ompA and ompAΩplz4. Lane 1, χ3761; lane 2, χ12518; lane 3, χ12619; lane 4, χ12808; lane 5, χ12809; lane 4, χ12810; lane 5, χ12811. (B) The growth of strains on LB, LB+ arabinose (0.1%), LB+DAP (50 ug/ml), LB+D-alanine (50 ug/ml) plates. (C) LPS gel profile of the strains. All strains are grown in LB media with 0.1% arabinose. (D) The percentage of attachment and invasion of strains in human bladder cancer cell line 5637 and mouse bladder cancer cell line MB49. *, P<0.05, **, P<0.01, *** P<0.001, ****, P<0.0001, compared with other LPS mutation strains

FIG. 24. Display of OmpAΩHer2 ScFV on the bacterial surface. Salmonella outer membrane proteins (OMPs) were isolated and subjected to SDS-PAGE gel and western blot with anti-His antibody. 1, χ12417; 2, χ12417(pG8R385); 3, χ12417(pG8R418); 4, χ12417(pG8R391).

FIG. 25. Strains carrying ompAΩher2 ScFV display higher attachment and invasion to Her2 over expression cell SKRB-3. ****, P<0.0001.

FIG. 26. Production of KillerRed in HEK293T cells transfected with plasmid pG8R327. The frames show the KillerRed red fluorescent signals from four different samples (EVOS FL, RFP channel)

FIG. 27. KillerRed kills HEK293T cells. Time zero is set immediately after irradiation with green light. Time 10 is set immediately after excitation for 10 min (EVOS FL, RFP channel)

FIG. 28. KillerRed kills HEK293T cell excitation for 10 and 20 minutes. Time zero is set immediately after irradiation with green light. Time 10 and 20 are set immediately after excitation for 10 min or 20 mins (EVOS FL, RFP channel)

FIG. 29. Salmonella adjuvant strain has higher abilities to active TLR4 and TLR5. HEK-Blue cells at 10⁵ cells/ml were mixed with bacterial cells at a MOI of 10 in a volume of 200 μl in 96 well plates. LPS and flagellin, 100 ng.

FIG. 30. Growth of χ3761 (wild type) and χ12419 (ΔP_{purA}) mutation in MOPs media with adenine, 0.2% or 0.5% rhamnose

FIG. 31. Growth of strains in M9 medium with 0.1% Arabinose or 0.1% Arabinose+0.1% Rhamnose.

FIG. 32. Growth of strains in M9 medium with Arabinose+Adenine or Arabinose+Guanine

FIG. 33. Growth of strains in M9 medium with Arabinose/Cytosine. Arabinose/Thymine or Arabinose/Uracil

FIG. 34. Plasmid maps of pG8R478 derived from pG8R320 by insertion of sequence encoding operon fusion of OmpA with RKOpep insertion and GFP and pG8R479 derived from pG8R320 by insertion of sequence encoding operon fusion of OmpA with TK/AG-32 insertion and GFP

FIG. 35. SDAAS strain χ12900 induces tumor recession in a melanoma model.

FIG. 36. Plasmid maps of pG8R481 derived from pG8R320 by insertion of sequence encoding operon fusion of OmpA with αMSH(1-13) insertion and GFP, pG8R482 derived from pG8R320 by insertion of sequence encoding operon fusion of OmpA with CY12-RP2 insertion and GFP, pG8R487 derived from pG8R320 by insertion of sequence encoding operon fusion of OmpA with Peptide C insertion and GFP, and pG8R488 derived from pG8R320 by insertion of sequence encoding operon fusion of OmpA with P20 insertion and GFP

FIG. 37. Plasmid maps of pG8R480 derived from pG8R320 by insertion of sequence encoding operon fusion of OmpA with A3 insertion and GFP, pG8R483 derived from pG8R320 by insertion of sequence encoding operon fusion of OmpA with EBIP-37 insertion and GFP, and pG8R486 derived from pG8R320 by insertion of sequence encoding operon fusion of OmpA with GE11 insertion and GFP.

FIG. 38. Plasmid maps of pG8R484 derived from pG8R320 by insertion of sequence encoding operon fusion of OmpA with FSHP(33-53) insertion and GFP, and pG8R485 derived from pG8R320 by insertion of sequence encoding operon fusion of OmpA with FSHP(81-95) insertion and GFP.

FIG. 39. Plasmid maps of pG8R489 derived from pG8R320 by insertion of sequence encoding operon fusion of OmpA with S36 insertion and GFP, pG8R490 derived from pG8R320 by insertion of sequence encoding operon fusion of OmpA with T7 insertion and GFP, and pG8R491 derived from pG8R320 by insertion of sequence encoding operon fusion of OmpA with T12 insertion and GFP

### DEFINITIONS

As used herein the specification, “a” or “an” may mean one or more, unless clearly indicated otherwise. As used herein in the claims, when used in conjunction with the word “comprising,” the words “a” or “an” may mean one or more than one.

The term “administering” or “administration” of an agent as used herein means providing the agent to a subject using any of the various methods or delivery systems for administering agents or pharmaceutical compositions known to those skilled in the art. Agents described herein may be administered by oral, intradermal, intravenous, intramuscular, intraocular, intranasal, intrapulmonary, epidermal, subcutaneous, mucosal, or transcutaneous administration.

The terms “animal host” or “subject” as used interchangeably hereinto refer to a human or nonhuman mammal or a vertebrate animal into which a genetically modified Salmonella cell has been administered. In a specific embodiment, the subject is a human.

The terms “attenuated” or “attenuation” as used herein refer to the process of rendering certain pathogen virulence attributes needed to cause diseases less able to cause such disease symptoms. In one example, attenuation involves imparting an attenuation mutation in the pathogen.

The term “attenuating mutation” refers to a mutation imparted into a pathogen that reduces infectivity, virulence, toxicity, induction of disease symptoms, and/or impairment of a subject upon administration of the pathogen (e.g. PIESV strain). Examples of attenuating mutations include those mutations that facilitate lysis in vivo (e.g. impairing synthesis of essential constituents of peptidoglycan layer), reduce or impair synthesis of LPS or other cell-surface components, and one or more mutations that provide auxotrophy (e.g. dependence on an amino acid, purine, pyrimidine, or vitamin for growth).

The term “balanced-lethal vector-host” refers to a host Salmonella cell into which a plasmid vector has been introduced such that survival of the host cell is dependent on the maintenance of the plasmid vector and loss of the plasmid vector results in death of the host Salmonella cell.

The term “biologically active fragment” or “biologically active variant” refers to a fragment or variant of a sequence that maintains its biological activity. In the context of H. pylori antigen sequences, a biologically active fragment or biologically active variant is a fragment or variant of an antigen amino acid sequence that elicits an immune response in a host.

The term “Cancer Cell Targeting Salmonella strain” or “CCTS strain” refers to a strain of Salmonella that has one or more attenuating mutations and expresses a gene product that causes selective localization and/or internalization of cells of the CCTS strain by a cancer cell.

As used herein, “codon” means, interchangeably, (i) a triplet of ribonucleotides in an mRNA which is translated into an amino acid in a polypeptide or a code for initiation or termination of translation, or (ii) a triplet of deoxyribonucleotides in a gene whose complementary triplet is transcribed into a triplet of ribonucleotides in an mRNA which, in turn, is translated into an amino acid in a polypeptide or a code for initiation or termination of translation. Thus, for example, 5′-TCC-3′ and 5′-UCC-3′ are both “codons” for serine, as the term “codon” is used herein.

The term “codon optimized” or “codon optimization” as used herein refers to enhancing the ability of the antigen encoding sequence to be expressed in the Salmonella strain by selecting codons that are used for highly expressed genes in Salmonella. Such codon optimization also includes changing the GC content of the antigen encoding sequence to be similar to that used for Salmonella (i.e., ˜52% GC). In addition, the codon optimization can also be used to enhance the stability of the mRNA encoded by the antigen encoding sequence so as to be less likely to be degraded by Rnases.

The term “delayed attenuation” as used herein refers to a means of gene regulation such that the attenuation attribute is not expressed during growth of the vaccine strain or during its administration to an animal host but is not expressed after the CCTS strain enters the animal host and is manifest as a consequence of vaccine cell division in vivo with gradual dilution of the virulence gene product by at least half at each cell division in vivo.

The term “gene product” refers to a transcript (RNA) or expressed polypeptide encoded by a heterologous gene or nucleic acid that has been introduced into a genetically modified Salmonella cell. In typical embodiments, the gene product causes selected localization to a target cell. The gene product may also cause cytotoxicity to the target cell upon internalization of the genetically modified Salmonella cell and/or cause a targeted immune response to target cells.

A “genetically modified Salmonella cell” or “GMSC” refers to a Salmonella cell that comprises an attenuating mutation and/or into which a heterologous gene or nucleic acid, e.g., an exogenous nucleic acid that is foreign to the Salmonella cell, has been introduced.

The term “operably linked” as used herein means that one nucleic acid sequence is linked to another nucleic acid sequence, and therefore the function or expression thereof is influenced by the linked nucleic acid sequence.

As used herein, the term “percentage of sequence identity” or “percent sequence identity” may refer to the value determined by comparing two optimally aligned sequences (e.g., nucleic acid sequences or polypeptide sequences) of a molecule over a comparison window, wherein the portion of the sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleotide or amino acid residue occurs in both sequences to yield the number of matched positions, dividing the number of matched positions by the total number of positions in the comparison window, and multiplying the result by 100 to yield the percentage of sequence identity. A sequence that is identical at every position in comparison to a reference sequence is said to be 100% identical to the reference sequence, and vice-versa. The term “about” with respect to a numerical value of a sequence length means the stated value with a +/−variance of up to 1-5 percent. For example, about 30 contiguous nucleotides means a range of 27-33 contiguous nucleotides, or any range in between. The term “about” with respect to a numerical value of percentage of sequence identity means the stated percentage value with a +/−variance of up to 1-3 percent rounded to the nearest integer. For example, about 90% sequence identity means a range of 87-93%. However, the percentage of sequence identity cannot exceed 100 percent. Thus, about 98% sequence identity means a range of 95-100%.

The term “regulated delayed lysis” refers to a construction in which the expression of one or more genes specifying synthesis of peptidoglycan precursors such as but not limited to diaminopimelic acid and muramic acid are regulated by a sugar-dependent process such that the genes are expressed in the presence of a sugar such as but not limited to arabinose or rhamnose supplied during cultivation of the strain and cease to be expressed in vivo since the sugar is absent to result in lysis as a consequence of cell division of the CCTS strain in vivo. The genes conferring the regulated delayed lysis phenotype may be either chromosomal and/or plasmid encoded.

The term “regulated delayed lysis plasmid” refers to a construction in which the expression of one or more genes specifying synthesis of peptidoglycan precursors such as but not limited to diaminopimelic acid and muramic acid that are regulated by a sugar-dependent process are located on a plasmid vector encoding synthesis of one or more foreign antigens or gene products.

The term “Salmonella cell” refers to a cell of a Salmonella species or serotype. Examples of a Salmonella serotype include Salmonella Typhimurium and Salmonella Enteritidis. In a more specific embodiment, the Salmonella serotype is S. typhimurium UK-1.

The term “sequence identity” or “identity,” as used herein in the context of two polynucleotides or polypeptides, refers to the residues in the sequences of the two molecules that are the same when aligned for maximum correspondence over a specified comparison window.

As used herein, the term “targeted immune response” refers to a response by a subject's immune system against target cells. Immune responses include both cell-mediated immune responses (responses mediated by antigen-specific T cells and non-specific cells of the immune system) and humoral immune responses (responses mediated by antibodies present in the plasma lymph, and tissue fluids and secreted onto mucosal surfaces).

The term “target cell” refers to a cell of a subject that is of a type to which a genetically modified Salmonella cell is designed for selective localization and/or internalization. Selective localization refers to increased migration of the genetically modified Salmonella cell to a target cell over other cells in a subject. Selective internalization refers to increased internalization of the genetically modified Salmonella cell in the target cell over other cells in the subject. Increased localization to and/or increased internalization means an increase of at least 5%, 10%, 20%, 30%, 40%, 50%, 60%, 70% or more respective to target cells as opposed to other cells in a subject. Typically, a target cell internalizes the genetically modified Salmonella cell by active invasion or endocytosis or phagocytosis. In exemplified embodiments, the target cell is a cancer cell and the genetically modified Salmonella cell is of a CCTS strain that is selectively internalized by the cancer cell over other cells in the subject. In alternative embodiments, the genetically modified Salmonella cell is engineered to localize at a tumor microenvironment where cancer cells are present without necessarily being internalized into a cancer cell.

The term “variant” as used herein refers to a nucleic acid sequence or amino acid sequence that possesses at least about 85, 90, 95, 96, 97, 98 or 99 percent sequence identity to another nucleic acid sequence or amino acid sequence, respectively.

Other relevant definitions are provided infra.

### DESCRIPTION

Disclosed herein are embodiments directed to designing, constructing and evaluating Cancer Cell Targeting Salmonella (CCTS) strains. CCTS strain embodiments have (i) ability to directly destroy tumor cells, (ii) deliver cargoes that cause tumor cells to self-destruct, (iii) deliver cargoes that enhance abilities to treat tumor cells, and/or (iv) directly and/or indirectly stimulate host immune responses to repress tumor cell growth, metastases and cell death. A potentially desirable feature involves rapid self-destruction of CCTS cells that enables their use for repeat treatments of subjects. A unique attribute of these newly designed and constructed CCTS strains is their ability to simultaneously synthesize and deliver protein cargoes to cancer cells but to also deliver DNA vaccines encoding other effective proteins to be synthesized by the tumor cells to their detriment.

The foregoing attributes are achieved by introducing numerous deletion and deletion-insertion mutations to enable and endow the desired phenotypic properties to the strains constructed. These mutations and their associated phenotypes are listed in Table 1 and the suicide vectors needed for their insertion into plasmids and the S. typhimurium chromosome are listed in Table 2. The distribution of genetic deletion and deletion-insertion mutations and the redundancy in critical modifications ensure both stability and safety of these CCTS strains.

Examples of genotypes of CCTS strains are listed in Table 3.

TABLE 1
Mutations and associated phenotypes in S. Typhimurium CCTS strains^{a} It is noted that the
genes can be inactivated or deleted in multiple ways to confer the same phenotypic traits.
Also, though certain allele numbers are indicated elsewhere herein for certain mutations, reference
to a certain allele is not limiting and the mutations can be executed in other alleles.

| Genotype | Phenotype |
|---|---|
| ΔaroA | encodes the first enzyme in the pathway to synthesize aromatic |
| | amino acids and derived vitamins |
| ΔasdA | deletes gene for aspartate semialdehyde dehydrogenase |
| | essential for synthesis of diaminopimelic acid (DAP) necessary |
| | for peptidoglycan synthesis |
| ΔP_{asdA}::TT araC P_{araBAD} asdA | makes synthesis of AsdA dependent on presence of |
| | arabinose |
| ΔP_{asdA}::TT rhaRS P_{rhaBAD} asdA | makes synthesis of AsdA dependent on presence of |
| | rhamnose |
| ΔasdA::TT araC P_{araBAD} c2 | inactivates asdA and makes synthesis of c2 repressor |
| | dependent on arabinose |
| Δalr and ΔdadB | deletes the genes for two alanine racemases essential for |
| | synthesis of D-alanine necessary for peptidoglycan synthesis |
| ΔP_{dadB}::TT araC P_{araBAD} dadB | makes synthesis of DadB dependent on presence of |
| | arabinose |
| ΔP_{dadB}::TT rhaRS P_{rhaBAD} dadB | makes synthesis of DadB dependent on presence of |
| | rhamnose |
| ΔP_{murA}::TT araC P_{araBAD} murA | makes synthesis of MurA, the first enzyme in the |
| | synthesis of muramic acid, dependent on arabinose in growth |
| | medium and ceases synthesis in vivo due to absence of |
| | arabinose |
| ΔP_{fur}::TT araC P_{araBAD} fur | makes synthesis of the Fur repressor protein |
| | dependent on arabinose in growth medium that ceases in vivo to |
| | result in high-level synthesis of all iron regulated proteins to |
| | result in attenuation |
| ΔmntR | eliminates gene for repressor MntR that regulates MntR- and |
| | some Fur-regulated genes for manganese and iron acquisition, |
| | respectively |
| ΔP_{mntR}::TT araC P_{araBAD} mntR | makes synthesis of the MntR repressor protein |
| | dependent on arabinose in growth medium that ceases in vivo to |
| | result in high-level synthesis of all manganese regulated proteins |
| | to contribute to attenuation |
| Δcya | encodes enzyme for adenylate cyclase |
| Δcrp | encodes adenylate cyclase catabolite represor protein |
| ΔaraBAD::TT | deletion of genes to eliminate arabinose catabolism with TT |
| | inserted to prevent transcription of downstream genes |
| ΔaraCBAD100::TT | Deletion of all genes in the ara operon |
| ΔrhaBADSR | deletion of genes to eliminate rhamnose catabolism |
| ΔpagP::Pₗₚₚ lpxE mutation | causes regulated delayed in vivo synthesis of the codon- |
| | optimized lpxE gene from Francisella tularensis to cause |
| | synthesis of the non-toxic adjuvant form of LPS lipid A lipid A |
| | (MPLA) |
| ΔlpxR::Pₗₚₚ lpxF mutation | causes regulated delayed in vivo synthesis of the codon- |
| | optimized lpxF gene from Francisella tularensis to cause |
| | synthesis of LPS with only the 1′-phosphoryl group |
| ΔpagL and ΔlpxR | eliminates two means by which Salmonella alters LPS |
| | components in vivo to decrease recruitment of innate immunity |
| | by interaction with TLR4 |
| ΔeptA | prevents addition of ethanolamine to lipid A |
| ΔarnT | prevents addition of 4-amino-4-deoxy-L-arabinose (L-Ara4N) |
| | groups to lipid A |
| ΔfliC | deletes gene specifying synthesis of the phase I flagellin FliC |
| ΔfljB | deletes gene specifying synthesis of the phase II flagellin FljB |
| ΔfliC180 | specifies a truncated FliC protein containing TLR5 recognition |
| | domain and CD4 epitope |
| Δ(hin-fljBA) | locks in expression of gene for phase I FliC flagellin and |
| | precludes synthesis of phase II FljB flagellin |
| Δ(agfG-agfC) | deletes two operons specifying thin aggregative fimbriae (curli) |
| | and an activator for synthesis and export of cellulose and other |
| | exopolysaccharides |
| ΔP_{saf5}::P_{murA} safA | causes constitutive synthesis of Saf fimbriae that facilitate spleen |
| | colonization |
| ΔP_{stc}::P_{murA} stcA | causes constitutive synthesis of Stc fimbriae that facilitate spleen |
| | colonization |
| ΔfimH | encodes the adhesin tip on Type 1 fimbriae |
| ΔompA | specifies synthesis of a very prevalent outer membrane protein |
| ΔompC | specifies synthesis of a prevalent outer membrane protein |
| | (58, 59) |
| ΔompD | specifies synthesis of a prevalent outer membrane protein |
| | (60-62) |
| ΔompF | specifies synthesis of a prevalent outer membrane protein (63) |
| ΔsopB | a protein secreted by the Salmonella SPI-I that can cause |
| | intestinal inflammation |
| ΔpabA & ΔpabB | Encode two enzyme subunits of the enzyme synthesizing p- |
| | amino benzoic acid |
| Δpmi | eliminates phosphomannose isomerase that precludes synthesis |
| | of GDP-mannose that is needed for LPS O-antigen synthesis |
| ΔwaaL & ΔpagL::TT | make synthesis of the Waal enzyme that couples O-antigen to |
| araC P_{araBAD} waaL | the LPS core synthesis dependent on presence of arabinose |
| (or ΔpagL::TT | (or rhamnose) |
| rhaRS P_{rhaBAD} waaL) | |
| ΔwbaP | encodes enzyme that couples LPS core to LPS O-antigen |
| ΔwaaC | encodes enzyme necessary for assembly of the LPS inner core |
| ΔwaaG | encodes enzymes essential fir assembly of the outer LPS core |
| Δ(wza-wcaM) | eliminates 20 genes encoding enzymes needed for synthesis of |
| | colanic acid, LPS capsular antigen and other polysaccharides to |
| | facilitate lysis, enhance immunogenicity and inhibit biofilm |
| | formation |
| ΔrelA | uncouples growth regulation from a dependence on protein |
| | synthesis |
| ΔrelA::araC P_{BAD} lacl TT | makes synthesis of Lacl hat represses gene expression controlled |
| and Δ(traM-traX)::araC | by P_{trc} dependent on presence of arabinose with either |
| P_{araBAD} lacl | inactivation of relA gene or deletion of genes encoding |
| | conjugational plasmid transfer in Salmonella virulence plasmid |
| ΔspoT | eliminates gene for synthesis of ppGpp |
| ΔspvRABCD | deletes Salmonella plasmid virulence genes encoding regulatory |
| | activator-repressor (R) and four genes conferring invasiveness |
| | and virulence, when the spvABCD genes are over expressed |
| | increase invasiveness and virulence |
| ΔcysG175::Pₛₚᵥ spvABCD | inserts spv operon without the R gene specifying the |
| | repressor-activator into a deletion of the cysG gene under control |
| | of a promoter not regulated by SpvR |
| ΔP_{hilA}::P_{traΔlacO} hilA | constitutive P_{trc} regulated synthesis of HilA that increases |
| | expression of SPI-1 genes for invasion of epithelial cells |
| ΔrecF | reduces inter- and intra-plasmidic recombination |
| ΔendA | deletes gene encoding endonuclease I to prevent degradation of |
| | released DNA vaccine |
| ΔsifA | enables Salmonella to escape from the SCV to enter the cytosol |
| ΔsseL | eliminates a gene that enables Salmonella to induce pyroptosis |
| ΔtlpA | eliminates a gene that enables Salmonella to induce pyroptosis |
| ΔpurA | deletion of purA encoding adenylosuccinate synthetase that |
| | catalyzes the first step toward the de novo synthesis of AMP, |
| | resulting in a purine-deficient autxotroph (64-67) |
| ΔP_{rhaBAD}::rhaRS P_{rhaBAD} purA | make synthesis of the PurA that catalyzes the first step |
| | toward the de novo synthesis of AMP dependent on presence of |
| | rhamnose. |
| ΔguaBA | deletion of guaB encoding IMP dehydrogenase (IMPDH)/inosine |
| | 5′-monophosphate dehydrogenase) catalyzes the NAD₊- |
| | dependent oxidation of IMP to XMP, the first committed, rate- |
| | limiting step in de novo guanine nucleotide biosynthesis and |
| | guaA encoding GMP synthetase catalyzes the glutamine- or |
| | ammonia-dependent synthesis of GMP from XMP. Both are |
| | needed to catalyze the de novo synthesis of GMP (68, 69). |
| ΔP_{guaBA}::rhaRS P_{rhaBAD} guaBA | make synthesis of the GuaB and GuaA that catalyzes |
| | the de novo synthesis of GMP dependent on presence of |
| | rhamnose. |
| ΔpyrF | deletion of pyrF encoding orotidine-5′-phosphate decarboxylase |
| | that catalyzes the last essential step in the de novo biosynthesis |
| | of pyrimidines, the synthesis of UMP by decarboxylation of |
| | orotidine-5′-phosphate (70, 71). |
| ΔP_{pyrF}::rhaRS P_{rhaBAD} pyrF | make synthesis of the PyrF that catalyzes the de |
| | novo synthesis of pyrimidines dependent on presence of |
| | rhamnose |
| ΔPₜₐᵣ::P_{trcΔlacO888} tar | constitutive expression of tar that encodes the aspartate and |
| | maltose receptor (72, 73) |
| ΔPₜₛᵣ:: P_{trcΔlacO888} tsr | constitutive expression of tsr that encodes the serine receptor |
| | (73) |
| Δtrg | deletion of gene encoding the ribose/galactose receptor (73, 74) |

^{a}Δ = deletion; TT = transcription terminator; P = promoter

TABLE 2
Suicide vectors for constructing the mutations in Table 1

| | | |
|---|---|---|
| | Suicide | |
| Genotype | Vector | Marker |

A. Deletion and deletion-insertion mutations
to facilitate regulated delayed lysis in vivo

| | | |
|---|---|---|
| ΔP_{murA25}::TT araC P_{araBAD} murA | pYA4686 | Cm |
| ΔasdA33 | pYA3736 | Cm |
| ΔP_{asdA55}::TT araC P_{araBAD} asdA | pG8R71 | Cm |
| ΔP_{asdA88}::TT rhaRS P_{rhaBAD1} asdA | pG8R354 | Cm |
| Δalr-3 | pYA3667 | Cm |
| ΔdadB4 | pYA3668 | Cm |
| ΔP_{dadB66}::TT araC P_{araBAD} dadB | pG8R73 | Cm |
| ΔP_{dadB22}::TT rhaRS P_{rhaBAD1} dadB | pG8R352 | Cm |
| Δ(wza-wcaM)-8 | pYA4368 | Cm |
| ΔrelA1123 | pYA3679 | Cm |

B. Mutations enabling regulation of genes that might
be present on plasmid vectors in conjunction with
strains undergoing regulated delayed lysis in vivo

| | | |
|---|---|---|
| ΔrelA197::araC P_{araBAD} lacl TT | pYA4064 | Cm |
| ΔasdA27::TT araC P_{araBAD} c2 | pYA4138 | Cm |
| Δ(traM-traX)-36::araC P_{araBAD} lacl TT | pG8R329 | Cm |
| Δ(traM-traX)-41::araC P_{araBAD} lacl TT | pG8R397 | Cm |

C. Mutations conferring attenuation of virulence

| | | |
|---|---|---|
| ΔaroA21419 | pYA3600 | Cm |
| Δcya-27 | pMEG080 | Tet |
| Δcrp-27 | pMEG084 | Tet |
| ΔpabA1516 | pMEG147 | Tet |
| ΔpabB232 | pYA3438 | Cm |

D. Mutations conferring regulated delayed attenuation
and over production of iron and manganese-regulated
proteins to confer cross-protective immunity

| | | |
|---|---|---|
| ΔP_{fur33}::TT araC P_{araBAD} fur | pYA3722 | Cm |
| ΔP_{mntR44}::TT araC P_{araBAD} mntR | pG8R227 | Cm |

E. Mutations altering synthesis of LPS components

| | | |
|---|---|---|
| Δpmi-2426 | pYA3546 | Tet |
| ΔpagP8 | pYA4288 | Cm |
| ΔpagP81::Pₗₚₚ lpxE | pYA4295 | Cm |
| ΔpagL7 | pYA4284 | Cm |
| ΔlpxR9 | pYA4287 | Cm |
| ΔlpxR93::Pₗₚₚ lpxF | pYA4289 | Cm |
| ΔarnT6 | pYA4286 | Cm |
| ΔeptA4 | pYA4283 | Cm |
| ΔwaaC41 | pYA5473 | Cm |
| ΔwaaG42 | pYA4896 | Cm |
| ΔwaaL46 | pYA4900 | Cm |
| ΔwbaP45 | pYA4899 | Cm |
| ΔpagL19::TT araC P_{araBAD1} waaL | pYA5468 | Cm |
| ΔpagL64::TT rhaRS P_{rhaBAD1} waaL1 | pYA5377 | Cm |
| ΔpagL38::TT rhaRS P_{rhaBAD1} waaL2 | pG8R296 | Cm |
| ΔpagL18::TT araC P_{araBAD1} waaC | pYA5458 | Cm |
| ΔpagL21::TT araC P_{araBAD1} waaG | pYA5462 | Cm |

F. Mutations blocking catabolism of sugars

| | | |
|---|---|---|
| ΔaraBAD65::TT | pYA4811 | Cm |
| ΔrhaBADSR515 | pG8R272 | Cm |
| ΔaraCBAD100::TT | pG8R392 | Cm |

G. Mutations altering synthesis of flagellar components

| | | |
|---|---|---|
| ΔfliC180 | pYA3729 | Cm |
| ΔfliC2426 | pYA3702 | Cm |
| ΔfljB217 | pYA3548 | Tet |
| Δ(hin-fljBA)-209 | pG8R306 | Cm |

H. Mutations altering synthesis of fimbrial components

| | | |
|---|---|---|
| Δ(agfG-agfC)-999 | pYA4941 | Cm |
| ΔP_{stc53}::P_{murA} stcA53 | pYA5053 | Cm |
| ΔstcABCD | pYA5007 | Tet |
| ΔP_{saf55}::P_{murA} safA55 | pYA5055 | Cm |
| ΔsafABCD | pYA4586 | Tet |
| ΔfimH1019 | pYA3545 | Tet |

I. Mutations eliminating or altering outer membrane proteins

| | | |
|---|---|---|
| ΔompA11 | pYA4757 | Tet |
| ΔompA3Ωplz4 | pG8R315 | Cm |

J. Mutations decreasing inflammation and enhancing mucosal immunity

| | | |
|---|---|---|
| ΔsopB1925 | pYA3733 | Cm |

K. Mutations eliminating or diminishing effective immunogenicity

| | | |
|---|---|---|
| ΔsifA26 | pYA3716 | Cm |

L. Mutations decreasing/delaying onset of pyroptosis

| | | |
|---|---|---|
| ΔsseL116 | pYA4621 | Cm |
| ΔtlpA181 | pYA4620 | Cm |

M. Mutations leading to degradation of DNA within Salmonella cells

| | | |
|---|---|---|
| ΔrecA62 | pYA4680 | Cm |
| ΔrecF126 | pYA3886 | Cm |
| ΔendA2311 | pYA3652 | Cm |

N. Mutations altering invasion

| | | |
|---|---|---|
| ΔP_{hilA}::P_{traΔlacO} hilA | pYA4681 | Cm |

O. Mutations leading to chemotaxis, purine and pyrimidine requirement

| | | |
|---|---|---|
| ΔpurA3114 | pG8R126 | Cm |
| ΔP_{purA185}::rhaRS P_{rhaBAD} purA | pG8R117 | Cm |
| ΔguaBA66 | pG8R449 | Cm |
| ΔP_{guaBA67}::rhaRS P_{rhaBAD} guaBA | pG8R450 | Cm |
| ΔpyrF68 | pG8R451 | Cm |
| ΔP_{pyrF69}::rhaRS P_{rhaBAD} pyrF | pG8R452 | Cm |
| ΔPₜₐᵣ::P_{trcΔlacO888} tar | pYA4946 | Cm |
| ΔPₜₛᵣ::P_{trcΔlacO888} tsr | pYA4947 | Cm |
| Δtrg-70 | pYA5077 | Cm |

^{a}Δ = deletion; TT = transcription terminator; P = promoter

TABLE 3
Genotypes of CCTS strains generated that have been
used in past research on anti-cancer therapies.

| Strain | genotype | Refs |
|---|---|---|
| χ4550 | Δcya-1 Δcrp-1 ΔasdA1 (Δzhf-4::Tn10) (from χ4064 STm SR-11) | (75-79) |
| χ8133 | Δcya-27 Δcrp-27 ΔasdA16 | (80) |
| χ11091 | ΔpabA1516 ΔpabB232 ΔasdA16 ΔmsbB48 ΔpagL7 ΔpagP81::Pₗₚₚ | (81) |
| | lpxE ΔlpxR93::Pₗₚₚ lpxF | |
| χ12342 | ΔwaaG42 ΔpagL21:TT araC P_{araBAD} waaG ΔlpxR9 ΔpagP8 (also | (16) |
| | with ΔaroA21419) | |
| BCT2 | ΔpabA1516 ΔpabB232 ΔasdA16 ΔmsbB48 ΔpagL7 ΔpagP81::Pₗₚₚ | (82) |
| | lpxE ΔlpxR93::Pₗₚₚ lpxF ΔfimH ΔfliC ΔfljB ΔrfaL (=waaL) ΔpgtEp | |
| | (from χ11091) | |

### Treatment Methods

The genetically modified Salmonella cells described herein and therapeutic compositions comprising the same may be used in methods to treat cancer, to attenuate the growth of a tumor or to regress a tumor. The methods described herein may be used to treat or attenuate the growth of any cancer or tumor type. Cancers and tumor types that may be treated or attenuated using the methods described herein include but are not limited to bone cancer, bladder cancer, brain cancer, breast cancer, cancer of the urinary tract, carcinoma, cervical cancer, colon cancer, esophageal cancer, gastric cancer, head and neck cancer, hepatocellular cancer, liver cancer, lung cancer, lymphoma and leukemia, melanoma, ovarian cancer, pancreatic cancer, pituitary cancer, prostate cancer, rectal cancer, renal cancer, sarcoma, testicular cancer, thyroid cancer, and uterine cancer. In addition, the methods may be used to treat tumors that are malignant (e.g., primary or metastatic cancers) or benign (e.g., hyperplasia, cyst, pseudocyst, hematoma, and benign neoplasm).

In some embodiments, a method for treating cancer may include administering a therapeutically effective amount of genetically modified Salmonella cells described herein or therapeutic compositions comprising the same to a subject who has cancer.

“Treating” or “treatment” of a condition may refer to preventing the condition, slowing the onset or rate of development of the condition, reducing the risk of developing the condition, preventing or delaying the development of symptoms associated with the condition, reducing or ending symptoms associated with the condition, generating a complete or partial regression of the condition, or some combination thereof.

A “therapeutically effective amount,” “effective amount” or “effective dose” is an amount of a composition (e.g., a therapeutic composition or cells) that produces a desired therapeutic effect in a subject, such as preventing or treating a target condition or alleviating symptoms associated with the condition. The precise therapeutically effective amount is an amount of the composition that will yield the most effective results in terms of efficacy of treatment in a given subject. This amount will vary depending upon a variety of factors, including but not limited to the characteristics of the therapeutic compound (including activity, pharmacokinetics, pharmacodynamics, and bioavailability), the physiological condition of the subject (including age, sex, disease type and stage, general physical condition, responsiveness to a given dosage, and type of medication), the nature of the pharmaceutically acceptable carrier or carriers in the formulation, and the route of administration. One skilled in the clinical and pharmacological arts will be able to determine a therapeutically effective amount through routine experimentation, namely by monitoring a subject's response to administration of a compound and adjusting the dosage accordingly. For additional guidance, see Remington: The Science and Practice of Pharmacy 21^{st} Edition, Univ. of Sciences in Philadelphia (USIP), Lippincott Williams & Wilkins, Philadelphia, Pa., 2005.

The therapeutic compositions described herein may be administered by any suitable route of administration. A “route of administration” may refer to any administration pathway known in the art, including but not limited to aerosol, enteral, nasal, ophthalmic, oral, parenteral, rectal, transdermal (e.g., topical cream or ointment, patch), or vaginal. “Parenteral” refers to a route of administration that is generally associated with injection, including infraorbital, infusion, intraarterial, intracapsular, intracardiac, intradermal, intramuscular, intraperitoneal, intrapulmonary, intraspinal, intrasternal, intrathecal, intrauterine, intravenous, subarachnoid, subcapsular, subcutaneous, transmucosal, or transtracheal. In one embodiment, the tumor antigen vaccines described herein (e.g., an SVN or CO-SVN Salmonella-based vaccine and associated expression plasmids) are administered orally and the compositions that disrupt tumor-derived immune suppression described herein (e.g., YS1646-shSTAT3 Y51646-shIDO1, YS1646-shArg1 or YS1646-shiNOS) are administered intravenously.

### EXAMPLES

### Example 1. Materials and Methods

a. Bacterial strains, media and bacterial growth. All CCTS strains are derived from the highly virulent S. typhimurium UK-1 strain since attenuated S. typhimurium UK-1 strains induce protective immunity to challenge with all S. typhimurium strains whereas other S. typhimurium strains attenuated with the same mutations often cannot induce protective immunity to some S. typhimurium strains and definitely not to virulent UK-1. LB broth and agar and Purple broth (PB) (Difco), which is devoid of arabinose (Ara), mannose (Man) and rhamnose (Rha), are used as complex media for propagation, phenotypic analyses and plating. MacConkey agar with 0.5% lactose (Lac) and 0.1% Ara, 0.1% rhamnose and 0.1% mannose (if needed) are used to enumerate bacteria recovered from mice or other animals. Bacterial growth is monitored spectrophotometrically and by plating for colony counts.

b. Molecular and genetic procedures. Methods for DNA isolation, restriction enzyme digestion, DNA cloning and use of PCR for construction and verification of bacterial strains and vectors are standard. DNA sequence analyses are performed commercially. All oligonucleotide and/or gene syntheses are done commercially with codon optimization to enhance translational efficiency in humans or Salmonella and stabilize mRNA to “destroy” RNase E cleavage sites to prolong mRNA half-life. Plasmids are evaluated by DNA sequencing and ability to specify synthesis of proteins using gel electrophoresis and western blot analyses. Expression of sequences encoded in DNA vaccine vectors is monitored after electroporation into Vero cells and using antibodies specific to DNA vaccine encoded proteins. Methods for generating mutant strains are described in previous publications and in Examples below using the suicide vector delivery strain χ7213 (thi-1 thr-1 leuB6 glnV44 fhuA21 lacY1 recA1 RP4-2-Tc::Mu λpir ΔasdA4 Δzhf-2::Tn 10). Recombinant plasmid constructs are transformed into E. coli χ6212(F⁻ λ⁻ φ80 Δ(lacZYA-argF) endA1 recA1 hsdR17 deoR thi-1 glnV44 gyrA96 relA1 ΔasdA4) with selection for AsdA⁺ for initial characterization prior to electroporation into CCTS strains.

c. Selection of targeting and effector proteins. Selection of proteins that facilitate targeting to cancer cells or constitute cargo proteins with desired biological effects to be encoded on regulated lysis plasmid vectors for synthesis and delivery by CCTS strains or to be encoded on DNA vaccine vectors for expression in the inoculated animal host are based on prior discoveries and evidence of well-established activities in the published literature.

d. CCTS strain characterization. CCTS constructs are evaluated in comparison with vector-control strains for stability of plasmid maintenance, integrity and protein synthesis ability when CCTSs are grown in the presence of arabinose and DAP and with and without IPTG for 50 generations. The IPTG dependence of protein synthesis to overcome the Lac repression of the P_{trc} promoter is also verified. IPTG-induced cultures are incubated with chloramphenicol to arrest protein synthesis to determine whether plasmid-specified proteins are stable during the next 4 h. If not, the nucleotide sequence is altered to eliminate protease cleavage sites (with subsequent comparison of both constructs for induction of immune responses). Measurement of LPS core and O-antigen is performed after electrophoresis using silver-stained gels. Final CCTS constructs are evaluated for bile sensitivity, acid tolerance and ability to survive in sera with and without complement and for sensitivity to antibiotics used to treat Salmonella infections.

e. Cell culture methods. Some tumors are caused by cancer cells with specific targetable receptors or that possess phenotypic properties that can be used to attract specially designed CCTS strains with specific targeting attributes. For example, bladder tumor cells uniquely display a receptor that can bind to a targeting peptide termed PLZ4 (amino acid sequence: CQDGRMGFC)(SEQ ID NO: 168) that is absent on normal uroepithelial cells and other cell types throughout the body. Nanoparticles coated with PLZ4 specifically target bladder tumor cells but not to other cancer cell types. This targeting is observed for bladder tumor cells from mice, dogs and humans. CCTS strains displaying PLZ4 can be evaluated by their differential ability to attach to and invade the bladder tumor cell lines 5637, TCCSUP, and T24. Methods for evaluating the abilities of Salmonella cells to attach to, invade into and survive in cells in culture are well established. These methods can be modified as needed for CCTS strains targeting other tumor cell types.

f. Cell imaging. Some plasmids have genes encoding fluorescent proteins enabling synthesis of GFP in Salmonella or EGFP or mCherry in animal cells. The fluorescent protein in bacteria or cells can be visualized using the EVOS Automated Cell Imaging System (ThermoFisher Scientific). The Cell Plasma Membrane Staining Kit—Orange Fluorescence—Cytopainter (ab219941, Abcam) was used to label cell membranes. The acquired image was processed using ImageJ software.

### Example 2. Construction of Mutant S. typhimurium Strains with Deletions of the ompA Gene to Enable Display of Altered OmpA Proteins with Inserted Peptides Enabling Targeting Specific Tumor Cells

Pan and associates have defined a nine amino acid peptide CQDGRMGFC (SEQ ID NO: 168) termed PLZ4 (U.S. Pat. No. 10,335,365) that targets a specific receptor present on bladder tumor cells. A number of S. typhimurium strains with anti-tumor attributes have been constructed to display PLZ4 to preferentially and specifically target bladder tumor cells. The objective was to insert the sequence for PLZ4 into one of the exposed outer loops of the OmpA protein. The OmpA protein was selected since it is the most abundant OMP in the Salmonella outer membrane and could be specified on a plasmid replicon to increase its relative quantity in relation to other OMPs.

To construct a strain to test the validity and feasibility of our approach, we generated a derivative of χ12341 to insert the ΔompA11 deletion mutation using the suicide vector pYA4757 (Table 2) to yield the strain χ12417 (Table 4). The ΔompA11 mutation deletes the entire ompA open reading frame including the start to stop codon sequence. χ12341 was selected since its viability and virulence are dependent on the supply of three sugars that can be supplied during culture but that are totally absent in animal tissues and since it cannot synthesize LPS O-antigen in vivo thus exposing the outer membrane proteins to enable better and more efficient interactions with eukaryotic cell surfaces in the in vivo environment.

After demonstrating that χ12417 harboring a multi-copy plasmid encoding the ompAΩplz4 fusion could adhere to bladder tumor cells displaying the receptor for PLZ4 (see below), studies were commenced to evaluate S. typhimurium strains with a diversity of properties for use with a diversity on new plasmid vectors encoding for synthesis of attributes that contribute to tumor therapy, tumor cell destruction and/or to recruit host immunity to target tumor antigens, etc., in addition to tumor cell adherence. All these S. typhimurium strains listed in Table 4 were constructed using the suicide vectors listed in Table 2 to introduce the mutations described in Table 1. Many were derived from Protective Immunity Enhanced Salmonella Vaccine (PIESV) strains of Self-Destructing Attenuated Adjuvant Salmonella (SDAAS) strains that have been described (PCT/US21/61814 and WO 2021/222696 A1, respectively), which are incorporated herein in their entirety.

TABLE 4
S. Typhimurium strains constructed and evaluated as CCTS strains.

χ12414 ΔwaaG42 ΔpagL21::TT araC P_{araBAD} waaG ΔlpxR9 ΔpagP8 ΔeptA4 ΔarnT6
Δpmi-2426 ΔrelA197::araC P_{araBAD} lacl TT ΔompA11
χ12417 ΔP_{murA25}::TT araC P_{BAD} murA ΔwaaL46 Δpmi-2426
ΔasdA27::TT araC P_{BAD} c2 ΔpagL64::TT rhaRS P_{rhaBAD} waaL Δ(wza-wcaM)-8
ΔrelA197::araC P_{BAD} lacl TT ΔrecF126 ΔsifA26 ΔompA11 (from χ12341)
χ12447 ΔP_{murA25}::TT araC P_{araBAD} murA ΔasdA27::TT araC P_{araBAD} c2 Δpmi-2426 Δ(wza-
wcaM)-8 ΔrecF126 ΔsifA26 ΔwaaL46 ΔpagL64::TT rhaRS P_{rhaBAD} waaL ΔendA2113
ΔrelA1123 ΔsseL116 ΔtlpA181 ΔompA11 (from χ12388)
χ12452 ΔP_{murA25}::TT araC P_{BAD} murA ΔwaaL46 ΔpagL64::TT rhaRS P_{rhaBAD} waaL Δpmi-
2426 ΔasdA27::TT araC P_{BAD} c2 ΔpagL64::TT rhaRS P_{rhaBAD} waaL Δ(wza-wcaM)-8
ΔrelA197::araC P_{BAD} lacl TT ΔrecF126 ΔsifA26 ΔompA11 ΔsopB1925 (from χ12417)
χ12485 ΔwaaL46 ΔpagL64::TT rhaRS P_{rhaBAD} waaL Δpmi-2426 ΔP_{fur33}::TT araC P_{araBAD}
fur ΔasdA33 ΔrelA197::araC P_{BAD} lacl TT Δ(wza-wcaM)-8 ΔP_{tolR67::}::TT araC P_{araBAD} tolR
ΔompA11 (from χ12473)
χ12494 ΔP_{murA25}::TT araC P_{araBAD} murA ΔasdA27::TT araC P_{araBAD} c2 Δpmi-2426 Δ(wza-
wcaM)-8 ΔrecF126 ΔsifA26 ΔwaaL46 ΔpagL64::TT rhaRS P_{rhaBAD} waaL ΔendA2113
ΔrelA1123 ΔsseL116 ΔtlpA181 ΔompA11 ΔsopB1925 (from χ12447)
χ12508 ΔP_{murA25}::TT araC P_{araBAD} murA ΔasdA27::TT araC P_{araBAD} c2 Δpmi-2426 Δ(wza-
wcaM)-8 ΔrelA197::araC P_{araBAD} lacl TT ΔrecF126 ΔsifA26 ΔwbaP45 ΔpagL14::TT araC
P_{araBAD} wbaP ΔlpxR9 ΔpagP8 ΔompA11 (from χ12449)
χ12525 ΔP_{murA25}::TT araC P_{BAD} murA ΔwaaL46 Δpmi-2426
ΔasdA27::TT araC P_{BAD} c2 ΔpagL64::TT rhaRS P_{rhaBAD} waaL Δ(wza-wcaM)-8
ΔrelA197::araC P_{BAD} lacl TT ΔrecF126 ΔsifA26 ΔompA11 ΔaraBAD65::TT
(from χ12417)
χ12526 ΔP_{murA25}::TT araC P_{BAD} murA ΔwaaL46 Δpmi-2426
ΔasdA27:TT araC P_{BAD} c2 ΔpagL64:TT rhaRS P_{rhaBAD} waaL Δ(wza-wcaM)-8
ΔrelA197::araC P_{BAD} lacl TT ΔrecF126 ΔsifA26 ΔompA11 ΔrhaDABSR515
(from χ12417)
χ12529 ΔP_{murA25}::TT araC P_{araBAD} murA ΔasdA27::TT araC P_{araBAD} c2 Δpmi-2426
ΔwaaL46 ΔpagL64::TT rhaRS P_{rhaBAD1} waaL1 Δ(wza-wcaM)-8
ΔrelA197::araC P_{araBAD} lacl TT ΔrecF126 ΔsifA26 ΔompA11 ΔaraBAD65::TT
ΔrhaBADSR515 (from χ12425)
χ12614 ΔasdA33 ΔompA11 (from χ8958)
χ12627 ΔwaaL46 ΔpagL64::TT rhaRS P_{rhaBAD} waaL Δpmi-2426 ΔP_{fur33}::TT araC P_{araBAD}
fur ΔasdA33 ΔrelA197::araC P_{BAD} lacl TT Δ(wza-wcaM)-8 ΔP_{tolR67::}::TT araC P_{araBAD} tolR
ΔompA11 ΔpagP81::Pₗₚₚ lpxE (from χ12485)
χ12628 ΔwaaL46 ΔpagL64::TT rhaRS P_{rhaBAD} waaL Δpmi-2426 ΔP_{fur33}::TT araC P_{araBAD}
fur ΔasdA33 ΔrelA197::araC P_{BAD} lacl TT Δ(wza-wcaM)-8 ΔP_{tolR67::}::TT araC P_{araBAD} tolR
ΔompA11 ΔpagP81::Pₗₚₚ lpxE ΔlpxR9 (from χ12627)
χ12632 ΔwaaL46 ΔpagL64::TT rhaRS P_{rhaBAD} waaL Δpmi-2426 ΔP_{fur33}::TT araC P_{araBAD}
fur ΔasdA33 ΔrelA197::araC P_{BAD} lacl TT Δ(wza-wcaM)-8 ΔP_{tolR67::}::TT araC P_{araBAD} tolR
ΔompA11 ΔpagP81::Pₗₚₚ lpxE ΔlpxR9 ΔeptA4 ΔarnT6 (from χ12631)
χ12654 ΔP_{murA25}::TT araC P_{BAD} murA ΔwaaL46 Δpmi-2426
ΔasdA27::TT araC P_{BAD} c2 ΔpagL64::TT rhaRS P_{rhaBAD} waaL Δ(wza-wcaM)-8
ΔrelA197::araC P_{BAD} lacl TT ΔrecF126 ΔsifA26 ΔompA11 ΔendA2311 (from χ12417)
χ12655 ΔP_{murA25}::TT araC P_{araBAD} murA ΔasdA27::TT araC P_{araBAD} c2 Δ(wza-wcaM)-8
ΔrecF126 ΔsifA26 ΔwaaL46 ΔpagL64::TT rhaRS P_{rhaBAD} waaL ΔendA2113 ΔrelA1123
ΔsseL116 ΔtlpA181 ΔompA11 (from χ12563)
χ12656 ΔP_{murA25}::TT araC P_{araBAD} murA ΔasdA27::TT araC P_{araBAD} c2 Δ(wza-wcaM)-8
ΔrelA197::araC P_{araBAD} lacl TT ΔrecF126 ΔsifA26 ΔwaaL46 ΔpagL38::TT rhaRS P_{rhaBAD2}
waaL2 ΔaraBAD65::TT ΔrhaBADSR515 ΔpagP8 ΔlpxR9 ΔompA11 (from χ12569)
χ12657 ΔP_{murA25}::TT araC P_{araBAD} murA ΔasdA27::TT araC P_{araBAD} c2 Δ(wza-wcaM)-8
ΔrecF126 ΔsifA26 ΔwaaL46 ΔpagL38::TT rhaRS P_{rhaBAD2} waaL2 ΔendA2113 ΔrelA1123
ΔsseL116 ΔtlpA181 ΔompA11 (from χ12601)
χ12658 ΔP_{murA25}::TT araC P_{araBAD} murA ΔasdA27::TT araC P_{araBAD} c2 Δ(wza-wcaM)-8
ΔrecF126 ΔsifA26 ΔwaaL46 ΔpagL38::TT rhaRS P_{rhaBAD2} waaL2 ΔaraBAD65::TT
ΔrhaBADSR515 ΔpagP8 ΔlpxR9 ΔrelA1123 ΔompA11 (pSTUK201 Δ(traM-traX)-
36::araC P_{araBAD} lacl TT) (from χ12615)
χ12667 ΔP_{murA25}::TT araC P_{araBAD} murA ΔasdA27::TT araC P_{araBAD} c2 Δ(wza-wcaM)-8
ΔrecF126 ΔsifA26 ΔwaaL46 ΔpagL38::TT rhaRS P_{rhaBAD2} waaL2 ΔaraBAD65::TT
ΔrhaBADSR515 ΔpagP8 ΔlpxR9 ΔrelA1123 ΔompA11(pSTUK206 Δ(traM-traX)-
41::araC P_{araBAD} lacl TT) (from χ12663)
χ12733 ΔP_{murA25}::TT araC P_{BAD} murA ΔwaaL46 Δpmi-2426
ΔasdA27::TT araC P_{BAD} c2 ΔpagL64::TT rhaRS P_{rhaBAD} waaL Δ(wza-wcaM)-8 ΔrecF126
ΔsifA26 ΔompA11 ΔendA2311 ΔrelA1123 (from χ12654)
χ12734 ΔP_{murA25}::TT araC P_{BAD} murA ΔwaaL46 ΔasdA27::TT araC P_{BAD} c2 ΔpagL64::TT
rhaRS P_{rhaBAD} waaL Δ(wza-wcaM)-8 ΔrelA197::araC P_{BAD} lacl TT ΔrecF126 ΔsifA26
ΔompA11 ΔendA2311 pmi₊ (from χ12654)
χ12735 ΔP_{murA25}::TT araC P_{araBAD} murA ΔasdA33 ΔwaaL46 ΔpagL38::TT rhaRS P_{rhaBAD2}
waaL2 Δ(wza-wcaM)-8 ΔrelA1123 ΔrecF126 ΔsifA26 ΔendA2113 ΔsseL116 ΔtlpA181
ΔrhaBADSR515 ΔaraBAD65::TT ΔompA11 (from χ12729)
χ12736 ΔP_{murA25}::TT araC P_{araBAD} murA ΔasdA33 ΔwaaL46 ΔpagL64::TT rhaRS P_{rhaBAD1}
waaL1 Δ(wza-wcaM)-8 ΔrelA1123 ΔrecF126 ΔsifA26 ΔendA2113 ΔsseL116 ΔtlpA181
ΔrhaBADSR515 ΔaraBAD65::TT ΔompA11 (from χ12730)
χ12748 ΔP_{murA25}::TT araC P_{araBAD} murA ΔasdA27::TT araC P_{BAD} c2 Δ(wza-wcaM)-8
ΔrelA197::araC P_{araBAD} lacl TT ΔrecF126 ΔsifA26 ΔwaaL46 ΔpagL64::TT rhaRS P_{rhaBAD1} waaL
ΔompA11 ΔsopB1925 (from χ12452)
χ12750 ΔP_{murA25}::TT araC P_{araBAD} murA ΔasdA33 ΔwaaL46 Δ(wza-wcaM)-8 ΔrecF126 ΔsifA26
ΔaraBAD65:TT ΔrhaBADSR515 ΔpagL38:TT rhaRS P_{rhaBAD2} waaL2 ΔpagP8 ΔlpxR9
ΔrelA1123 (pSTUK206 Δ(traM-traX)-41::araC P_{araBAD} lacl TT) ΔompA11 (from χ12688)
χ12751 ΔP_{murA25}::TT araC P_{araBAD} murA ΔasdA33 ΔwaaL46 Δ(wza-wcaM)-8 ΔrecF126 ΔsifA26
ΔaraBAD65::TT ΔrhaBADSR515 ΔpagL38::TT rhaRS P_{rhaBAD2} waaL2 ΔpagP8 ΔlpxR9
ΔrelA1123 (pSTUK206 Δ(traM-traX)-41::araC P_{araBAD} lacl TT) ΔompA11 ΔsopB1925 (from
χ12750)
χ12753 ΔP_{murA25}::TT araC P_{araBAD} murA ΔasdA33 ΔwaaL46 Δ(wza-wcaM)-8 ΔrecF126 ΔsifA26
ΔaraBAD65:TT ΔrhaBADSR515 ΔpagL38:TT rhaRS P_{rhaBAD2} waaL2 ΔpagP81: Pₗₚₚ lpxE
ΔlpxR9 ΔrelA1123 (pSTUK206 Δ(traM-traX)-41::araC P_{araBAD} lacl TT) ΔompA11 (from χ12702)
χ12754 ΔP_{murA25}::TT araC P_{araBAD} murA ΔasdA33 ΔwaaL46 Δ(wza-wcaM)-8 ΔrecF126 ΔsifA26
ΔaraBAD65::TT ΔrhaBADSR515 ΔpagL38::TT rhaRS P_{rhaBAD2} waaL2 ΔpagP81::Pₗₚₚ lpxE
ΔlpxR9 ΔrelA1123 (pSTUK206 Δ(traM-traX)-41::araC P_{araBAD} lacl TT) ΔompA11 ΔsopB1925
(from χ12753)
χ12755 ΔP_{murA25}::TT araC P_{BAD} murA ΔwaaL46 ΔasdA27::TT araC P_{BAD} c2 ΔpagL64::TT
rhaRS P_{rhaBAD} waaL Δ(wza-wcaM)-8 ΔrecF126 ΔsifA26 ΔompA11 ΔendA2311
ΔrelA1123 (from χ12734)
χ12756 ΔwaaG42 ΔpagL21::TT araC P_{araBAD} waaG ΔlpxR9 ΔpagP8 ΔeptA4 ΔarnT6
Δpmi-2426 ΔrelA197::araC P_{araBAD} lacl TT ΔompA11 ΔrelA1123 (from χ12414)
χ12775 ΔP_{murA25}::TT araC P_{BAD} murA ΔwaaL46 ΔasdA27::TT araC P_{BAD} c2 ΔpagL64::TT
rhaRS P_{rhaBAD} waaL Δ(wza-wcaM)-8 ΔrecF126 ΔsifA26 ΔompA11 ΔendA2311
ΔrelA1123 (pSTUK206 Δ(traM-traX)-41::araC P_{araBAD} lacl TT) (from χ12755)
χ12776 ΔwaaG42 ΔpagL21:TT araC P_{araBAD} waaG ΔlpxR9 ΔpagP8 ΔeptA4 ΔarnT6
Δpmi-2426 ΔrelA197::araC P_{araBAD} lacl TT ΔompA11 ΔrelA1123 (pSTUK206 Δ(traM-
traX)-41::araC P_{araBAD} lacl TT) (from χ12756)
χ12838 ΔP_{murA25}::TT araC P_{BAD} murA ΔwaaL46 ΔpagL64::TT rhaRS P_{rhaBAD} waaL1
Δ(wza-wcaM)-8 ΔrecF126 ΔsifA26 ΔompA11 ΔendA2311 ΔrelA1123 ΔasdA33 (from
χ12775)
χ12846 ΔwaaG42 ΔpagL21::TT araC P_{araBAD} waaG ΔlpxR9 ΔpagP8 ΔeptA4 ΔarnT6
Δpmi-2426 ΔrelA197::araC P_{araBAD} lacl TT ΔompA11 ΔrelA1123 (pSTUK206 Δ(traM-
traX)-41::araC P_{araBAD} lacl TT) ΔsifA26 (from χ12776)

We also constructed strains in which the ompAΩplz4 fusion replaced the wild-type chromosomal ompA gene to use as comparative controls with but one copy of the fusion. These strains constructed using the suicide vector pG8R315 (Table 2) are listed in Table 5. One of the examples is χ12619. The mutations ΔwaaL46, ΔwaaG42 and ΔwaaC41 were introduced into strain χ12619 to generate a family of strains differing in the presence of the LPS O-antigen, LPS O-antigen and outer LPS core and O-antigen and outer and inner LPS core, respectively.

We also constructed strain χ12614 with the ΔasdA33 ΔompA11 deletion mutations. This strain can be transformed with a plasmid encoding ompAΩplz4 to compare the effects of surface modification in Salmonella that affect the targeting ability of Salmonella. The plasmids could be pG8R341, or any plasmid carrying ompAΩplz4 fusion, or other ompA fused with varied targeting peptide sequences. The mutations ΔwaaL46, ΔwaaG42 and ΔwaaC41 were introduced into χ12614 to generate a series of strains analogous to those generated in χ12619 resulting in defects in O-antigen, outer core and inner core, respectively. These strains are also listed in Table 5.

TABLE 5
S. Typhimurium strains constructed with the ompAΩplz4 fusion

χ12518 Δalr-3 ΔP_{dadB66}::TT araC P_{araBAD} dadB ΔP_{asdA55}::TT araC P_{araBAD} asdA ΔfliC180
ΔpagP81::Pₗₚₚ lpxE ΔpagL7 ΔlpxR9 (from χ12516)
χ12542 Δalr-3 ΔP_{dadB66}::TT araC P_{araBAD} dadB ΔP_{asdA55}::TT araC P_{araBAD} asdA ΔfliC180
ΔpagP81::Pₗₚₚ lpxE ΔpagL7 ΔlpxR9 ΔwaaC41
χ12543 Δalr-3 ΔP_{dadB66}::TT araC P_{araBAD} dadB ΔP_{asdA55}::TT araC P_{araBAD} asdA ΔfliC180
ΔpagP81::Pₗₚₚ lpxE ΔpagL7 ΔlpxR9 ΔwaaG42
χ12544 Δalr-3 ΔP_{dadB66}::TT araC P_{araBAD} dadB ΔP_{asdA55}::TT araC P_{araBAD} asdA ΔfliC180
ΔpagP81::Pₗₚₚ lpxE ΔpagL7 ΔlpxR9 ΔwaaL46
χ12617 ompAΩplz4 (from χ3761)
χ12618 ΔrelA4 ΔspoT1 ΔasdA27:TT araC P_{araBAD} c2 ompAΩplz4 (from χ11001)
χ12619 Δalr-3 ΔP_{dadB66}::TT araC P_{araBAD} dadB ΔP_{asdA55}::TT araC P_{araBAD} asdA ΔfliC180
ΔpagP81::Pₗₚₚ lpxE ΔpagL7 ΔlpxR9 ompAΩplz4 (from χ12518)
χ12808 Δalr-3 ΔP_{dadB66}::TT araC P_{araBAD} dadB ΔP_{asdA55}::TT araC P_{araBAD} asdA ΔfliC180
ΔpagP81::Pₗₚₚ lpxE ΔpagL7 ΔlpxR9 ompAΩplz4 ΔwaaL46 (from χ12619)
χ12809 Δalr-3 ΔP_{dadB66}::TT araC P_{araBAD} dadB ΔP_{asdA55}::TT araC P_{araBAD} asdA ΔfliC180
ΔpagP81::Pₗₚₚ lpxE ΔpagL7 ΔlpxR9 ompAΩplz4 ΔwaaG42 (from χ12619)
χ12810 Δalr-3 ΔP_{dadB66}::TT araC P_{araBAD} dadB ΔP_{asdA55}::TT araC P_{araBAD} asdA ΔfliC180
ΔpagP81::Pₗₚₚ lpxE ΔpagL7 ΔlpxR9 ompAΩplz4 ΔwaaC41 (from χ12619)
χ12811 Δalr-3 ΔP_{dadB66}::TT araC P_{araBAD} dadB ΔP_{asdA55}::TT araC P_{araBAD} asdA ΔfliC180
ΔpagP81::Pₗₚₚ lpxE ΔpagL7 ΔlpxR9 ΔwaaC41 ompAΩplz4 (from χ12542)
χ12614 ΔasdA33 ΔompA11 (from χ8958)
χ12812 ΔasdA33 ΔompA11 ΔwaaL46 (from χ12614)
χ12813 ΔasdA33 ΔompA11 ΔwaaG42 (from χ12614)
χ12814 ΔasdA33 ΔompA11 ΔwaaC41 (from χ12614)

### Example 3. Construction of Plasmid Vectors for Use and Evaluation in Candidate CCTS Strains

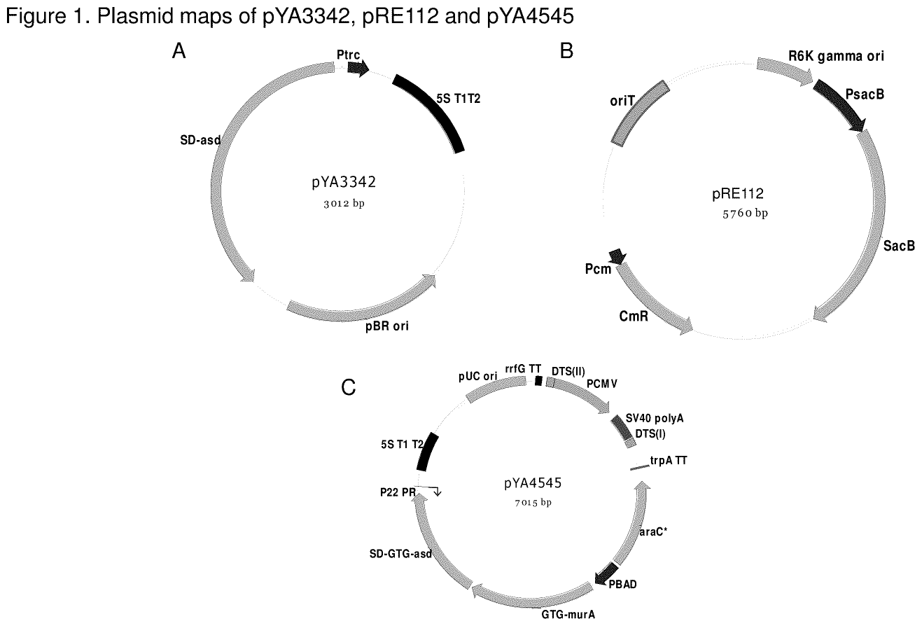

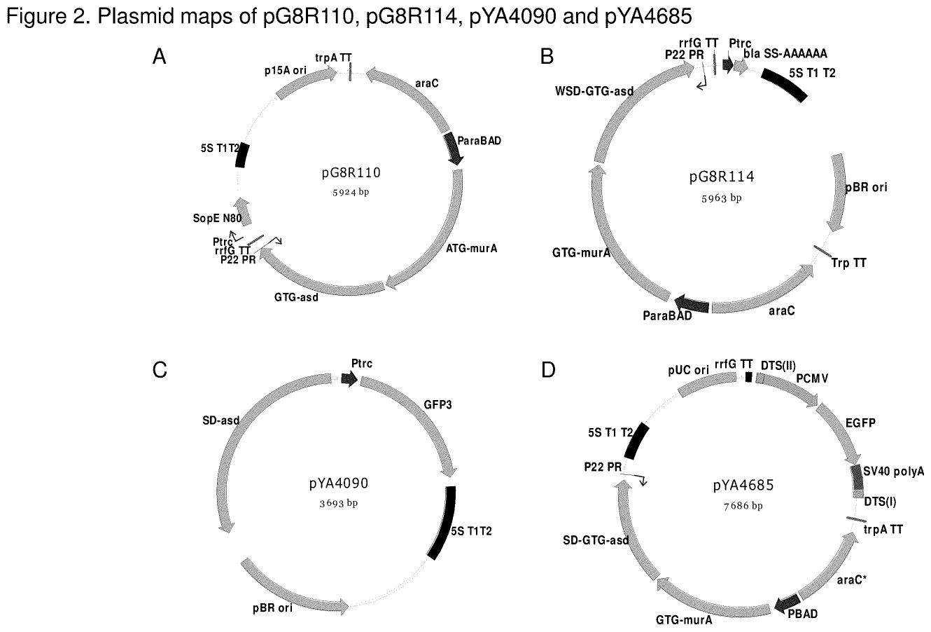

FIGS. 1 and 2 depict the plasmids used as parents or for component segments of derived and constructed plasmids. The derived and constructed plasmids are listed and described in Table 6 with their use and evaluation described in subsequent Examples. Table 7 lists all the nucleotide primers used to construct the plasmids listed in Table 6. A unique and original feature of many of the plasmids designed and constructed is the ability to encode proteins that are synthesized by the CCTS strain to be displayed during targeting and attaching to, invading into and acting within tumor cells in vivo prior to display of regulated lysis within the tumor cell to release the plasmid now serving as a DNA vaccine with unique features to be directed to the nucleus for transcription of encoded sequences that yield products after mRNA translation that exhibit anti-tumor activities. These products with their features as described in later Examples might kill the tumor cell, cause the tumor cell to kill itself (i.e., commit suicide) and/or attract host immune responses that inhibit tumor cell growth and metastases.

The ability to design and effectively use these newly designed dual function hybrid plasmids is dependent on using CCTS delivery strains that are engineered to express a regulated delayed lysis in vivo phenotype that is a composite function of regulated expression of both chromosomal and plasmid encoded genes.

TABLE 6
Lists of plasmids generated for use in anti-tumor research^{a}.

| | | | | Gene | | | |
|---|---|---|---|---|---|---|---|
| | | | | Prokaryotic | Eukaryotic | | Parent |
| Plasmid | Replicon | Marker | Promoter | expression | expression | Linker | plasmid |
| pG8R314 | pBR | asd | P_{trc} | ompAΩPLZ4 | | AC-PLZ4- | pYA3342 |
| | | | | | | CG | |
| pG8R315 | R6K | Cm | | ompAΩPLZ4 | | AC-PLZ4- | pRE112 |
| | | | | | | CG | |
| pG8R319 | pBR | asd | P_{trc} | ompAΩPLZ4 | | AC-PLZ4- | pG8R314 |
| | | | P_{CMV} | | | CG | |
| pG8R320 | pUC | araC | P_{trc} | ompAΩPLZ4 | | AC-PLZ4- | pYA4545 |
| | | P_{BAD} | P_{CMV} | | | CG | |
| | | murA | | | | | |
| | | asd | | | | | |
| pG8R321 | pBR | asd | P_{trc} | ompAΩPLZ4 | human | AC-PLZ4- | pG8R319 |
| | | | P_{CMV} | | CXCL11 | CG | |
| pG8R322 | pBR | asd | P_{trc} | ompAΩPLZ4 | mouse | AC-PLZ4- | pG8R319 |
| | | | P_{CMV} | | CXCL11 | CG | |
| pG8R323 | pBR | asd | P_{trc} | ompAΩPLZ4 | KillerRed | AC-PLZ4- | pG8R319 |
| | | | P_{CMV} | | Mem | CG | |
| pG8R324 | pBR | asd | P_{trc} | ompAΩPLZ4 | KillerRed | AC-PLZ4- | pG8R319 |
| | | | P_{CMV} | | Mito | CG | |
| pG8R325 | pUC | araC | P_{trc} | ompAΩPLZ4 | human | AC-PLZ4- | pG8R320 |
| | | P_{BAD} | P_{CMV} | | CXCL11 | CG | |
| | | murA | | | | | |
| | | asd | | | | | |
| pG8R326 | pUC | araC | P_{trc} | ompAΩPLZ4 | mouse | AC-PLZ4- | pG8R320 |
| | | P_{BAD} | P_{CMV} | | CXCL11 | CG | |
| | | murA | | | | | |
| | | asd | | | | | |
| pG8R327 | pUC | araC | P_{trc} | ompAΩPLZ4 | KillerRed | AC-PLZ4- | pG8R320 |
| | | P_{BAD} | P_{CMV} | | Mem | CG | |
| | | murA | | | | | |
| | | asd | | | | | |
| pG8R328 | pUC | araC | P_{trc} | ompAΩPLZ4 | KillerRed | AC-PLZ4- | pG8R320 |
| | | P_{BAD} | P_{CMV} | | Mito | CG | |
| | | murA | | | | | |
| | | asd | | | | | |
| pYA4090 | pBR | asd | P_{trc} | GFP3 | | | pYA3342 |
| pYA4685 | pUC | araC | P_{CMV} | | EGFP | | pYA4545 |
| | | P_{BAD} | | | | | |
| | | murA | | | | | |
| | | asd | | | | | |
| pG8R341 | pBR | Asd | P_{trc} | ompAΩPLZ4 SD | | | pG8R314 |
| | | | | GFP | | | |
| pG8R342 | pUC | araC | P_{trc} | ompAΩPLZ4 | EGFP | | pG8R320 |
| | | P_{BAD} | P_{CMV} | | | | |
| | | murA | | | | | |
| | | asd | | | | | |
| pG8R343 | pUC | araC | P_{trc} | ompAΩPLZ4 | KillerRED- | GSG P2A | pG8R320 |
| | | P_{BAD} | P_{CMV} | | memo- | | |
| | | murA | | | Human | | |
| | | asd | | | CXCL11 | | |
| pG8R344 | pUC | araC | P_{trc} | ompAΩPLZ4 | KillerRED- | GSG P2A | pG8R320 |
| | | P_{BAD} | P_{CMV} | | memo- | | |
| | | murA | | | Mouse | | |
| | | asd | | | CXCL11 | | |
| pG8R345 | pUC | araC | P_{trc} | ompAΩPLZ4 | HLAB | | pG8R320 |
| | | P_{BAD} | P_{CMV} | | leading and | | |
| | | murA | | | tail peptide | | |
| | | asd | | | | | |
| pG8R346 | pUC | araC | P_{trc} | ompAΩPLZ4 | HLAB- | | pG8R345 |
| | | P_{BAD} | P_{CMV} | | leading and | | |
| | | murA | | | tail peptide | | |
| | | asd | | | EGFP | | |
| pG8R347 | pUC | araC | P_{trc} | ompAΩPLZ4 | HLAB | | pG8R320 |
| | | P_{BAD} | P_{CMV} | | leading and | | |
| | | murA | | | tail peptide | | |
| | | asd | | | with 5′ and 3′ | | |
| pG8R348 | pUC | araC | P_{trc} | ompAΩPLZ4 | HLAB | | pG8R347 |
| | | P_{BAD} | P_{CMV} | | leading and | | |
| | | murA | | | tail peptide | | |
| | | asd | | | with 5′ and 3′ + | | |
| | | | | | EGFP | | |
| pG8R349 | pUC | araC | P_{trc} | ompAΩPLZ4 | HLAB | | pG8R347 |
| | | P_{BAD} | P_{CMV} | | leading and | | |
| | | murA | | | tail peptide | | |
| | | asd | | | with 5′ and 3′ | | |
| | | | | | BBN963 | | |
| pG8R350 | pUC | araC | P_{trc} | ompAΩPLZ4 | HLAB | | pG8R347 |
| | | P_{BAD} | P_{CMV} | | leading and | | |
| | | murA | | | tail peptide | | |
| | | asd | | | with 5′ and 3′ + | | |
| | | | | | MB49 | | |
| pG8R361 | pUC | araC | P_{trc} | ompAΩPLZ4 | | | pG8R320 |
| | | P_{BAD} | P_{EF1α} | | | | |
| | | murA | | | | | |
| | | asd | | | | | |
| pG8R362 | pUC | araC | P_{trc} | ompAΩPLZ4 | HAC-PD1- | 36 AA | pG8R320 |
| | | P_{BAD} | P_{CMV} | | hCXCL11 | Linker | |
| | | murA | | | | | |
| | | asd | | | | | |
| pG8R363 | pUC | araC | P_{trc} | ompAΩPLZ4 | HAC-PD1- | 36 AA | pG8R320 |
| | | P_{BAD} | P_{CMV} | | mCXCL11 | Linker | |
| | | murA | | | | | |
| | | asd | | | | | |
| pG8R364 | pUC | araC | P_{trc} | ompAΩPLZ4 | HAC-PD1- | 36 AA | pG8R361 |
| | | P_{BAD} | P_{EF1α} | | hCXCL11 | Linker | |
| | | murA | | | | | |
| | | asd | | | | | |
| pG8R365 | pUC | araC | P_{trc} | ompAΩPLZ4 | HAC-PD1- | 36 AA | pG8R361 |
| | | P_{BAD} | P_{EF1α} | | mCXCL11 | Linker | |
| | | murA | | | | | |
| | | asd | | | | | |
| pG8R366 | pUC | araC | Ptrc | ompAΩPLZ4 | IL2 SS | | pG8R320 |
| | | P_{BAD} | P_{CMV} | | | | |
| | | murA | | | | | |
| | | asd | | | | | |
| pG8R367 | pUC | araC | P_{trc} | ompAΩPLZ4 | IL2 SS-HAC- | 36 AA | pG8R366 |
| | | P_{BAD} | P_{CMV} | | PD1- | Linker | |
| | | murA | | | hCXCL11 | | |
| | | asd | | | | | |
| pG8R368 | pUC | araC | P_{trc} | ompAΩPLZ4 | IL2 SS-HAC- | 36 AA | pG8R366 |
| | | P_{BAD} | P_{CMV} | | PD1- | Linker | |
| | | murA | | | mCXCL11 | | |
| | | asd | | | | | |
| pG8R372 | pUC | araC | P_{trc} | ompAΩPLZ4 | IL2 SS-HAC- | | pG8R320 |
| | | P_{BAD} | P_{CMV} | | PD1 | | |
| | | murA | | | | | |
| | | asd | | | | | |
| pG8R373 | pUC | araC | P_{trc} | ompAΩPLZ4 | IL2 SS-HAC- | PPVAT | pG8R320 |
| | | P_{BAD} | P_{CMV} | | PD1-EGFP | (SEQ ID | |
| | | murA | | | | NO: 323) | |
| | | asd | | | | linker | |
| pG8R374 | pUC | araC | P_{trc} | ompAΩPLZ4 | hCXCL11- | PPVAT(SEQ | pG8R320 |
| | | P_{BAD} | P_{CMV} | | EGFP | ID NO: 323) | |
| | | murA | | | | linker | |
| | | asd | | | | | |
| pG8R375 | pUC | araC | P_{trc} | ompAΩPLZ4 | mCXCL11- | PPVAT(SEQ | pG8R320 |
| | | P_{BAD} | P_{CMV} | | EGFP | ID NO: 323) | |
| | | murA | | | | linker | |
| | | asd | | | | | |
| pG8R380 | pUC | araC | P_{trc} | ompAΩLHRH- | | | pG8R320 |
| | | P_{BAD} | P_{CMV} | GFP | | | |
| | | murA | | | | | |
| | | asd | | | | | |
| pG8R381 | pUC | araC | P_{trc} | ompAΩLHRH | | | pG8R380 |
| | | P_{BAD} | P_{CMV} | | | | |
| | | murA | | | | | |
| | | asd | | | | | |
| pG8R382 | pUC | araC | P_{trc} | ompAΩPLZ4 | haPD1-IgG | | pG8R320 |
| | | P_{BAD} | P_{CMV} | | | | |
| | | murA | | | | | |
| | | asd | | | | | |
| pG8R383 | pUC | araC | P_{trc} | ompAΩPLZ4 | haPD1-IgG- | GSG P2A | pG8R320 |
| | | P_{BAD} | P_{CMV} | | KillerRed- | peptide | |
| | | murA | | | memo | | |
| | | asd | | | | | |
| pG8R384 | pUC | araC | P_{trc} | ompAΩPLZ4 | KillerRed- | GSG P2A | pG8R320 |
| | | P_{BAD} | P_{CMV} | | memo | peptide | |
| | | murA | | | haPD1-IgG- | | |
| | | asd | | | | | |
| pG8R385 | pUC | araC | P_{trc} | ompAΩHer2 scFv- | | | pG8R320 |
| | | P_{BAD} | P_{CMV} | GFP | | | |
| | | murA | | | | | |
| | | asd | | | | | |
| pG8R386 | pUC | araC | P_{trc} | ompAΩHer2 scFv | | | pG8R385 |
| | | P_{BAD} | P_{CMV} | | | | |
| | | murA | | | | | |
| | | asd | | | | | |
| pG8R388 | pUC | araC | P_{trc} | | | | pG8R320 |
| | | P_{BAD} | P_{CMV} | | | | |
| | | murA | | | | | |
| | | asd | | | | | |
| pG8R389 | pUC | araC | P_{trc} | Bla AAAAAA | | | pG8R320 |
| | | P_{BAD} | P_{CMV} | | | | |
| | | murA | | | | | |
| | | asd | | | | | |
| pG8R390 | pUC | araC | P_{trc} | ompAΩLHRH | KillerRed- | | pG8R381 |
| | | P_{BAD} | P_{CMV} | | mem | | |
| | | murA | | | | | |
| | | asd | | | | | |
| pG8R391 | pUC | araC | P_{trc} | ompAΩHer2 scFv | KillerRed- | | pG8R386 |
| | | P_{BAD} | P_{CMV} | | mem | | |
| | | murA | | | | | |
| | | asd | | | | | |
| pG8R418 | pUC | araC | P_{trc} | ompAΩHer2 scFv- | KillerRed- | | pG8R385 |
| | | P_{BAD} | P_{CMV} | GFP | mem | | |
| | | murA | | | | | |
| | | asd | | | | | |
| pG8R478 | pUC | araC | P_{trc} | ompAΩRKOpep | | | pG8R320 |
| | | P_{BAD} | P_{CMV} | GFP | | | |
| | | murA | | | | | |
| | | asd | | | | | |
| pG8R479 | pUC | araC | P_{trc} | ompAΩTK | | | pG8R320 |
| | | P_{BAD} | P_{CMV} | GFP | | | |
| | | murA | | | | | |
| | | asd | | | | | |
| pG8R480 | pUC | araC | P_{trc} | ompAΩA3 | | | pG8R320 |
| | | P_{BAD} | P_{CMV} | GFP | | | |
| | | murA | | | | | |
| | | asd | | | | | |
| pG8R481 | pUC | araC | P_{trc} | ompAΩaMSH(1- | | | pG8R320 |
| | | P_{BAD} | P_{CMV} | 13) GFP | | | |
| | | murA | | | | | |
| | | asd | | | | | |
| pG8R482 | pUC | araC | P_{trc} | ompAΩCy12-RP2 | | | pG8R320 |
| | | P_{BAD} | P_{CMV} | GFP | | | |
| | | murA | | | | | |
| | | asd | | | | | |
| pG8R483 | pUC | araC | P_{trc} | ompAΩEBIP37 | | | pG8R320 |
| | | P_{BAD} | P_{CMV} | GFP | | | |
| | | murA | | | | | |
| | | asd | | | | | |
| pG8R484 | pUC | araC | P_{trc} | ompAΩFSHβ(33- | | | pG8R320 |
| | | P_{BAD} | P_{CMV} | 53) GFP | | | |
| | | murA | | | | | |
| | | asd | | | | | |
| pG8R485 | pUC | araC | P_{trc} | ompAΩFSHβ(81- | | | pG8R320 |
| | | P_{BAD} | P_{CMV} | 95) GFP | | | |
| | | murA | | | | | |
| | | asd | | | | | |
| pG8R486 | pUC | araC | P_{trc} | ompAΩGE11 GFP | | | pG8R320 |
| | | P_{BAD} | P_{CMV} | | | | |
| | | murA | | | | | |
| | | asd | | | | | |
| pG8R487 | pUC | araC | P_{trc} | ompAΩPeptideC | | | pG8R320 |
| | | P_{BAD} | P_{CMV} | GFP | | | |
| | | murA | | | | | |
| | | asd | | | | | |
| pG8R488 | pUC | araC | P_{trc} | ompAΩP20 GFP | | | pG8R320 |
| | | P_{BAD} | P_{CMV} | | | | |
| | | murA | | | | | |
| | | asd | | | | | |
| pG8R489 | pUC | araC | P_{trc} | ompAΩS36 GFP | | | pG8R320 |
| | | P_{BAD} | P_{CMV} | | | | |
| | | murA | | | | | |
| | | asd | | | | | |
| pG8R490 | pUC | araC | P_{trc} | ompAΩT7 GFP | | | pG8R320 |
| | | P_{BAD} | P_{CMV} | | | | |
| | | murA | | | | | |
| | | asd | | | | | |
| pG8R491 | pUC | araC | P_{trc} | ompAΩT12 GFP | | | pG8R320 |
| | | P_{BAD} | P_{CMV} | | | | |
| | | murA | | | | | |
| | | asd | | | | | |

^{a}The sizes of all plasmids in number of nucleotide bases is indicated for each plasmid in the accompanying Figures diagramming the plasmids listed in this Table 6.

TABLE 7
List of all the nucleotide primers used to construct
the plasmids listed in Table 6.

| SEQ ID | | |
|---|---|---|
| NO: | Name | Sequence |
| 54 | OmpA-s | 5′ |
| | | GATAACAATTTCACACAGGAAACAGACCATGAAAAAGACAG |
| | | CTATCGC 3′ |
| 55 | OmpA-PLZ4-a | 5′ |
| | | GAAACCCATACGACCGTCCTGGCACGCGCCAGGGACGTTA |
| | | GACTTG 3′ |
| 56 | OmpA-PLZ4-s | 5′ |
| | | CCAGGACGGTCGTATGGGTTTCTGCGGTGGCCCGTCTACTA |
| | | AAGACCAC 3′ |
| 57 | OmpA-SacIHindIII- | 5′ |
| | a | GCCAAAACAGCCAAGCTTGAGCTCATTAAGCCTGCGGCTGA |
| | | GTTAC 3′ |
| 58 | OmpA-XbaI-s | 5′ CCCAGCAGTCTAGAATGAAAAAGACAGCTATCGC 3′ |
| 59 | rrfGTT-s | 5′ |
| | | CTGCAAAGAGATGTGOGGATCTCTAGATTATGCGAAAGGC |
| | | 3′ |
| 60 | trpTT-a | 5′ |
| | | CAACAGCTCATTTCAGAATGGAAGAAAAAAAAGCCCGCTCA |
| | | TTAG 3′ |
| 61 | trpTT-s | 5′ |
| | | CTAATGAGCGGGCTTTTTTTTCTTCCATTCTGAAATGAGCTG |
| | | TTG 3′ |
| 62 | rrfGTT-a | 5′ GCCTTTCGCATAATCTAGAGATCCGCACATCTCTTTGCAG |
| | | 3′ |
| 63 | pYA4545-TT- | 5′ |
| | BstBI-s | GTAACTCAGCCGCAGGCTTAATGAGCTTCGAAACAGATTAA |
| | | ATCAGAACGCAGAAGCG 3′ |
| 64 | pYA4545-TT-a1 | 5′ |
| | | AAAAAAAACCCCGCCCTGTCAGGGGGGGGGTTTTTTTTTCC |
| | | TACGCTCACCCATCAATTG 3′ |
| 65 | pYA4545-TT-BcII- | 5′ |
| | a | GATTAATTGTCAACAGCTCATTTCAGAATGATCAAAAAAAAC |
| | | CCCGCCCTGTCAGGGGC 3′ |
| 66 | Ptrc-BcII-s | 5′ |
| | | CGCCCCTGACAGGGGGGGGTTTTTTTTGATCATTCTGAAAT |
| | | GAGCTGTTGAC 3′ |
| 67 | ompA-BstEI-a | 5′ |
| | | CTTCTGOGTTCTGATTTAATCTGTTTCGAAGCTCATTAAGCC |
| | | TGCGGCTGAG 3′ |
| 68 | Human-CXCL11- | 5′ |
| | KpnI-s | CGTTTAAACTTAAGCTTGGTACCGCCATGAGTGTGAAGGGC |
| | | ATGGC 3′ |
| 69 | Human-CXCL11- | 5′ |
| | Not-a | GTCTGCTCGAAGCATTCTCGAGCGGCCGCTTAAAAATTCTTT |
| | | CTTTCAAC 3′ |
| 70 | Mouse-CXCL11- | 5′ |
| | KpnI-s | GCGTTTAAACTTAAGCTTGGTACCGCCATGAACAGGAAGGT |
| | | CACAGC 3′ |
| 71 | Mouse-CXCL11- | 5′ |
| | NotI-a | CTCGAAGCATTCTCGAGCGGCCGCTTACATGTTTTGACGCC |
| | | TTAAAAAATTC 3′ |
| 72 | KiIIerRed-Mem- | 5′ |
| | KpnI-s | GCGTTTAAACTTAAGCTTGGTACCGCCACCATGCTGTGCTG |
| | | TATGAGAAGAACCAAAC 3′ |
| 73 | KiIIerRed- | 5′ |
| | NotIXhoI-a | GCTCGAAGCATTCTCGAGCGGCCGCTTTAATCCTCGTCGCT |
| | | ACCG 3′ |
| 74 | KillerRed-Mito- | 5′ |
| | KpnI-s | GCGTTTAAACTTAAGCTTGGTACCGCCACCATGTCCGTCCT |
| | | GACGCCGCTGC 3′ |
| 75 | SD-GFP-SacI-gs | 5 |
| | | CGCAGGCTTAATGAGCTCAAGGAACAGTCAATGAGTAAAGG |
| | | AGAAGAAC 3′ |
| 76 | GFP-HindIII-ga | 5′ |
| | | CATCCGCCAAAACAGCCAAGCTTATTATTTGTATAGTTCATC |
| | | CATGC 3′ |
| 77 | EGFP-KpnI-gs | 5′ |
| | | GTTTAAACTTAAGCTTGGTACCACCAAAATGGTGAGCAAGG |
| | | GCGAG 3′ |
| 78 | EGFP-XhoI-ga | 5 |
| | | CTGCTCGAAGCATTCTCGAGTTACTTGTACAGCTCGTCCAT |
| | | G 3′ |
| 79 | KillerRed-C-P2A- | 5′ |
| | a1 | GCCTGCTTCAGCAGGCTGAAGTTAGTAGCTCCGCTTCCATC |
| | | CTCGTCGCTACCGATGG 3′ |
| 80 | KillerRed-C-P2A- | 5′ |
| | a2 | AGGTCCAGGGTTCTCCTCCACGTCGCCAGCCTGCTTCAGCA |
| | | GGCTGAAG 3′ |
| 81 | P2A-Mouse | 5′ |
| | CXCL11-s | CGACGTGGAGGAGAACCCTGGACCTATGAACAGGAAGGTC |
| | | ACAGC 3′ |
| 82 | P2A-Human | 5 |
| | CXCL11-s | CGACGTGGAGGAGAACCCTGGACCTATGAGTGTGAAGGGC |
| | | ATGGC 3′ |
| 83 | HLAB-Leading-gs | 5′ |
| | | CTTAAGCTTGGTACGCCGCCACCATGCTGGTCATGGCGCCC |
| | | CG 3′ |
| 84 | HLAB-Leading- | 5′ |
| | MCS-ga | CCTAGGCCCGGGCCCGGTACCGGAGCCGGCCCAGGTCTC |
| | | GG 3′ |
| 85 | HLAB-tail-MCS-gs | 5′ |
| | | GGTACCGGGCCCGGGCCTAGGGGCCTGGCTGTCCTGGCA |
| | | G 3′ |
| 86 | HLAB-tail-XhoI-ga | 5 |
| | | CTGCTCGAAGCATTCTCGAGTCAAGCTGTGAGAGACACATC |
| | | 3′ |
| 87 | EGFP(HLAB)- | 5′ |
| | KpnI-gs | CTGGGCCGGCTCCGGTACCATGGTGAGCAAGGGCGAGGA |
| | | G 3′ |
| 88 | EGFP(HLAB)- | 5′ |
| | AvrII-ga | CTAGGACAGCCAGGCCCCTAGGCTTGTACAGCTCGTCCATG |
| | | CCG 3′ |
| 89 | HLAB-5′ Leading- | 5′ |
| | gs | GGCTAGCGTTTAAACTTAAGCTTGGTACAATTTGTAATACGA |
| | | CTCACTATAGGGCGGCCG 3′ |
| 90 | HLAB-3′ tail-XhoI- | 5′ |
| | ga | CTGCTCGAAGCATTCTCGAGGTACGACTATGGAACCGCGGC |
| | | CG 3′ |
| 91 | EGFP(HLAB)- | 5′ CTGGGCCGGCTCCGGTACCATGGTGAGCAAGGGCGAGG |
| | KpnI-gs | AG 3′ |
| 92 | EGFP(HLAB)- | 5′ |
| | AvriI-ga | CTAGGACAGCCAGGCCCCTAGGCTTGTACAGCTCGTCCATG |
| | | CCG 3′ |
| 93 | 4545- | 5′ |
| | (ForPEF1a)KpnIX | GAGGTACCTGCAGGCCCGGGGCGGCCGCTCGAGAATGCTT |
| | maINotIXhoI-s | CG 3′ |
| 94 | 4545(ForPEF1a)- | 5′ CGGGCACCGGAGCGGAAAGTCCCCGGAAAGTCCCCGCC |
| | a2 | 3′ |
| 95 | PEF1a-s | 5′ CTTTCCGGGGACTTTCCGCTCCGGTGCCCGTCAGTGG 3′ |
| 96 | PEF1a-KpnI-a | 5′ |
| | | CCCGGGCCTGCAGGTACCTCACGACACCTGAAATGGAAG 3′ |
| 97 | HAC-PD1-KpnI-s | 5′ |
| | | GCGTTTAAACTTAAGCTTGGTACCGCCATGGATTCCCCAGA |
| | | TAGACCATG 3′ |
| 98 | HAC-PD1-Linker- | 5′ |
| | a1 | CCTCGCTTCCTCCGCCTTCACTTCCACCGCCCTCACTGCCG |
| | | CCGCCGGAGCCGCCTCTTTCAGTGACTCTCAATTC 3′ |
| 99 | HAC-PD1-Linker- | 5′ |
| | a2 | GCTTCCGCCGCCGCTGCCTCCACCCTCAGACCCGCCTCCT |
| | | TCGGAGCCTCCTCCCTCGCTTCCTCCGCCTTCAC 3′ |
| 100 | Linker-mCXCL11- | 5′ CAGCGGCGGCGGAAGCATGAACAGGAAGGTCACAGC 3′ |
| | S | |
| 101 | Linker-hCXCL 11-s | 5′ CAGCGGCGGCGGAAGCATGAGTGTGAAGGGCATGGC 3′ |
| 102 | IL-2-s2 | 5′ |
| | | GTTTAAACTTAAGCTTGGTACGCCACCATGTACAGGATGCAA |
| | | CTCCTGTCTTGCATTGCACTAAGTCTTGCAC 3′ |
| 103 | IL-2-a2 | 5′GACTAGTGGATCCGAGCTCGGTACCTGCACTGTTTGTGAC |
| | | AAGTGCAAGACTTAGTGCAATGCAAGACAGGAG 3′ |
| 104 | HAC-PD1-a | 5′ |
| | | CTCGAAGCATTCTCGAGCGGCCGCTTATCTTTCAGTGACTC |
| | | TCAATTC 3′ |
| 105 | HAC-PD1-Iinker- | 5′ GGTGGCGACCGGTGGTCTTTCAGTGACTCTCAATTC 3′ |
| | EGFP-a | |
| 106 | HAC-PD1-Iinker- | 5′ CCACCGGTCGCCACCATGGTGAGCAAGGGCGAGGAG 3′ |
| | EGFP-s | |
| 107 | C terminaI EGFP- | 5′ |
| | XhoINotI-a | CTCGAAGCATTCTCGAGCGGCCGCTTACTTGTACAGCTCGT |
| | | CCATGCC 3′ |
| 108 | hCXCL11-EGFP-a | 5′ GGTGGCGACCGGTGGAAAATTCTTTCTTTCAACTTTTTTG |
| | | 3′ |
| 109 | mCXCL11-EGFP- | 5′ |
| | a | GGTGGCGACCGGTGGCATGTTTTGACGCCTTAAAAAATTC |
| | | 3′ |
| 110 | LHRH-a | 5′ |
| | | CGCAGGCCATAGCTCCAATGTTCGCACGCGCCAGGGACGT |
| | | TAG 3′ |
| 111 | LHRH-s | 5′ |
| | | GAGCTATGGCCTGCGTCCGGGCTGCGGTGGCCCGTCTACT |
| | | AAAG 3′ |
| 112 | OmpAGFP-BstBI- | 5′ |
| | a | CTCATGGTGACGAGCCTTCGAATCATTAAGCCTGCGGCTG |
| | | 3′ |
| 113 | GFP-s | 5′ GGCTCGTCACCATGAGTAAAGGAGAAGAACTTTTC 3′ |
| 114 | GFP-BstBI-a | 5′ |
| | | CGTTCTGATTTAATCTGTTTCGAATTATTTGTATAGTTCATC |
| | | 3′. |
| 115 | haPD1IgG-KpnI-s | 5′ |
| | | CGTTTAAACTTAAGCTTGGTACCGCCATGGGCTGGTCCTGT |
| | | ATCATC 3′ |
| 116 | haPD1IgG-NotI-a | 5′ |
| | | GTCTGCTCGAAGCATTCTCGAGCGGCCGCTTATTTACCTGG |
| | | AGTCCGG 3′. |
| 117 | P2A-KillerRed- | 5 |
| | Mem-s | CGACGTGGAGGAGAACCCTGGACCTATGCTGTGCTGTATGA |
| | | GAAG 3′ |
| 118 | P2A-gs | 5′ |
| | | CTAACTTCAGCCTGCTGAAGCAGGCTGGCGACGTGGAGGA |
| | | GAACCCTG 3′ |
| 119 | P2A-haPD1IgG-s | 5′ |
| | | GGAGAACCCTGGACCTATGGGCTGGTCCTGTATCATCGTG |
| | | 3′ |
| 120 | OmpA-Her2-a | 5′ |
| | | CCTCTGCCCCAGACTGGCCAGGGACGTTAGACTTGGTGTC |
| | | 3′ |
| 121 | OmpA-Her2-s | 5′ CATCACCATCACCATGGCCCGTCTACTAAAGACCAC 3′ |
| 122 | Her2-s | 5′ CAGTCTGGGGCAGAGGTGAAAAAG 3′ |
| 123 | Her2His-a | 5′ ATGGTGATGGTGATGATGAGATCC 3′ |
| 124 | haPD1IgG-C- | 5′ |
| | P2A-a | GCAGGCTGAAGTTAGTAGCTCCGCTTCCTTTACCTGGAGTC |
| | | CGGGAGAAG 3′. |
| 125 | 4545Ptrc-a | 5′ |
| | | CTGATTTAATCTGTTTCGAACCTGCAGGGGCCCGGGCCGCG |
| | | GAGTACTCCTAGGGTCTGTTTCCTGTGTGAAATTG 3′ |
| 126 | 4545PtrcBIaAAA-a | 5′ |
| | | CTGATTTAATCTGTTTCGAACCTGCAGGGGCCCGGGCCGCG |
| | | GAGTACTCCTAGGTTCAGCATCTTTTACTTTCAC 3′ |
| 140 | RKOPEP-A | 5′ |
| | | CCGTTGTTGCTTTTCGGGCACGCGCCAGGGACGTTAG |
| | | AC 3′ |
| 141 | RKOPEP-S | 5′ |
| | | GAAAAGCAACAACGGCGTGTGCGGTGGCCCGTCTACT |
| | | AAAG 3′ |
| 142 | TK-A | 5′ |
| | | CGCTGAAAAGCAATTTTATACCAGGTGCACGCGCCAG |
| | | GGACGTTAG 3′ |
| 143 | TK-S | 5′ |
| | | AAATTGCTTTTCAGCGTAACCGTAAATGCGGTGGCCCG |
| | | TCTACTAAAG 3′ |
| 144 | S36-A | 5′ |
| | | ATTCATGTGGTGCAACTGGACGAGTTTCCATGCACGC |
| | | GCCAGGGACGTTAG 3′ |
| 145 | S36-S | 5′ |
| | | GTTGCACCACATGAATTTCGTTGCGGTGGCCCGTCTAC |
| | | TAAAG 3′ |
| 146 | T7-A | 5′ |
| | | CGCAATGACGCGGATAAATTGCATGGCACGCGCCAGG |
| | | GACGTTAG 3′ |
| 147 | T7-S | 5′ |
| | | ATTTATCCGCGTCATTGCGGTGGCCCGTCTACTAAAG |
| | | 3′ |
| 148 | T12-A | 5′ |
| | | AAACTGGAGACCACATCGGTGGACGATGAGTGCACGC |
| | | GCCAGGGACGTTAG 3′ |
| 149 | T12-S | 5′ |
| | | GATGTGGTCTCCAGTTTGGCCATGCGGTGGCCCGTCT |
| | | ACTAAAG 3′ |
| 150 | GE11-A | 5′ |
| | | AATAACATTTTGCGGAGTATAACCATACCAATGATAGC |
| | | ACGCGCCAGGGACGTTAG 3′ |
| 15- | GE11-S | 5′ |
| | | CTCCGCAAAATGTTATTTGCGGTGGCCCGTCTACTAAA |
| | | G 3′ |
| 152 | A3-A | 5′ |
| | | GTTTGGACCCCAGCCATCGCACGCGCCAGGGACGTTA |
| | | G 3′ |
| 153 | A3-S | 5′ |
| | | GGCTGGGGTCCAAACTGCGGTGGCCCGTCTACTAAAG |
| | | 3′ |
| 154 | FSHB(33-53)-A | 5′ |
| | | TTTGAATTTTTGGACGAGCTGGATCTTTATAAACCAGAT |
| | | CACGAGTATAGCACGCGCCAGGGACGTTAG 3′ |
| 155 | FSHB(33-53)-S | 5′ |
| | | CGTCCAAAAATTCAAAAAACTTGTACTTTTTGCGGTGG |
| | | CCCGTCTACTAAAG 3′ |
| 156 | FSHB(81-95)-A | 5′ |
| | | ATCAGTAGAATCAGAATCACATTTACCACAATGACACT |
| | | GGCACGCGCCAGGGACGTTAG 3′ |
| 157 | FSHB(81-95)-S | 5′ |
| | | GATTCTGATTCTACTGATTGTACTTGCGGTGGCCCGTC |
| | | TACTAAAG 3′ |
| 158 | EBIP37-A | 5′ |
| | | ATTCAGCAGATGCAGCAGCAGAGAAACACCGCCACCA |
| | | GTGCACGCGCCAGGGACGTTAG 3′ |
| 159 | EBIP37-S | 5′ |
| | | GCTGCATCTGCTGAATACTGAACAAGGTGAATCTTGCG |
| | | GTGGCCCGTCTACTAAAG 3′ |
| 160 | P20-A | 5′ |
| | | CCGCAATGATGCATAGTACGAGAAGAGCACGCGCCAG |
| | | GGACGTTAGACTTG 3′ |
| 161 | P20-S | 5′ CTATGCATCATTGCGGTGGCCCGTCTACTAAAG 3′ |
| 162 | CY12RP2-A | 5′ |
| | | GCAATGATGAGTATAATCACAAGCAGTACACAGATAAG |
| | | TGCACGCGCCAGGGACGTTAG 3′ |
| 163 | CY12RP2-S | 5′ |
| | | GATTATACTCATCATTGCGGTGGCCCGTCTACTAAAG |
| | | 3′ |
| 164 | AMSH(1-13)-A | 5′ |
| | | AACTGGTTTACCCCAACGAAAATGTTCCATAGAATAAG |
| | | AGCACGCGCCAGGGACGTTAG 3′ |
| 165 | AMSH(1-13)-S | 5 |
| | | GTTGGGGTAAACCAGTTTGCGGTGGCCCGTCTACTAA |
| | | AG 3′ |
| 166 | PEPTIDEC-A | 5′ |
| | | CACCGCAAGCAAATGCTGGATGATTAACGCACGCGCC |
| | | AGGGACGTTAG 3′ |
| 167 | PEPTIDEC-A | 5′ GCATTTGCTTGCGGTGGCCCGTCTACTAAAG 3′ |

### Example 4. Insertion of the Nucleotide Sequence Encoding the Nine Amino Acids of PLZ4 into the Third Exposed Loop of the S. typhimurium ompA Gene and Construction of Plasmids to Encode Synthesis of this Fusion or Insert it into the S. typhimurium Chromosome

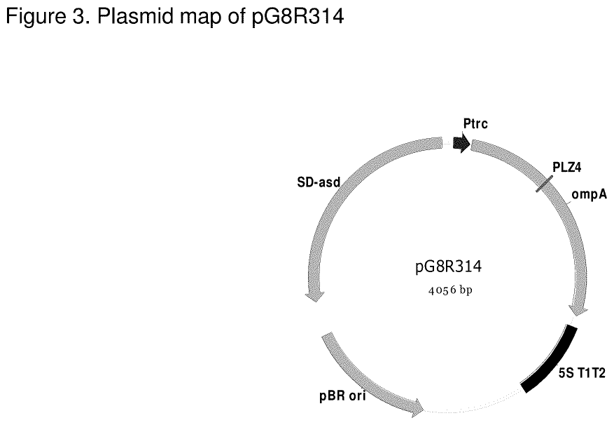

The pG8R314 plasmid (FIG. 3) with the ompAΩplz4 fusion was constructed by amplifying a 454 bp fragment of the S. typhimurium UK-1 (χ3761) chromosome using primers OmpA-s and OmpA-PLZ4-a and a 707 bp fragment with primers OmpA-PLZ4-s and OmpA-SacIHindIII-a. These two fragments were cloned into plasmid pYA3342 (FIG. 1A) cut with NcoI/HindIII to generate plasmid pG8R314. Note that the sequence encoding the PLZ4 peptide was introduced by both primers OmpA-PLZ4-a and OmpA-PLZ4-s. This plasmid has a gene encoding the PLZ4 peptide inserted into the third exposed loop of the OmpA protein enabling expression of the ompAΩplz4 insertion mutated gene in Salmonella. The PLZ4 peptide was flanked with 2 cysteines forming a disulfide linkage to facilitate its exposure on loop 3.

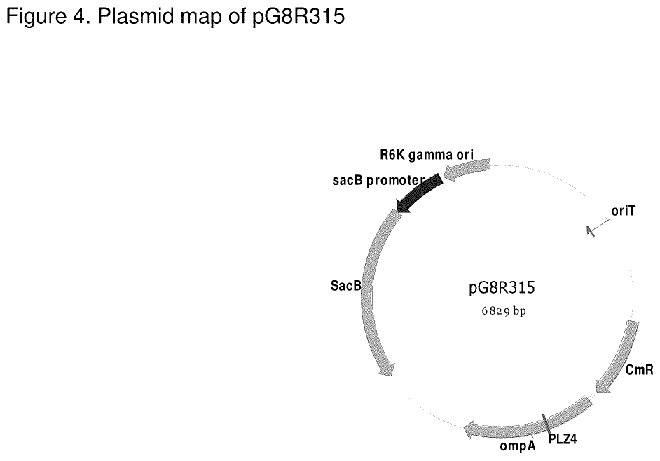

To construct the suicide vector pG8R315 (FIG. 4), the plasmid pG8R314 was used as the template to generate a 1.1 kb fragment encoding synthesis of OmpAΩPLZ4. This fragment was amplified with primers OmpA-XbaI-s and OmpA-SacIHindIII-a and cut with XbaI/SacI. The fragment was then inserted into suicide plasmid pRE112 (FIG. 1B) cut with XbaI/SacI to generate plasmid pG8R315. This suicide vector is then used to introduce the ompAΩplz4 mutation into the chromosome of the S. typhimurium strains listed in Table 5.

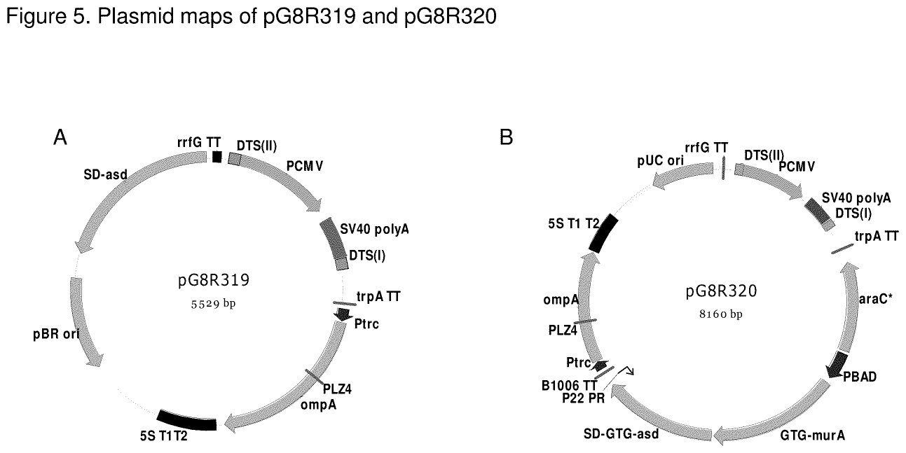

For the construction of pG8R319 (FIG. 5A), we fused two DNA fragments. With plasmid pYA4545 (FIG. 1C) as a template, a 1,549 bp fragment containing rrfG TT-PCMV-SV40 polyA Trp TT was amplified with primers rrfGTT-s and trpTT-a. With plasmid pG8R314 (FIG. 3) as a template, a 4,085 bp fragment that includes the whole pG8R314 plasmid was amplified with primers trpTT-s and rrfGTT-a. The two fragments were assembled to generate plasmid pG8R319. The balanced-lethal plasmid has a pBR on and could express ompAΩplz4 in Salmonella and has a P_{cmv} promoter to be used for gene expression in eukaryotic cells. The P_{cmv} and P_{trc} ompAΩplz4 are separated by the trpA TT.

For the construction of pG8R320 (FIG. 5B), we used plasmid pYA4545 (FIG. 1C) as a template to generate a 8 kb fragment containing the entire pYA4545 plasmid that was amplified with primers pYA4545-TT-BstBI-s and pYA4545-TT-a1 and then extended by PCR with primers pYA4545-TT-BstBI-s and pYA4545-TT-BcII-a. Then with plasmid pG8R314 (FIG. 3) as a template, a 1,242 bp fragment containing P_{trc}-ompA was amplified with primers Ptrc-BcII-s and ompA-BstEI-a. The two fragments were then assembled to generate plasmid pG8R320 (FIG. 5B). This regulated delayed lysis plasmid has a pUC on and can express ompAΩplz4 in Salmonella to display the synthesized OmpAΩPLZ4 on the cell surface and after lysis of the cell employs the P_{cmv} promoter to express an inserted gene sequence in eukaryotic cells. The P_{cmv} and P_{trc} ompAΩplz4 are separated in the dual plasmid vector pG8R320 by the regulated delayed lysis cassette araC P_{araBAD} GTG murA GTG asdA.

### Example 5. Display of OmpAΩPLZ4 on the Bacterial Cell Surface Enables S. typhimurium Cells to Preferentially Attach to Bladder Tumor Cells

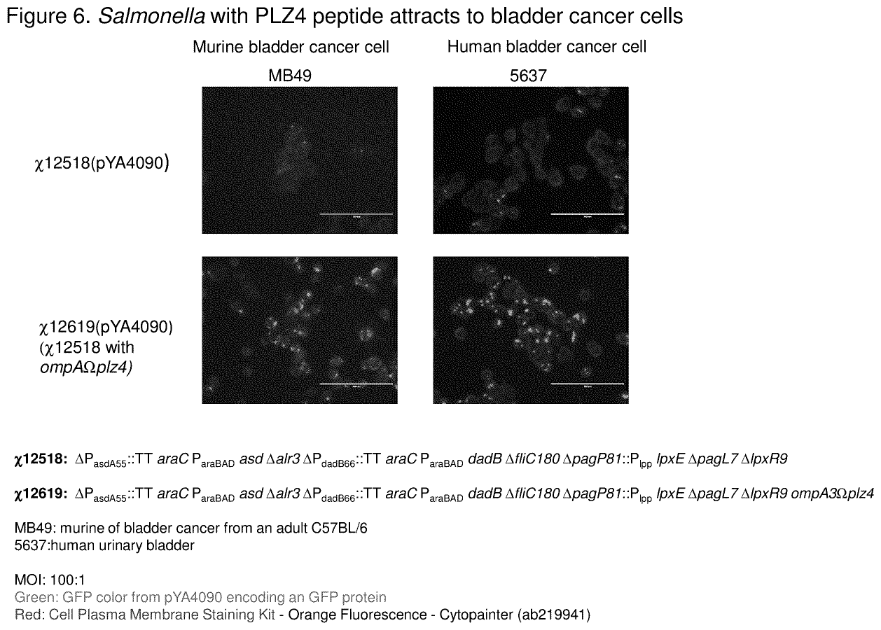

The ompAΩplz4 mutation was introduced into strain χ12518 to generate strain χ12619 using suicide vector χ7213(pG8R315). Both strains were transformed with plasmid pYA4090 to enable tagging the bacteria with the GFP protein. Overnight cultures of χ12518(pYA4090) and χ12619(pYA4090) were diluted into LB broth with 0.1% arabinose and grown until OD600 reached 0.9. The bacteria were washed once with PBS and then used to infect MB49 murine bladder cancer cells and 5637 human bladder cancer cells at MOI 1:100 for 1 hour. The MB49 membrane was stained with Cell Plasma Membrane Staining Kit—Orange Fluorescence—Cytopainter (ab219941, Abcam). As shown in FIG. 6, the strain χ12619 with OmpAΩPLZ4 is more attracted to and invades the bladder cancer cells much better than the strain χ12518 without the OmpAΩPLZ4 fusion.

### Example 6. Construction of Dual Plasmids to Cause CCTS Strains to Target Bladder Tumor Cells and then Express Proteins that Synthesize the CXCL11 Chemokine that Attract Cells of the Immune System

Although the quantity of immune cells in the bladder is not well studied, the bladder has γδ, CD4 and CD8 T cells, macrophages, dendritic cells and NK cells. Notably, there is no report of CD8 T cells in the mouse bladder. Thus, it is important to recruit T cells to the bladder to potentiate immunotherapy of bladder cancer. CXCL11 functions by binding to the receptors CXCR3 predominantly, as well as CXCR7. CXCR3 is expressed on immune cells, such as activated T cells, NK and NKT cells, DCs, but not on naive T cells, and a variety of non-immune cells, such as astrocytes, fibroblasts, endothelial cells, epithelial muscle cells, and cancer cells. CXCR7 is expressed on multiple immune cells, such as T cells, monocytes, DC cells, B cell and NK cells. CXCL11 has diverse functions including inhibiting angiogenesis, increasing immune cell migration, affecting proliferation of different cell types, stimulation of IFN-γ production by immune cells, suppressing M2 macrophage polarization, playing a role in fibroblast directed carcinoma invasion, increasing adhesion and invasion properties, facilitating the migration of certain immune cells, and serving as an adjuvant to anti-cancer therapies. Although CXCL11 mainly works for immune cell migration, differentiation and activation, it could promote cancer cell proliferation and metastasis. Intratumor delivery of CXCL11 has been shown to enhance the efficacy of T-cell infiltration, adoptive T-cell therapy and vaccine efficacy. Locally produced CXCL11 in tumor cells can mediate the recruitment of T cells and NK cells to the tumor site to combat tumor development and growth. This can reduce the global toxicity related to overproduction of CXCL11 in non-tumor sites. For these reasons, we determined that the synthesis of CXCL11 by CCST cells would be optimal if the chemokine was synthesized by tumor cells rather than into the environment if synthesized and delivered by the CCST cells being used for combatting bladder cancer.

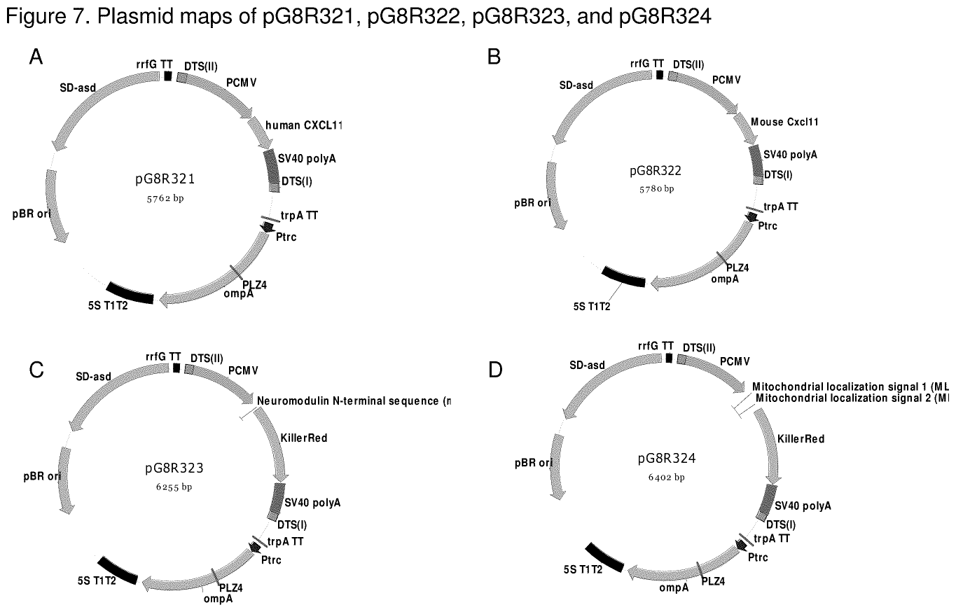

FIG. 7 displays diagrams of pG8R321 (A) and pG8R322 (B) that express ompAΩplz4 in Salmonella to display the synthesized OmpAΩPLZ4 on the cell surface and after plasmid release of the plasmid in tumor cells employs the P_{cmv} promoter to express the human and mouse CXCL11 chemokines, respectively. To construct pG8R321, we used the CXCL11 (NM_005409) Human Tagged ORF Clone (human CXCL11 (Myc-DDK-tagged), ORIGENE Cat #RC210320) as a template to amplify the gene encoding human CXCL11 with primers Human-CXCL11-KpnI-s and Human-CXCL11-Not-a, which was inserted into plasmid pG8R319 cut with KpnI/NotI. The balanced-lethal vector-host combination specifically targets human bladder cancer cells due to the display of the OmpAΩPLZ4 surface protein fusion to induce synthesis of the human CXC11 after invasion into tumor cells to release pG8R321. pG8R322 was similarly constructed using CXCL11 (NM_019494) Mouse Tagged ORF Clone (mouse CXCL11 (Myc-DDK-tagged), ORIGENE Cat #MR222244) as the template to amplify the gene encoding mouse CXCL11 with primers Mouse-CXCL11-KpnI-s and Mouse-CXCL11-NotI-a. This sequence was then inserted into plasmid pG8R319 cut with KpnI/NotI to generate plasmid pG8R322. The balanced-lethal vector-host targets mouse bladder cancer cells due to the display of the OmpAΩPLZ4 surface protein fusion to induce synthesis of the murine CXC11 after invasion into tumor cells to release pG8R322.

### Example 7. Construction of Dual Plasmids to Cause CCTS Strains to Target Bladder Tumor Cells and then Express a Gene Sequence Encoding for Synthesis of KillerRed to Potentiate Tumor Cell Killing

Photodynamic therapy is an important therapeutic treatment for cancer and other diseases. KillerRed is the first engineered photosensitizer with light-induced cytotoxicity that could be used for precise light-induced cell killing and target protein inactivation. Upon light activation, KillerRed can produce toxic reactive oxygen species to use for photodynamic therapy against cancer. Plasmid pG8R323 (FIG. 7C) carries the gene encoding a membrane-targeting KillerRed by fusing with Neuromodulin N-terminal sequence (KillerRed mem thereafter) while plasmid pG8R324 (FIG. 7D) carries the gene encoding a mitochondria targeting KillerRed by fusion with mitochondrial location signals (KillerRed mito thereafter). Both plasmids are balanced-lethal plasmids. These vectors specify expression of the ompAΩplz4 gene in Salmonella to display the synthesized OmpAΩPLZ4 on the cell surface and after release from the CCST cell in the tumor cell employs the P_{cmv} promoter to express the KillerRed encoding genes to kill tumor cell when induced with light.

To construct pG8R323 (FIG. 7C), we used plasmid pCS2-NXE+mem-KillerRed (Addgene Cat #45761) as a template to amplify the gene encoding KillerRed mem using primers KillerRed-Mem-KpnI-s and KillerRed-NotIXhoI-a. The sequence was then inserted into plasmid pG8R319 (FIG. 5A) cut with KpnI/NotI to generate plasmid pG8R323. The balanced-lethal vector-host construct with pG8R323 specifies synthesis of a membrane-targeted KillerRed.

To construct pG8R324 (FIG. 7D) specifying KillerRed-dMito, we used pKillerRed-dMito (EVROGEN cat #FP964) as a template by amplifying the gene encoding KillerRed mito with primers KillerRed-Mito-KpnI-s and KillerRed-NotIXhoI-a and inserting into plasmid pG8R319 (FIG. 5A) cut with KpnI/NotI to generate plasmid pG8R324. The balanced-lethal vector-host construct carries a mitochondria-targeted KillerRed. KillerRed localized on cellular membranes can be used for effective light-induced cell killing by light-induced production of reactive oxygen species and can also be used to detect tumor cells infected with the CCST cells.

### Example 8. Construction of CCST Strains with Regulated Delayed Lysis with Regulated Delayed Lysis High Copy Number Plasmid Vectors Encoding Display of the OmpAΩPLZ4 Surface Protein Fusion and In Situ Synthesis of CXCL11 and KillerRed

The plasmids pG8R321, pG8R322, pG8R323 and pG8R324 (FIG. 7) all have the moderate copy number pBR on and specify the balanced-lethal phenotype using plasmid encoded expression of the asdA gene. We previously determined during the development of means for DNA vaccine delivery by Salmonella that use of high copy number plasmids with pUC on acting as DNA vaccines were more effectively delivered by Salmonella cells undergoing lysis after invasion into host cells. To further validate this belief in developing optimal means for using CCST strains, we constructed versions of the pG8R321, pG8R322, pG8R323 and pG8R324 plasmids with the high copy number pUC on and with regulated delayed lysis attributes to use in Salmonella vector strains also displaying the regulated delayed lysis phenotype (see Table 4).

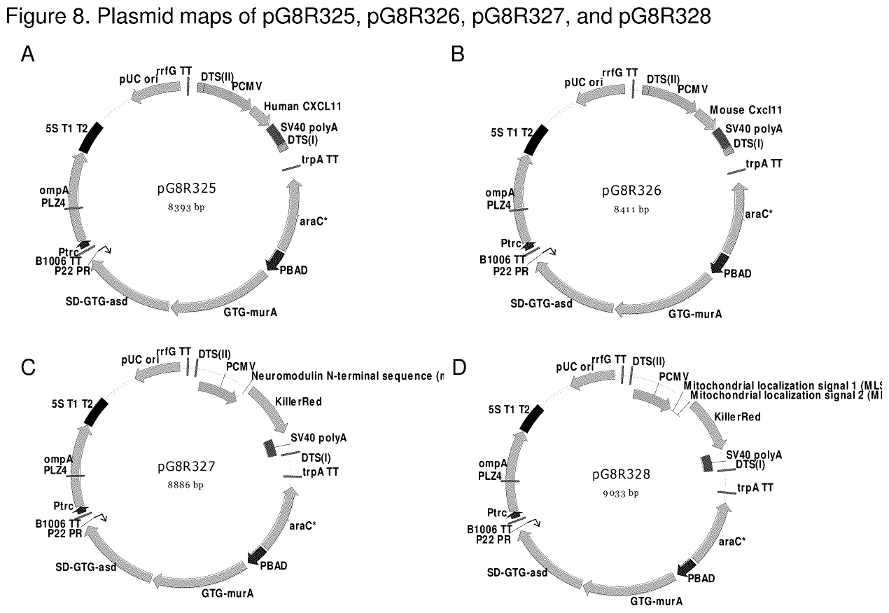

To construct pG8R325 (FIG. 8A) encoding human CXCL11, we amplified a sequence from CXCL11 (NM_005409) Human Tagged ORF Clone (human CXCL11 (Myc-DDK-tagged), ORIGENE Cat #RC210320) as a template with primers Human-CXCL11-KpnI-s and Human-CXCL11-NotI-a and cloned into plasmid pG8R320 (FIG. 5B) cut with KpnI/NotI to generate plasmid pG8R325. The lysis vector carries a human CXC11 gene.

To construct pG8R326 (FIG. 8B) encoding mouse CXCL11, we amplified a sequence from CXCL11 (NM_019494) Mouse Tagged ORF Clone (mouse CXCL11 (Myc-DDK-tagged), ORIGENE Cat #MR222244) as a template with primers Mouse-CXCL11-KpnI-s and Mouse-CXCL11-NotI-a to insert into plasmid pG8R320 (FIG. 5B) cut with KpnI/NotI. The pG8R326 lysis vector carries a mouse CXC11 gene. CXCL11 is chemotactic for interleukin-activated T-cells but not unstimulated T-cells, neutrophils or monocytes. It is the dominant ligand for CXCR3.

To construct pG8R327 (FIG. 8C), we used pCS2-NXE+mem-KillerRed (Addgene Cat #45761) as a template to amplify the gene encoding KillerRed mem with primers KillerRed-Mem-KpnI-s and KillerRed-NotIXhoI-a to insert into plasmid pG8R320 (FIG. 5B) cut with KpnI/NotI. The pG8R327 lysis vector carries a membrane-targeted KillerRed.

To construct pG8R328 (FIG. 8D), we used pKillerRed-dMito (EVROGEN cat #FP964) as a template to amplify a sequence encoding KillerRed mito using primers KillerRed-Mito-KpnI-s and KillerRed-NotIXhoI-a to insert into plasmid pG8R320 (FIG. 5B) cut with KpnI/NotI. The lysis vector pG8R328 specifies synthesis of the mitochondria-targeted KillerRed. KillerRed localized on cellular membranes can be used for effective light-induced cell killing by light-induced production of reactive oxygen species.

### Example 9. Construction of Plasmid Vectors Encoding Synthesis of GFP or EGFP to Track Salmonella Extracellularly and Intracellularly to Evaluate the Targeting Ability of CCTS Strains to Bladder Tumors

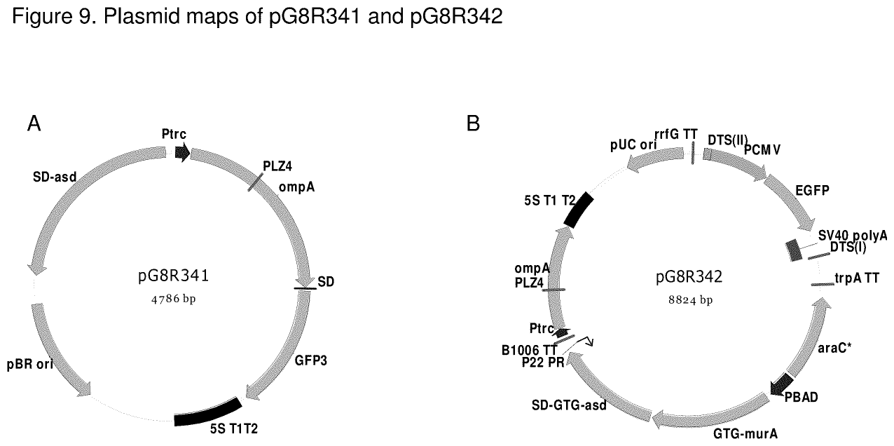

FIG. 9 displays the diagrams of plasmids pG8R341 (FIG. 9A) and pG8R342 (FIG. 9B) used to tag Salmonella with fluorescent proteins. The balanced-lethal plasmid pG8R341 carries a P_{trc} promoter which can express the operon fusion of ompAΩplz4 and gfp that enables GFP production in the Salmonella cytosol and displays the synthesized OmpAΩPLZ4 on the cell surface of Salmonella. P_{trc} is a prokaryotic promoter that can express at high level protein synthesis under both anaerobic and aerobic conditions and is repressed by Lac. Salmonella strains carrying this plasmid in vivo in the absence of arabinose to preclude synthesis of Lac produce GFP in Salmonella cells present extracellularly and intracellularly. This regulated delayed lysis plasmid pG8R342 (FIG. 9B) has a pUC on and can express the ompAΩplz4 in Salmonella to display the synthesized OmpAΩPLZ4 on the cell surface and after lysis of the cell within host cells employs the P_{cmv} promoter to express an inserted egfp in eukaryotic cells. EGFP is only produced when Salmonella is inside the mammalian cells.

To construct pG8R341 (FIG. 9A), we used plasmid pYA4090 as a template to amplify the gene encoding GFP3 using primers SD-GFP-SacI-gs and GFP-HindIII-ga. The sequence was inserted into plasmid pG8R314 (FIG. 3) cut with Sac/HindIII to generate plasmid pG8R341. The balanced-lethal plasmid uses GFP to track Salmonella with OmpAΩPLZ4.

To construct pG8R342 (FIG. 9B), we used plasmid pYA4685 as a template to amplify the gene encoding EGFP using primers EGFP-KpnI-gs and EGFP-XhoI-ga. The sequence was inserted into plasmid pG8R320 (FIG. 5B) cut with KpnI/XhoI to generate plasmid pG8R342. The lysis plasmid uses EGFP to track Salmonella within mammalian cells.

### Example 10. Construction of Dual Plasmids to Cause CCTS Strains to Target Bladder Tumor Cells and then Express Gene Sequences Encoding KillerRed to Potentiate Tumor Cell Killing and CXCL11 to Attract Immune Cells

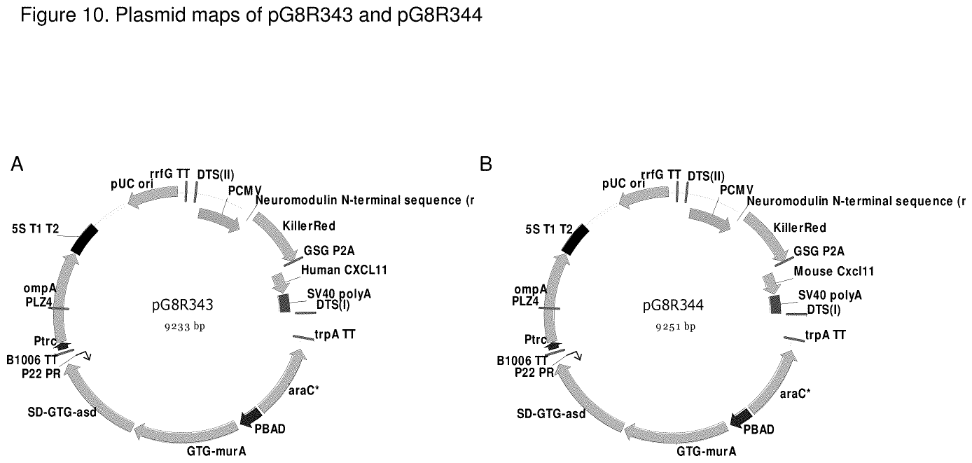

A construction that can kill cancer cells and recruit immune cells to tumor cells can have synergic effect to benefit bladder cancer therapy. FIG. 10 displays diagrams of such constructions, balanced-lethal plasmids with regulated delayed lysis attributes pG8R343 (FIG. 9A) and pG8R344 (FIG. 9B) with pUC on that express ompAΩplz4 in Salmonella to display the synthesized OmpAΩPLZ4 on the cell surface and after lysis of the bacterial cell employs the P_{cmv} promoter to express genes encoding KillerRed mem that kill tumor cells and human or mouse CXCL11 that recruit immune cells to combat bladder tumors. To enable co-expression of genes encoding KillerRed mem and human or mouse CXCL11 in mammalian cells, a P2A peptide was introduced between the genes encoding KillerRed mem and human or mouse CXCL11 to enable ribosome skip to enable synthesis of a peptide bond at the C-terminus of a 2A element leading to cleavage between the end of the 2A sequence and the CXCL11 peptide downstream.

To construct pG8R343 (FIG. 10A), we fused two fragments encoding KillerRed mem P2A and human CXCL11. We used plasmid pCS2-NXE+mem-KillerRed (Addgene plasmid #45761) as a template to amplify the gene encoding KillerRed mem using primers KillerRed-Mem-KpnI-s and KillerRed-C-P2A-a1. The fragment was used as a template and amplified with primers KillerRed-Mem-KpnI-s and KillerRed-C-P2A-a2 to include the sequence encoding P2A. We then used CXCL11 (NM_005409) Human Tagged ORF Clone (human CXCL11 (Myc-DDK-tagged), ORIGENE Cat #RC210320) as a template to amplify the gene encoding human CXCL11 using primers P2A-Human CXCL11-s and Human-CXCL11-NotI-a. The KillerRed Mem-P2A and human CXCL11 fusion sequence was inserted into plasmid pG8R320 (FIG. 5B) cut with KpnI/NotI to generate plasmid pG8R343. The lysis plasmid carries genes encoding KillerRed mem and human CXCL11. The two genes were separated by a P2A peptide which can induce cleavage at the C-terminal of the P2A peptide to enable production of both KillerRed mem and human CXCL11 after Salmonella invasion into tumor cells and lysis to release pG8R343 and enable synthesis of KillerRed mem and human CXCL11.

To construct pG8R344 (FIG. 10B), we fused two fragments for KillerRed mem P2A and mouse CXCL11. The KillerRed mem P2A was generated as above for plasmid pG8R343. We used CXCL11 (NM_019494) Mouse Tagged ORF Clone (mouse CXCL11 (Myc-DDK-tagged), ORIGENE Cat #MR222244) as a template to amplify the gene encoding mouse CXCL11 using primers P2A-Mouse CXCL11-s and Mouse-CXCL11-NotI-a. The KillerRed Mem-P2A and mouse CXCL11 fusion were inserted into plasmid pG8R320 (FIG. 5B) cut with KpnI/NotI to generate plasmid pG8R344. The lysis plasmid carries genes encoding KillerRed mem and mouse CXCL11. The two genes were separated by a P2A peptide which can induce cleavage at the C-terminal of the P2A peptide to enable production of both KillerRed mem and mouse CXCL11 after Salmonella invasion into tumor cells and lysis to release pG8R344 and enable synthesis of KillerRed mem and mouse CXCL11.

### Example 11. Construction of Dual Plasmids to Cause CCTS Strains to Target Bladder Tumor Cells and then after Invasion Express a Gene Sequence Fused with an HLA Peptide Encoding Sequence to Potentiate Immune Responses to Tumor Cells

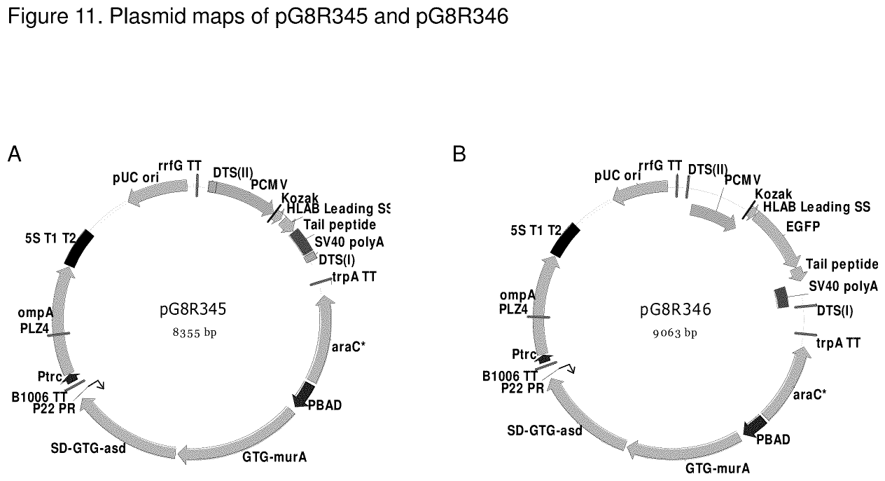

Many factors can affect vaccine-induced immune responses. Antigens can be linked to lysosomal or endosomal targeting signals to route the antigen into an MHC class II processing compartment to improve CD4⁺ T cell responses. A chimeric protein fused with the N-terminal leader peptide with an MHC class I trafficking signal (tail peptide) attached to the C-terminal end of an antigen can strongly improve the presentation of MHC class I and class II epitopes in human and murine dendritic cells, leading to efficient expansion of antigen specific CD4⁺ and CD8⁺ T cells and their effector functions. We thus generated plasmid pG8R345 (FIG. 11 A) specifying HLAB leading and tail peptides to enable an inserted antigen to be presented to MHC class I and class II pathways.

To construct pG8R345 (FIG. 11A), we used HLAB (HLA-B) (NM_005514) Human Untagged Clone (ORIGENE Cat #SC124484) as a template to amplify the gene encoding the HLA leading peptide using primers HLAB-Leading-gs and HLAB-Leading-MCS-ga and HLA tail peptide using primers HLAB-tail-MCS-gs and HLAB-tail-XhoI-ga. The sequence generated encoding the above two fragments were inserted into plasmid pG8R320 (FIG. 5B) cut with KpnI/XhoI to generate plasmid pG8R345. This plasmid has sequences for HLAB leading and tail peptides which could be used to coupling a selected antigen to MHC Class I Trafficking Signals to increase antigen presentation efficiency. This regulated delayed lysis plasmid pG8R345 has a pUC on and can express ompAΩplz4 in Salmonella to display the synthesized OmpAΩPLZ4 on the cell surface and after tumor cell invasion and lysis of the CCST cell employs the P_{cmv} promoter to express the HLA-antigen encoding gene in eukaryotic tumor cells.

We then inserted egfp into plasmid pG8R345 to generate plasmid pG8R346 (FIG. 11 B). The egfp is under the control of PCMV which can be expressed in eukaryotic cells. To construct pG8R346, we used plasmid pYA4685 (FIG. 2D) as a template to amplify the gene encoding EGFP using primers EGFP(HLAB)-KpnI-gs and EGFP(HLAB)-AvrII-ga. The sequence was inserted into plasmid pG8R345 (FIG. 11A) cut with KpnI/AvrII to generate plasmid pG8R346. The lysis plasmid carries a gene encoding EGFP and could be used to track the CCTS strain in mammalian cells.

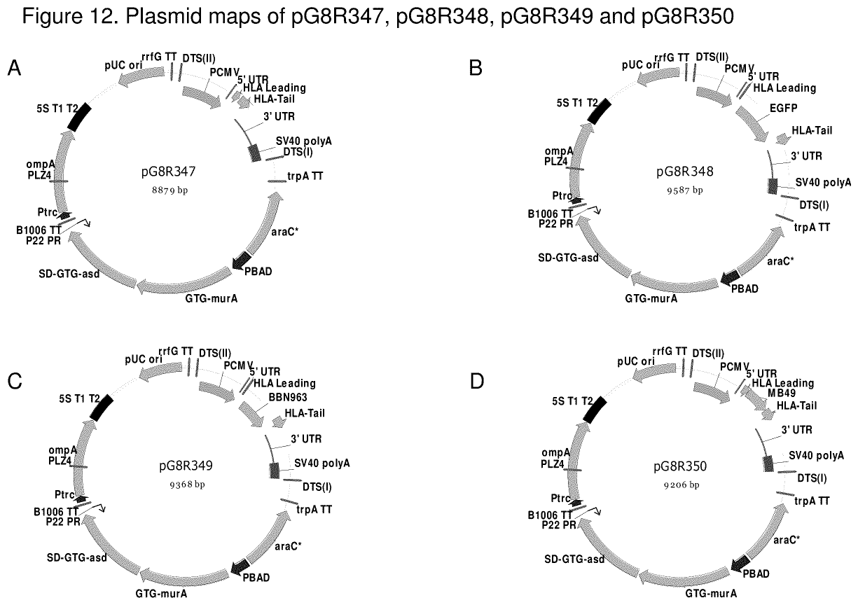

The 5′ and 3′ terminal nucleotide sequences of eukaryotic genes affect the translation of the gene. Thus, we generated plasmid pG8R347 (FIG. 12A) that includes the 5′ and 3′ terminal nucleotide sequence of HLAB gene.

To construct pG8R347, we used HLAB (HLA-B) (NM_005514) Human Untagged Clone (ORIGENE Cat #SC124484) as a template to amply the sequence of the 5′ terminal of HLAB using primers HLAB-5′ Leading-gs and HLAB-Leading-MCS-ga and the 3′ terminal of HLAB using primers HLAB-tail-MCS-gs and HLAB-3′ tail-XhoI-ga. The above two fragments were cloned into pG8R320(FIG. 5B) cut with KpnI/XhoI to generate pG8R347. The lysis plasmid has sequences for HLAB 5′ terminus, leading and tail peptides and 3′ terminus which could be used to coupling antigen to MHC Class I Trafficking Signals to increase antigen presentation efficiency. The inclusion of 5′ and 3′ termini of HLAB increases the transcript stability and translational efficiency.

Similar, we inserted the egfp gene into plasmid pG8R347 to generate plasmid pG8R348 (FIG. 12B). To construct plasmid pG8R348, we used plasmid pYA4685 as a template to amplify the gene encoding EGFP using primers encoding EGFP was amplified with primers EGFP(HLAB)-KpnI-gs and EGFP(HLAB)-AvrII-ga and cloned into plasmid pG8R347 (FIG. 12A) cut with KpnI/AvrII to generate plasmid pG8R348. The lysis plasmid carries a gene encoding EGFP and could be used to track the Salmonella in mammalian cells, although this requires replacing the ompAΩplz4 construction that enables targeting to bladder tumor cells with sequences specifying a protein to target other cell types.

Tumor neoantigens can be presented by major histocompatibility complex proteins and recognized by T cells to induce anti-tumor immune responses. This approach has been used as therapeutic vaccines in preclinical models to promote tumor specific T-cell responses. Tumor neoantigens are derived from mutated proteins that lead to the generation of novel immune epitopes that are foreign to the body. Vaccines targeting tumor neoantigens are a promising strategy for personalized cancer immunotherapy. Due to the complex immune tolerance mechanisms in tumors, neoantigen based tumor vaccines are normally combined with immune checkpoint inhibitors. Clinical trials with this combination therapy demonstrated that the induction of neoantigen-specific CD4⁺ and CD8⁺ T cell responses and cytotoxic vaccine-induced T cells, had some efficacy in treating bladder cancer. Mouse derived BBN963 and MB49 cell lines are commonly used as an in vitro and in vivo model of bladder cancer. Neoantigens have been identified in these two cell lines. These neoantigens were cloned into vector pG8R347 to generate plasmid pG8R349 (FIG. 12C) and pG8R350 (FIG. 12D) to be used in vaccine trials.

To construct pG8R349, the gene encoding neo-antigen BBN963 was cut from with plasmid pUC57-BBN963 with KpnI/AvrII and cloned into plasmid pG8R347 (FIG. 12A) cut with same enzymes to generate plasmid pG8R349. The lysis plasmid carries a gene encoding neo-antigen BBN963.

To construct pG8R350, the gene encoding neo-antigen MB49 was cut from with plasmid pJET1.2-MB49 with KpnI/AvrII and cloned into plasmid pG8R347 (FIG. 12A) cut with same enzymes to generate plasmid pG8R350. The lysis plasmid carries a gene encoding neo-antigen MB49.

### Example 12. Construction of Dual Plasmids to Cause CCTS Strains to Target Bladder Tumor Cells and then Express a Gene Sequence Under the Control of P_{EF1α} Promoter

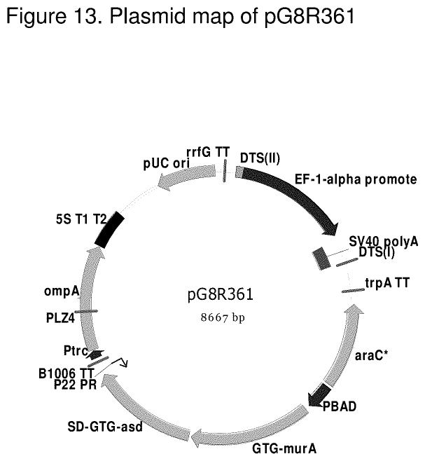

The viral derived PCMV promoter is a strong promoter that has been widely used to express genes in eukaryotic cells as in DNA or viral vectors, such as adenovirus. However, it could be silenced in certain cell types due to methylation, leading to considerable variability in gene expression in different cell types. Human elongation factor-1α is a constitutive human promoter that can drive ectopic gene expression homogeneously and persistently in vivo and in vitro. It can replace PCMV when P_{CMV} has diminished activity due to being silenced. We therefore generated the regulated delayed lysis plasmid pG8R361 (FIG. 13) that has a pUC on and can express ompAΩplz4 in Salmonella to display the synthesized OmpAΩPLZ4 on the cell surface and after lysis of the CCTS cell employs the P_{EF1α} promoter to express inserted genes in eukaryotic cells.

For the construction of pG8R361 (FIG. 13), we fused two fragments. We used plasmid pG8R320 (FIG. 5B) as a template to generate a 7.4 kb fragment with primers 4545-(ForPEF1a) KpnIXmalNotIXhoI-s and 4545(ForPEF1a)-a2. Then, with plasmid pLVX EF1α IRES Puro N (BEI NR52973) as a template, a fragment containing the P_{EF1α} promoter was amplified with primers PEF1a-s and PEF1a-KpnI-a. The two fragments were then assembled into plasmid pG8R361. The regulated delayed lysis plasmid has a pUC on and can express ompAΩplz4 in Salmonella to display the synthesized OmpAΩPLZ4 on the cell surface to target bladder cancer cells and after lysis of the CCTS cell employs the P_{EF1α} promoter to express an inserted gene sequence in eukaryotic cells, in this case into bladder tumor cells. The P_{EF1α} and P_{trc} ompAΩplz4 in the dual plasmid vector pG8R361 are separated by the regulated delayed lysis cassette araC P_{araBAD} GTG murA GTG asdA.

### Example 13. Construction of Dual Plasmids to Cause CCTS Strains to Target Bladder Tumor Cells and then Express a Gene Sequence Encoding HAC-PD1 to Block PD1L1 and Activate T Cells and CXCL11 to Recruit Immune Cells

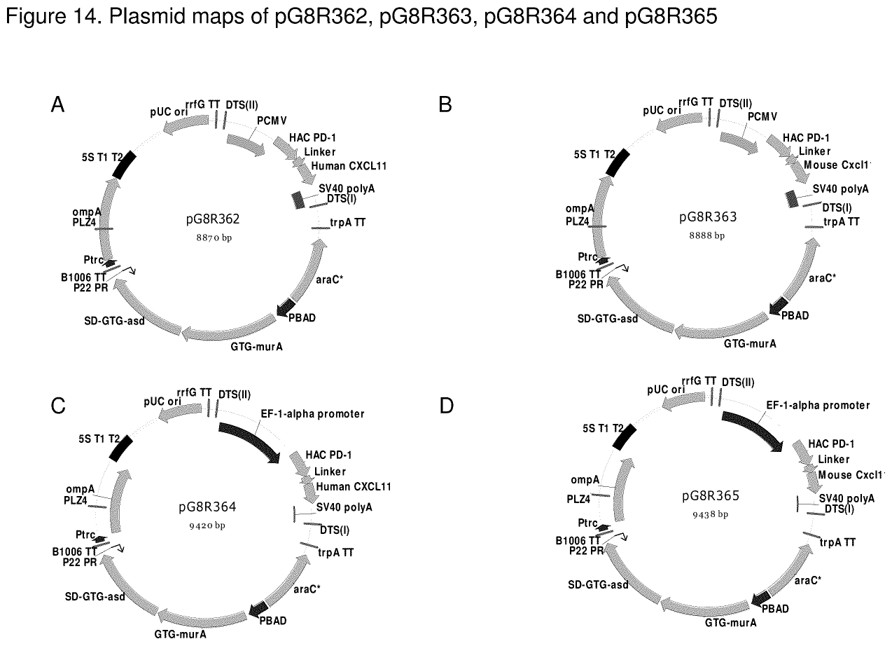

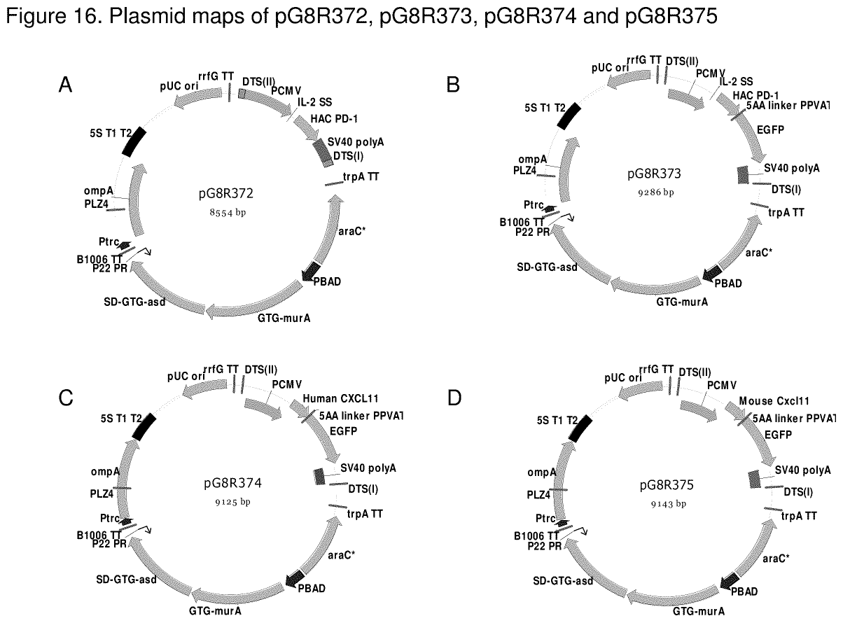

The interaction between the Programmed cell death protein-1 (PD-1) and programmed cell death ligand-1 (PD-L1) functions as a T cell checkpoint to regulate T cell responses. Cancer cells upregulate the levels of PD-L1 to evade immune detection and elimination. Monoclonal antibodies blocking PD1 and PDL1 have been approved as effective immunotherapies against different tumors. However, the use of antibodies has inherent limitations that include poor and slow distribution within hypoxic regions of large tumors and immune-related adverse events, such as Fc-mediated cytotoxic immune responses and severe cytokine associated inflammatory and immunological process. For monoclonal antibodies against PD1/PLD1, they can also reduce circulation of T cell numbers in patients. To overcome these shortcomings, a soluble fragment of the PD1 ectodomain, the high-affinity consensus (HAC)-PD1, was identified as an alternative agent that exhibits improved antitumor responses and avoids antibody limitations. The HAC-PD1 has an over 40,000-fold higher affinity for PD-L1 than native PD1 and 32- and 12-times higher affinity than the FDA-approved anti-PD-L1 antibodies atezolizumab and durvalumab, respectively. At the same dose and schedule through intratumoral injection, it is also more effective than an anti-PD1 antibody in inducing anti-cancer immunity. Multiple vectors carrying the gene encoding HAC-PD1 are depicted in FIGS. 14, 15 and 16. FIG. 14 depicts plasmids that contain sequences encoding HAC-PD1 combined with CXCL11 for combinational immunotherapy. FIG. depicts plasmids that contain sequences encoding IL2 SS-HAC-PD1 and CXCL11 for combinational immunotherapy. FIG. 16 depicts plasmids that carry sequences encoding HAC-PD1, HAC-PD1 and EGFP, CXCL11 and EGFP. All these plasmids display the synthesized OmpAΩPLZ4 on the cell surface to target bladder cancer cells and after lysis of the CCTS cell employ the P_{CMV} or PEF1a promoter to express the inserted eukaryotic genes in bladder tumor cells.

To construct plasmid pG8R362 (FIG. 14A), we used plasmid pMaI-HAC-PD1 as a template to amplify the gene encoding HAC-PD1 using primers HAC-PD1-KpnI-s and HAC-PD1-Linker-a1 and then extended by PCR with primers HAC-PD1-KpnI-s and HAC-PD1-Linker-a2. Then with CXCL11 (NM_005409) Human Tagged ORF Clone (human CXCL11 (Myc-DDK-tagged), ORIGENE Cat #RC210320) as a template, the gene encoding human CXCL11 was amplified with primers Linker-hCXCL11-s and Human-CXCL11-NotI-a. The two fragments were inserted into plasmid pG8R320 (FIG. 5B) cut with KpnI/NotI to generate plasmid pG8R362. The lysis plasmid carries the gene encoding HAC-PD1 fused with human CXCL11 with a 36 amino acid linker under the control of a P_{CMV} promoter. The 36 aa linker GGS(GGGSE)₅(GGGS)₂ (SEQ ID NO: 169) was inserted to enable fusion of HAC-PD1 and human CXCL11. The regulated delayed lysis plasmid has a pUC on and can express ompAΩplz4 in Salmonella to display the synthesized OmpAΩPLZ4 on the cell surface and after lysis of the CCTS cell employs the P_{cmv} promoter to express the fused gene sequence.

To construct plasmid pG8R363 (FIG. 14B), we used plasmid pMaI-HAC-PD1 as a template to amplify the gene encoding HAC-PD1 using primers HAC-PD1-KpnI-s and HAC-PD1-Linker-a1 and then extended by PCR with primers HAC-PD1-KpnI-s and HAC-PD1-Linker-a2). Then with Cxcl11 (NM_019494) Mouse Tagged ORF Clone (mouse CXCL11 (Myc-DDK-tagged), ORIGENE Cat #MR222244) as a template, the gene encoding mouse CXCL11 was amplified with primers Linker-mCXCL11-s and Mouse-CXCL11-NotI-a. The two fragments were inserted into plasmid pG8R320 (FIG. 5B) cut with KpnI/NotI to generate plasmid pG8R363. The lysis plasmid carries a gene encoding HAC-PD1 and fused with mouse CXCL11 with a 36 amino acid linker under the control of a P_{CMV} promoter. The 36 aa linker GGS(GGGSE)₅(GGGS)₂ (SEQ ID NO: 169) was inserted to enable fusion of HAC-PD1 and mouse CXCL11. The regulated delayed lysis plasmid has a pUC on and can express ompAΩplz4 in Salmonella to display the synthesized OmpAΩPLZ4 on the cell surface and after lysis of the CCTS cell employs the P_{cmv} promoter to express the fused gene sequence.

To generate plasmid pG8R364 (FIG. 14C), a fragment encoding HAC-PD1-hCXCL11 was cut from plasmid pG8R362 (FIG. 14A) with KpnI/NotI and cloned into plasmid pG8R361 (FIG. 13) cut with the same enzymes to generate plasmid pG8R364. The lysis plasmid carries a gene encoding HAC-PD1 that was fused with human CXCL11 using a 36 amino acid linker under the control of the P_{EF1α} promoter. The regulated delayed lysis plasmid has a pUC on and can express ompAΩplz4 in Salmonella to display the synthesized OmpAΩPLZ4 on the cell surface and after lysis of the CCTS cell employs the P_{EF1α} promoter to express the fused gene sequence.

To generate plasmid pG8R365 (FIG. 14D), a fragment encoding HAC-PD1-mCXCL11 was cut from plasmid pG8R363 (FIG. 14B) with KpnI/NotI and cloned into plasmid pG8R361 (FIG. 13) cut with the same enzymes to generate plasmid pG8R365. The lysis plasmid carries a gene encoding HAC-PD1 that was fused with mouse CXCL11 using a 36 amino acid linker under the control of the P_{EF1α} promoter. The regulated delayed lysis plasmid has a pUC on and can express ompAΩplz4 in Salmonella to display the synthesized OmpAΩPLZ4 on the cell surface and after lysis of the CCTS cell employs the P_{EF1α} promoter to express the fused gene sequence.

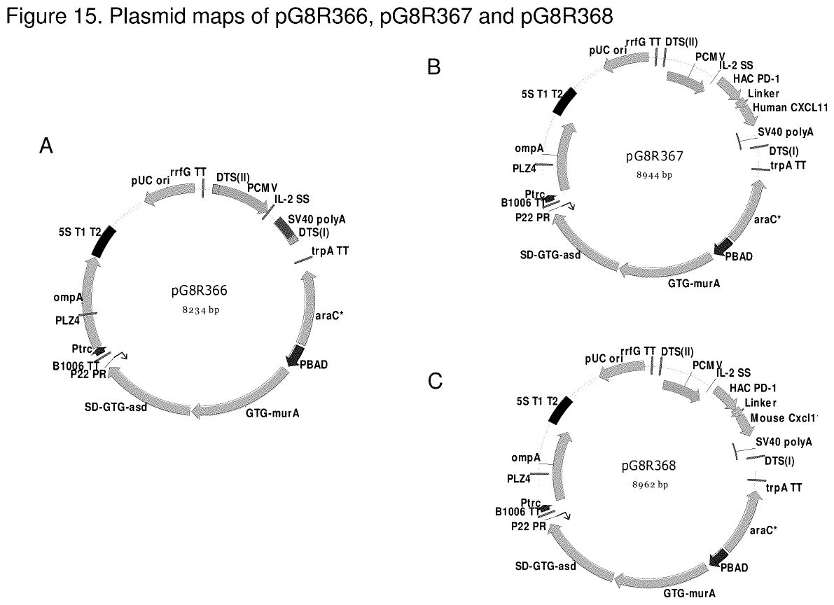

Secretion of proteins increases the levels of therapeutic molecules that can significantly enhance the efficacy of therapy at the site of the disease. The IL2 signal peptide is one of the most commonly used secretion facilitating sequences used for protein production in gene therapy research. To increase the secretion of HAC-PD1, we first generated plasmid pG8R366 (FIG. 15A) that carries the sequence encoding the IL2 secretion signal. We amplified the IL2 SS using primers IL2-s2 and IL2-a2. The sequence was inserted into plasmid pG8R320 (FIG. 5B) cut with KpnI to generate plasmid pG8R366. The regulated delayed lysis plasmid has a pUC on and can express ompAΩplz4 in Salmonella to display the synthesized OmpAΩPLZ4 on the cell surface and after lysis of the CCTS cell employs the P_{cmv} promoter to express any gene sequence fused to the IL2 secretion signal sequence. We then inserted HAC-PD1 with human and mouse CXCL11 into pG8R366 to generate plasmids pG8R367 (FIG. 15B) and pG8R368 (FIG. 15C), respectively.

To generate plasmid pG8R367, the fragment encoding HAC-PD1-hCXCL11 was cut from plasmid pG8R362 (FIG. 14A) with KpnI/NotI and inserted into plasmid pG8R366 (FIG. 15A) cut with the same enzymes to generate plasmid pG8R367. The regulated delayed lysis plasmid has a pUC on and can express ompAΩplz4 in Salmonella to display the synthesized OmpAΩPLZ4 on the cell surface and after lysis of the CCTS cell employs the P_{cmv} promoter to express the gene sequence encoding HAC-PD1-human CXCL11 fused to the IL2 secretion signal.

To generate plasmid pG8R368, the fragment encoding HAC-PD1-mCXCL11 was cut from plasmid pG8R363 (FIG. 14B) with KpnI/NotI and inserted into plasmid pG8R366 (FIG. 15A) cut with the same enzymes to generate plasmid pG8R368. The regulated delayed lysis plasmid has a pUC on and can express ompAΩplz4 in Salmonella to display the synthesized OmpAΩPLZ4 on the cell surface and after lysis of the CCTS cell employs the P_{cmv} promoter to express the gene sequence encoding HAC-PD1-mouse CXCL11 fused to the IL2 secretion signal.

We also generated plasmid pG8R372 (FIG. 16A) which only has HAC-PD1 fused to the IL2 secretion signal. To generated plasmid pG8R372, we used plasmid pG8R367 (FIG. 15B) as a template to amplify the fragment encoding IL2 SS and HAC-PD1 with primers IL2-s2 and HAC-PD1-a. The fragment was inserted into plasmid pG8R320 (FIG. 5B) cut with KpnI/NotI to generate plasmid pG8R372. The regulated delayed lysis plasmid has a pUC on and can express ompAΩplz4 in Salmonella to display the synthesized OmpAΩPLZ4 on the cell surface and after lysis of the CCTS cell employs the P_{cmv} promoter to express the gene sequence encoding HAC-PD1-fused to the IL2 secretion signal.

To facilitate tracking of HAC-PD1 in mammalian cells, we tagged HAC-PD1 with EGFP to generate plasmid pG8R373 (FIG. 16B). For the construction of plasmid pG8R373, we used plasmid pG8R367 (FIG. 15B) as a template to amplify the fragment encoding IL2 SS-HAC-PD1 fusion using primers IL2-s2 and HAC-PD1-linker-EGFP-a. And then with plasmid pYA4685 (FIG. 2D) as a template, the gene encoding EGFP was amplified with primers HAC-PD1-linker-EGFP-s and C terminal EGFP-XhoINotI-a. The two fragments were inserted into plasmid pG8R320 (FIG. 5B) cut with KpnI/NotI to generate plasmid pG8R373. The regulated delayed lysis plasmid has a pUC on and can express ompAΩplz4 in Salmonella to display the synthesized OmpAΩPLZ4 on the cell surface and after lysis of the CCTS cell employs the P_{cmv} promoter to express the gene encoding fusion of IL2 secretion signal-HAC-PD1-fused and EGFP.

Similarly, we generate pG8R374 (FIG. 16C) and pG8R375 (FIG. 16D) in which CXCL11 fused with EGFP to track the behavior of CXCL11. To generate plasmid pG8R374, we used CXCL11 (NM_005409) Human Tagged ORF Clone (human CXCL11 (Myc-DDK-tagged), ORIGENE Cat #RC210320) as a template to amplify the gene encoding human CXCL11 using primers Human-CXCL11-KpnI-s and hCXCL11-EGFP-a. And then with plasmid pYA4685 (FIG. 2D) as a template, the gene encoding EGFP was amplified with primers HAC-PD1-linker-EGFP-s and C-terminal EGFP-XhoINotI-a. The two fragments were inserted into plasmid pG8R320 (FIG. 5B) cut with KpnI/NotI to generate plasmid pG8R374. The regulated delayed lysis plasmid has a pUC on and can express ompAΩplz4 in Salmonella to display the synthesized OmpAΩPLZ4 on the cell surface and after lysis of the CCTS cell employs the P_{cmv} promoter to express the gene encoding the fusion of human CXCL11 and EGFP.

To generate plasmid pG8R375 (FIG. 16D), we used CXCL11 (NM_019494) Mouse Tagged ORF Clone (mouse CXCL11 (Myc-DDK-tagged), ORIGENE Cat #MR222244) as a template to amplify the gene encoding mouse CXCL11 using primers Mouse-CXCL11-KpnI-s and mCXCL11-EGFP-a. And then with plasmid pYA4685 (FIG. 2D) as a template, the gene encoding EGFP was amplified with primers HAC-PD1-linker-EGFP-s and C-terminal EGFP-XhoINotI-a. The 2 fragments were inserted into plasmid pG8R320 (FIG. 5B) cut with KpnI/NotI to generate plasmid pG8R375. The regulated delayed lysis plasmid has a pUC on and can express ompAΩplz4 in Salmonella to display the synthesized OmpAΩPLZ4 on the cell surface and after lysis of the CCTS cell in situ employs the P_{cmv} promoter to express the gene encoding fusion of mouse CXCL11 and EGFP.

### Example 14. Insertion of the Nucleotide Sequence Encoding the Ten Amino Acids of the Luteinizing Hormone-Releasing Hormone (LHRH) Peptide Binding to the LHRH Receptor into the Third Exposed Loop of the S. typhimurium ompA Gene to Cause CCTS Strains to Target Endometrial, Bladder, Ovarian, Prostate and Breast Tumors with Overexpression of LHRH Receptors

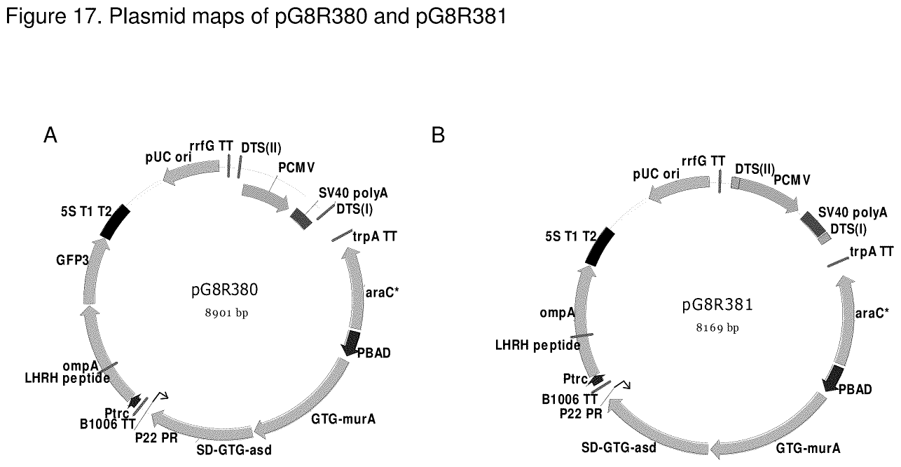

Luteinizing hormone-releasing hormone (LHRH) receptors are overexpressed in many cancers, including endometrial, bladder, ovarian, prostate and breast cancers, while limited in normal healthy tissues. LHRH has been employed to efficiently guide anticancer and imaging agents to cancer cells, thereby increasing the amount of these substances in tumors, but limiting delivery to normal tissues to reduce unnecessary exposure and toxicity. We thus generated regulated delayed lysis plasmids pG8R380 (FIG. 17A) and pG8R381 (FIG. 17B) that have a pUC on and can express ompAΩlhrh in Salmonella to display the synthesized OmpAΩLHRH on the cell surface to target endometrial, bladder, ovarian, prostate and breast cancers and after lysis of the CCTS cell in the invaded tumor cell employ the P_{cmv} promoter to express a selected gene of importance to tumor therapy or identification.

To construct plasmid pG8R380 (FIG. 17A), we used plasmid pG8R320 (FIG. 5B) as a template to amplify a 540 bp fragment using primers P_{trc}-BcII-s and LHRH-a and a 707 bp fragment using primes LHRH-s and OmpAGFP-BstBI-a. And then with plasmid pYA4090 (FIG. 2C) as a template, the gene encoding GFP was amplified with primer GFP-s and GFP-BstBI-a. The above 3 fragments were then inserted into plasmid pG8R320 (FIG. 5B) cut with BcII/BstBI to generate plasmid pG8R380. The regulated delayed lysis plasmid has a pUC on and can express the ompAΩlhrh and gfp gene sequences in Salmonella to display the synthesized OmpAΩLHRH on the cell surface and GFP in the cytosol and after lysis of the CCTS cell employs the P_{cmv} promoter to express a selected gene to specify synthesis of a desired gene product.

To construct plasmid pG8R381 (FIG. 17B), the plasmid pG8R380 was cut with BstBI to remove the gene encoding GFP. The 8.1 kb fragment was self-ligated to generate plasmid pG8R381. The regulated delayed lysis plasmid has a pUC on and can express ompAΩlhrh and gfp in Salmonella to display the synthesized OmpAΩLHRH on the cell surface and after lysis of the CCTS cell employs the P_{cmv} promoter to express a selected gene to specify synthesis of a desired gene product.

### Example 15. Construction of Dual Plasmids to Cause CCTS Strains to Target Bladder Tumor Cells and then Express a Gene Sequence Encoding haPD1-IgG to Block PD1L1 Inactivation of T Cell Functions and a Gene Sequence Encoding KillerRed Mem to Kill Tumor Cells

The HAC-PD1 has a 40,000-fold higher affinity for inactivating PD-L1 than native PD1 and a 32- and 12-times higher inactivating ability than the FDA-approved anti-PD-L1 antibodies atezolizumab and durvalumab, respectively. HAC-PD1 is also more potent than an anti-PD1 antibody in inducing anti-cancer immunity using the same dose and schedule for intratumor injection. However, HAC-PD1, due to its small size, can leak from cells and thus elicit undesired immune responses against normal tissues. Furthermore, HAC-PD1 has a relatively short half-life and thus requires daily intratumoral injections. Thus, a HAC-PD1-IgG chimeric protein (haPD1-IgG, thereafter) can retain HAC-PD1 activity in tumors with a prolonged half-life and enhanced efficacy.

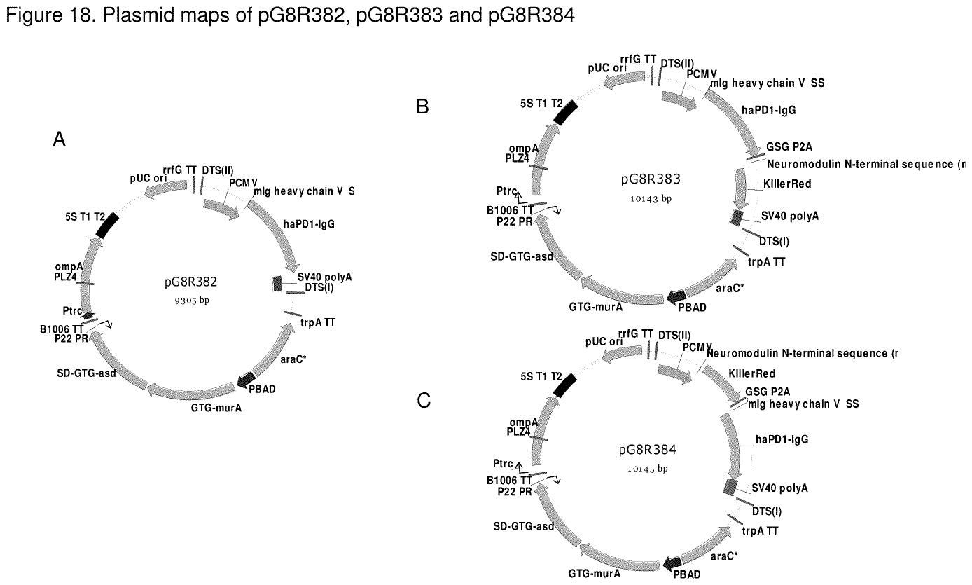

Since IgG has a long half-life, use of the chimeric fusion protein can reduce the need for frequent administration. We therefore generated plasmids pG8R382 (FIG. 18A), pG8R383 (FIG. 18B) and pG8R384 (FIG. 18C) carrying a sequence encoding haPD1-IgG.

To construct plasmid pG8R382 (FIG. 18A), we used plasmid pCMV3-haPD1-IgG (Sinobiological, Project number BWH2-P) as a template to amplify the gene encoding haPD1-IgG using primers haPD1IgG-KpnI-s and haPD1IgG-NotI-a. The gene was inserted into plasmid pG8R320 (FIG. 5B) cut with KpnI/NotI to generate plasmid pG8R382. The regulated delayed lysis plasmid has a pUC on and can express ompAΩplz4 in Salmonella to display the synthesized OmpAΩPLZ4 on the CCTS cell surface to target bladder cancer and after lysis of the CCTS cell in tumor cells employs the P_{cmv} promoter to express the haPD1-IgG to prevent inactivation of T cells.

To construct plasmid pG8R383 (FIG. 18B), we used plasmid pCMV3-haPD1-IgG (Sinobiological, Project number BWH2-P) as a template to amplify the gene encoding haPD1-IgG using primers and haPD1IgG-C-P2A-a. Then with plasmid pCS2-NXE+mem-KillerRed (Addgene plasmid #45761) as a template, the gene encoding KillerRed mem was amplified using primers P2A-KillerRed-Mem-s and KillerRed-NotIXhoI-a and then extended by PCR using primers P2A-gs and KillerRed-NotIXhoI-a. The two fragments were then inserted into plasmid pG8R320 (FIG. 5B) cut with KpnI/NotI to generate plasmid pG8R383. The lysis plasmid carries genes encoding haPD1-IgG and KillerRed mem separated by the P2A peptide under the control of the P_{cmv} promoter to enable synthesis of haPD1-IgG and KillerRed mem in tumor cells invaded by the CCTS strain. The regulated delayed lysis plasmid has a pUC on and can express ompAΩplz4 in Salmonella to display the synthesized OmpAΩPLZ4 on the cell surface and after lysis of the CCTS cell employs the P_{cmv} promoter to express synthesis of haPD1-IgG to prevent inactivation of T cells and KillerRed mem to kill the tumor cells.

To construct plasmid pG8R384 (FIG. 18C), we used plasmid pG8R343 (FIG. 10A) as a template to amplify the gene encoding KillerRed mem using primers KillerRed-Mem-KpnI-s and KillerRed-C-P2A-a2. And then with plasmid pCMV3-haPD1-IgG (Sinobiological, Project number BWH2-P) as a template, the gene encoding haPD1-IgG was amplified with primers P2A-haPD1IgG-s and haPD11 gG-NotI-a. The two fragments were then inserted into plasmid pG8R320 (FIG. 5B) cut with KpnI/NotI to generate plasmid pG8R384. The lysis plasmid carries genes encoding KillerRed mem and haPD1-IgG separated by P2A peptide under the control of a P_{cmv} promoter to enable synthesis of KillerRed mem and haPD1-IgG. The regulated delayed lysis plasmid has a pUC on and can express ompAΩplz4 in Salmonella to display the synthesized OmpAΩPLZ4 on the cell surface and after lysis of the CCTS cell employs the P_{cmv} promoter to express KillerRed mem to kill the tumor cell and haPD1-IgG to prevent the inactivation of T cells. Note that the only difference between pG8R343 and pG8R344 is the order of the sequences encoding the synthesis of haPD1-IgG and KillerRed.

### Example 16. Insertion of the Nucleotide Sequence Encoding the Single-Chain Fragment Variable (scFv) Targeting HER2 into the Third Exposed Loop of the S. typhimurium ompA Gene to Cause CCTS Strains to Target Bladder, Prostate and Breast Tumors

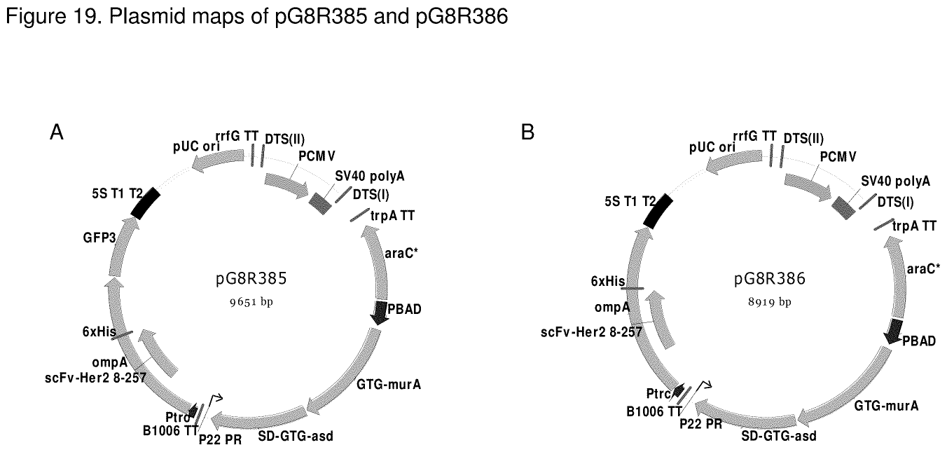

Human epidermal growth factor receptor 2 (HER2) is overexpressed in bladder, gastric, prostate and breast cancers. Single-chain fragment variables (scFv, ˜25 kDa) penetrate tumors better than large IgG and have faster clearance rates from the circulation to provide greater efficacy. The scFv targets Her2 to bind to ErB2+ cells to potentiate delivery of exogenous DNA and siRNA into ErB2+ cells. We thus generated regulated delayed lysis plasmids pG8R385 (FIG. 19A) and pG8R386 (FIG. 19B) that have a pUC onto synthesize scFv targeting Her2 on the Salmonella cell surface to target attaching to and invading bladder, gastric, prostate and breast cancers and after lysis of the CCTS cell in cancer cells employs the P_{cmv} promoter to drive expression of desired gene sequences important for tumor therapy.

To construct plasmid pG8R385 (FIG. 19A), we used plasmid pG8R320 (FIG. 5B) as a template to amplify a 536 bp fragment using primers P_{trc}-BcII-s and OmpA-Her2-a and a 694 bp fragment using primers OmpA-Her2-s and ompAGFP-BstBI-a. And then with plasmid pACgp67B-Her2 (Addgene Plasmid #10794) as a template, the gene encoding Her2 scFv was amplified with primers Her2-s and Her2His-a. With plasmid pYA4090 (FIG. 2C) as a template, the gene encoding GFP was amplified with primer GFP-s and GFP-BstBI-a. The 4 fragments were inserted into plasmid pG8R320 cut with BcII/BstBI to generate plasmid pG8R385. The regulated delayed lysis plasmid has a pUC on and can express ompAΩHer2 scFv and gfp in Salmonella to display the synthesized OmpAΩHer2 scFv on the CCTS cell surface and GFP in the cytosol and after lysis of the CCTS cell in a cancer cell employs the P_{cmv} promoter to express an inserted gene sequence of importance for cancer cell/tumor therapy.

To construct plasmid pG8R386 (FIG. 19B), the plasmid pG8R385 was cut with BstBI to remove the gene encoding GFP. The 8.9 kb fragment was self-ligated to generate plasmid pG8R386. The regulated delayed lysis plasmid has a pUC on and can express ompAΩHer2 scFv in Salmonella to display the synthesized OmpAΩHer2 scFv on the cell surface and after lysis of the CCTS cell employs the P_{cmv} promoter to express an inserted gene sequence of importance for cancer cell/tumor therapy.

### Example 17. Construction of Universal Vaccine Vectors to Enable Expression of Both Bacterial and Eukaryotic Genes by Insertion of Selected Nucleotide Sequences after the P_{trc} or P_{trc} Bla SSopt Promoter and the P_{CMV} Promoter, Respectively

The regulated delayed lysis plasmid pG8R320 (FIG. 5B) has a pUC on and can express the bacterial gene ompAΩplz4 under the control of the P_{trc} promoter in Salmonella to display the synthesized OmpAΩPLZ4 on the cell surface and after lysis of the cell employs the P_{cmv} promoter to express an inserted gene in eukaryotic cells. It is mainly used with Salmonella strains possessing the ΔompA mutation and mainly targeting bladder cancer cells with PLZ4 peptide. This mutation may not be required in all situations. The targeting to bladder cancer cells limits its usage for other cancer cells. Thus, a universal vector without the OmpAΩPLZ4 can be used for other purposes, such as targeting other cancer types or as a dual antigen delivery system. We generated plasmids pG8R388 (FIG. 19A) and pG8R389 (FIG. 19B) as two universal vectors to enable their use to express and deliver proteins of both bacterial and eukaryotic origins.

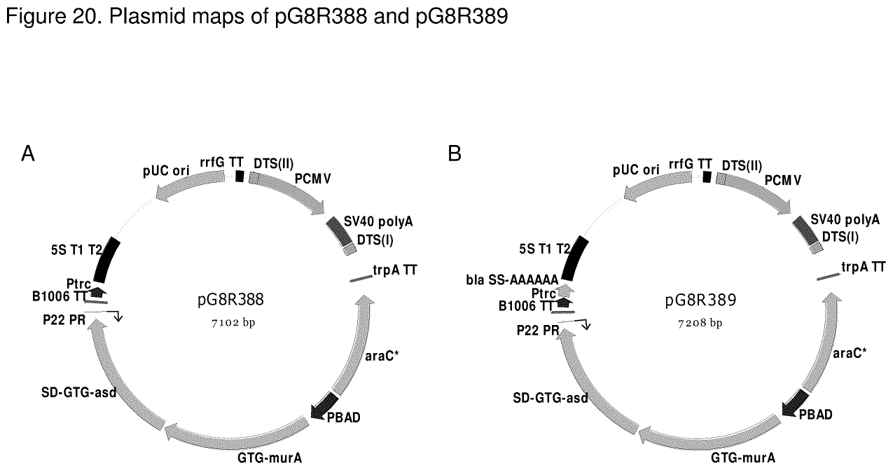

To construct pG8R388 (FIG. 20A), we used plasmid pYA3342 (FIG. 1A) as a template to amplify a 145 bp fragment containing the P_{trc} promoter using primers Ptrc-BcII-s and 4545Ptrc-a. The fragment was inserted into plasmid pG8R320 (FIG. 5B) cut with BcII/BstBI to generate plasmid pG8R388. The regulated delayed lysis plasmid has a pUC on and can express bacterial genes in the Salmonella cytosol and after invasion into a eukaryotic cell and lysis can employ the P_{cmv} promoter to express the eukaryotic gene in animal cells.

To construct pG8R389 (FIG. 20B), we used plasmid pG8R114 (FIG. 2B) as a template to amplify a 237 bp fragment containing the P_{trc} promoter with an optimized bla secretion signal (SS) using primers Ptrc-BcII-s and 4545PtrcBlaAAA-a. The fragment was inserted into plasmid pG8R320 (FIG. 5B) cut with BcII/BstBI to generate plasmid pG8R389. The regulated delayed lysis plasmid has a pUC on and can express bacterial gene fused with bla SSopt in Salmonella and secrete the synthesized protein into the periplasm and invasion into a eukaryotic cell and lysis can employ the P_{cmv} promoter to express the eukaryotic gene in animal cells.

### Example 18. Construction of Dual Plasmids to Cause CCTS Strains to Target Bladder Tumor Cells with LHRH Peptide or HER2 scFv and then Express a Gene Sequence Encoding KillerRed to Potentiate Tumor Cell Killing

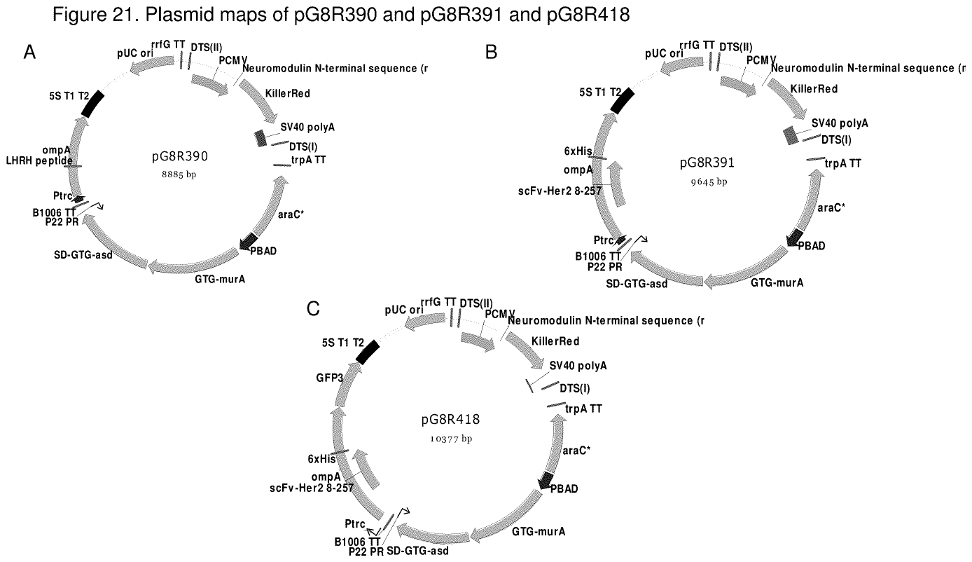

To validate the function of the LHRH peptide and HER2 scFv to target bladder tumor cells, we generated plasmids pG8R390 (FIG. 21A), pG8R391 (FIG. 21B) and pG8R418 (FIG. 21C) with KillerRed mem to potentiate tumor cell killing.

To construct pG8R390 (FIG. 20A), the gene encoding KillerRed mem was cut from pG8R327 (FIG. 8C) with KpnI/Not and cloned into plasmid pG8R381 (FIG. 17B) cut with KpnI/NotI to generate plasmid pG8R390. The regulated delayed lysis plasmid has a pUC on and can express ompAΩlhrh in Salmonella to display the synthesized OmpAΩLHRH on the cell surface and after lysis of the CCTS cell in situ employs the P_{cmv} promoter to express the gene encoding KillerRed mem to potentiate tumor cell killing.

To construct pG8R391 (FIG. 20B), the gene encoding KillerRed mem was cut from pG8R327 (FIG. 8C) with KpnI/Not and cloned into plasmid pG8R386 (FIG. 19B) cut with KpnI/NotI to generate plasmid pG8R391. The regulated delayed lysis plasmid has a pUC on and can express ompAΩher2 scFv in Salmonella to display the synthesized OmpAΩHer2 scFv on the CCTS cell surface and after lysis of the CCTS cell after invading into a cancer cell employs the P_{cmv} promoter to express the gene encoding KillerRed mem to potentiate tumor cell killing.

To construct pG8R391 (FIG. 21C), the gene encoding KillerRed mem was cut from pG8R327 (FIG. 8C) with KpnI/Not and cloned into plasmid pG8R385 (FIG. 19A) cut with KpnI/NotI to generate plasmid pG8R418. The regulated delayed lysis plasmid has a pUC on and can express ompAΩher2 scFv in Salmonella to display the synthesized OmpAΩHer2 scFv on the CCTS cell surface and after lysis of the CCTS cell after invading into a cancer cell employs the P_{cmv} promoter to express the gene encoding KillerRed mem to potentiate tumor cell killing.

### Example 19. Construction of Recombinant Plasmid CCTS Strains with Potential to Attach to and Invade into Bladder Tumor Cells to Deliver Desired Cargo to Directly and/or Indirectly Reduce Tumor Survival

All of the candidate CCTS strains listed in Table 4 possess the ΔompA11 mutation which can be substituted by a chromosomal ompAΩplz4 fusion allele and deletion of the asdA gene to enable establishment of a balanced-lethal vector-host strain after introduction of any of the recombinant plasmid vectors displayed in FIGS. 3 to 5 and 7 to 21, all of which encode synthesis of a receptor ligand facilitating specific targeting to bladder tumor cells. Most strains have the ΔP_{murA25}::TT araC P_{araBAD} murA mutation with a ΔasdA mutation to enable the display of the regulated delayed lysis in vivo phenotype that is desirable for delivery of cargoes synthesized by the CCTS strain and also for the delivery of plasmids serving as DNA vaccines to enable synthesis of a desired gene product by tumor cells. The inclusion of the re/A mutation facilitates the completeness of lysis by uncoupling the dependance of growth on continued protein synthesis. Most CCTS strains in Table 4 display a means to cause a regulated delayed attenuation in vivo by cessation in the synthesis of the LPS outer core or the LPS O-antigen. This phenotype also enhances the efficiency of CCTS strains to attach to and invade eukaryotic cells and in this case better display the modified outer membrane protein OmpAΩPLZ4 that is the means for targeting specific attachment to bladder tumor cells. These CCTS strains also have a means to display a regulated delayed synthesis of P_{trc} regulated gene insertions by the araC P_{araBAD} regulated expression of the lac/gene, an attribute that enhances the efficiency and frequency of in vivo colonization of the CCTS strains in target tissues. The presence of the Δ(wza-wcaM)-8 mutation facilitates complete lysis of strains with the regulated delayed lysis phenotype, enhances levels of plasmid encoded protein synthesis and precludes synthesis of exopolysaccharides that contribute to biofilm formation. Most strains have a ΔrecF mutation to reduce inter- and intra-plasmid recombination to enhance construct stability and a ΔendA mutation to eliminate the endonuclease that could degrade the plasmid vector upon lysis of the CCTS cell. Also present, is a ΔsifA mutation that enables the CCTS strain after invasion into a cell to escape the Salmonella containing vesicle (SCV) or endosome. This is important for release of DNA vaccines by CCTS cells since the DNA vaccine must be free to be directed to the tumor cell nucleus to enable transcription of the inserted gene sequence under the control of the plasmid encoded P_{CMV} or P_{EF1α} promoter. In this regard, the DNA vaccine components of all the plasmids containing sequence from pYA4545 (FIG. 1C) contain multiple sequences that direct the plasmid DNA to the cell nucleus (45). Since Salmonella infection into host cells induces pyroptosis that acts to destroy the nuclear organization and function, it is important that CCTS cells delivering DNA vaccines possess mutations such as the ΔsseL and ΔtlpA mutations to delay onset of pyroptosis and thus enhance expression of DNA vaccine encoded genes. Thus, many of the strains listed in Table 4 have such mutations and these mutations can be added to other strains using the suicide vectors listed in Table 2. Some of the strains listed in Table 4 have deletion mutations in the pagL, pagP, lpxR, eptA and arnT genes that alter the structure and activities of the LPS lipid A. These mutations may or may not contribute to the efficacy of CCTS constructs by altering the degree of inflammation in interacting with TLR4. Further modification of these activities can be accomplished by inclusion of the ΔpagP81::Pₗₚₚ lpxE and/or the ΔlpxR93::Pₗₚₚ lpxF deletion-insertion mutations that cause expression of codon-optimized Francisella tularensis genes to delete the 1′ of 4′ phosphates from lipid A to render it non-toxic but retain ability to bind to and activate TLR4. Since many CCTS gene activities are regulated by the sugars arabinose and rhamnose that must be supplied during in vitro cultivation but are absent in animal tissues, the timing of shut off of sugar-regulated gene expression after CCTS cell entry into an animal (human) host can be modulated by whether the sugars supplied during in vitro growth and retained in the cells are or are not quickly metabolized. Thus the mutations ΔaraBAD65::TT and ΔrhaBADSR515 are sometimes added to CCTS strains to delay shut off of the sugar regulated genes for several cell divisions. Additional refinements of strains can be achieved by inclusion of mutations such as ΔP_{tolR67}::::TT araC P_{araBAD} to/R that acts to increase production and release of outer membrane vesicles and other mutations that alter display or non-display of flagellar and fimbrial appendages or component parts to alter recruitment of innate immunity. These activities are well described in WO 2020/096994 A1 and WO 2021/222696 A1.

### Example 20. Evaluation of CCTS Constructs for Ability to Attach to and Invade Bladder Tumor Cells

CCTS strains with pG8R341 specifying synthesis of GFP by the CCTS strain and pG8R342 in which the EGFP activity must be synthesized by the bladder tumor cell after invasion by the CCTS strain. Based on the discussion in Example 19, we can compare constructs in 112417 (Δ_{PmurA25}::TT araC P_{BAD} murA ΔwaaL46 Δpmi-2426 ΔasdA27::TT araC P_{BAD} c2 ΔpagL64::TT rhaRS P_{rhaBAD} waaL Δ(wza-wcaM)-8 ΔrelA197::araC P_{BAD} lacI TT ΔrecF126ΔsifA26ΔompA11) that was used for our initial work and the much improved strains χ12735 (ΔPmurA25::TT araC P_{araBAD} murA ΔasdA33 ΔwaaL46 ΔpagL38::TT rhaRS P_{rhaBAD2} waaL2 Δ(wza-wcaM)-8 ΔrelA1123 ΔrecF126 ΔsifA26 ΔendA2113 ΔsseL116 ΔtlpA181 ΔrhaBADSR515 ΔaraBAD65::TT ΔompA11) and χ12736 (ΔP_{murA25}::TT araC P_{araBAD} murA ΔasdA33 ΔwaaL46 ΔpagL64::TT rhaRS P_{rhaBAD1} waaL1 Δ(wza-wcaM)-8 ΔrelA1123 ΔrecF126 ΔsifA26 ΔendA2113 ΔsseL116 ΔtlpA181 ΔrhaBADSR515 ΔaraBAD65::TT ΔompA11) (see Table 4). Strains can be grown in LB broth with 0.1% arabinose and with and without 0.1% rhamnose and evaluated for ability to attach to and invade bladder tumor cells as described in Example 1 and FIG. 6. It is expected based on the information provided in forgoing Examples that strains grown without rhamnose to prevent synthesis of the LPS O-antigen can be most proficient in attaching to bladder tumor cells. It is also expected that CCTS strains χ12735 and χ12736 can be most proficient in inducing synthesis of EGFP.

### Example 21. Exemplary Sequences

Sequences and SEQ ID NOs related to embodiments described herein are provided in a sequence listing in ST26 format.

pG8R314 (SEQ ID NO: 134)
ompA sequence (SEQ ID NO:1 (DNA) and SEQ ID NO: 2 (amino acid))
PLZ4 peptide (SEQ ID NO:3 (DNA) and SEQ ID NO:4 (amino acid))
Linker (SEQ ID NO:5 (DNA) and SEQ ID NO:6 (amino acid))
asd sequence (SEQ ID NO:7 (DNA) and SEQ ID NO:8 (amino acid))
   pG8R320 (SEQ ID NO: 135) (Includes SEQ ID NOs: 1, 3, 5, and 7)
murA sequence (SEQ ID NO: 9 (DNA) and SEQ ID NO: 10 (amino acid))
araC sequence (SEQ ID NO: 11 (DNA) and SEQ ID NO: 12 (amino acid))
Human CXCL11 (SEQ ID NO: 13 (DNA) and SEQ ID NO:14 (amino acid))
Mouse CXCL11 (SEQ ID NO: 15 (DNA) and SEQ ID NO:16 (amino acid))
KillerRed-memo (SEQ ID NO: 17 (DNA) and SEQ ID NO: 18 (amino acid))
KillerRed-mito (SEQ ID NO: 19 (DNA) and SEQ ID NO: 20 (amino acid))
HLAB in pG8R345 (SEQ ID NO: 21 (DNA) and SEQ ID NO:22 (amino acid))
OmpA-LHRH peptide (SEQ ID NO: 136) in pG8R380 and pG8R381
LHRH peptide (SEQ ID NO: 23 (DNA) and SEQ ID NO:24 (amino acid))
LHRH peptide (SEQ ID NO:25)
   KillerRed memo-P2A-HumanCXCL11 (SEQ ID NO: 52)
Neuromodulin N-terminal sequence (mem)(SEQ ID NO: SEQ ID NO: 26 (DNA) and SEQ ID NO: 27 (amino acid))
KillerRed sequence (SEQ ID NO: 28 (DNA) and SEQ ID NO: 29 (amino acid))
GSG P2A sequence (SEQ ID NO: 30 (DNA) and SEQ ID NO: 31 (amino acid))
Human CXCL11 sequence (SEQ ID NO: 32 (DNA) and SEQ ID NO: 33 (amino acid))

### KillerRed Memo-P2A-Mouse CXCL11 (SEQ ID NO: 53)

Neuromodulin N-terminal sequence (mem)(SEQ ID NO: 34 (DNA) and SEQ ID NO: 35 (amino acid))
KillerRed sequence (SEQ ID NO: 36 (DNA) and SEQ ID NO: 37 (amino acid))
GSG P2A sequence (SEQ ID NO: 38 (DNA) and SEQ ID NO: 39 (amino acid))
Mouse CXCL11 sequence (SEQ ID NO: 40 (DNA) and SEQ ID NO: 41 (amino acid))
GSG P2A sequence (SEQ ID NO: 42 (DNA) and (SEQ ID NO: 43 (amino acid))
   Her2 ScFV Sequence (SEQ ID NO: 137), Includes ompA and Her2ScFV DNA
ompA sequence (mem) (SEQ ID NO: 44 (DNA) and SEQ ID NO: 45 (amino acid))
Her2 ScFV sequence (SEQ ID NO: 46 (DNA) and SEQ ID NO: 47 (amino acid))
HAC-PD1 sequence (SEQ ID NO: 48 (DNA) and SEQ ID NO: 49 (amino acid))
haPD1-IgG sequence (SEQ ID NO: 50 (DNA) and SEQ ID NO: 51 (amino acid))
   pG8R388 (SEQ ID NO: 138), which Includes SEQ ID NO: 7, 9, and 11
   pG8R389 (SEQ ID NO: 139), which Includes SEQ ID NOs: 127, 7, 9 and 11
optimal bla sequence (SEQ ID NO: 127 (DNA) and SEQ ID NO: 128 (amino acid))
GSG T2A sequence (derived from thoseaasigna virus 2A) (SEQ ID NO: 129 (amino acid))
GSG E2A sequence (derived from equine rhinitis A virus) (SEQ ID NO: 130 (amino acid))
GSG F2A sequence (derived from foot-and-mouth disease virus) (SEQ ID NO: 131 (amino acid))
Linker sequence in pG8R362-pG8R365 (SEQ ID NO:132 (DNA)) (SEQ ID NO: 133 (amino acid))

### Example 22. Evaluation of the Effect of O-Antigen on the CCTS Constructs for Ability to Attach to and Invade Bladder Tumor Cells

Clinical trials showed only 3 out of 25 patients had Salmonella colonization at the tumor sites after intravenous injection, indicating that targeting efficiency of Salmonella should be increased. The OmpAΩPLZ4 fusion enables Salmonella displaying a bladder cancer targeting peptide on the surface of Salmonella to target bladder cancer cells. Lipopolysaccharide (LPS) is the structure that covers the Salmonella surface. Modifications of LPS in Salmonella can potentially affect Salmonella tumor targeting. LPS comprises Kdo-lipid A, inner core, outer core and O-antigen side chain. Kdo-lipid A is essential to the survival of Salmonella. The LPS mutations, ΔwaaL46, ΔwaaG42, and ΔwaaC41, which enable Salmonella to display defects in the synthesis of O-antigen, outer core and inner core, respectively, were introduced into Salmonella strains. It should be noted that waaC mutant defective in synthesis of the inner core are unable to synthesize and assemble the outer core and O-antigen whereas mutants unable to synthesize the outer LPS core also unable to display the O-antigen. These mutations were introduced into χ12614 to yield strain χ12812, χ12813, and χ12814, respectively (Table 5). A plasmid pG8R341 carrying multiple copies of ompAΩplz4 was introduced into these strains.

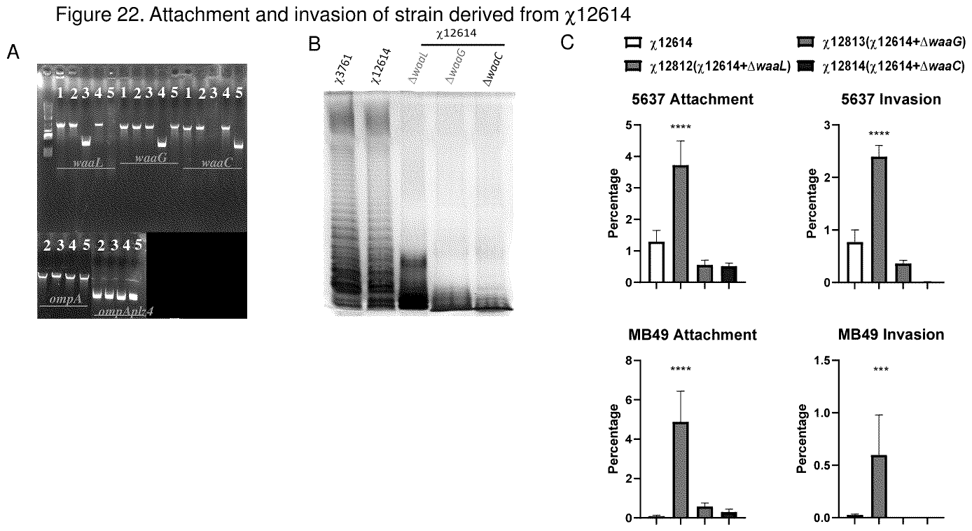

PCR with correspondent primers proved that strains derived from χ12614 have the correct expected genotype and the LPS gel proved that each strain has the right LPS phenotype human bladder cancer cell 5637 and mouse bladder cancer cells MB49 and BBN967 were performed in 24-well culture plates as described previously. The χ12614 lineage strains were grown in LB media until OD600 reached 0.85˜0.9. The bacteria were collected and resuspended in DMEM media with 10% fetal bovine serum. A MOI 10:1 was used to infect cells for 1 hour. After infection, half of the monolayers were washed with PBS and lysed with PBS containing 0.1% sodium deoxycholate to assess the total number of attached bacteria. The other half of cells were incubated for 1 h with DMEM media containing 100 μg/ml gentamicin to eliminate extracellular bacteria. Monolayers were then lysed with PBS containing 0.1% sodium deoxycholate to assess the total number of internalized bacteria. As shown in FIG. 22C, strain χ12812 with ΔwaaL46 mutation displays the highest abilities in attachment and invasion compared to the other strains abilities to attach to and invade both human and mouse bladder cells.

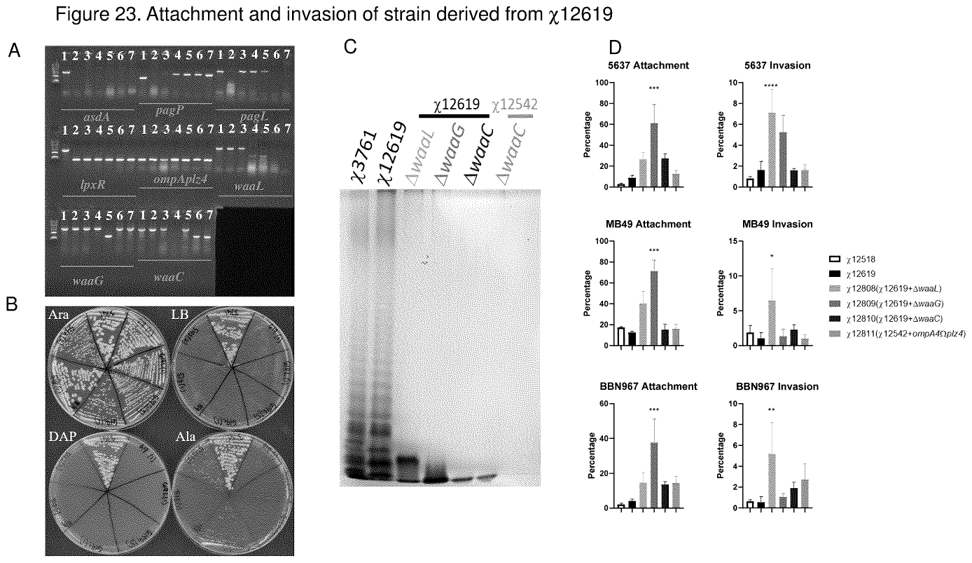

The LPS mutations, ΔwaaL46, ΔwaaG42, and ΔwaaC41 were also introduced into strain χ12619 to generate χ12808, χ12809, and χ12810, respectively. The ompAΩplz4 mutation (Table 2) was also introduced into strain χ12542 (Table 5) to yield strain χ12811 which has the identical genotypes as strain χ12810. These strains only have one copy of ompAΩplz4 in the chromosome. PCR with correspondent primers proved that strains derived from χ12619 have the correct expected genotype and the LPS gel proved that each strain had the right LPS phenotype (FIGS. 23 A and B). All the strains grew on LB with arabinose plates, but not on LB, LB with DAP and LB with alanine plates (FIG. 23C). The evaluation of the abilities of Salmonella cells to attach to and invade into human bladder cancer cell 5637 and mouse bladder cancer cells MB49 and BBN967 were performed in 24-well culture plates as described previously. The χ12619 lineage strains were grown in LB media with 0.1% arabinose until OD600 reaches 0.85˜0.9. The bacteria were collected and resuspended in DMEM media with 10% fetal bovine serum. A MOI of 10:1 was used to infect cells for 1 hour. After infection, half of the monolayers were washed with PBS and lysed with PBS containing 0.1% sodium deoxycholate to assess the total number of attached bacteria. The other half of the monolayers was incubated for 1 h with DMEM media containing 100 μg/ml gentamicin to eliminate extracellular bacteria. Monolayers were then lysed with PBS containing 0.1% sodium deoxycholate to assess the total number of internalized bacteria. As shown in FIG. 23C, strain χ12809 with ΔwaaG46 mutation displayed the highest abilities in attachment, however, strain χ12808 with ΔwaaL46 displays highest abilities of invasion to both human and mouse bladder cells.

### Example 24. Display of OmpAΩHer2 ScFV on the Bacterial Surface Enables S. typhimurium Cells to Preferentially Attach to Tumor Cells with Higher Her2 Production Levels

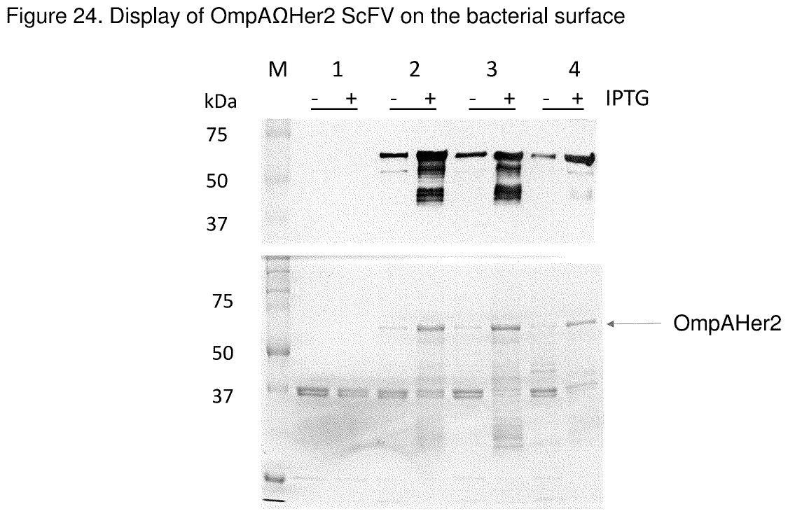

To evaluate the surface display of OmpAΩHer2 ScFV, strain χ12417 was transformed with plasmids pG8R385, pG8R418 and pG8R391 that carry the ompAΩher2 ScFV gene. The strains were grown in LB with 0.1% arabinose. 1 mM IPTG was added to induce the production of OmpAΩHer2 ScFV for 4 hours. The Salmonella outer member proteins (SOMPs) were prepared as described previously. The OmpAΩHer2 in strain χ12417 carrying any of the above plasmids can be detected in SOMPs portion of the SDS PAGE gel by Coomassie blue staining and western blot using anti-His6 antibody as an expected band around 65.1 kDa, but not the SOMPs from strain χ12417 (with no plasmid) (FIG. 24).

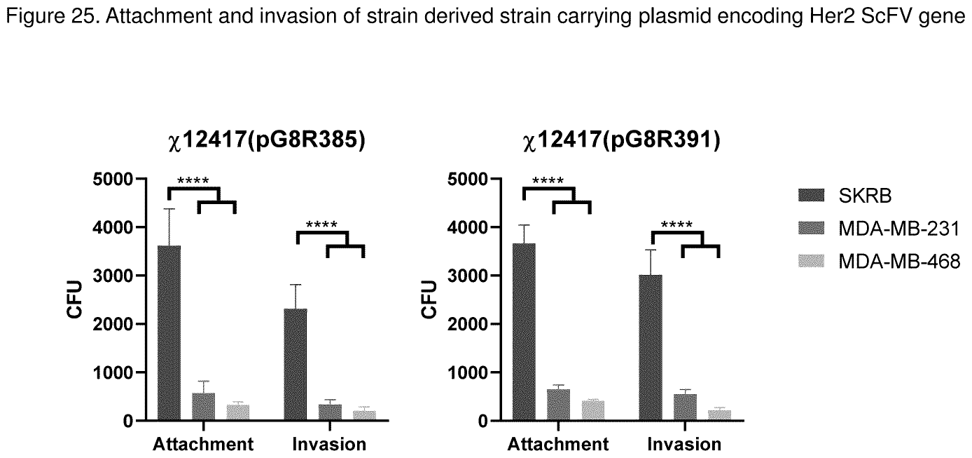

Different cell lines have different HER2 expression status. Her2 overexpression (SKBR-3, ATCC®HTB30) and low expression cell lines (MDA-MB-231 (ATCC® CRM-HTB-26) and MDA-MB-468 (ATCC® HTB-132)) were used to detect the attachment and invasion of the χ12417(pG8R385) and χ12417(pG8R391) strains (FIG. 25). Overnight cultures of χ12417(pG8R385) and χ12417(pG8R391) were diluted into LB broth with 0.1% arabinose and grown until OD600 reached 0.9. The bacteria were washed once with PBS and then used to infect SKBR-3, MDA-MB-431 and MDA-MB-468 breast cancer cells at an MOI 10:1 for 1 hour. After infection, half of the monolayers were washed with PBS and lysed with PBS containing 0.1% sodium deoxycholate to assess the total number of attached bacteria. The other half of the monolayers were treated for 1 h with DMEM media containing 100 μg/ml gentamicin to eliminate extracellular bacteria. Monolayers were then lysed with PBS containing 0.1% sodium deoxycholate to assess the total number of internalized bacteria. The strain χ12417(pG8R385) and χ12417(pG8R391) producing OmpAΩPLZ4 attached to and invaded to the highest levels the Her2 overexpression cell line SKBR-3, but not to the Her2 low expression cell lines MDA-MB-431 and MDA-MB-468.

### Example 25. KillerRed Kills HEK293T Cells

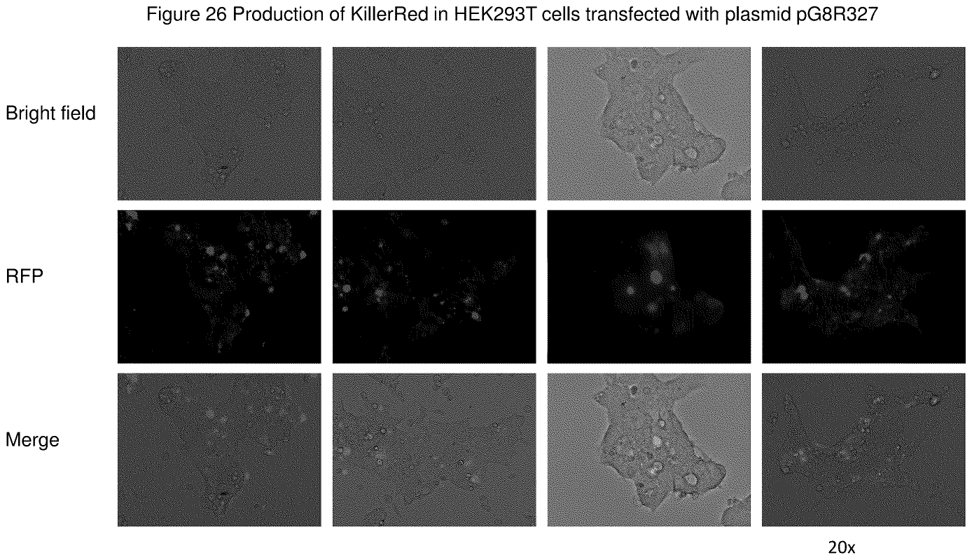

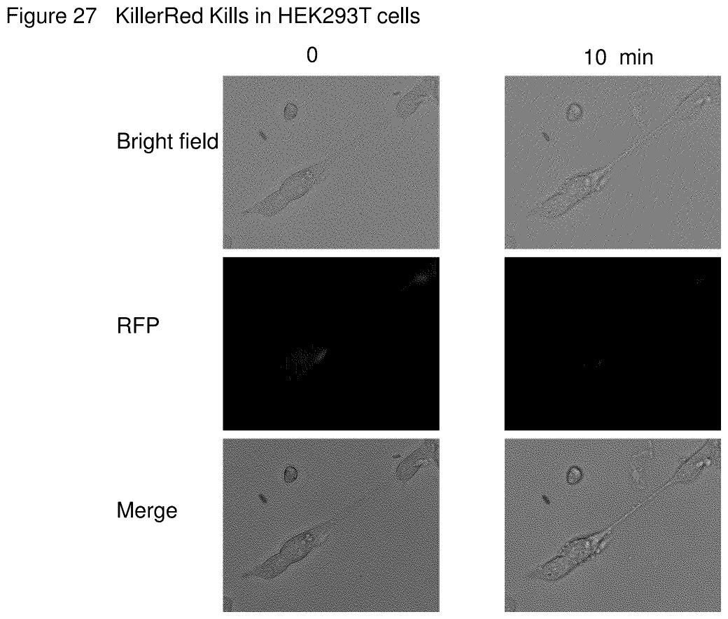

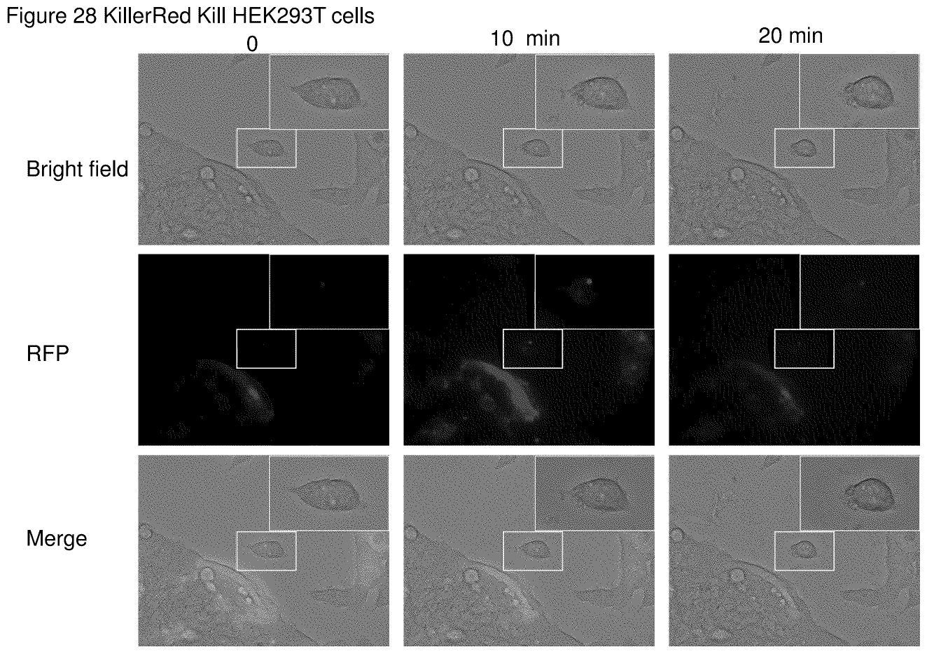

HEK293T cells were transfected with plasmid pG8R327 (FIG. 8C). The fluorescence was observed using EVOS Fl with RFP channel (Thermofisher). The RFP channel in EVOS Fl has 531/40 nm excitation and 593/40 nm émission. KillerRed can absorb 540˜580 nm wavelength light and emit an longer 610 nm red light with maximum fluorescence excitation/emission at 585/610 nm. Although the RFP channel is not the optimal wavelength for KillerRed, strong red fluorescence signals were observed in HEK293T cells transfected with plasmid pG8R327 (FIG. 26). Before RFP channel excitation, the cells showed spindle shapes with smooth surfaces. After excitation for 10 min, membrane blebs appear on the surface of the cells (FIG. 27). The phenomena was also observed in other samples. Further excitation for an additional 10 min led to the cell morphology changing to round instead of spindle shaped (FIG. 28). This observation is at a lower level but consistent with previous reports.

### Example 26. Evaluation of the CCTS Strains with Targeting Peptide In Vivo

The safety of CCTS strains uses oral and intravenous routes. Groups of 5 mice are inoculated with CCTS strains at varying doses ranging from 104 to 109 CFU. The mice are closely monitored for one month to see if any disease symptoms are observed.

Studies are conducted using multiple subcutaneous syngeneic tumor models to evaluate the distribution of CCTS strains based on the specific targeting peptide utilized. To establish the subcutaneous syngeneic tumor, approximately 1-5×10⁶ tumor cells are injected into the right flank of 6-8 week old mice. When tumor sizes reach approximately 100 mm³, the mice are administrated with the CCTS strains intravenously and orally using the highest safe dose determined from the previous experiment. At 24, 48, 72 and 96 hours, the spleen, liver, heart, lung, kidney, and tumors are harvested, weighted, homogenized and plated on LB agar with supplements. Fluorescence detection is performed on the tumors and organs to identify the presence of CCTS strains carry plasmids with the KillerRed or EGFP genes.

To test the target ability of the CCTS strain displayed PLZ4 peptide, BBN963 or MB49 murine bladder cancer cells are injected into the right flank of C57BL/6 mice. Mice are treated with CCTS strains with plasmids carrying the ompA3Ωplz4 gene and monitored as above. To test the target ability of LHRH peptide, high LHRH receptor expression cell lines, such as A2780 human ovarian cancer cells (1-5×10⁶) or human breast cancer cells MCF-7 (ATCC® HTB-22), MDA-MB-231 (ATCC® HTB-26), HCC1806 (ATCC® CRL-2335) are used to generate xenographs in athymic nu/nu mice. Mice are treated with a CCTS strain with a plasmid specifying the LHRH peptide and monitored as above. To test the target ability of Her2 ScFV, Her2 overexpression (SKBR-3, ATCC®HTB30) and low expression cell lines (MDA-MB-231 (ATCC® CRM-HTB-26) and MDA-MB-468 (ATCC® HTB-132)), respectively, are used to generate xenographs in athymic nu/nu mice. Mice are treated with a CCTS strain with a plasmid specifying Her2 SCFV and monitored as above.

Over 60% of bladder tumors have little immune cell infiltration inside tumors. CXCL11 is a cytokine that can attract CD8 cytotoxic T cells. The abilities of CCTS strains delivering plasmids pG8R319 (pBR ori, ompAΩplz4), pG8R320 (pUC ori, ompA3Ωplz4), pG8R322 (pBR ori, ompA3Ωplz4, cxcl11) and pG8R326 (pUC ori, ompA3Ωplz4, cxc/11) are compared to convert an immune “cold” to immune “hot” tumor. When tumors in C57BL/6 mice reach around 100 mm³, mice are fed with PBS, CCTS(pG8R319), CCTS(pG8R320), CCTS(pG8R322), or CCTS(pG8R326). Tumors can be harvested 24, 48, 72 or 96 hours later. Flow cytometry is used to compare the amount of CD8+ cells in different groups at these time points.

To further evaluate the abilities of CCTS strains to destroy tumors, C57BL6 mice carrying subcutaneous bladder tumors are used. When tumors reach the size of around 100 mm³, groups of 5 tumor-bearing mice are treated with a safe dose of CCTS strains with different cargos, including CXCL11, haPD1, haPD1-IgG. The treatment could be once or multiple times. Mice are monitored for weight, tumor growth and survival. If tumors regress, mice are maintained and monitored to see if the regrowth of tumors does or does not occur. Mice are euthanized once tumor sizes reach 1,500 mm³ or at the humane endpoint. Tumors are excised at the endpoint and either frozen directly in liquid nitrogen for storage or fixed in 10% formalin for histology or immunohistochemistry. In another experiment, tumors are collected before humane endpoint and split into 3 portions: formalin-fixation for immunohistochemical staining, fresh frozen for RNA and DNA extraction and deep sequencing; and single cell suspension for single cell sequencing.

Based on the characterizations of all the plasmid constructs in the previous Examples, we expect to observe specific tumor cell targeting in ectopic tumors and the expression of the encoded payloads to exhibit the desired effects.

### Example 27. Evaluation of the CCTS Strains with KillerRed In Vivo

KillerRed has been used in Photodynamic therapy (PDT). Mice carrying subcutaneous tumors are used. When tumors reach the size of around 100 mm³, tumor-bearing mice are treated with a safe dose of CCTS strains specifying synthesis of KillerRed. Fluorescence imagings of tumors are acquired daily in vivo using an IVIS-Spectrum (PerkinElmer, USA) with excitation wavelength of 570 nm and emission wavelength of 620 nm.

A suitable wavelength is used for PDT. When tumors reach the size of around 100 mm³, tumor-bearing mice are treated with a safe dose of CCTS strains delivering KillerRed. Tumors are either treated with a continuous wave or pulsed laser without causing excessive temperature effect on the skin surface. A validation parameter is described by Shirmanova et. al (83). The PDT is carried at 593 nm, 150 mW/cm², 270 J/cm² for the continuous laser wave daily for 7 days, or at 584 nm, 225 mW/cm², 337 J/cm² for the pulsed laser on the days 6, 7, and 8 of tumor growth. Skin surface temperature is monitored using an Infrared thermograph. After the treatment, randomly selected tumors in treated and untreated groups are collected and split into 3 portions: formalin-fixation for immunohistochemical staining, fresh frozen for RNA and DNA extraction and deep sequencing; and single cell suspension for single cell sequencing. During these studies, tumors are monitored with the size measured with a caliper twice a week until the mice reach humane endpoint.

### Example 28. Universal Vaccine Vectors to Enable Expression of Both Bacterial and Eukaryotic Genes by Insertion of Selected Nucleotide Sequences after the P_{trc} or P_{trc} Bla SSopt Promoter and the P_{CMV} Promoter, Respectively

A universal vaccine vector is a single vector that enables expression of genes both in prokaryotic and eukaryotic cells, even though it only specifies expression of gene only either in prokaryotic or eukaryotic cells. The regulated delayed lysis plasmids pG8R388 (FIG. 19A) and pG8R389 (FIG. 19B) are examples of two universal vectors to enable expression and delivery of proteins of both bacterial and eukaryotic origins. Plasmid pG8R388 has a pUC on and can express bacterial genes in the cytosol or on the surface of Salmonella and after invasion into a eukaryotic cell and lysis can employ the P_{cmv} promoter to express the eukaryotic gene(s) in animal cells. Plasmid pG8R389 has a pUC on and can express a bacterial gene fused with bla SSopt in Salmonella and secrete the synthesized protein into the periplasm and upon invasion into a eukaryotic cell and lysis can employ the P_{cmv} promoter to express the eukaryotic gene in animal cells. The prokaryotic promoter is not limited to P_{trc} and could be any promoter that functions in prokaryotic cells. Other prokaryotic promoters can be used. Multiple prokaryotic promoters can be used to drive the expression of multiple genes. Other secretion signals can be used to replace the bla SSopt. The eukaryotic promoter is not limited to P_{cmv} or P_{EF1α} and could be any promoter that functions in eukaryotic cells. Other eukaryotic promoters can be used. Multiple eukaryotic promoters can also be used to drive the expression of multiple genes.

Under the control of a prokaryotic promoter, a single gene can be expressed and multiple genes can also be expressed as an operon or protein fusion with or without suitable linkers. These linkers could be flexible, rigid, cleavable or dipeptide linkers. Some examples are listed by Chen et. al (84, 85). An unexhaustive list includes

(SEQ ID NO: 170)
(GGGS)n,
(SEQ ID NO: 171)
(GGGGS)n,
(G)n,
(SEQ ID NO: 172)
(EAAAK)n,
(XP)n,
(SEQ ID NO: 173)
GCT KESGSVSSEQLAQFRSLD,
(SEQ ID NO: 174)
EGKSSGSGSESKST,
and
(SEQ ID NO: 175)
GSAGSAAGSGEF.

FIG. 17A illustrates plasmid pG8R380, in which an operon fusion links ompAΩlhrh and gfp. FIG. 19A and FIG. 21C illustrates plasmids pG8R385 and pG8R418, in which an operon fusion links ompAΩher2 SCFV and gfp.

Under the control of a eukaryotic promoter, a single gene can be expressed and multiple genes can also be expressed separated by one or multiple 2A cleavage peptides. The 2A peptide could be P2A, E2A, T2A, F2A or other 2A-like sequences and thus form bi-, tri-, and quad-, penta- or multiple cistronic vectors. Plasmid pG8R343 (FIG. 10), pG8R344 (FIG. 10), pG8R383 (FIG. 18), pG8R384 (FIG. 18) are examples of bicistrion vectors with the P2A peptide. One or multiple IRES sequences could be used to generate multicistronic constructions for simultaneous expression of multiple genes. Multiple genes can be linked by a linker described above. FIG. 14 and FIGS. 15B and 15C illustrate examples in which HAC PD1 is linked to CXCL11 through a linker. FIG. 16B-D illustrate examples in which HAC PD1 is linked to EGFP through a 5A linker PPVAT (SEQ ID NO: 323).

### Example 29. Mutations to Activate Innate Immunity

MPLA is a potent activator of innate immune responses. However, natural lipid A in Salmonella, containing two phosphate groups and six acyl chains, is most efficient in activating pro-inflammatory responses through the TLR4-MD2-CD14 pathway (86-90), which is divided into a MyD88-dependent pathway contributing to reactogenicity and a TRIF-dependent pathway contributing to immunogenicity. Native Salmonella lipid A triggers an inflammatory response, whereas lipid A can be modified to induce diverse innate immune responses (91). MPLA (92), an extract from S. Minnesota by chemical method with low endotoxic activity and enhanced immunostimulatory activity, has been used as adjuvant for vaccines. Different enzymes have been identified to modify lipid A by reducing the number of phosphate groups or altering the length or number of the acyl chains (93). A phosphatase LpxE from Francisella tularensis can remove the 1-phosphate group of lipid A resulting in reduced toxicity of natural lipid A in Salmonella (88, 94). We thus generated the mutation ΔpagP::Pₗₚₚ lpxEto detoxify lipid A (95) (Tables 1, 2 and 3). When combined with mutations ΔpagL7ΔlpxR9, a strain with the mutation ΔpagP81::Pₗₚₚ lpxE mainly synthesizes a 4′-monophosphoryl-hexa-acylated lipid A under the range of conditions experienced in the host. The lpxE expression from this chromosomal location (pagP) attenuates Salmonella about five orders of magnitude without compromising the wild type-like ability of the strain to colonize lymphoid tissues nor the strain's immunogenicity (95), ensure the safety of the strains. Lipid A structure can be further modified by LpxF derived from Francisella that can remove the 4′-phosphate group to enhance its safety (Tables 1, 2 and 3). Lipid A structure can also be modified by mutations in arnT and eptA (Tables 1, 2 and 8).

We also generated the mutation ΔfliC180 that keeps the conserved N- and C-terminal ends of flagellin to interact with TLR5, but lack the hypervariable domain in the middle to reduce the adaptive immunity response to flagellin (Tables 1 and 2).

Both ΔpagP::Pₗₚₚ lpxE and ΔfliC180 mutation are important components of SDAAS strains to stimulate innate immune responses (Table 8). One of the examples of strain having both mutation is χ12518 (Table 8). The abilities of χ12518 to activate TLR4 and TLR5 are described in Example 30. To implement the abilities of activation of innate immunity and fully execute the advantages of SDAAS strains, they need to be improved for effective tumor and/or cancer cell targeting to ensure its activity in tumor/cancer and reduce the unneeded or unwanted activation in normal tissues. One improvement is to integrate the mutations for chemotaxis, purine and pyrimidine requirements. Another improvement is to induce an ΔompA mutation as described in Example 2, which enables the strain to display target peptide on the surface of Salmonella (Table 9). Consider Salmonella has at least 69 known outer membrane proteins (96), tumor target peptide can be inserted into these outer membrane proteins, or other surface protein enabling targeting to specific tumor cells.

TABLE 8
S. Typhimurium strains constructed and evaluated
as CCTS strains for increasing tumor colonization
by chemotaxis, purine and pyrimidine requirement.

χ12419 ΔP_{purA185}::rhaRS P_{rhaBAD} purA (from χ3761)
χ12492 ΔP_{murA25}::TT araC P_{BAD} murA ΔwaaL46 Δpmi-2426
ΔasdA27::TT araC P_{BAD} c2 ΔpagL64::TT rhaRS P_{rhaBAD} waaL Δ(wza-wcaM)-8
ΔrelA197::araC P_{BAD} lacI TT ΔrecF126 ΔsifA26 ΔompA11 ΔP_{purA185}::rhaRS P_{rhaBAD} purA
(from χ12417)
χ12493 ΔP_{murA25}::TT araC P_{BAD} murA ΔwaaL46 Δpmi-2426
ΔasdA27::TT araC P_{BAD} c2 ΔpagL64::TT rhaRS P_{rhaBAD} waaL Δ(wza-wcaM)-8
ΔrelA197::araC P_{BAD} lacI TT ΔrecF126 ΔsifA26 ΔompA11 ΔP_{purA185}::rhaRS P_{rhaBAD} purA
ΔsopB1925 (from χ12492)
χ12497 ΔpurA3114 (from χ3761)
χ12518 Δalr-3 ΔP_{dadB66}::TT araC P_{araBAD} dadB ΔP_{asdA55}::TT araC P_{araBAD} asdA ΔfliC180
ΔpagP81::Pₗₚₚ lpxE ΔpagL7 ΔlpxR9 (from χ12516)
χ12570 Δalr-3 ΔP_{dadB66}::TT araC P_{araBAD} dadB ΔP_{asdA55}::TT araC P_{araBAD} asdA ΔfliC180
ΔpagP81::Pₗₚₚ lpxE ΔpagL7 ΔlpxR9 ΔarnT (from χ12518)
χ12583 Δalr-3 ΔP_{dadB66}::TT araC P_{araBAD} dadB ΔP_{asdA55}::TT araC P_{araBAD} asdA ΔfliC180
ΔpagP81::Pₗₚₚ lpxE ΔpagL7 ΔlpxR9 ΔeptA (from χ12518)
χ12586 Δalr-3 ΔP_{dadB66}::TT araC P_{araBAD} dadB ΔP_{asdA55}::TT araC P_{araBAD} asdA ΔfliC180
ΔpagP81::Pₗₚₚ lpxE ΔpagL7 ΔlpxR9 ΔarnT ΔeptA (from χ12570)
χ12872 Δalr-3 ΔP_{dadB66}::TT araC P_{araBAD} dadB ΔP_{asdA55}::TT araC P_{araBAD} asdA ΔfliC180
ΔpagP81::Pₗₚₚ lpxE ΔpagL7 ΔlpxR9 ΔguaBA66 (from χ12518)
χ12873 Δalr-3 ΔP_{dadB66}::TT araC P_{araBAD} dadB ΔP_{asdA55}::TT araC P_{araBAD} asdA ΔfliC180
ΔpagP81::Pₗₚₚ lpxE ΔpagL7 ΔlpxR9 ΔP_{guaBA67}::rhaRS P_{rhaBAD} guaBA (from χ12518)
χ12874 Δalr-3 ΔP_{dadB66}::TT araC P_{araBAD} dadB ΔP_{asdA55}::TT araC P_{araBAD} asdA ΔfliC180
ΔpagP81::Pₗₚₚ lpxE ΔpagL7 ΔlpxR9 ΔpyrF68 (from χ12518)
χ12875 Δalr-3 ΔP_{dadB66}::TT araC P_{araBAD} dadB ΔP_{asdA55}::TT araC P_{araBAD} asdA ΔfliC180
ΔpagP81::Pₗₚₚ lpxE ΔpagL7 ΔlpxR9 ΔP_{pyrF69}::rhaRS P_{rhaBAD} pyrF (from χ12518)
χ12900 Δalr-3 ΔP_{dadB66}::TT araC P_{araBAD} dadB ΔP_{asdA55}::TT araC P_{araBAD} asdA ΔfliC180
ΔpagP81::Pₗₚₚ lpxE ΔpagL7 ΔlpxR9 ΔP_{purA185}::rhaRS P_{rhaBAD} purA (from χ12518)
χ12927 Δalr-3 ΔP_{dadB66}::TT araC P_{araBAD} dadB ΔP_{asdA55}::TT araC P_{araBAD} asdA ΔfliC180
ΔpagP81::Pₗₚₚ lpxE ΔpagL7 ΔlpxR9 ΔP_{purA185}::rhaRS P_{rhaBAD} purA Δtrg-70 (from χ12900)
χ12928 Δalr-3 ΔP_{dadB66}::TT araC P_{araBAD} dadB ΔP_{asdA55}::TT araC P_{araBAD} asdA ΔfliC180
ΔpagP81::Pₗₚₚ lpxE ΔpagL7 ΔlpxR9 ΔP_{purA185}::rhaRS P_{rhaBAD} purA Δtrg-70
ΔPₜₐᵣ::P_{trcΔlacO888} tar (from χ12927)
χ12929 Δalr-3 ΔP_{dadB66}::TT araC P_{araBAD} dadB ΔP_{asdA55}::TT araC P_{araBAD} asdA ΔfliC180
ΔpagP81::Pₗₚₚ lpxE ΔpagL7 ΔlpxR9 ΔP_{purA185}::rhaRS P_{rhaBAD} purA Δtrg-70
ΔPₜₛᵣ::P_{trcΔlacO888} tsr (from χ12927)
χ12930 Δalr-3 ΔP_{dadB66}::TT araC P_{araBAD} dadB ΔP_{asdA55}::TT araC P_{araBAD} asdA ΔfliC180
ΔpagP81::Pₗₚₚ lpxE ΔpagL7 ΔlpxR9 ΔP_{purA185}::rhaRS P_{rhaBAD} purA Δtrg-70
ΔPₜₛᵣ::P_{trcΔlacO888} tsr ΔPₜₐᵣ::P_{trcΔlacO888} tar (from χ12929)

TABLE 9
S. Typhimurium strains constructed and evaluated
as CCTS strains for increasing tumor colonization
by surface modification.

χ12869 Δalr-3 ΔP_{dadB66}::TT araC P_{araBAD} dadB ΔP_{asdA55}::TT araC P_{araBAD} asdA ΔfliC180
ΔpagP81::Pₗₚₚ lpxE ΔpagL7 ΔlpxR9 ΔompA11 (from χ12518)
χ12862 Δalr-3 ΔP_{dadB66}::TT araC P_{araBAD} dadB ΔP_{asdA55}::TT araC P_{araBAD} asdA ΔfliC180
ΔpagP81::Pₗₚₚ lpxE ΔpagL7 ΔlpxR9 ompAΩplz4 ΔwaaL46 ΔpagL19::TT araC P_{araBAD1}
waal (from χ12808)
χ12863 Δalr-3 ΔP_{dadB66}::TT araC P_{araBAD} dadB ΔP_{asdA55}::TT araC P_{araBAD} asdA ΔfliC180
ΔpagP81::Pₗₚₚ lpxE ΔpagL7 ΔlpxR9 ompAΩplz4 ΔwaaL46 ΔpagL38::TT rhaRS P_{rhaBAD1}
waaL2 (from χ12808)
χ12864 Δalr-3 ΔP_{dadB66}::TT araC P_{araBAD} dadB ΔP_{asdA55}::TT araC P_{araBAD} asdA ΔfliC180
ΔpagP81::Pₗₚₚ lpxE ΔpagL7 ΔlpxR9 ompAΩplz4 ΔwaaG42 ΔpagL21::TT araC P_{araBAD1}
waaG (from χ12809)
χ12865 Δalr-3 ΔP_{dadB66}::TT araC P_{araBAD} dadB ΔP_{asdA55}::TT araC P_{araBAD} asdA ΔfliC180
ΔpagP81::Pₗₚₚ lpxE ΔpagL7 ΔlpxR9 ompAΩplz4 ΔwaaC41 ΔpagL18::TT araC P_{araBAD1}
waaC (from χ12810)
χ12866 Δalr-3 ΔP_{dadB66}::TT araC P_{araBAD} dadB ΔP_{asdA55}::TT araC P_{araBAD} asdA ΔfliC180
ΔpagP81::Pₗₚₚ lpxE ΔpagL7 ΔlpxR9 ompAΩplz4 ΔwaaL46 ΔpagL19::TT araC P_{araBAD1}
waaL ΔsifA26 (from χ12862)
χ12867 Δalr-3 ΔP_{dadB66}::TT araC P_{araBAD} dadB ΔP_{asdA55}::TT araC P_{araBAD} asdA ΔfliC180
ΔpagP81::Pₗₚₚ lpxE ΔpagL7 ΔlpxR9 ompAΩplz4 ΔwaaL46 ΔpagL38::TT rhaRS P_{rhaBAD1}
waaL2 ΔsifA26 (from χ12863)
χ12870 Δalr-3 ΔP_{dadB66}::TT araC P_{araBAD} dadB ΔP_{asdA55}::TT araC P_{araBAD} asdA ΔfliC180
ΔpagP81::Pₗₚₚ lpxE ΔpagL7 ΔlpxR9 ompAΩplz4 ΔwaaL46 ΔpagL19::TT araC P_{araBAD1}
waaL ΔsifA26 ΔompA11 (from χ12866)
χ12871 Δalr-3 ΔP_{dadB66}::TT araC P_{araBAD} dadB ΔP_{asdA55}::TT araC P_{araBAD} asdA ΔfliC180
ΔpagP81::Pₗₚₚ lpxE ΔpagL7 ΔlpxR9 ompAΩplz4 ΔwaaL46 ΔpagL38::TT rhaRS P_{rhaBAD1}
waaL2 ΔsifA26 ΔompA11 (from χ12867)

### Example 30. Construction of CCTS Constructs with Super Activities to Activate Innate Immunity

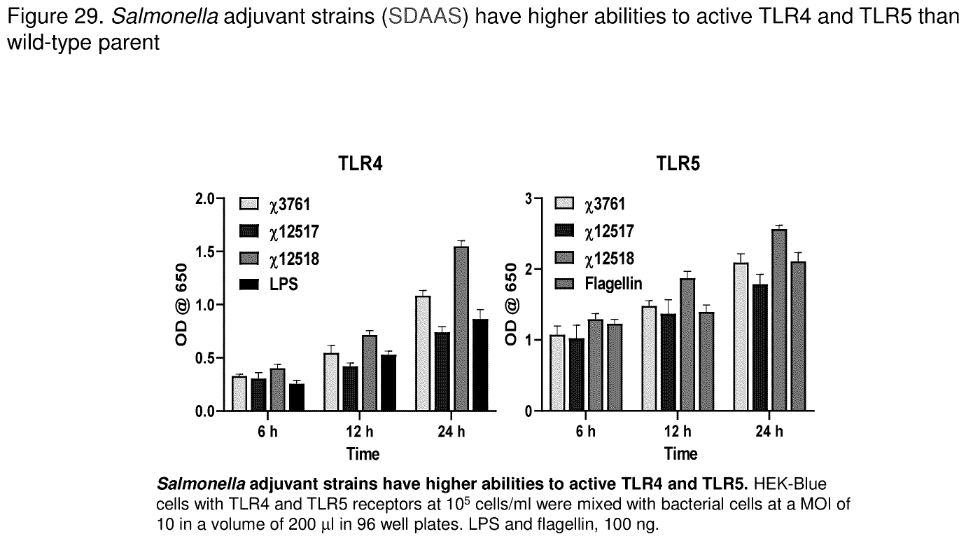

Strain χ12518 (Table 8) has mutations in lipid A and flagellin to increase their ability to activate the innate immune system. It is derived from the highly-virulent S. typhimurium UK-1 which is superior to the commonly used ATCC 14028 strain in destroying CT26 colorectal tumors in BALB/c mice (16). χ12518 has the genotype Δalr ΔP_{dadB}::TT araC P_{araBAD} dadB ΔP_{asdA}::TT araC P_{araBAD} asd ΔfliC180 ΔpagP::Pₗₚₚ lpxE ΔpagL7ΔlpxR9. The Δalr ΔP_{dadB} and ΔP_{asdA} mutations, based on our regulated delayed attenuation and lysis concept (97, 98), govern the arabinose-dependent synthesis of D-alanine and diaminopimelic acid, two essential constituents of peptidoglycan. They collectively provide regulated delayed attenuation and lysis in the absence of arabinose in vivo to enable total attenuation and safety. The regulated delayed attenuation strategy has been shown to better enhance inducing tumor regression than constitutive attenuation (16). The strain χ12518, with the mutations ΔfliC180 and ΔpagP::Pₗₚₚ lpxE, has higher activities to activate TLR4 and TLR5 than the wild-type parent χ3761 (FIG. 29), even higher than the natural agonists, LPS and flagellin. The ΔfliC180 mutation keeps the conserved N- and C-terminal ends of flagellin to interact with TLR5, which stimulates higher levels of TLR5 activity than its parent strain (FIG. 29B). The deletion of the hypervariable domain leads to higher adjuvancy and reduces the adaptive immunity response to flagellin (99). Strains with the triple mutations ΔpagP8 ΔpagL7ΔlpxR9 have been showed to induce tumor regression in a CT26 mouse model (16). We used the mutation ΔpagP::Pₗₚₚ lpxE replacing ΔpagP, which enables the strain to display adjuvant activity of MPLA with low toxicity (81, 95) to stimulate IFN-γ and induces higher levels of TLR4 activity than the parent strain (FIG. 29A)

### Example 31. Construction of CCTS Constructs to Colonize in Tumors Through the Attributes of Purine and/or Pyrimidine Requirement, Regulated Delayed Attenuation and Regulated Delayed Lysis to Facilitate Maximal Colonization of Tumor Tissues

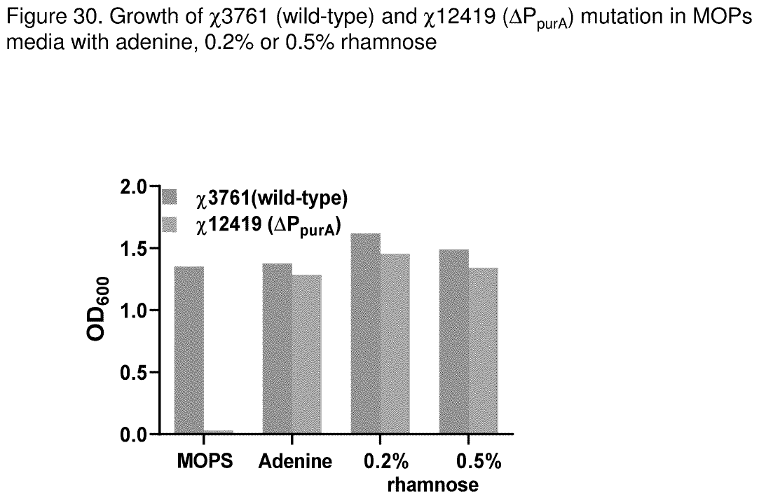

Our preliminary data showed that the mutations in χ12518 confer super activities to interact with TLR4 and TLR5 (FIG. 29). To further improve the strain to effectively killing tumor and/or cancer cells, we modified the strain to enhance its ability to home to and selectively colonize tumors in addition to the natural ability of Salmonella to colonize tumors. We therefore constructed a ΔP_{purA}::rhaRS P_{rhaBAD} purA mutation such that the PurA enzyme is synthesized during the growth of the SDAAS in medium with rhamnose, which is absent in vivo (FIG. 30). PurA(AdeK) catalyzes the first step toward the de novo synthesis of AMP dependent on presence of rhamnose. Non-phosphorylated purines are very scarce in normal tissues such that S. typhimurium strains with purA mutations fail to multiply when administered orally (65, 66) and parenterally (64, 65). They are therefore not immunogenic (65, 66). However, purines and other nucleosides are abundant in solid tumors due to cell necrosis and breakdown of nucleic acids (100). Adenine and uridine nucleotide are found in the extracellular space of tumors in millimolar concentrations, while only nanomolar concentration in nonpathological conditions (101, 102). Compared with uridine nucleotides, adenine nucleotides and nucleosides are a more important signaling pathway in cancer (102, 103). A strain χ12900 with the ΔP_{purA} mutation can be invasive but gradually become unable to grow unless accessing a tumor to multiply because of the abundant purines due to cell necrosis and breakdown of nucleic acids, but fail to multiply in normal tissues where purines are scarce (64-66) (Table 8). The usage of two sugars, rhamnose and arabinose, to regulate gene activities increases the safety of SDAAS strains.

We also constructed a ΔguaBA mutation (Tables 1 and 2) that deletes guaB(guaR) encoding IMP dehydrogenase (IMPDH)/inosine 5′-monophosphate dehydrogenase that catalyzes the NAD+-dependent oxidation of IMP to XMP, the first committed, rate-limiting step in de novo guanine nucleotide biosynthesis and guaA encoding GMP synthetase that catalyzes the glutamine- or ammonia-dependent synthesis of GMP from XMP. Both are needed to catalyze the de novo synthesis of GMP. Similarly, we generated a rhamnose regulated guaBA mutation ΔP_{guaBA}::rhaRS P_{rhaBAD} guaBA that enables the strain be invasive but gradually become unable to grow unless accessing a tumor to multiply because of the abundant guanine due to cell necrosis and breakdown of nucleic acids, but fail to multiply in normal tissues where guanine are scarce (64-66).

We further constructed a Δpyr Fmutation (Tables 1 and 2) that deletes pyrF encoding orotidine-5′-phosphate decarboxylase that catalyzes the last essential step in the de novo biosynthesis of pyrimidines. Similarly, we generate a rhamnose regulated pyrF mutation ΔP_{pyrF}::rhaRS P_{rhaBAD} pyrF that enables the strain be invasive but gradually become unable to grow unless accessing a tumor to multiply because of the abundant pyrimidine due to cell necrosis and breakdown of nucleic acids, but fail to multiply in normal tissues where non-phosphylated pyrimidines are scarce (64-66).

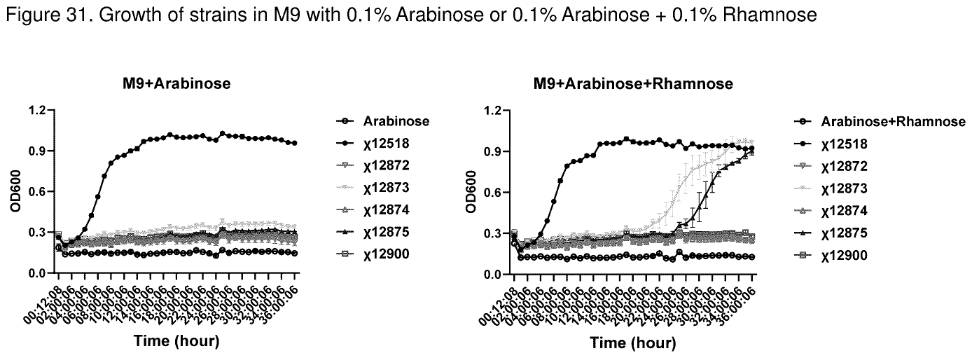

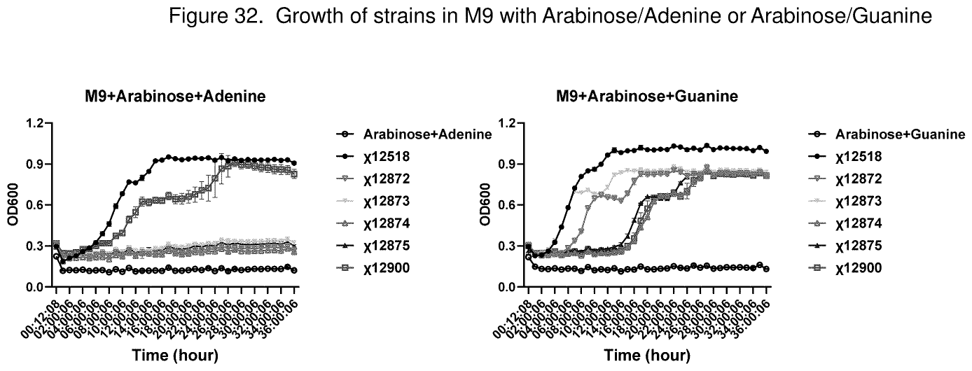

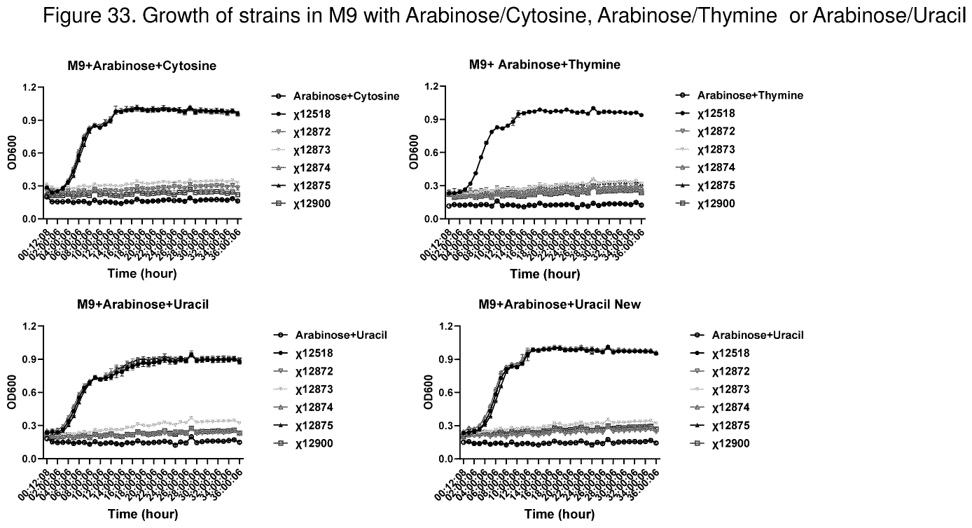

These mutations were introduced into strain χ12518 to generate strains χ12872(ΔguaBA66), χ12873(ΔP_{guaBA}), χ12874(ΔpyrF), χ12875(ΔP_{pyrF}), and χ12900 (ΔP_{purA}) (Table 8). The growth of these strains were evaluated in M9 medium supplemented with arabinose, rhamnose, adenine, guanine, cytosine, thymine and uracil as appropriate (FIGS. 31, 32 and 33). The OD600s of overnight cultures of strains grown in LB media with arabinose and rhamnose were recorded. The cultures were adjusted to the same OD600. The same volumes of bacteria were washed 3 times with BSG and diluted 1:100 into M9 media with different supplements. The cultures were checked on a growth curve machine in triplicates hourly for 36 hours. When these strains were grown in M9 media with arabinose, all strains could not grow except the parent strain χ12518 (FIG. 31). When grown in M9 with arabinose and rhamnose, there was no growth of χ12900 after 36 hours (FIG. 31). There was growth of χ12873 starting at 20 hours and χ12875 at 26 hours (FIG. 31), indicating that there are alternative pathways to scavenge the defects in the guaBA and pyrF mutations. When these strains were grown in M9 media with arabinose and adenine, only χ12900 grows as expected. When in M9 media with arabinose and guanine, χ12873 has a shorter lag phase than χ12872, whereas all other stains, χ12874(ΔpyrF), χ12875(ΔP_{pyrF}), and χ12900 (ΔP_{purA}) have very long lag phases around 18 hours (FIG. 32). When in M9 media with arabinose and cytosine or urcail, χ12874(ΔpyrF) and χ12875(ΔP_{pyrF}) can grow as expected, but no strain could grow with arabinose and thymine (FIG. 33).

Beside the above genes mentioned above, other genes, such as purF(ade, purC), purD(adth(a), adth), purN(_{ade(c), ade}), purT, purS, purQ, purL(purI), purM(purG, purI), purK(purE2), purE(ade(f), pur2, ade, ade3), purC(ade(g), ade), purB(ade(h), ade), purH, gmk(spoR), adk(dnaW, plsA), ndk, nrdD, nrdB(ftsB), nrdA(dnaF), nrdF(ygaD), nrdE, pyk, pykA(pyk), pykF(pyk1), pyrl, pyrB, pyrC, pyrD, pyrE, pyrF, pyrH(umk, smbA), pyrG, nudl(yagO), dut(dnaS, sot), mazG, thyA, tmk(ycfG), and comEB, that affect the purine and pyrimidine synthesis, can be used either as a deletion or regulated production by arabinose, rhamnose, xylose or other sugar regulated promoters can be used to achieve tumor-specific bacteria colonization.

### Example 32. Construction of CCTS Constructs for Ability to Colonize in Tumors Through the Over-Synthesis of the Serine and Aspartate Chemoreceptors and Deletion of the Ribose/Galactose Receptor to Facilitate Maximal Colonization of Tumor Tissues

In using SDAAS strains to eliminate tumors, it is critically important to kill tumor cells but without damage to normal cells. The effectiveness of most immunotherapeutic and chemotherapeutic drugs is also limited by their inability to penetrate deeply into tumor tissues and their ineffectiveness against quiescent cells (104, 105). Motile S. typhimurium can be attracted to compounds produced by cancer cells to overcome this therapeutic barrier to kill tumor cells (23, 106, 107). S. typhimurium has been shown to accumulate within the necrotic regions of tumors in vitro and in vivo by the mechanism of chemotaxis, which is essential to initiate bacterial accumulation (19, 108). Among the five chemotaxis-specific transmembrane receptors in S. typhimurium (109), four of them bind specific chemical ligands including aspartate/maltose, serine, citrate, and ribose/galactose (72, 74, 110-112). These compounds attract bacteria with chemoreceptors to home to tumors (19, 111, 113). Tar (aspartate and maltose receptor) initiates aspartate chemotaxis toward tumor cylindroids, Tsr (serine receptor) initiates serine chemotaxis for cylindroid penetration, and the Trg (ribose/galactose receptor) directs S. typhimurium toward the necrosis area and induces more apoptosis than a wild-type strain (19, 113). We constructed strains with the ΔPₜₐᵣ::P_{trcΔlacO} tar and ΔPₜₛᵣ::P_{trc ΔlacO} tsr mutations to enhance the accumulation of Salmonella within quiescent cells in tumors (Tables 1 and 2)(73). In these mutations, the promoters of tar and tsr genes were replaced with a P_{trc ΔlacO888} promoter that lacks the operator lacO sequence to enable constitutive synthesis of Tar and Tsr even when the lacI gene in the host strain is expressed. The constitutive synthesis of the Tar and Tsr attracts SDAAS strains toward the tumor microenvironment with aspartate and serine (72, 111, 113), but not to healthy tissues. The Δtrg mutation enables SDAAS strains to accumulate in tumor quiescence cells and induce apoptosis (113). A strain with these 3 mutations could effectively induce tumor regression in a breast cancer mice model (73). The strain χ12900 was further modified with ΔPₜₐᵣ::P_{trc ΔlacO} tar and ΔPₜₛᵣ::P_{trc ΔlacO} tsr mutations to generate strain χ12930 to display chemotaxis for effective cancer treatment (Table 8).

### Example 33. Quantify Bacterial Chemotaxis by Capillary Assay or Motility Test

The ability of the CCTS strains to migrate toward chemoattractant molecules could be quantified using the needle-syringe capillary assay (114). Briefly, the CCTS strains χ12900, χ12927, χ12929, χ1293 can be grown to mid-logarithmic phase, centrifuged, washed, and suspended in motility buffer to a final concentration of 3×10⁷ CFU/ml bacteria. Hypodermic needles (25 gauge) attached to 1 ml syringes can be filled with 0.1 ml of chemoattractant solution containing 0.1 mM serine or 1 mM aspartate or 1 mM galactose or 1 mM ribose. The needle-syringe assemblies can be inserted into 200 μl pipette tips containing the bacterial suspension and incubated for 1 h at 37° C. After incubation, the contents of the needles can be removed, diluted, and plated to quantify the bacterial numbers (CFU). Chemotactic ability can be reported as the ratio of the average number of bacteria that accumulated in the chemoattractant capillaries to the average number of bacteria that accumulated in the chemoattractant-free controls.

The ability of the CCTS strains to migrate toward chemoattractant molecules can also be quantified using the swimming test Briefly, the CCTS strains χ12900, χ12927, χ12929, χ12930 can be grown to mid-logarithmic phase, centrifuged, washed, and suspended in BSG to a final concentration of 3×10⁷ CFU/ml bacteria. A 5 ul sample can be spotted on a M9 minimal agar plate with 10-100μ mM serine, aspartate, galactose or ribose. The swarm ability can be checked after incubation overnight at 30° C.

### Example 34. Accumulation of Bacteria in Cylindroid

Human colon cancer cells, LS174T, HCT116, Human Melanoma cancer cell, A375, G361 or SK-Mel-1, mouse Melanoma cells, B16/F10, may be obtained from the American Type Culture Collection (Manassas, Va.) and cultured in Advanced Dulbecco's modified Eagle's medium (ADMEM) with 10% fetal bovine serum (FBS) and 26 mM HEPES buffer at 37° C. and 5% CO2. Cell aggregates can be grown in tissue culture flasks coated with 20 mg/ml poly (2-hydroxyethyl methacrylate) for 9 days to form spheroids. Formation of tumor cylindroids can be done as described by Kasinskas (19). Briefly, cylindroids can be formed by constraining spheroids between the bottom surface of a 96-well plate and the top surface of a set of polycarbonate cylindrical plugs attached to a polycarbonate lid. The diameter of each cylindroid is dependent on the initial size of the spheroid used in its formation. Spheroids ranging from 150 to 1,000 μm in diameter can be selected based on their size, symmetry, and overall integrity. After being constrained, cylindroids can be allowed to equilibrate for 22 h in 100 μL DMEM to relieve mechanical stress and establish oxygen and metabolic gradients before subjected to further experimentation (19).

Before inoculation into cylindroid cultures, the CCTS strains χ12900(pYA4685), χ12927(pYA4685), χ12929(pYA4685), χ12930(pYA4685) can be grown to mid-logarithmic phase (OD₆₀₀ 0.3-0.5) from single colony cultures at 37° C. Individual colonies can be chosen from agar plates following confirmation of GFP expression using fluorescence microscopy. These bacterial cultures can be collected and resuspended in Advanced DMEM to a final concentration of 500 CFU/ml. Equilibrated cylindroid cultures can be inoculated with 100 μl of 500 CFU/ml S. typhimurium. Time-lapse fluorescent images can be acquired at 10-min intervals up to 300 h after inoculation using time-lapse microscopy (Nikon Eclipse TE300 Inverted Microscope). Excitation light may be shuttered between acquisitions to prevent photobleaching. To test the influence of aspartate and serine on the accumulation of CCTS strains χ12900(pYA4685), χ12927(pYA4685), χ12929(pYA4685), χ12930(pYA4685), cylindroids can be prepared as described above and equilibrated in the medium containing 1 to 100 mM of added aspartate, serine, ribose, or galactose. Bacteria added to the cylindroids can be suspended in medium containing corresponding concentrations (1 to 100 mM) of aspartate, serine, ribose, or galactose. The accumulation of bacteria and fluorescent dyes in cylindroids can be quantified as described by (19).

### Example 35. Evaluation of the Safety of SDAAS Strains

Our strains are completely safe when administered by the oral route. We can test the attenuation and safety of SDAAS strains to determine the highest safe dose by the intravenous route which is normally used in Salmonella-cancer research. SDAAS can be grown in LB broth with 0.1% arabinose and/or 0.1% rhamnose. Groups of 5 mice can be inoculated intravenously with 104 to 109 CFU of SDAAS strains and the mice monitored for one month to see if any disease symptoms are observed. We can modify strains if adverse symptoms are observed though we do not expect any disease symptoms. The safety can also be tested using intranasal, intramuscular, and intraperitoneal routes.

### Example 36. Evaluation of the SDAAS Strains to Induce Tumor Regression and Ablation in a CT26 Colorectal Tumor Model

Colorectal cancer (CRC) is the 4th most common cancer diagnosed in the United States. In 2023, an estimated 153,020 new cases can be diagnosed with CRC in the U.S. As the 2nd leading cause of cancer-related deaths, it annually causes approximately 52,550 in the U.S. The CT26 CRC model is the most used mouse model and has been used in our previous studies (16, 115, 116). TLR4 expression is upregulated in CRC (117) and TLR5 activation leads to human colon tumor regression (54). Both MPLA and flagellin have been shown to enhance CRC immunotherapy (52, 55, 118). The abilities of the CCTS strains listed in Table 8 can be evaluated to target and destroy CT26 tumor in BALB/c mice (H-2d).

a. Animal model. Six to eight weeks old BALB/c mice can receive a subcutaneous injection into the right flank with 5×10³ CT26 colon carcinoma cells in 100 μl PBS. After 8 to 10 days of development for CT26, reaching tumor sizes of 100-150 mm³, mice can be grouped and standardized for mean and variance in tumor volume prior to injection treatments. Tumor development can be calculated via volume calculations based on caliper measurements in two dimensions: V=π/6×h×w2, wherein “h”=height and “w”=width (119, 120).

b. Evaluation of the abilities of SDAAS strains to home to tumors. Groups of mice with tumors can be inoculated intravenously with doses determined by Example 35 of SDAAS strains grown in LB broth with 0.1% arabinose and/or 0.1% rhamnose. Three mice for each group can be euthanized on days 1, 3, 6 and 10 to determine strain titers in blood, mesenteric lymph nodes, spleen, liver, lungs, heart, kidneys, bladder, ovary, pancreases, and tumors. These data can be expressed as c.f.u. per gram of tissue. We then calculate the differential abilities of the SDAAS strain to target and multiply in tumors but are unable to infect or persist in normal tissues. In all studies, we can observe excessive inflammatory responses or other signs of potential adverse occurrences by observation of the levels of TNF-α, IFN-γ, aspartate transaminase, and alanine aminotransferase in serum, and the numbers of leukocytic infiltration spots in histologic lung, liver and spleen and tumor sections. Repeating studies with lower/higher doses or possible second doses or alternate routes can be also conducted with SDAAS strains.

c. Evaluation of the abilities of SDAAS strains to destroy tumors. Groups of 5 tumor-bearing mice can be treated with SDAAS strains. If tumors regress, mice can be maintained and monitored to see if the regrowth of tumors does or does not occur. If new tumors are observed, we can inoculate mice with the same SDAAS strain. The body weight, tumor size, and mass can be recorded. Tumors can be exercised at the endpoint and either frozen directly in liquid nitrogen for storage or fixed in 10% formalin for histology or immunohistochemistry. Two pathologists blinded to the experiment can independently review histology sections.

d. Evaluation of treatment with SDAAS strains on innate and adaptive immune cells. After treatment with a SDAAS strain, tumor tissue can be collected at different times to characterize the ratio of different innate cell types in tumors with a focus on NK cells (121-123), neutrophils (124, 125), dendritic cells (DC) (126) and monocyte/macrophages (127-129) as well as T and B cells. The markers to screen for are CD3/CD4/CD8 (T cells), B220/CD22 (B cells), CD335/Nkp46/CD49b (NK cells), CD15/CD16 (neutrophils), Vα24Vβ11 (NKT cells), CD11c/MHCII (DCs), CD11b/Mac-1/Ly-71 (macrophages/monocytes) and CD31/CD106 (endothelial cells).

### Example 37. Augment of Melanoma Targeting SDAAS Strains by Adopting Colorectal Cancer Targeting Peptides

Colorectal cancer targeting peptide can be used to augment the targeting ability of SDAAS strain to colorectal cancer. Many colorectal cancer targeting peptides have been identified by different methods (Table 10). These peptides are in different categories. Some peptides have a general target, like RGD targeting αᵥβ₃. αᵥβ₃ involves in cell adhesion and signal transduction for tumor progression and angiogenesis, especially for tumors with the invasive and metastatic potential, including colorectal cancer. Targeting αᵥβ₃ could inhibit the angiogenesis and tumor spread. These peptides could target multiple tumors, not limited to including colorectal cancer. There are also other peptides, such as CP15 and COLO320HSR, that target specific colorectal cancer cells. Some peptides, like EBP and Pep11, have identified target, but have not been tested in colorectal cancer cells. SDAAS can deliver all these peptides thorough fusion of these peptides with outer membrane protein(s) to augment the target ability of SDAAS. The outer membrane protein is not limited to OmpA, but could be OmpC, OrnpD, OmpF, OmpN, OmpW, OmpX, ApeE/EstA, BtuB, CirA, FepA, FhuA, IroN, PagC, Rck or any other proteins that could display these peptides on the surface of Salmonella.

OmpC
>AEF08184.1 outer membrane porin protein C [Salmonella
enterica subsp. enterica serovar Typhimurium str. UK-1,
378 AA, complement (CP002614.1:2365188..2366324),
STMUK_2298]
(SEQ ID NO: 176)
MKVKVLSLLVPALLVAGAANAAEIYNKDGNKLDLFGKVDGLHYFSDDKGSDGDQTYM
RIGFKGETQVNDQLTGYGQWEYQIQGNQTEGSNDSWTRVAFAGLKFADAGSFDYGR
NYGVTYDVTSWTDVLPEFGGDTYGADNFMQQRGNGYATYRNTDFFGLVDGLDFALQ
YQGKNGSVSGENTNGRSLLNQNGDGYGGSLTYAIGEGFSVGGAITTSKRTADQNNTA
NARLYGNGDRATVYTGGLKYDANNIYLAAQYSQTYNATRFGTSNGSNPSTSYGFANK
AQNFEVVAQYQFDFGLRPSVAYLQSKGKDISNGYGASYGDQDIVKYVDVGATYYFNK
NMSTYVDYKINLLDKNDFTRDAGINTDDIVALGLVYQF
OmpD
>AEF07442.1 putative outer membrane porin precursor ompD
[Salmonella enterica subsp. enterica serovar Typhimurium
str. UK-1, 362 AA, CP002614.1:1613342..1614430, STMUK_1541]
(SEQ ID NO: 177)
MKLKLVAVAVTSLLAAGVVNAAEVYNKDGNKLDLYGKVHAQHYFSDDNGSDGDKTYA
RLGFKGETQINDQLTGFGQWEYEFKGNRTESQGADKDKTRLAFAGLKFADYGSFDYG
RNYGVAYDIGAWTDVLPEFGGDTWTQTDVFMTGRTTGVATYRNTDFFGLVEGLNFAA
QYQGKNDRDGAYESNGDGFGLSATYEYEGFGVGAAYAKSDRTNNQVKAASNLNAAG
KNAEVWAAGLKYDANNIYLATTYSETLNMTTFGEDAAGDAFIANKTQNFEAVAQYQFD
FGLRPSIAYLKSKGKNLGTYGDQDLVEYIDVGATYYFNKNMSTFVDYKINLLDDSDFTK
AAKVSTDNIVAVGLNYQF
OmpF
>AEF06873.1 outer membrane protein F precursor [Salmonella
enterica subsp. enterica serovar Typhimurium str. UK-1,
363 AA, complement (1,047,430..1,048,521) STMUK_0965]
(SEQ ID NO: 178)
MMKRKILAAVIPALLAAATANAAEIYNKDGNKLDLYGKAVGRHVWTTTGDSKNADQTY
AQIGFKGETQINTDLTGFGQWEYRTKADRAEGEQQNSNLVRLAFAGLKYAEVGSIDYG
RNYGIVYDVESYTDMAPYFSGETWGGAYTDNYMTSRAGGLLTYRNSDFFGLVDGLSF
GIQYQGKNQDNHSINSQNGDGVGYTMAYEFDGFGVTAAYSNSKRTNDQQDRDGNG
DRAESWAVGAKYDANNVYLAAVYAETRNMSIVENTVTDTVEMANKTQNLEVVAQYQF
DFGLRPAISYVQSKGKQLNGAGGSADLAKYIQAGATYYFNKNMNVWVDYRFNLLDEN
DYSSSYVGTDDQAAVGITYQF
OmpN
>AEF07339.1 outer membrane protein N precursor [Salmonella
enterica subsp. enterica serovar Typhimurium str. UK-1,
377 AA, complement (CP002614.1:1507844..1508977), STMUK_1437]
(SEQ ID NO: 179)
MKRKVLALVIPALLAAGAAHAAEIYNKDGNKLDLYGKVDGLHYFSDDSSKDGDQTYMR
VGFKGETQINDQLTGYGQWEYNVQANTTEGEGANSWTRLAFAGLKFGDYGSFDYGR
NYGVLYDVEGWTDMLPEFGGDSYTYADNFMTGRANGVATYRNTDFFGLVDGLNFAL
QYQGANENQVSHEQEGTNNGGDRNVKNANGDGFGISSTYDLGMGVSFGTAYTTSDR
TNGQVNYSTAGGDKADAWTVGAKYDANNIYLATMYSETRNMTPYGNDDCRSCVANK
TQNFEVTAQYQFDFGLRPAVSFLMSKGKDLTYNNVNGDDKDLVKYADVGATYYFNKN
FSTYVDYKINLLDDDDQFYKDAGISTDDIVALGMVYQF
OmpW
>AEF07604.1 outer membrane protein W [Salmonella enterica
subsp. enterica serovar Typhimurium str. UK-1, 212 AA,
complement (CP002614.1:1786774..1787412), STMUK_1704]
(SEQ ID NO: 180)
MKKFTVAALALTTLLSGSAFAHEAGEFFMRAGPATVRPTEGAGGTLGHLNGFDVSNN
TQLGLTFTYMATDNIGVELLAATPFRHKVGTGATGDIATVHLLPPTLMAQWYFGDSSS
KVRPYVGVGVNYTTFFDNDFNDNGKNAGLSDLSFKDSWGAAGQVGVDYLINRDWLIG
ASVWYMDIDTTANYKMGGVQHHDSVRLDPWVFMFSAGYRF
OmpX
>AEF06746.1 outer membrane protein X [Salmonella enterica
subsp. enterica serovar Typhimurium str. UK-1, 171 AA,
CP002614.1:899875..900390 STMUK_0837]
(SEQ ID NO: 181)
MKKIACLSALAAVLAFSAGTAVAATSTVTGGYAQSDAQGVANKMSGFNLKYRYEQDD
NPLGVIGSFTYTEKDRTNGAGDYNKGQYYGITAGPAYRLNDWASIYGVVGVGYGKFQ
TTDYPTYKHDTSDYGFSYGAGLQFNPMENVALDFSYEQSRIRSVDVGTWIAGVGYRF
ApeE/EstA
>AEF06499.1 outer membrane esterase [Salmonella enterica
subsp. enterica serovar Typhimurium str. UK-1, 656 AA,
CP002614.1:627886..629856 STMUK_0575]
(SEQ ID NO: 182)
MTQKRTLLKYGILSLALAAPLSACAFDSLTVIGDSLSDTGNNGRWTWDSGQNKLYDEQ
LAERYGLELSPSSNGGSNYAAGGATATPELNPQDNTADQVRQWLAKTGGKADHNGL
YIHWVGGNDLAAAIAQPTMAQQIAGNSATSAAAQVGLLLDAGAGLVVVPNVPDISATP
MILEAVITAGLGAAAPPALKAALDALAEGATPDFASRQQAIRKALLAAAATVSSNPFIQQ
LLVEQLLAGYEAAAGQASALTDYYNQMEEKGLEQHGGNIARADINGLFKEILANPQAF
GLTNTVGMACPPGVSASACSSAMPGFNASQDYLFADHLHPGPQVHTIIAQYIQSIIAAP
VQATYLNQSVQSMAQGSRTTLDSRYQQLRQGENPVGSLGMFGGYSGGYQRYDNNE
ADGNGNHNNLTVGVDYQLNEQVLLGGLIAGSLDKQHPDDNYRYDARGFQAAVFSHLR
AGQAWLDSDLHFLSAKFSNIQRSITLGALRRVEEGETNGRLWGARLTSGYDFVMVPW
LTTGPMLQYAWDYSHVNGYSEKLNTSTSMRFGDQNAHSQVGSAGWRLDLRHSIIHS
WAQINYRRQFGDDTYVANGGLKSTALTFSRDGKTQDKNWVDIAIGADFPLSATVSAFA
GLSQTAGLSDGNQTRYNVGFSARF
BtuB
>AEF09941.1 vitamin B12/cobalamin outer membrane transporter
[Salmonella enterica subsp. enterica serovar Typhimurium str.
UK-1, 614 AA, CP002614.1:4309301..4311145 STMUK_4114]
(SEQ ID NO: 183)
MIKKATLLTAFSVTAFSAWAQDTSPDTLVVTANRFQQPRSAVLAPVTIVTRQDIERWQS
TSVNDVLRRLPGVDIAQSGGAGQNSSIFIRGTNSSHVLVLIDGVRLNLAGVSGSADLSQ
FPVSLVQRIEYIRGPRSAIYGSDAIGGVVNIITTRDNPGTELTAGWGSNSYQNYDISTQQ
QLGENTRATLIGDYEYTKGFDVVAKGGTGMQAQPDRDGFLSKTLYGALEHTFSDRWS
GFVRGYGYDNRTDYDAYYSPGSPLIDTRKLYSQSWDAGLHFNGERIQSQLVSSYSHS
KDYNYDPHYGRYDTSATLDEMKQYNVQWTNSVVVGHGNVGAGVDWQKQTTTPGTG
YVPEGYDQRNTGVYLTGLQQLGDFTLEAAARSDDNSQFGRHGTWQTSAGWEFIEGY
RFIASYGTSYKAPNLGQLYGYYGNPNLNPEKSKQWEGAFEGLTAGVSWRISGYRNDI
NDMIDYDDHLQKYYNEGKARIKGIEATANFDTGPLTHTVSYDYVDARNAITDTPLPRRS
KQMAKYQLDWDVYDFDWGMTYQYLGSRYDSDYSAYPYRTVKMGGVSLWDLTVAYP
VTSHLTVRGKIANLFDKDYETVYGYQTAGREYTLSGSYTF
CirA
>AEF08116.1 colicin I receptor [Salmonella enterica subsp.
enterica serovar Typhimurium str. UK-1, 663 AA, complement
(CP002614.1:2296297..2298288) STMUK_2229]
(SEQ ID NO: 184)
MFRFNPFVRVGLCMSAVTLAWPVAAATDDGETMVVTASAIEQNLKDAPASISVITQQD
LQRRPVQNLKDVLKEVPGVQLTNEGDNRKGVSIRGLDSSYTLILIDGKRVNSRNAVFR
HNDFDLNWIPVDAIERIEVVRGPMSSLYGSDALGGVVNIITKKIGQKWHGSVTVDSTIQ
EHRDRGDTYNGQFFTSGPLIDGVLGMKAYGSLAKREKDEQQSSATTATGETPRIEGFT
SRDGNVEFAWTPNENHDVTAGYGFDRQDRDSDSLDKNRLERQNYALSHNGRWDLG
NSELKFYGEKVENKNPGNSSPITSESNSIDGKYVLPLASVNQFLTFGGEWRHDKLSDA
VNLTGGSSTKTSASQYALFLEDEWRIFEPLALTTGIRMDDHETYGDHWSPRAYLVYNA
TDTLTVKGGWATAFKAPSLLQLSPDWATNSCRGGCRIVGSPDLKPETSESWELGLYY
RGEEGILEGVEASVTTFRNDVDNRISISRTPDVNAAPGYSNFVGFETNSRGQRVPVFR
YYNVNKARIQGVETELKVPFNEAWKLSLNYTYNDGRDVSNGGNKPLSDLPFHTANGT
LDWKPVQLEDWSFYVSGNYTGRKRADSATAKTPGGYVVWDTGAAWQATKNVKLRA
GVLNVGDKDLKRDDYGYTEDGRRYFMAVDYRF
FepA
>AEF06514.1 outer membrane receptor FepA [Salmonella
enterica subsp. enterica serovar Typhimurium str. UK-1,
751 AA, complement (CP002614.1:642251..644506),
STMUK_0590]
(SEQ ID NO: 185)
MNKKIHSLTLLVNLGIYGAALPVMAEDKTDSAALTNEDTIVVTAAQQNLQAPGVSTITAD
EIRKNPPARDVSEIIRTMPGVNLTGNSTSGQRGNNRQIDIRGMGPENTLILIDGKPVTSR
NSVRLGWRGERDTRGDTAWVPPEMIERIEVLRGPAAARYGNGAAGGVVNIITKKGGS
EWHGSWNTYFNAPEHKDEGATKRTNFSLNGPLGGDFSFRLYGNLDKTQADARNINQ
GHQSERTGSYADTLPAGREGVINKDINGVVRWDFAPLQSLELEAGYSRQGNLYAGDT
QNTNTNQLVKDNYGKETNRLYRQNYSLTWNGGWNNGVTTSNWVQYEHTRNSRMPE
GLAGGTEGIFDPKASQKYADADLNDVTLHSEVSLPFDLLVNQNLTLGTEWAQQRMKD
QLSNSQTFMGGNIPGYSSTNRSPYSKAEIFSLFAENNMELTDSTMLTPGLRFDHHSIV
GDNWSPSLNLSQGLGDDFTLKMGIARAYKAPSLYQTNPNYILYSKGQGCYATGAGTGI
GCYMMGNDDLKAETSINKEIGLEFKRDGWLAGVTWFRNDYRNKIEAGTVPLQRINNG
KTDVYQWENVPKAVVEGLEGTLNVPVSDTVNWTNNVTYMLQSKNKETGERLSIIPQYT
LNSTLSWQVRQDVSLQSTFTWYGKQEPKKYDYQGNPVTGTDKQAVSPYSIVGLSAT
WDVTKNVSLTGGVDNLFDKRLWREGNAQTVRDTQTGAYMAGAGAYTYNEPGRTWY
MSINTHF
FhuA
>AEF06126.1 ferrichrome outer membrane transporter
[Salmonella enterica subsp. enterica serovar Typhimurium
str. UK-1, 729 AA, CP002614.1:223733..225922, STMUK_0193]
(SEQ ID NO: 186)
MARLKTAQPNSSLRKIAVVVATAVSGMSVYAQAAVQPKEETITVTAAPAAQESA
WGPAPTIAAKRSATTTKTDTPIEKTPQSVSVVTNEEMQMHQFQSVKEALGYTPGVTVS
SRGASNTYDFVIIRGFSSVGLSQNNYLDGLKLQGNFYNDAVIDPYMLERVELMRGPTS
VLYGKSNPGGIISMVSKRPTTEPLKEIQFKMGTDNLFQTGFDFSDSLDDNGEFSYRLTG
LARSTNEQQKSSESQRYAIAPSFTWRPDEKTNFTFLSYFQNEPETGYYGWLPKEGTV
EPLPNGKRLPTDFNEGASNNTYSRNEKMVGYSFEHGFNDTFTVRQNLRFVEMKTAQK
SVYGTGIAADGHTLNRGTIVDNERLQNFSVDTQLESKFATGDIDHTLLTGVDFMRMRN
DINATFGSAPSIDLYNNYHPEYFAFGGAEPYQMNESKQTGLYVQDQAEWNKWVFTLG
GRYDWSKQATTVRQNSTTPTEGYIERNDHQFTWRGGVNYVFDNGISPYFSYSQSFEP
SAFDLWSTPRVSYKPSKGEQYEAGVKYVPKDMPVVVTGAVYQLTKTNNLTADPTNPL
AQVPAGEIRARGVELEAKAALTANINMTASYTYTDAEYTKDTNLKGNTPEQVPEHMAS
LWGDYTFNEGPLSGLTLGTGGRFIGSSYGDPANSFKVGSAAVMDAVVKYDLARFGMA
GSSIAVNVNNLLDREYVASCFQTYGCFWGAERQVVATATFRF
IroN
>AEF08636.1 outer membrane receptor [Salmonella enterica
subsp. enterica serovar Typhimurium str. UK-1, 726 AA,
complement (CP002614.1:2892077..2894257), STMUK_2765]
(SEQ ID NO: 187)
MGMRVKKFIWLITVVSTGVNSPLSAAESTDDNGETMVVESTAEQVLKQQPGVSIITRD
DIQKNPPVNDLADIIRKMPGVNLTGNSASGTRGNNRQIDIRGLGPENTLVLIDGVPVTS
RNSVRYSWRGERDTRGDTNWVPPEMVERIEVIRGPAAARYGSGAAGGVVNIITKRPT
NDWHGSLSLYTNQPESSKEGDTRRGNFSLSGPLAGDTLTMRLYGNLNRTDADSWDIN
SSAGTKNAAGREGVTNKDINSVFSWKMTPQQILDFEAGYSRQGNIYAGDTQNSTSNA
VTKSLAQSGRETNRLYRQNYGLTHNGIWDWGQSRLGFYYEKTDNTRMNEGLSGGGE
GRITNDQTFTTNRLTSYRTSGEVNVPVIWLFEQTLTVGAEWNRDELNDPSSTSLTVKD
SNIAGIPGSAANRSSKNKSEISALYVEDNIEPMAGTNIIPGLRFDYLSESGSNFSPSLNLS
QELGEYVKVKAGIARAFKAPNLYQTSEGYLLYSKGNGCPKDITSGGCYLVGNKNLDPEI
SINKEIGLEFTVDDYHASVTYFRNDYQNKIVAGDQIIGRSASGAYVLQWQNGGKALIEGI
EASMAVPLMPDRLNWNTNATYMIASEQKDTGNPLSIIPKYTVNTFLDWTITSALSANVN
WTLYGKQKPRTHAESRSEETKGLSGKALGAYSLVGANVNYDINKNLRLNVGISNIFDK
QIYRSAEGANTYNEPGRAYYAGVTASF
PagC
>AEF07120.1 virulence membrane protein PagC precursor
[Salmonella enterica subsp. enterica serovar Typhimurium
str. UK-1, 185 AA, CP002614.1:1290416..1290973, STMUK_1214]
(SEQ ID NO: 188)
MKNIILSTLVITTSVLVVNVAQADTNAFSVGYAQSKVQDFKNIRGVNVKYRYEDDSPVS
FISSLSYLYGDRQASGSVEPEGIHYHDKFEVKYGSLMVGPAYRLSDNFSLYALAGVGT
VKATFKEHSTQDGDSFSNKISSRKTGFAWGAGVQMNPLENIVVDVGYEGSNISSTKIN
GFNVGVGYRF
Rck
>AEF10395.1 resistance to complement killing (plasmid)
[Salmonella enterica subsp. enterica serovar Typhimurium
str. UK-1, 185 AA, complement (CP002615.1:80634..81191),
STMUK_p088]
(SEQ ID NO: 189)
MKKIVLSSLLLSAAGLAAVPVAQADTHSVSVGYAQSRIEHFKDIRGVNLKYRYEAQTPL
GLMASFSWQSGKRGESGGIPGGMSWRDDVKATYWSLMAGPAVRVNELVSLYALAG
AGTGRAEVKERISMPGYNGRFTGSERRTGFAWGAGVQFNPVENVVIDLGYEGSKVG
AAKLNGVNVGVGYRF

One example of these peptides is targeting peptide RKOpep (130) that can bind membrane protein monocarboxylate transporter 1 (MCT1/SLC16A1) on the colorectal cancer cells (131, 132). MCT1 plays a crucial role in transporting lactate and other monocarboxylates (like pyruvate) across membranes, which is involved in the colon cancer and other tumors with altered metabolic pathways. MCT1 is overexpressed in colon cancer and associated with poor prognosis (133-135). Another example is integrin α6β1, a laminin receptor overexpressed in many cancers, including colorectal cancer (CRC), and associated with poor patient prognosis (136). We generated plasmids pG8R478 and pG8R479 to target colonrectal cancer through targeting MCT1 and α6β1, respectively.

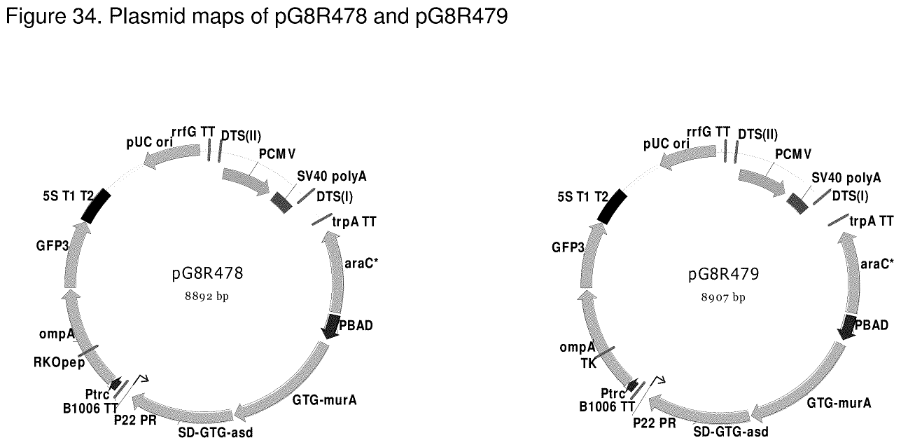

To construct plasmid pG8R478 encoding RKOpep that targets MCT1 (FIG. 34), we used plasmid pG8R320 (FIG. 5B) as a template to amplify a 534 bp fragment using primers Ptrc-BcII-s and RKOpep-a and a 704 bp fragment using primers RKOpep-s and ompAGFP-BstBI-a. With plasmid pYA4090 (FIG. 2C) as a template, the gene encoding GFP was amplified with primer GFP-s and GFP-BstBI-a. The 3 fragments were inserted into plasmid pG8R320 cut with BcII/BstBI to generate plasmid pG8R478. The regulated delayed lysis plasmid has a pUC on and can express ompA)RKOpep and gfp in Salmonella to display the synthesized OmpAΩRKOpep on the CCTS cell surface and GFP in the cytosol and after lysis of the CCTS cell in a cancer cell employs the P_{cmv} promoter to express an inserted gene sequence of importance for cancer cell/tumor therapy. The plasmid can target colonrectal cancer and other cancer cells that overexpress MCT1.

To construct plasmid pG8R479 encoding TK that targets asp, (FIG. 34), we used plasmid pG8R320 (FIG. 5B) as a template to amplify a 543 bp fragment using primers Ptrc-BcII-s and TK-a and a 711 bp fragment using primers TK-s and ompAGFP-BstBI-a. With plasmid pYA4090 (FIG. 2C) as a template, the gene encoding GFP was amplified with primer GFP-s and GFP-BstBI-a. The 3 fragments were inserted into plasmid pG8R320 cut with BcII/BstBI to generate plasmid pG8R479. The regulated delayed lysis plasmid has a pUC on and can express ompAΩTK and gfp in Salmonella to display the synthesized OmpAΩTK on the CCTS cell surface and GFP in the cytosol and after lysis of the CCTS cell in a cancer cell employs the P_{cmv} promoter to express an inserted gene sequence of importance for cancer cell/tumor therapy. The plasmid can target colonrectal cancer and other cancer cells that overexpress α₆β₁.

TABLE 10
Peptide used for colorectal cancer targeting

| | | SEQ | | Ovarian |
|---|---|---|---|---|
| Target | | ID | | Cancer |
| GM2 | Peptide | NO | Sequence | Cell |
| | Peptide with identified target/receptor | | | |
| α₆β₁ | TK/AG-32 (137) | 190 | TWYKIAFQRNRK | Caco-2 |
| | RWY (138) | 191 | CRWYDENAC | |
| | S5 (139) | 192 | CRWYDANAC | |
| HER2/Erb2 | KSP (140) | 193 | KSPNPRF | HT29 |
| | | | | SW480 |
| EGFR | QRH (141) | 194 | QRHKPRE | HT29, |
| | | | | SW480, and |
| | | | | SW620 |
| EGFR | GE11 (142) | 195 | YHWYGYTPQNVI | SW480, |
| | | | | SSW620, |
| | | | | Caco-2, |
| | | | | HCT116 |
| | | | | and SW620 |
| | D4 (143) | 196 | LARLLT | HT-29, |
| | | | | SW480 |
| | EBP (144) | 197 | CMYIEALDKYAC | HT29 |
| | QRH (141) | 194 | QRHKPRE | |
| | EGBP (145) | 198 | FPMFNHWEQWPP | |
| | EGFRi/S3 (146) | 199 | RCSHGYTGIRCQAV | |
| | | | VL | |
| | FAL (147) | 200 | FALGEA | |
| | PEPHC1 (148), | 201 | HFLIIGFMRRALCGA | |
| | A12PEPHC1 (149) | 202 | HFLIIGFMRRAACGA | |
| | P75 (150) | 203 | KYFPPLALYNPTEYF | |
| | | | Y | |
| | --- (151) | 204 | VLGREEWSTSYW | |
| | Pep 11 (152) | 205 | WSGENGPGFYDYE | |
| | | | A | |
| | AEYLR (153) | 206 | AEYLR | |
| | --- (154) | 207 | EEEEYFELV | |
| | | 208 | DEDEYFELV | |
| FGFR | P7 (155) | 209 | PLLQATLGGGS | HT-29, |
| | | | | LoVo, |
| | | | | and Caco2 |
| GRPR | BBN7-14 (156) | 210 | QWAVGHLM | Caco-2 |
| VEGFR1 | anti-Flt1 (157) | 211 | GNQWFI | W480, M7 |
| vimentin | STP (158) | 212 | VATANST | CT26 |
| Patatin-like | CCBP1 (159) | 213 | HAMRAQP | SW480 |
| phospholipase | | | | |
| domain-containing | | | | |
| protein | | | | |
| Monocarboxylate | RKOpep (130) | 214 | CPKSNNGVC | RKO, |
| transporter 1 | | | | Caco-2, |
| (MCT1) | | | | HCT 116 |
| | | | | and HCT-15 |
| P32 | LyP-1 (160) | 215 | CGNKRTRGC | |
| neuropilin-1/2 | LyP-1b (161) | 216 | CNKRTRGGC | |
| vascular | TCP-1 (162) | 217 | CTPSPFSHC | HCT116 and |
| endothelia | | | | HT-29 |
| Anxa1 | IF7 (163) | 218 | IFLLWQR | HCT116 |
| | Peptide without identified target/receptor | | | |
| | CP15 (164) | 219 | VHLGYAT | SW480 and |
| | | | | HT29 |
| | COLO320HSR (165) | 220 | DWSSWVYRDPQT | COLO320HSR, |
| | | | | HCT116, |
| | | | | SW480, |
| | | | | HT29, LoVo |
| | RPMrel (166) | 221 | CPIEDRPMC | HCT116 |
| | L20 (167) | 222 | ANLNLWTDYIRW | SW480, |
| | | | | HCT116, |
| | | | | HT29, and |
| | | | | LoVo |
| | HEW (168) | 223 | HEWSYLAPYPWF | WiDr |
| | SPT (169) | 224 | SPTKSNS | DLD-1 and |
| | | | | HCT-15 |

### Example 38. Evaluation of the SDAAS Strains to Induce Tumor Regression and Ablation in a B16F10 Melanoma Mouse Model

Tumors are very heterogeneous. The efficacy of SDAAS strains might be different in different tumor types. We therefore tested our SDAAS strains in a different B16F10 melanoma model. Skin cancer is the most common of all cancers. Melanoma causes most skin cancer deaths. The American Cancer Society estimated that 100,640 new melanomas will be diagnosed and 8,290 individuals are expected to die of melanoma in 2024. Melanoma cells have a higher levels of TLR4 compared with normal tissues (170, 171) and TLR5 agonists enhance antitumor activity against melanoma (172, 173). Both MPL and flagellin have been shown to enhance melanoma immunotherapy (118, 119, 172-175).

Animal model. Six- to eight-week-old C57BL/6 mice (H-2b) receive a subcutaneous injection into the right flank with 1×10⁶ B16F10 melanoma cells or 316-F10-Red-F-luc (Perkinelmer) in 100 μl PBS. After 8 to 10 days of development for B16F10, reaching tumor sizes of 100-150 mm³, mice can be grouped and standardized for mean and variance in tumor volume prior to injection treatments. Tumor development is calculated as described in Example 35 (119).

Evaluation of the abilities of SDAAS strains to home to tumor, destroy tumor, and the treatment of SDAAS on immune cells. The SDAAS stains can be tested as described in Example 36.

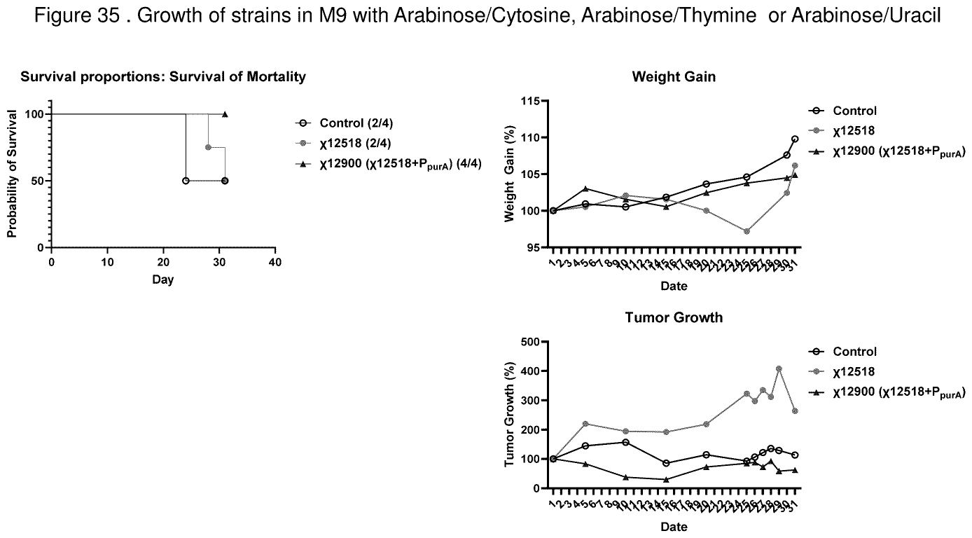

We have tested the abilities of SDAAS strain and its derivatives in BALB/c mice (Figure. 35). Six-week-old BALB3/c mice (H-2^{d}) receive a subcutaneous injection with 1×10⁶ B16F10 melanoma cells in 100 μl PBS. After 10 days of development for B16F10, reaching tumor sizes of around 100 mm³, mice were grouped and standardized for mean and variance in tumor volume prior to treatments. Mice were then treated with 10⁶ of strain χ12518 and χ12900 intravenously. C57BL/6 is a syngeneic model for 816 melanoma, while BALB/c has a different genetic background that could generate immune rejection responses against: B16. Thus, the tumor is not grown robustly and consistently. Although SDAAS Strain χ12518 did not induce tumor regress, the SDAAS strain χ12900 with a ΔP_{purA} mutation inhibits the tumor growth (FIG. 35). The mice in groups without treatment and treated with χ12518 have 50% mortality, while the group of mice treated with χ12900 has no mortality. These data prove that SDAAS strains need to target tumors to be effective, while unregulated activation might promote tumor growth.

### Example 39. Augment of Melanoma Targeting of SDAAS Strains by Adopting Melanomas Targeting Peptides

Melanoma targeting peptide can be used to augment the targeting ability of SDAAS strain to melanoma. Many melanoma targeting peptides have been identified by different methods (Table 11). These peptides are in different categories. Some peptides have a general target, like RGD targeting αᵥβ₃. αᵥβ₃ involves in cell adhesion and signal transduction for tumor progression and angiogenesis, especially for tumors with the invasive and metastatic potential, such as melanoma, breast and glioblastoma. Targeting αᵥβ₃ could inhibit the angiogenesis and tumor spread. These peptides could target multiple tumors, not limited to melanoma. Other peptides, like aMSH1-13 targeting MC1R, only target melanoma. There are also other peptides, such as WDC-2 and IP, that target specific melanoma cells. Some peptides, like P1 and phage 2, have identified target, but have't been tested in melanoma cells. SDAAS can deliver all these peptides thorough fusion of these peptides with outer membrane protein(s) to augment the target ability of SDAAS. The outer membrane protein is not limited to OmpA, but could be OmpC, OmpD, OmpF, OmpN, OmpW, OrnpX, ApeE/EstA, BtuB, CirA, FepA, FhuA, IroN, PagC, Rck or any other proteins that could display these peptides on the surface of Salmonella.

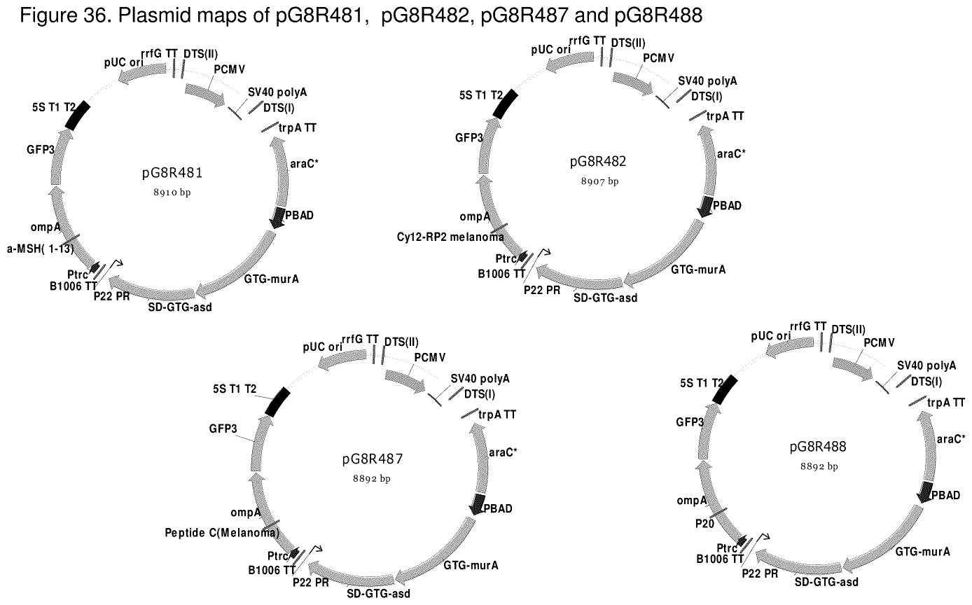

We generated plasmids pG8R481, pG8R482, and pG8R487 to target melanoma. To construct plasmid pG8R481 encoding αMSH(1-13) that targets MC1R (FIG. 36), we used plasmid pG8R320 (FIG. 5B) as a template to amplify a 565 bp fragment using primers Ptrc-BcII-s and aMSH1-13-a. With plasmid pG8R380 (FIG. 17A) as a template, the gene encoding c-terminal of OmpA and GFP was amplified with primer αMSH(1-13)-s and GFP-BstBI-a. The 2 fragments were inserted into plasmid pG8R320 cut with BcII/BstBI to generate plasmid pG8R481. The regulated delayed lysis plasmid has a pUC ori and can express ompAΩaMSH1-13 and gfp in Salmonella to display the synthesized OmpAΩaMSH1-13 on the CCTS cell surface and GFP in the cytosol and after lysis of the CCTS cell in a cancer cell employs the P_{cmv} promoter to express an inserted gene sequence of importance for cancer cell/tumor therapy. This plasmid can target melanoma.

To construct plasmid pG8R482 encoding peptide CY12RP2 that targets podoplanin (FIG. 36), we used plasmid pG8R320 (FIG. 5B) as a template to amplify a 565 bp fragment using primers Ptrc-BcII-s and CY12RP2-a. With plasmid pG8R380 (FIG. 17A) as a template, the gene encoding c-terminal of OmpA and GFP was amplified with primer CY12RP2-s and GFP-BstBI-a. The 2 fragments were inserted into plasmid pG8R320 cut with BcII/BstBI to generate plasmid pG8R482. The regulated delayed lysis plasmid has a pUC on and can express ompAΩcy12rp2 and gfp in Salmonella to display the synthesized OmpAΩCY12RP2 on the CCTS cell surface and GFP in the cytosol and after lysis of the CCTS cell in a cancer cell employs the P_{cmv} promoter to express an inserted gene sequence of importance for cancer cell/tumor therapy. This plasmid can target melanoma.

Both Peptide C and P20 targets sonic hedgehog protein. To construct plasmid pG8R487 encoding Peptide C (FIG. 36), we used plasmid pG8R320 (FIG. 5B) as a template to amplify a 554 bp fragment using primers Ptrc-BcII-s and PeptideC-a. With plasmid pG8R380 (FIG. 17A) as a template, the gene encoding c-terminal of OmpA and GFP was amplified with primer PeptideC-s and GFP-BstBI-a. The 2 fragments were inserted into plasmid pG8R320 cut with BcII/BstBI to generate plasmid pG8R487. The regulated delayed lysis plasmid has a pUC on and can express ompA)PeptideC and gfp in Salmonella to display the synthesized OmpAΩPeptideC on the CCTS cell surface and GFP in the cytosol and after lysis of the CCTS cell in a cancer cell employs the P_{cmv} promoter to express an inserted gene sequence of importance for cancer cell/tumor therapy. This plasmid can target melanoma.

To construct plasmid pG8R488 encoding P20 (FIG. 36), we used plasmid pG8R320 (FIG. 5B) as a template to amplify a 552 bp fragment using primers Ptrc-BcII-s and PeptideC-a. With plasmid pG8R380 (FIG. 17A) as a template, the gene encoding c-terminal of OmpA and GFP was amplified with primer PeptideC-s and GFP-BstBI-a. The 2 fragments were inserted into plasmid pG8R320 cut with BcII/BstBI to generate plasmid pG8R488. The regulated delayed lysis plasmid has a pUC on and can express ompAΩP20 and gfp in Salmonella to display the synthesized OmpAΩP20 on the COTS cell surface and GFP in the cytosol and after lysis of the CCTS cell in a cancer cell employs the P_{cmv} promoter to express an inserted gene sequence of importance for cancer cell/tumor therapy. This plasmid can target melanoma.

TABLE 11
Peptide used for melanoma targeting

| | | | | |
|---|---|---|---|---|
| | | SEQ | | Melanoma |
| Target | Peptide | ID NO | Sequence | Cancer Cell |

Peptide with identified target/receptor

| αᵥβ₃ | RGD (176) | 225 | RGD | SK-MEL-110. |
|---|---|---|---|---|
| αᵥβ₅ | RGD-4C (177) | | CDCRGDCFC | A375, M21, |
| | | | | WM164, A375, |
| | | | | SKMel-28, |
| | | | | B16F10, MDA- |
| | | | | MB-435 |
| | ALOS4 (178) | 226 | CSSAGSLFC | B16F10, WM- |
| | | | | 266-4 |
| α₆β₁ | TK/AG-32 | 190 | TWYKIAFQRNRK | LOX, B16F10 |
| | AG-10 (137) | 227 | NPWHSIYITRFG | |
| FGFR | P7 (155) | 228 | PLLQATL GGGS | B16F10 |
| | | 229 | | |
| | P9 (179) | 230 | LSPPRYP | B16F10 |
| | P1 (180) | 231, | VYMSPF, | |
| | | 232 | CAVYMSPFAC | |
| | P12 (181) | 233 | HSQAAVP | |
| Glucose- | Pep42 (182) | 234 | CTVALPGGYVRVC | Me6652/4, |
| regulated | | | | Me6652/56B1 |
| protein 78 | | | | 6F10 |
| (GRP78) | | | | |
| | WIFPWIQL, | 235, | WIFPWIQL, | |
| | WDLAWMFRL | 236 | WDLAWMFRLPVG | |
| | PVG (183) | | | |
| GD2 | phage 2 (184) | 237 | WHWRLPS | |
| (Gangliosides) | | | | |
| P32 | LyP-1 (160) | 215 | CGNKRTRGC | MDA-MB-435, |
| neuropilin- | LyP-1b (161) | 216 | CNKRTRGGC | S180 |
| 1/2 | | | | |
| Podoplanin | CY12-RP2 | 238 | TYLCTACDYTHH | A375, A875, |
| (PDPN) | (185) | | | B16F10 |
| MC1R | αMSH1-13 | 239 | SYSMEHFRWGKPV | D10, LSD 22, |
| (Melanocortin-1 | (186) | | | Me8, 205, |
| receptor) | | | | IGR3, A375, |
| | | | | Me1477, |
| | | | | Me34, GLL19, |
| | | | | Mel Juso, B16- |
| | | | | F1, Cloudman |
| | | | | S91 |
| | ReMSH (187) | 240 | CEHFRWCKPV | B16-F1, |
| | ReCCMSH | 241 | CCEHFRWCKPV | TXM13 |
| | (187) | | | |
| | MSH2.0 (188) | 242 | AMEHFRWGRPVGSGSGSG | B16-F1 |
| | | | SVWYAG | |
| | MS05 (189) | 243 | SSIISHFRWGKPV | |
| γ-MSH | γ-MSH | 244 | YVLGHFLFDRFG | |
| | compound 5 | | | |
| | (190) | | | |
| Melanin | 4B4 (191) | 245 | YERKFWHGRH | SK-MEL-28, |
| | MBP | | | MNT1, |
| | | | | A2058 |
| | 3D (192) | 246 | NPNWGPR | MNT1, A2058 |
| | 1C (192) | 247 | TTHQFPF | |
| Sonic | Peptide C | 248 | CVNHPAFAC | B16F10 |
| hedgehog | (193) | | | |
| protein, | | | | |
| SCUBE2 | | | | |
| ligand | | | | |
| | KK11 (194) | 249 | VPWXEPAYQRFL | A375, |
| | | | | WM115, SK- |
| | | | | Mel-28, |
| | | | | WM3211 |
| | P20 (195) | 250 | CSSRTMHHC | B16F10 |
| Neural/glial | TAA/T10, | 251 | TAASGVRSMH | B16F10 |
| antigen 2 | LTL/LS10 | 252 | LTLRWVGLMS | |
| (NG2)/ | (196) | | | |
| melanoma- | | | | |
| associated | | | | |
| chondroitin | | | | |
| sulfate | | | | |
| proteoglycan | | | | |
| (MCSP) | | | | |
| Mimotopes | | | | |
| BAT | Peptide A | 253 | PRRIKPRKIMLQ | B16F10 |
| | Peptide B | 254 | QRILQQINLPRI | |
| | (197) | | | |
| P763.74 | P763.74 (198) | 255 | QCTGPNVATNCR | MV3 |
| | Peptide without identified target/receptor | | | |
| | WDC-2 (199) | 256 | TRTKLPRLHLQS | B16F10 |
| | IP (200) | 257 | INQDARTMVMVP | B16F10 |

### Example 40. Evaluation of SDAAS Strains to Induce Tumor Regression and Ablation in Ovary Cancer Mouse Model

Unmet medical needs in ovarian cancer. Ovarian cancer is a devastating disease characterized by late-stage diagnosis, limited treatment options, and high mortality rates. Conventional therapies, surgery, radiation and chemotherapy often fail to eradicate tumors completely, leading to relapse, metastasis or resistance to chemotherapy (201, 202). Currently, there is no approved precision medicine or immune therapy for ovarian cancer. There is an urgent public health need for conceptually new treatments for ovarian cancer. It is necessary to probe new methods to treat ovarian cancer. Specially, improving the overall survival and disease control in this critical population with an oral drug that has a manageable toxicity profile and easy administration deserves to be considered a therapeutically successful achievement. Considering the urgent need for innovative treatment strategies to improve outcomes for ovarian cancer patients, we can harness the tumor-targeting capabilities of an extensively modified Salmonella as a novel therapeutic approach for ovarian cancer.

Salmonella based immunotherapy. Salmonella has shown promising potential as an anti-tumor agent due to its intrinsic tumor-targeting ability and capacity to induce immune responses (203), either as a delivery system for cancer therapy alone (204), or as an integrated part of cancer combination therapy. A live Salmonella vaccine has been approved as an oral vaccine for typhoid fever in humans. Oral (205-208), intravenous, or intratumoral routes (20, 209-211) have been adopted in cancer clinical trials. However, these previous trials showed that lower abilities to colonize in tumor tissues and lower anti-tumor activities were observed even with intravenous or intratumoral injection (20, 209-211), indicating that both the abilities of tumor targeting and killing need to be increased. We hypothesize that the lack of colonization and antitumor efficacy in these human clinical trials could be overcome by increasing the ability of Salmonella (i) to specifically sense, invade into and replicate in the ovarian cancer cells, and (ii) to further enhance the ability of Salmonella to selectively kill ovarian cancer cells by increasing innate immune responses without harming healthy cells.

We can develop an oral ovarian cancer-specific microbiotherapy platform named SOCTI (Salmonella Ovarian Cancer Targeted Immunotherapy) that combines multiple ways to increase the abilities of Salmonella to target and directly kill ovarian cancer by microbiotherapy and local immunotherapy in a single platform. It has three attributes: 1) Salmonella: It naturally homes to tumors as an anaerobic, nutrient rich environment to directly lyse and kill cancer cells (microbiotherapy) and stimulate anti-cancer immunity; 2) Dual-peptide to target ovarian cancer: Both epithelial cell adhesion molecule (EpCAM) and folate receptor alpha (FRα) receptors are known to be overexpressed on the surface of ovarian cancer cells. Salmonella can display peptides targeting EpCAM and FRα that enable Salmonella to specifically adhere to and internalize into ovarian cancer cells; 3) Adjuvant Augmented Salmonella: Salmonella can be modified to enhance its adjuvant activities to induce innate immune responses to enhance immunotherapy against the ovarian cancer cells. Furthermore, SOCTI can be delivered by the oral route (as well as other routes). Although intravenous and intraperitoneal routes are used in ovarian cancer therapy, oral is the most commonly accepted route for drug administration due to the benefits of ease of administration, patient compliance, and cost-effectiveness. Salmonella has the advantage since it can conquer the harsh environment of the gastrointestinal tract. The single-agent SOCTI platform with dual-specificity targeting and killing ovarian cancer cells can be an effective strategy for treating ovarian cancer. After oral inoculation, SOCTI specifically targets and amplifies in ovarian cancer cells for local immunotherapy without systemic toxicity through the activation of innate immune responses, and recruitment of T cells to kill tumor cells. In addition, Salmonella microbiotherapy can further overcome multiple chemotherapeutic drug resistance mechanisms and potentiate other immunotherapy regimens. We test the hypothesis that Salmonella with enhanced specific targeting to ovarian cancer and adjuvant abilities can enhance immunotherapy and mediate potent and safe eradication of tumor cells through oral inoculation.

Peptides targeting EpCAM and FRα can be used. Both EpCAM and FRα are well-characterized targets for ovarian cancer. EpCAM is overexpressed in more than 70% of ovarian cancer cells, especially in late stage and metastasis. Its expression in the peritoneal cavity is tumor specific (212). Up to 97% of ovarian cancer constitutively express FRα, but is rarely expressed in non-malignant ovarian tissues (213, 214). The first FRα-targeting antibody-drug conjugate was just approved by the FDA to treat platinum-resistant ovarian cancer (215). Peptide EP114 can bind to EpCAM (216). The peptide C7 can target FRα (217). Both peptides can be expressed on the surface of Salmonella to increase the targeting ability of SOCTI (217). Combined targeting of EpCAM and FRα enable SOCTI to identify both malignant and non-malignant ovarian cancer cells. Once inside cancer cells, Salmonella can directly kill cancer cells to release tumor antigens, induce local inflammation, create an immune stimulatory milieu and mitigate the immunosuppressive tumor microenvironment. Besides EP114 and C7, other peptides that can be used for targeting are listed in Table 12. Salmonella can be further engineered to increase its ability to recruit innate immune responses, including, but not limited to, TLR4, TLR5 and TLR9 responses. The expression of LpxE can convert Salmonella lipid A into 1-dephosphorylated lipid A (TLR4 agonist) (95), similar to the adjuvant MPLA approved for human use (218), which eliminate the toxicity of Salmonella and ensure safety. Heterologous flagellin secreting in Salmonella can increase the anti-tumor effect through TLR5 (55).

Increasing the lysis of Salmonella within cancer cells can enable its degraded genomic DNA to activate TLR9 and increase the attribute of biocontainment and safety (219). Through the activation of innate immune responses, the SDAAS can increase local inflammation and harness host immune cells. The host immune cells are thus attracted to the cancer and effectively kill cancer cells even if only some tumor cancer cells are targeted. The means to modify SDAAS strains to incorporate all these modifications alone or in combination to achieve safe and efficacious SOCTI strains are described in multiple of the foregoing Examples.

The SOCTI strains can be tested to effectively target and kill ovarian cancer cells and not harm healthy tissues, To accomplish this, SOCTI with EpCAM and FRα targeting peptides and its parents are evaluated for their abilities to attach to and invade into ovarian cancer cell lines including metastatic and non-invasive cell lines, followed by in vivo characterization of the tumor/organ (spleen, liver, heart, lung, kidney) ratios after oral delivery to mice with ectopic syngeneic and xenograft ovarian cancers.

### Example 41. Augment of Targeting Ability of SDAAS Strains by Adopting Ovarian Cancer Targeting Peptides

Besides the EpCAM and FRα targeting peptide, other peptides listed in Table 12 could be used instead of EpCAM and FRα to target various receptors. Some peptides, such as PC3-1 (220, 221) and Peptide 1 (222), have dual functions that can target ovarian cancer and also suppress the cell adhesion and invasion. These peptides can be used to augment the targeting ability of SDAAS strain to ovarian cancer. Many ovarian cancer targeting peptides have been identified by different methods (Table 12). These peptides are in different categories. Some peptides have a general target, like RGD targeting αvβ3. αvβ3 involves in cell adhesion and signal transduction for tumor progression and angiogenesis, especially for tumors with the invasive and metastatic potential, including ovarian cancer. Targeting αvβ3 could inhibit the angiogenesis and tumor spread. These peptides could target multiple tumors, not limited to ovarian cancer. There are also other peptides, such as Peptide 1 and S36, that target specific ovarian cancer cells. Some peptides, like APY and H8, have identified target, but have't been tested in ovarian cancer cells, and ASDAAS can deliver all these peptides thorough fusion of these peptides with outer membrane protein(s) to augment the target ability of SDAAS. The outer membrane protein is not limited to OmpA, but could be OmpC, OmpD, OmpF, OmpN, OmpW, OmpX, ApeE/EstA, BtUB, CirA, FepA, FhuA, IroN, PagC, Rck or any other proteins that could display these peptides on the surface of Salmonella.

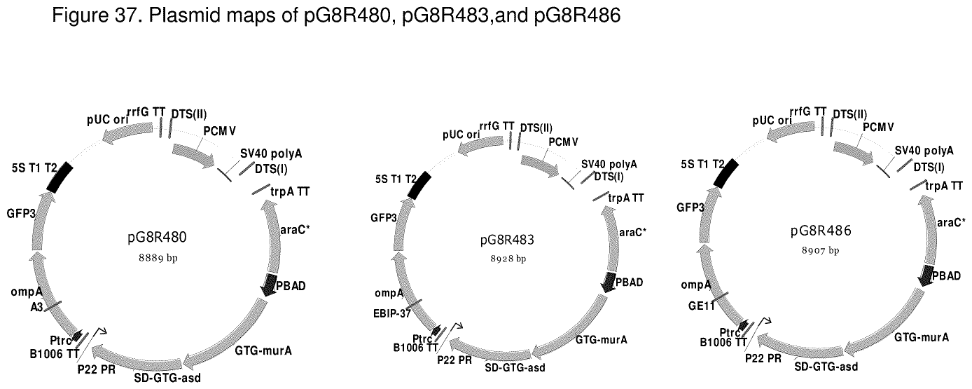

To construct plasmid pG8R480 encoding peptide A3 that targets a3 (FIG. 37), we used plasmid pG8R320 (FIG. 5B) as a template to amplify a 544 bp fragment using primers Ptrc-BcII-s and A3-a. With plasmid pG8R380 (FIG. 17A) as a template, the gene encoding c-terminal of OmpA and GFP was amplified with primer A3-s and GFP-BstBI-a. The 2 fragments were inserted into plasmid pG8R320 cut with BcII/BstBI to generate plasmid pG8R480. The regulated delayed lysis plasmid has a pUC on and can express ompAΩa3 and gfp in Salmonella to display the synthesized OmpAΩA3 on the CCTS cell surface and GFP in the cytosol and after lysis of the CCTS cell in a cancer cell employs the P_{cmv} promoter to express an inserted gene sequence of importance for cancer cell/tumor therapy. This plasmid can ovarian cancer and other cancer that overexpress a3.

To construct plasmid pG8R483 encoding peptide EBIP-37 that targets ERβ (FIG. 37), we used plasmid pG8R320 (FIG. 5B) as a template to amplify a 565 bp fragment using primers Ptrc-BcII-s and EBIP-37-a. With plasmid pG8R380 (FIG. 17A) as a template, the gene encoding c-terminal of OmpA and GFP was amplified with primer EBIP-37-s and GFP-BstBI-a. The 2 fragments were inserted into plasmid pG8R320 cut with BcII/BstBI to generate plasmid pG8R483. The regulated delayed lysis plasmid has a pUC on and can express ompAΩebip-37 and gfp in Salmonella to display the synthesized OmpAΩEBIP-37 on the CCTS cell surface and GFP in the cytosol and after lysis of the CCTS cell in a cancer cell employs the P_{cmv} promoter to express an inserted gene sequence of importance for cancer cell/tumor therapy. This plasmid can target ovarian cancer and other cancers that overexpress ERβ.

To construct plasmid pG8R486 encoding peptide GE11 that targets EGFR (FIG. 37), we used plasmid pG8R320 (FIG. 5B) as a template to amplify a 562 bp fragment using primers Ptrc-BcII-s and GE11-a. With plasmid pG8R380 (FIG. 17A) as a template, the gene encoding c-terminal of OmpA and GFP was amplified with primer GE11-s and GFP-BstBI-a. The 2 fragments were inserted into plasmid pG8R320 cut with BcII/BstBI to generate plasmid pG8R486. The regulated delayed lysis plasmid has a pUC on and can express ompAΩge11 and gfp in Salmonella to display the synthesized OmpAΩGE11 on the CCTS cell surface and GFP in the cytosol and after lysis of the CCTS cell in a cancer cell employs the P_{cmv} promoter to express an inserted gene sequence of importance for cancer cell/tumor therapy. This plasmid can target ovarian cancer and other cancers that overexpress EGFR.

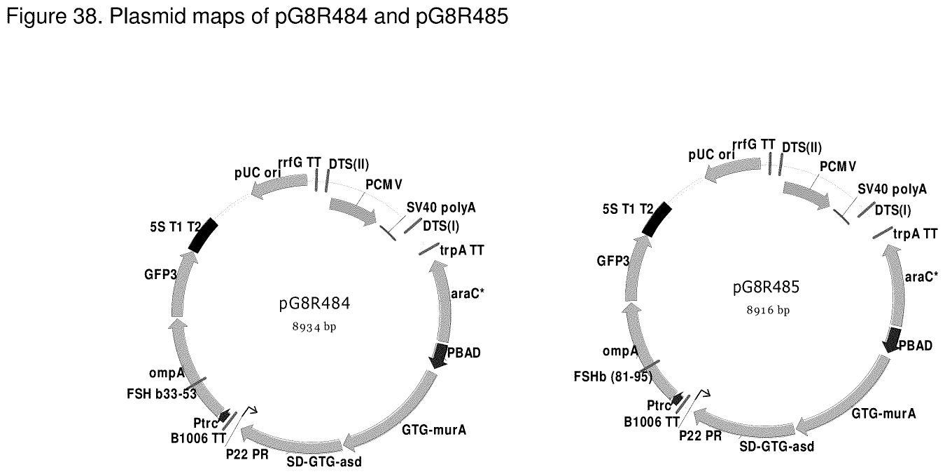

To construct plasmid pG8R484 encoding peptide FSHβ(33-53) that targets FSHR (FIG. 38), we used plasmid pG8R320 (FIG. 5B) as a template to amplify a 575 bp fragment using primers Ptrc-BcII-s and FSHβ(33-53)-a. With plasmid pG8R380 (FIG. 17A) as a template, the gene encoding c-terminal of OmpA and GFP was amplified with primer FSHβ(33-53)-s and GFP-BstBI-a. The 2 fragments were inserted into plasmid pG8R320 cut with BcII/BstBI to generate plasmid pG8R484. The regulated delayed lysis plasmid has a pUC on and can express ompAΩfshβ(33-53) and gfp in Salmonella to display the synthesized OmpAΩFSHβ(33-53) on the CCTS cell surface and GFP in the cytosol and after lysis of the CCTS cell in a cancer cell employs the P_{cmv} promoter to express an inserted gene sequence of importance for cancer cell/tumor therapy. This plasmid can target ovarian cancer and other cancers that overexpress FSHR.

To construct plasmid pG8R485 encoding peptide FSHβ(81-95) that targets FSHR (FIG. 38), we used plasmid pG8R320 (FIG. 5B) as a template to amplify a 565 bp fragment using primers Ptrc-BcII-s and FSHβ(81-95)-a. With plasmid pG8R380 (FIG. 17A) as a template, the gene encoding c-terminal of OmpA and GFP was amplified with primer FSHβ(81-95)-s and GFP-BstBI-a. The 2 fragments were inserted into plasmid pG8R320 cut with BcII/BstBI to generate plasmid pG8R485. The regulated delayed lysis plasmid has a pUC on and can express ompAΩfshp(81-95) and gfp in Salmonella to display the synthesized OmpAΩFSHβ(81-95) on the CCTS cell surface and GFP in the cytosol and after lysis of the CCTS cell in a cancer cell employs the P_{cmv} promoter to express an inserted gene sequence of importance for cancer cell/tumor therapy. This plasmid can target ovarian cancer and other cancers that overexpress FSHR.

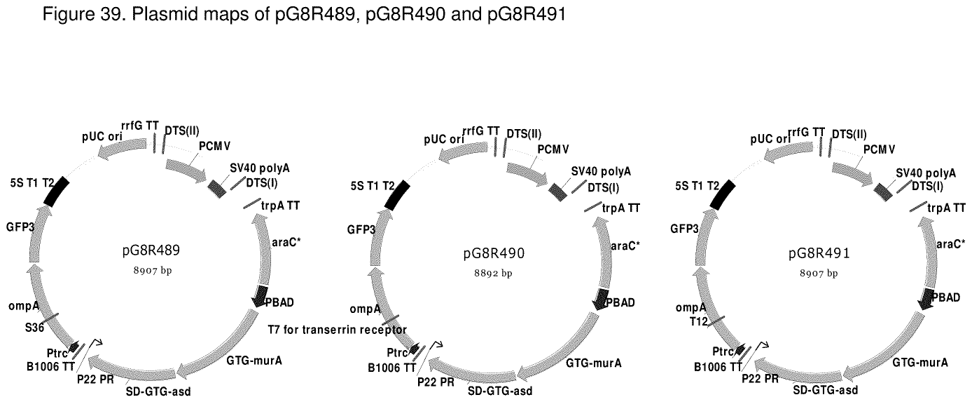

To construct plasmid pG8R489 encoding peptide S36 that targets ovarian cancer (FIG. 39), we used plasmid pG8R320 (FIG. 5B) as a template to amplify a 557 bp fragment using primers Ptrc-BcII-s and S36-a. With plasmid pG8R380 (FIG. 17A) as a template, the gene encoding c-terminal of OmpA and GFP was amplified with primer S36-s and GFP-BstBI-a. The 2 fragments were inserted into plasmid pG8R320 cut with BcII/BstBI to generate plasmid pG8R489. The regulated delayed lysis plasmid has a pUC on and can express ompA)s36 and gfp in Salmonella to display the synthesized OmpAΩS36 on the CCTS cell surface and GFP in the cytosol and after lysis of the CCTS cell in a cancer cell employs the P_{cmv} promoter to express an inserted gene sequence of importance for cancer cell/tumor therapy. This plasmid can treat ovarian cancer.

To construct plasmid pG8R490 encoding peptide T7 that targets TFR (FIG. 39), we used plasmid pG8R320 (FIG. 5B) as a template to amplify a 551 bp fragment using primers Ptrc-BcII-s and T7-a. With plasmid pG8R380 (FIG. 17A) as a template, the gene encoding c-terminal of OmpA and GFP was amplified with primer T7-s and GFP-BstBI-a. The 2 fragments were inserted into plasmid pG8R320 cut with BcII/BstBI to generate plasmid pG8R490. The regulated delayed lysis plasmid has a pUC on and can express ompAΩt7 and gfp in Salmonella to display the synthesized OmpAΩT7 on the CCTS cell surface and GFP in the cytosol and after lysis of the CCTS cell in a cancer cell employs the P_{cmv} promoter to express an inserted gene sequence of importance for cancer cell/tumor therapy. This plasmid can treat ovarian cancer and other cancers that overexpress TFR.

To construct plasmid pG8R491 encoding peptide T12 that targets TFR (FIG. 39), we used plasmid pG8R320 (FIG. 5B) as a template to amplify a 557 bp fragment using primers Ptrc-BcII-s and T12-a. With plasmid pG8R380 (FIG. 17A) as a template, the gene encoding c-terminal of OmpA and GFP was amplified with primer T12-s and GFP-BstBI-a. The 2 fragments were inserted into plasmid pG8R320 cut with BcII/BstBI to generate plasmid pG8R491. The regulated delayed lysis plasmid has a pUC on and can express ompAΩt12 and gfp in Salmonella to display the synthesized OmpAΩT12 on the CCTS cell surface and GFP in the cytosol and after lysis of the CCTS cell in a cancer cell employs the P_{cmv} promoter to express an inserted gene sequence of importance for cancer cell/tumor therapy. This plasmid can treat ovarian cancer and other cancers that overexpress TFR.

We can then establish the ability of SOCTI to induce tumor regression. The ability of oral SOCTI to induce tumor regression and reduce metastasis can be evaluated in ovarian cancer tumor-bearing mice. The innate immune responses in the tumor-bearing mice pre and after SOCTI treatment are evaluated in the syngeneic ID8 and HSG mouse models.

TABLE 12
Peptide used for ovarian cancer targeting

| | | SEQ | | |
|---|---|---|---|---|
| | | ID | | Ovarian |
| Target | Peptide | NO | Sequence | Cancer Cell |
| | | | Peptide with identified target/receptor | |
| α₃ | cDGXGXXc, | 258, | cDGLGDDc, | ES-2, CaOV-3, |
| | A3 (223) | 259, | cDGWGPNc, | SKOV-3, and |
| | | 260, | cDGLGDAc, | OVCAR-3 |
| | | 261, | cDGWGPAc, | |
| | | 262 | cLDWDAIc, | |
| α₉β₁ | PC3-1 (220, | 263 | WSGPGVWGASVK | SKOV3, A2780 |
| | 221) | | | |
| αᵥβ₃ | RGD (224) | | RGD | SKOV3, |
| | | | | OvCAR-3, |
| | | | | OVCAR4, |
| | RGD analog | 264 | GRGDYV | |
| | (225) | | | |
| CD13 | NGR (226) | 265 | NGR | ES-2, OVCAR- |
| | | 266 | CNGRCVSGCAGRC | 3, Patient |
| | | | CNGRC | |
| EGFR | GE11 (142) | 195 | YHWYGYTPQNVI | SKOV3, |
| | | | | OVCAR3 |
| | Peptide 22 | 267 | YHWYGYTPENVI | |
| | (227) | | | |
| | P1 (228) | 268 | SYPIPDT | SKOV3, |
| | P2 (228) | 269 | HTSDQTN | COV362 |
| EpCAM | EP114 (216) | 270 | KHLQCVRNICWS | |
| | EP133 (216) | 271 | EHLHCLGSLCWP | |
| | ep6 (229) | 272 | NNLRCIGNICWS | |
| | Epi-1/Ep-3 | 273/ | cWRPTRYRLLPWWIC/ | SKOV3 |
| | (230) | 274 | cWSRIALRLGLIC | |
| | SNF (231)/ | 275 | SNFYMPL | |
| | YQ-S2 (232) | 276 | SNFYMPLK | |
| | EP1 (233) | 277 | YEVHTYYLDc | |
| | Pep-14 | 278 | FALKGDY | |
| | Pep-9 (234) | 279 | FAAKGDY | |
| EphA | YSA | 280 | YSAYPDSVPMMS | SKOV-3, Hey |
| (epithelial | SWL (235) | 282 | SWLAYPGAVSYR | |
| cell kinase) | | | | |
| | APY | 282 | APYCVYRGSWSC | |
| | KYL | 283 | KYLPYWPVLSSL | |
| | VTM | 284 | VTMEAINLAFPG | |
| | NHW (236) | 285 | NHWLDTLFPMHM | |
| ERβ | EBIP-37 (237) | 286 | TGGGVSLLLHLLNTEQGES | |
| FRα | C7 (217) | 287 | MHTAPGWGYRLS | SKOV3 |
| | TVR (238) | 288 | CTVRTSAEC, | SKVO3 |
| | DWS (238) | 220 | DWSSWVYRDPQT, | |
| | IGN (238) | 289 | CIGNSNTLC | |
| FSHR | FSHβ (33-53) | 290 | YTRDLVYKDPARPKIQKTCTF | A2780, ES-2, |
| | (239) | | | OVCAR-3, |
| | | | | Caov-3, SKOV-3 |
| | FSHβ (81-95) | 291 | QCHCGKCDSDSTDCT | Caov-3 |
| | (240) | | | |
| FGFR | P7 (155) | 209 | PLLQATLGGGS | SKOV3 |
| | MQL (241) | 292 | MQLPLAT | SKOV3 |
| HB-EGF | Peptide 7 | 293 | DRWVARDPASIF | SKOV3, A2780 |
| | Peptide 29 | 294 | TVGLPMTYYMHT | |
| | (242) | | | |
| hTS (Human | LR (243) | 295 | LSCQLYQR | A2780, 2008 |
| thymidylate | | | | |
| synthase) | | | | |
| Her2/ErbB-2 | --- (244) | 296 | KCCYSL | |
| | H8 | 297 | ACSLQDPNCDWWGHYCG | |
| | H33 | 298 | ACYLQDPNCDWWGPYCG | |
| | H30 (245) | 299 | ACGLQGYGCWGMYGKCG | |
| | MAR or MSR | 300, | MARSGL, MARAKE, or | |
| | (246) | 301, | MSRTMS | |
| | | 302 | | |
| | APT_{HER2} | 303 | PFTPLAGSWTWENGKWTWK | |
| | (247) | | GHYRGKT | |
| | EC-1 | 304 | WTGWCLNPEESTWGFCTGSF | |
| | (248) | | | |
| | Ph7-2 (249) | 305 | LTVSPWY | |
| LAPTM4B | AP2H (250) | 306 | IHGHHIISVG | |
| LHRHR | LHRH | 307 | EHWSYGLRPG | A2780, |
| | | | | OVCAR-3, |
| | | | | SKOV3, |
| | | | | SKOV3-TR30, |
| | | | | EFO-21, EFO- |
| | | | | 27 |
| TFR | T7 (251) | 308 | HAIYPRH | SKOV-3 |
| (transferrin | T12 (251) | 309 | THRPPMWSPVWP | |
| receptor) | | | | |
| VCAM | MCP (252) | 310 | CVHSPNKKC | |
| | MLP (253) | 311 | VHPKQHR | SKOV-3, A2780 |
| | | | Peptide without identified target/receptor | |
| | Peptide 1 | 312 | SWQIGGN | HO8910 |
| | (222) | | | |
| | S36 (254) | 313 | METRPVAPHEFR | SKOV3 |
| | PDHPS1 | 314 | IATTTASAATAAAIGATPRAK | SKOV3, A2780 |
| | (255) | | | |
| | OC-6 | 315 | LTPPGRLSSWPL | OC-3, ES2, |
| | OC-26 (256) | 316 | SVTLSLRLPFPS | SKOV-3 |
| | OSTP (257) | 317 | PHLTALF | A2780 |
| | P2 (258) | 318 | NPMIRRQ | HO-8910 |
| | pJ18/J18 | 319 | RSLWSDFYASASRGP | SKOV-3, |
| | (259) | 320 | RRLPHLMPFEGSVFL | OVCAR-3 |
| | pJ24 (260) | | | |
| | oligopeptides | 321 | MPHPTKNFDLYV | BG1, A2780 |
| | I (261) | | | |
| | ZP1 (262) | 322 | SVSVGMKPSPRP | SK-OV-3 |

### REFERENCES

1. Smyth M J, Thia K Y, Street S E, Cretney E, Trapani J A, Taniguchi M, Kawano T, Pelikan S B, Crowe N Y, Godfrey D I. 2000. Differential tumor surveillance by natural killer (N K) and NKT cells. J Exp Med 191:661-668.
2. Swann J B, Uldrich A P, van Dommelen S, Sharkey J, Murray W K, Godfrey D I, Smyth M J. 2009. Type I natural killer T cells suppress tumors caused by p53 loss in mice. Blood 113:6382-6385.
3. Demaria O, Cornen S, Daeron M, Morel Y, Medzhitov R, Vivier E. 2019.

Harnessing innate immunity in cancer therapy. Nature 574:45-56.
4. Vesely M D, Kershaw M H, Schreiber R D, Smyth M J. 2011. Natural innate and adaptive immunity to cancer. Annu Rev Immunol 29:235-271.
5. Dunn G P, Bruce A T, Ikeda H, Old L J, Schreiber R D. 2002. Cancer immunoediting: from immunosurveillance to tumor escape. Nat Immunol 3:991-998.
6. Hanahan D, Weinberg R A. 2011. Hallmarks of cancer: the next generation. Cell 144:646-674.
7. Liu Z, Han C, Fu Y X. 2019. Targeting innate sensing in the tumor microenvironment to improve immunotherapy. Cell Mol Immunol doi:10.1038/s41423-019-0341-y.
8. Corrales L, Matson V, Flood B, Spranger S, Gajewski T F. 2017. Innate immune signaling and regulation in cancer immunotherapy. Cell Res 27:96-108.
9. Rusakiewicz S, Semeraro M, Sarabi M, Desbois M, Locher C, Mendez R, Vimond N, Concha A, Garrido F, Isambert N, Chaigneau L, Le Brun-Ly V, Dubreuil P, Cremer I, Caignard A, Poirier-Colame V, Chaba K, Flament C, Halama N, Jager D, Eggermont A, Bonvalot S, Commo F, Terrier P, Opolon P, Emile J F, Coindre J M, Kroemer G, Chaput N, Le Cesne A, Blay J Y, Zitvogel L. 2013. Immune infiltrates are prognostic factors in localized gastrointestinal stromal tumors. Cancer Res 73:3499-3510.
10. Mahmoud S M, Paish E C, Powe D G, Macmillan R D, Grainge M J, Lee A H, Ellis 10, Green A R. 2011. Tumor-infiltrating CD8+ lymphocytes predict clinical outcome in breast cancer. J Clin Oncol 29:1949-1955.
11. Azimi F, Scolyer R A, Rumcheva P, Moncrieff M, Murali R, McCarthy S W, Saw R P, Thompson J F. 2012. Tumor-infiltrating lymphocyte grade is an independent predictor of sentinel lymph node status and survival in patients with cutaneous melanoma. J Clin Oncol 30:2678-2683.
12. Mlecnik B, Tosolini M, Kirilovsky A, Berger A, Bindea G, Meatchi T, Bruneval P, Trajanoski Z, Fridman W H, Pages F, Galon J. 2011.

Histopathologic-based prognostic factors of colorectal cancers are associated with the state of the local immune reaction. J Clin Oncol 29:610-618.
13. Nguyen V H, Kim H S, Ha J M, Hong Y, Choy H E, Min J J. 2010. Genetically engineered Salmonella typhimurium as an imageable therapeutic probe for cancer. Cancer Res 70:18-23.
14. Hoffman R M. 2011. Tumor-seeking Salmonella amino acid auxotrophs. Curr Opin Biotechnol 22:917-923.
15. Wang C Z, Kazmierczak R A, Eisenstark A. 2016. Strains, Mechanism, and Perspective: Salmonella-Based Cancer Therapy. Int J Microbiol 2016:5678702.
16. Feigner S, Kocijancic D, Frahm M, Curtiss R, III, Erhardt M, Weiss S. 2016. Optimizing Salmonella enterica serovar Typhimurium for bacteria-mediated tumor therapy. Gut Microbes 7:171-177.
17. Hernandez-Luna M A, Luria-Perez R. 2018. Cancer Immunotherapy: Priming the Host Immune Response with Live Attenuated Salmonella enterica. J Immunol Res 2018:2984247.
18. Liang K, Liu Q, Li P, Luo H, Wang H, Kong Q. 2019. Genetically engineered Salmonella Typhimurium: Recent advances in cancer therapy. Cancer Lett 448:168-181.
19. Kasinskas R W, Forbes N S. 2006. Salmonella typhimurium specifically chemotax and proliferate in heterogeneous tumor tissue in vitro. Biotechnol Bioeng 94:710-721.
20. Toso J F, Gill V J, Hwu P, Marincola F M, Restifo N P, Schwartzentruber D J, Sherry R M, Topalian S L, Yang J C, Stock F, Freezer L J, Morton K E, Seipp C, Haworth L, Mavroukakis S, White D, MacDonald S, Mao J, Sznol M, Rosenberg S A. 2002. Phase I study of the intravenous administration of attenuated Salmonella typhimurium to patients with metastatic melanoma. J Clin Oncol 20:142-152.
21. Cunningham C, Nemunaitis J. 2001. A phase I trial of genetically modified Salmonella typhimurium expressing cytosine deaminase (TAPET-C D, VNP20029) administered by intratumoral injection in combination with 5-fluorocytosine for patients with advanced or metastatic cancer. Protocol no: CL-017. Version: Apr. 9, 2001. Hum Gene Ther 12:1594-1596.
22. Hoffman R M. 2009. Tumor-targeting amino acid auxotrophic Salmonella typhimurium. Amino Acids 37:509-521.
23. Pawelek J M, Low K B, Bermudes D. 1997. Tumor-targeted Salmonella as a novel anticancer vector. Cancer Res 57:4537-4544.
24. Franchi L. 2011. Role of inflammasomes in Salmonella infection. Front Microbiol 2:8.
25. Broz P, Ohlson M B, Monack D M. 2012. Innate immune response to Salmonella typhimurium, a model enteric pathogen. Gut Microbes 3:62-70.
26. Kawai T, Akira S. 2010. The role of pattern-recognition receptors in innate immunity: update on Toll-like receptors. Nat Immunol 11:373-384.
27. Luo J L, Maeda S, Hsu L C, Yagita H, Karin M. 2004. Inhibition of NF-kappaB in cancer cells converts inflammation-induced tumor growth mediated by TNFalpha to TRAIL-mediated tumor regression. Cancer Cell 6:297-305.
28. Harmey J H, Bucana C D, Lu W, Byrne A M, McDonnell S, Lynch C, Bouchier-Hayes D, Dong Z. 2002. Lipopolysaccharide-induced metastatic growth is associated with increased angiogenesis, vascular permeability and tumor cell invasion. Int J Cancer 101:415-422.
29. Cen X, Liu S, Cheng K. 2018. The Role of Toll-Like Receptor in Inflammation and Tumor Immunity. Front Pharmacol 9:878.
30. Hagar J A, Powell D A, Aachoui Y, Ernst R K, Miao E A. 2013. Cytoplasmic LPS activates caspase-11: implications in TLR4-independent endotoxic shock. Science 341:1250-1253.
31. Kayagaki N, Wong M T, Stowe I B, Ramani S R, Gonzalez L C, Akashi-Takamura S, Miyake K, Zhang J, Lee W P, Muszynski A, Forsberg L S, Carlson R W, Dixit V M. 2013. Noncanonical inflammasome activation by intracellular LPS independent of TLR4. Science 341:1246-1249.
32. Shi J, Zhao Y, Wang Y, Gao W, Ding J, Li P, Hu L, Shao F. 2014.

Inflammatory caspases are innate immune receptors for intracellular LPS. Nature 514:187-192.
33. Rathinam V A K, Zhao Y, Shao F. 2019. Innate immunity to intracellular LPS. Nat Immunol 20:527-533.
34. Kortmann J, Brubaker S W, Monack D M. 2015. Cutting Edge: Inflammasome Activation in Primary Human Macrophages Is Dependent on Flagellin. J Immunol 195:815-819.
35. Kofoed E M, Vance R E. 2011. Innate immune recognition of bacterial ligands by NAIPs determines inflammasome specificity. Nature 477:592-595.
36. Zhao Y, Yang J, Shi J, Gong Y N, Lu Q, Xu H, Liu L, Shao F. 2011. The NLRC4 inflammasome receptors for bacterial flagellin and type III secretion apparatus. Nature 477:596-600.
37. Yang J, Zhao Y, Shi J, Shao F. 2013. Human NAIP and mouse NAIP1 recognize bacterial type III secretion needle protein for inflammasome activation. Proc Natl Acad Sci USA 110:14408-14413.
38. Lamkanfi M, Dixit V M. 2014. Mechanisms and functions of inflammasomes. Cell 157:1013-1022.
39. Mariathasan S, Newton K, Monack D M, Vucic D, French D M, Lee W P, Roose-Girma M, Erickson S, Dixit V M. 2004. Differential activation of the inflammasome by caspase-1 adaptors ASC and Ipaf. Nature 430:213-218.
40. Hu B, Elinav E, Huber S, Booth C J, Strowig T, Jin C, Eisenbarth S C, Flavell R A. 2010. Inflammation-induced tumorigenesis in the colon is regulated by caspase-1 and NLRC4. Proc Natl Acad Sci USA 107:21635-21640.
41. Sadasivam S, Gupta S, Radha V, Batta K, Kundu T K, Swarup G. 2005. Caspase-1 activator Ipaf is a p53-inducible gene involved in apoptosis. Oncogene 24:627-636.
42. Otto F, Schmid P, Mackensen A, Wehr U, Seiz A, Braun M, Galanos C, Mertelsmann R, Engelhardt R. 1996. Phase II trial of intravenous endotoxin in patients with colorectal and non-small cell lung cancer. Eur J Cancer 32A:1712-1718.
43. Engelhardt R, Mackensen A, Galanos C. 1991. Phase I trial of intravenously administered endotoxin (Salmonella abortus equi) in cancer patients. Cancer Res 51:2524-2530.
44. Mackensen A, Galanos C, Engelhardt R. 1991. Modulating activity of interferon-gamma on endotoxin-induced cytokine production in cancer patients. Blood 78:3254-3258.
45. Goto S, Sakai S, Kera J, Suma Y, Soma G I, Takeuchi S. 1996. Intradermal administration of lipopolysaccharide in treatment of human cancer. Cancer Immunol Immunother 42:255-261.
46. Vosika G J, Barr C, Gilbertson D. 1984. Phase-I study of intravenous modified lipid A. Cancer Immunol Immunother 18:107-112.
47. Kiani A, Tschiersch A, Gaboriau E, Otto F, Seiz A, Knopf H P, Stutz P, Farber L, Haus U, Galanos C, Mertelsmann R, Engelhardt R. 1997. Downregulation of the proinflammatory cytokine response to endotoxin by pretreatment with the nontoxic lipid A analog SDZ MRL 953 in cancer patients. Blood 90:1673-1683.
48. de Bono J S, Dalgleish A G, Carmichael J, Diffley J, Lofts F J, Fyffe D, Ellard S, Gordon R J, Brindley C J, Evans T R. 2000. Phase I study of ONO-4007, a synthetic analogue of the lipid A moiety of bacterial lipopolysaccharide. Clin Cancer Res 6:397-405.
49. Myers K, Truchot A, Ward J. 1990. A critical determinant of lipid A endotoxic activity. In Congress IES, Nowotny A, Spitzer J J, Ziegler E J (ed), Cellular and molecular aspects of endotoxin reactions: proceedings of the 1 st Congress of the International Endotoxin Society, San Diego, 9-12 May 1990. Elsevier Sciences Publishers, Amsterdam.
50. Evans J T, Cluff C W, Johnson D A, Lacy M J, Persing D H, Baldridge J R. 2003. Enhancement of antigen-specific immunity via the TLR4 ligands MPL adjuvant and Ribi.529. Expert Rev Vaccines 2:219-229.
51. MacLeod M K, McKee A S, David A, Wang J, Mason R, Kappler J W, Marrack P. 2011. Vaccine adjuvants aluminum and monophosphoryl lipid A provide distinct signals to generate protective cytotoxic memory CD8 T cells. Proc Natl Acad Sci USA 108:7914-7919.
52. Cluff C. 2013. Monophosphoryl Lipid A (MPL) as an Adjuvant for Anti-Cancer Vaccines: Clinical Results. In: Madame Curie Bioscience Database [Internet]. Austin (TX): Landes Bioscience; 2000-2013. Available from: https://www.ncbi.nlm.nih.gov/books/NBK7284/.
53. Nguyen C T, Hong S H, Sin J I, Vu H V, Jeong K, Cho K O, Uematsu S, Akira S, Lee S E, Rhee J H. 2013. Flagellin enhances tumor-specific CD8(+) T cell immune responses through TLR5 stimulation in a therapeutic cancer vaccine model. Vaccine 31:3879-3887.
54. Rhee S H, Im E, Pothoulakis C. 2008. Toll-like receptor 5 engagement modulates tumor development and growth in a mouse xenograft model of human colon cancer. Gastroenterology 135:518-528.
55. Zheng J H, Nguyen V H, Jiang S N, Park S H, Tan W, Hong S H, Shin M G, Chung I J, Hong Y, Bom H S, Choy H E, Lee S E, Rhee J H, Min J J. 2017. Two-step enhanced cancer immunotherapy with engineered Salmonella typhimurium secreting heterologous flagellin. Sci Transl Med 9.
56. Mills C D, Kincaid K, Alt J M, Heilman M J, Hill A M. 2000. M-1/M-2 macrophages and the Th1/Th2 paradigm. J Immunol 164:6166-6173.
57. Feigner S, Sporing I, Pawar V, Kocijancic D, Preusse M, Falk C, Rohde M, Häussler S, Weiss S, Erhardt M. 2019. The immunogenic potential of bacterial flagella for Salmonella-mediated tumor therapy. Int J Cancer doi:10.1002/ijc.32807.
58. Venegas A, Gomez I, Zaror I, Yudelevich A. 1988. The nucleotide sequence of the Salmonella typhi ompC porin gene. Nucleic Acids Res 16:7721.
59. Puente J L, Alvarez-Scherer V, Gosset G, Calva E. 1989. Comparative analysis of the Salmonella typhi and Escherichia coli ompC genes. Gene 83:197-206.
60. Meyer P N, Wilmes-Riesenberg M R, Stathopoulos C, Curtiss R, III. 1998.

Virulence of a Salmonella typhimurium OmpD mutant. Infect Immun 66:387-390.
61. Singh S P, Miller S, Williams Y U, Rudd K E, Nikaido H. 1996. Immunochemical structure of the OmpD porin from Salmonella typhimurium. Microbiology (Reading) 142 (Pt 11):3201-3210.
62. Singh S P, Upshaw Y, Abdullah T, Singh S R, Klebba P E. 1992. Structural relatedness of enteric bacterial porins assessed with monoclonal antibodies to Salmonella typhimurium OmpD and OmpC. J Bacteriol 174:1965-1973.
63. Balasubramaniam D, Arockiasamy A, Kumar P D, Sharma A, Krishnaswamy S. 2012. Asymmetric pore occupancy in crystal structure of OmpF porin from Salmonella typhi. J Struct Biol 178:233-244.
64. McFarland W C, Stocker B A. 1987. Effect of different purine auxotrophic mutations on mouse-virulence of a Vi-positive strain of Salmonella dublin and of two strains of Salmonella typhimurium. Microb Pathog 3:129-141.
65. O'Callaghan D, Maskell D, Liew F Y, Easmon C S, Dougan G. 1988. Characterization of aromatic- and purine-dependent Salmonella typhimurium: attention, persistence, and ability to induce protective immunity in BALB/c mice. Infect Immun 56:419-423.
66. Sigwart D F, Stocker B A, Clements J D. 1989. Effect of a purA mutation on efficacy of Salmonella live-vaccine vectors. Infect Immun 57:1858-1861.
67. Brown R F, Stocker B A. 1987. Salmonella typhi 205aTy, a strain with two attenuating auxotrophic characters, for use in laboratory teaching. Infect Immun 55:892-898.
68. Wang J Y, Pasetti M F, Noriega F R, Anderson R J, Wasserman S S, Galen J E, Sztein M B, Levine M M. 2001. Construction, genotypic and phenotypic characterization, and immunogenicity of attenuated ΔguaBA Salmonella enterica serovar Typhi strain CVD 915. Infect Immun 69:4734-4741.
69. Schafer M P, Hannon W H, Levin A P. 1974. In vivo and in vitro complementation between guaB and in vivo complementation between guaA auxotrophs of Salmonella typhimurium. J Bacteriol 117:1270-1279.
70. Theisen M, Neuhard J. 1990. Translational coupling in the pyrF operon of Salmonella typhimurium. Mol Gen Genet 222:345-352.
71. Theisen M, Kelln R A, Neuhard J. 1987. Cloning and characterization of the pyrF operon of Salmonella typhimurium. Eur J Biochem 164:613-619.
72. Blat Y, Eisenbach M. 1995. Tar-dependent and -independent pattern formation by Salmonella typhimurium. J Bacteriol 177:1683-1691.
73. Curtiss R, III, Kong W. Mar. 21, 2017 2011. Recombinant bacterium to decrease tumor growth patent U.S. Pat. No. 9,598,697 B2.
74. Zhang M, Forbes N S. 2015. Trg-deficient Salmonella colonize quiescent tumor regions by exclusively penetrating or proliferating. J Control Release 199:180-189.
75. Saltzman D A, Heise C P, Hasz D E, Zebede M, Kelly S M, Curtiss R, III, Leonard A S, Anderson P M. 1996. Attenuated Salmonella typhimurium containing interleukin-2 decreases MC-38 hepatic metastases: a novel anti-tumor agent. Cancer Biother Radiopharm 11:145-153.
76. Saltzman D A, Katsanis E, Heise C P, Hasz D E, Kelly S M, Curtiss R, III, Leonard A S, Anderson P M. 1997. Patterns of hepatic and splenic colonization by an attenuated strain of Salmonella typhimurium containing the gene for human interleukin-2: a novel anti-tumor agent. Cancer Biother Radiopharm 12:37-45.
77. Saltzman D A, Katsanis E, Heise C P, Hasz D E, Vigdorovich V, Kelly S M, Curtiss R, III, Leonard A S, Anderson P M. 1997. Antitumor mechanisms of attenuated Salmonella typhimurium containing the gene for human interleukin-2: a novel antitumor agent? J Pediatr Surg 32:301-306.
78. Schödel F, Kelly S M, Peterson D L, Milich D R, Curtiss R, III. 1994. Hybrid hepatitis B virus core-pre-S proteins synthesized in avirulent Salmonella typhimurium and Salmonella typhi for oral vaccination. Infect Immun 62:1669-1676.
79. Fritz S E, Henson M S, Greengard E, Winter A L, Stuebner K M, Yoon U, Wilk V L, Borgatti A, Augustin L B, Modiano J F, Saltzman D A. 2016. A phase I clinical study to evaluate safety of orally administered, genetically engineered Salmonella enterica serovar Typhimurium for canine osteosarcoma. Vet Med Sci 2:179-190.
80. Konjufca V, Jenkins M, Wang S, Juarez-Rodriguez M D, Curtiss R, III. 2008. Immunogenicity of recombinant attenuated Salmonella enterica serovar Typhimurium vaccine strains carrying a gene that encodes Eimeria tenella antigen S07. Infect Immun 76:5745-5753.
81. Kong Q, Six D A, Liu Q, Gu L, Wang S, Alamuri P, Raetz C R, Curtiss R, III. 2012. Phosphate groups of lipid A are essential for Salmonella enterica serovar Typhimurium virulence and affect innate and adaptive immunity. Infect Immun 80:3215-3224.
82. Augustin L B, Milbauer L, Hastings S E, Leonard A S, Saltzman D A, Schottel J L. 2021. Salmonella enterica Typhimurium engineered for nontoxic systemic colonization of autochthonous tumors. J Drug Target 29:294-299.
83. Shirmanova M, Yuzhakova D, Snopova L, Perelman G, Serebrovskaya E, Lukyanov K, Turchin I, Subochev P, Lukyanov S, Kamensky V, Zagaynova E. 2015. Towards PDT with Genetically Encoded Photosensitizer KillerRed: A Comparison of Continuous and Pulsed Laser Regimens in an Animal Tumor Model. PLoS One 10:e0144617.
84. Chen X, Zaro J L, Shen W C. 2013. Fusion protein linkers: property, design and functionality. Adv Drug Deliv Rev 65:1357-1369.
85. Reddy Chichili V P, Kumar V, Sivaraman J. 2013. Linkers in the structural biology of protein-protein interactions. Protein Sci 22:153-167.
86. Miller S I, Ernst R K, Bader M W. 2005. LPS, TLR4 and infectious disease diversity. Nat Rev Microbiol 3:36-46.
87. Takeda K, Akira S. 2004. TLR signaling pathways. Semin Immunol 16:3-9.
88. Mata-Haro V, Cekic C, Martin M, Chilton P M, Casella C R, Mitchell T C. 2007. The vaccine adjuvant monophosphoryl lipid A as a TRIF-biased agonist of TLR4. Science 316:1628-1632.
89. Kagan J C, Su T, Horng T, Chow A, Akira S, Medzhitov R. 2008. TRAM couples endocytosis of Toll-like receptor 4 to the induction of interferon-β. Nat Immunol 9:361-368.
90. Takeuchi O, Akira S. 2010. Pattern recognition receptors and inflammation. Cell 140:805-820.
91. Park B S, Song D H, Kim H M, Choi B S, Lee H, Lee J O. 2009. The structural basis of lipopolysaccharide recognition by the TLR4-MD-2 complex. Nature 458:1191-1195.
92. Reed S G, Bertholet S, Coler R N, Friede M. 2009. New horizons in adjuvants for vaccine development. Trends Immunol 30:23-32.
93. Wang S, Kong Q, Curtiss R, III. 2013. New technologies in developing recombinant attenuated Salmonella vaccine vectors. Microb Pathog 58:17-28.
94. Wang X, Karbarz M J, McGrath S C, Cotter R J, Raetz C R. 2004. MsbA transporter-dependent lipid A 1-dephosphorylation on the periplasmic surface of the inner membrane: topography of Francisella novicida LpxE expressed in Escherichia coli. J Biol Chem 279:49470-49478.
95. Kong Q, Six D A, Roland K L, Liu Q, Gu L, Reynolds C M, Wang X, Raetz C R, Curtiss R, III. 2011. Salmonella synthesizing 1-dephosphorylated lipopolysaccharide exhibits low endotoxic activity while retaining its immunogenicity. J Immunol 187:412-423.
96. Chooneea D, Karlsson R, Encheva V, Arnold C, Appleton H, Shah H. 2010. Elucidation of the outer membrane proteome of Salmonella enterica serovar Typhimurium utilising a lipid-based protein immobilization technique. BMC Microbiol 10:44.
97. Kong W, Wanda S Y, Zhang X, Bollen W, Tinge S A, Roland K L, Curtiss R, III. 2008. Regulated programmed lysis of recombinant Salmonella in host tissues to release protective antigens and confer biological containment. Proc Natl Acad Sci USA 105:9361-9366.
98. Curtiss R, III., Wanda S Y, Gunn B M, Zhang X, Tinge S A, Ananthnarayan V, Mo H, Wang S, Kong W. 2009. Salmonella enterica serovar Typhimurium strains with regulated delayed attenuation in vivo. Infect Immun 77:1071-1082.
99. Liu F, Yang J, Zhang Y, Zhou D, Chen Y, Gai W, Shi W, Li Q, Tien P, Yan H. 2010. Recombinant flagellins with partial deletions of the hypervariable domain lose antigenicity but not mucosal adjuvancy. Biochem Biophys Res Commun 392:582-587.
100. Traut T W. 1994. Physiological concentrations of purines and pyrimidines. Mol Cell Biochem 140:1-22.
101. Alvarez C L, Troncoso M F, Espelt M V. 2022. Extracellular ATP and adenosine in tumor microenvironment: Roles in epithelial-mesenchymal transition, cell migration, and invasion. J Cell Physiol 237:389-400.
102. Di Virgilio F, Sarti A C, Falzoni S, De Marchi E, Adinolfi E. 2018. Extracellular ATP and P2 purinergic signalling in the tumour microenvironment. Nat Rev Cancer 18:601-618.
103. Di Virgilio F, Adinolfi E. 2017. Extracellular purines, purinergic receptors and tumor growth. Oncogene 36:293-303.
104. Kim J J, Tannock I F. 2005. Repopulation of cancer cells during therapy: an important cause of treatment failure. Nat Rev Cancer 5:516-525.
105. Minchinton A I, Tannock I F. 2006. Drug penetration in solid tumours. Nat Rev Cancer 6:583-592.
106. Jain R K, Forbes N S. 2001. Can engineered bacteria help control cancer? Proc Natl Acad Sci USA 98:14748-14750.
107. Avogadri F, Martinoli C, Petrovska L, Chiodoni C, Transidico P, Bronte V, Longhi R, Colombo M P, Dougan G, Rescigno M. 2005. Cancer immunotherapy based on killing of Salmonella-infected tumor cells. Cancer Res 65:3920-3927.
108. Forbes N S, Munn L L, Fukumura D, Jain R K. 2003. Sparse initial entrapment of systemically injected Salmonella typhimurium leads to heterogeneous accumulation within tumors. Cancer Res 63:5188-5193.
109. Bren A, Eisenbach M. 2000. How signals are heard during bacterial chemotaxis: protein-protein interactions in sensory signal propagation. J Bacteriol 182:6865-6873.
110. Yamamoto K, Imae Y. 1993. Cloning and characterization of the Salmonella typhimurium-specific chemoreceptor Tcp for taxis to citrate and from phenol. Proc Natl Acad Sci USA 90:217-221.
111. Adler J. 1966. Chemotaxis in bacteria. Science 153:708-716.
112. Kondoh H, Ball C B, Adler J. 1979. Identification of a methyl-accepting chemotaxis protein for the ribose and galactose chemoreceptors of Escherichia coli. Proc Natl Acad Sci USA 76:260-264.
113. Kasinskas R W, Forbes N S. 2007. Salmonella typhimurium lacking ribose chemoreceptors localize in tumor quiescence and induce apoptosis. Cancer Res 67:3201-3209.
114. Mazumder R, Phelps T J, Krieg N R, Benoit R E. 1999. Determining chemotactic responses by two subsurface microaerophiles using a simplified capillary assay method. J Microbiol Methods 37:255-263.
115. Frahm M, Feigner S, Kocijancic D, Rohde M, Hensel M, Curtiss R, III, Erhardt M, Weiss S. 2015. Efficiency of conditionally attenuated Salmonella enterica serovar Typhimurium in bacterium-mediated tumor therapy. MBio 6.
116. Felgner S, Frahm M, Kocijancic D, Rohde M, Eckweiler D, Bielecka A, Bueno E, Cava F, Abraham W, Curtiss R, III, Haussler S, Erhardt M, Weiss S. 2016. AroA deficient Salmonella Typhimurium—more than a metabolically attenuated mutant. MBio Accepted.
117. Doan H Q, Bowen K A, Jackson L A, Evers B M. 2009. Toll-like receptor 4 activation increases Akt phosphorylation in colon cancer cells. Anticancer Res 29:2473-2478.
118. Kay C, Wang R, Kirkby M, Man S M. 2020. Molecular mechanisms activating the NAIP-NLRC4 inflammasome: Implications in infectious disease, autoinflammation, and cancer. Immunol Rev 297:67-82.
119. Kocijancic D, Leschner S, Feigner S, Komoll R M, Frahm M, Pawar V, Weiss S. 2017. Therapeutic benefit of Salmonella attributed to LPS and TNF-alpha is exhaustible and dictated by tumor susceptibility. Oncotarget 8:36492-36508.
120. Tomayko M M, Reynolds C P. 1989. Determination of subcutaneous tumor size in athymic (nude) mice. Cancer Chemother Pharmacol 24:148-154.
121. Morvan M G, Lanier L L. 2016. N K cells and cancer: you can teach innate cells new tricks. Nat Rev Cancer 16:7-19.
122. Guillerey C, Huntington N D, Smyth M J. 2016. Targeting natural killer cells in cancer immunotherapy. Nat Immunol 17:1025-1036.
123. Lopez-Soto A, Gonzalez S, Smyth M J, Galluzzi L. 2017. Control of Metastasis by N K Cells. Cancer Cell 32:135-154.
124. Treffers L W, Hiemstra I H, Kuijpers T W, van den Berg T K, Matlung H L. 2016. Neutrophils in cancer. Immunol Rev 273:312-328.
125. Nicolas-Avila J A, Adrover J M, Hidalgo A. 2017. Neutrophils in Homeostasis, Immunity, and Cancer. Immunity 46:15-28.
126. Vo M C, Nguyen-Pham T N, Lee H J, Jaya Lakshmi T, Yang S, Jung S H, Kim H J, Lee J J. 2017. Combination therapy with dendritic cells and lenalidomide is an effective approach to enhance antitumor immunity in a mouse colon cancer model. Oncotarget 8:27252-27262.
127. Ruffell B, Coussens L M. 2015. Macrophages and therapeutic resistance in cancer. Cancer Cell 27:462-472.
128. Mills C D, Lenz L L, Harris R A. 2016. A Breakthrough: Macrophage-Directed Cancer Immunotherapy. Cancer Res 76:513-516.
129. Mantovani A, Marchesi F, Malesci A, Laghi L, Allavena P. 2017. Tumour-associated macrophages as treatment targets in oncology. Nat Rev Clin Oncol 14:399-416.
130. Ferreira D, Silva A P, Nobrega F L, Martins I M, Barbosa-Matos C, Granja S, Martins S F, Baltazar F, Rodrigues L R. 2019. Rational Identification of a Colorectal Cancer Targeting Peptide through Phage Display. Sci Rep 9:3958.
131. Amorim R, Pinheiro C, Miranda-Goncalves V, Pereira H, Moyer M P, Preto A, Baltazar F. 2015. Monocarboxylate transport inhibition potentiates the cytotoxic effect of 5-fluorouracil in colorectal cancer cells. Cancer Lett 365:68-78.
132. Ferro S, Azevedo-Silva J, Casal M, Corte-Real M, Baltazar F, Preto A. 2016. Characterization of acetate transport in colorectal cancer cells and potential therapeutic implications. Oncotarget 7:70639-70653.
133. Silva A, Cerqueira M C, Rosa B, Sobral C, Pinto-Ribeiro F, Costa M F, Baltazar F, Afonso J. 2023. Prognostic Value of Monocarboxylate Transporter 1 Overexpression in Cancer: A Systematic Review. Int J Mol Sci 24.
134. Cuff M A, Lambert D W, Shirazi-Beechey S P. 2002. Substrate-induced regulation of the human colonic monocarboxylate transporter, MCT1. J Physiol 539:361-371.
135. Payen V L, Mina E, Van Hee V F, Porporato P E, Sonveaux P. 2020. Monocarboxylate transporters in cancer. Mol Metab 33:48-66.
136. Qin Y, Shembrey C, Smith J, Paquet-Fifield S, Behrenbruch C, Beyit L M, Thomson B N J, Heriot A G, Cao Y, Hollande F. 2020. Laminin 521 enhances self-renewal via STAT3 activation and promotes tumor progression in colorectal cancer. Cancer Lett 476:161-169.
137. Nakahara H, Nomizu M, Akiyama S K, Yamada Y, Yeh Y, Chen W T. 1996. A mechanism for regulation of melanoma invasion. Ligation of alpha6beta1 integrin by laminin G peptides. J Biol Chem 271:27221-27224.
138. Feng G K, Zhang M Q, Wang H X, Cai J, Chen S P, Wang Q, Gong J, Leong K W, Wang J, Zhang X. 2019. Identification of an integrin α6-targeted peptide for nasopharyngeal carcinoma-specific nanotherapeutics. Advanced Therapeutics 2:1900018.
139. Mei Y, Li Y H, Yang X C, Zhou C, Li Z J, Zheng X B, Ye J C, Li C, Zhang X H, Yuan J M, Huang H Q, Fan W, Zhang W G, Zeng M S, Feng G K. 2020. An optimized integrin alpha6-targeted peptide for positron emission tomography/magnetic resonance imaging of pancreatic cancer and its precancerous lesion. Clin Transl Med 10:e157.
140. Joshi B P, Zhou J, Pant A, Duan X, Zhou Q, Kuick R, Owens S R, Appelman H, Wang T D. 2016. Design and Synthesis of Near-Infrared Peptide for in Vivo Molecular Imaging of HER2. Bioconjug Chem 27:481-494.
141. Zhou J, Joshi B P, Duan X, Pant A, Qiu Z, Kuick R, Owens S R, Wang T D. 2015. EGFR Overexpressed in Colonic Neoplasia Can be Detected on Wide-Field Endoscopic Imaging. Clin Transl Gastroenterol 6:e101.
142. Li Z, Zhao R, Wu X, Sun Y, Yao M, Li J, Xu Y, Gu J. 2005. Identification and characterization of a novel peptide ligand of epidermal growth factor receptor for targeted delivery of therapeutics. FASEB J 19:1978-1985.
143. Song S, Liu D, Peng J, Deng H, Guo Y, Xu L X, Miller A D, Xu Y. 2009. Novel peptide ligand directs liposomes toward EGF-R high-expressing cancer cells in vitro and in vivo. FASEB J 23:1396-1404.
144. Ai S, Duan J, Liu X, Bock S, Tian Y, Huang Z. 2011. Biological evaluation of a novel doxorubicin-peptide conjugate for targeted delivery to EGF receptor-overexpressing tumor cells. Mol Pharm 8:375-386.
145. Jeong M H, Kim K, Kim E M, Cheong S J, Lee C M, Jeong H J, Kim D W, Lim S T, Sohn M H, Chung J. 2012. In vivo and in vitro evaluation of Cy5.5 conjugated epidermal growth factor receptor binding peptide. NucI Med Biol 39:805-812.
146. Chen W W, Zhang H R, Huang Z G, Zhou Z Y, Lou Q W, Jiang X Y, Zhu Z H. 2021. Expression and purification of a recombinant ELRL-MAP30 with dual-targeting anti-tumor bioactivity. Protein Expr Purif 185:105893.
147. Denholt C L, Hansen P R, Pedersen N, Poulsen H S, Gillings N, Kjaer A. 2009. Identification of novel peptide ligands for the cancer-specific receptor mutation EFGRvIII using a mixture-based synthetic combinatorial library. Biopolymers 91:201-206.
148. Campa M J, Kuan C T, O'Connor-McCourt M D, Bigner D D, Patz E F, Jr. 2000. Design of a novel small peptide targeted against a tumor-specific receptor. Biochem Biophys Res Commun 275:631-636.
149. Hansen C L, Hansen P R, Pedersen N, Poulsen H S, Gillings N, Kjaer A. 2008. Identification of amino acid residues in PEPHC1 important for binding to the tumor-specific receptor EGFRvIII. Chem Biol Drug Des 72:273-278.
150. Xiang Z, Yang X, Xu J, Lai W, Wang Z, Hu Z, Tian J, Geng L, Fang Q. 2017. Tumor detection using magnetosome nanoparticles functionalized with a newly screened EGFR/HER2 targeting peptide. Biomaterials 115:53-64.
151. Mansour S, Adhya I, Lebleu C, Dumpati R, Rehan A, Chall S, Dai J, Errasti G, Delacroix T, Chakrabarti R. 2022. Identification of a novel peptide ligand for the cancer-specific receptor mutation EGFRvIII using high-throughput sequencing of phage-selected peptides. Sci Rep 12:20725.
152. Sachdeva S, Joo H, Tsai J, Jasti B, Li X. 2019. A Rational Approach for Creating Peptides Mimicking Antibody Binding. Sci Rep 9:997.
153. Han C Y, Yue L L, Tai L Y, Zhou L, Li X Y, Xing G H, Yang X G, Sun M S, Pan W S. 2013. A novel small peptide as an epidermal growth factor receptor targeting ligand for nanodelivery in vitro. Int J Nanomedicine 8:1541-1549.
154. Sobral D V, Fuscaldi L L, Durante A C R, Rangel M G, Oliveira L R, Mendonca F F, Miranda A C C, Cabeza J M, Montor W R, Cabral F R, Barboza M F F, Malavolta L. 2020. Radiochemical and biological properties of peptides designed to interact with EGF receptor: Relevance for glioblastoma. NucI Med Biol 88-89:14-23.
155. Wu X, Yan Q, Huang Y, Huang H, Su Z, Xiao J, Zeng Y, Wang Y, Nie C, Yang Y, Li X. 2010. Isolation of a novel basic FGF-binding peptide with potent antiangiogenetic activity. J Cell Mol Med 14:351-356.
156. Smith C J, Gali H, Sieckman G L, Hayes D L, Owen N K, Mazuru D G, Volkert W A, Hoffman T J. 2003. Radiochemical investigations of ¹⁷⁷Lu-DOTA-8-Aoc-BBN[7-14]NH2: an in vitro/in vivo assessment of the targeting ability of this new radiopharmaceutical for PC-3 human prostate cancer cells. NucI Med Biol 30:101-109.
157. Bae D G, Kim T D, Li G, Yoon W H, Chae C B. 2005. Anti-flt1 peptide, a vascular endothelial growth factor receptor 1-specific hexapeptide, inhibits tumor growth and metastasis. Clin Cancer Res 11:2651-2661.
158. Liu G, Wang M, He H, Li J. 2021. Doxorubicin-Loaded Tumor-Targeting Peptide-Decorated Polypeptide Nanoparticles for Treating Primary Orthotopic Colon Cancer. Front Pharmacol 12:744811.
159. Bakhshinejad B, Sadeghizadeh M. 2022. Identification of a novel colon adenocarcinoma cell targeting peptide using phage display library biopanning. Biotechnol Appl Biochem 69:2753-2765.
160. Laakkonen P, Porkka K, Hoffman J A, Ruoslahti E. 2002. A tumor-homing peptide with a targeting specificity related to lymphatic vessels. Nat Med 8:751-755.
161. Laakkonen P, Akerman M E, Biliran H, Yang M, Ferrer F, Karpanen T, Hoffman R M, Ruoslahti E. 2004. Antitumor activity of a homing peptide that targets tumor lymphatics and tumor cells. Proc Natl Acad Sci USA 101:9381-9386.
162. Li Z J, Wu W K, Ng S S, Yu L, Li H T, Wong C C, Wu Y C, Zhang L, Ren S X, Sun X G, Chan K M, Cho C H. 2010. A novel peptide specifically targeting the vasculature of orthotopic colorectal cancer for imaging detection and drug delivery. J Control Release 148:292-302.
163. Hatakeyama S, Sugihara K, Shibata T K, Nakayama J, Akama T O, Tamura N, Wong S M, Bobkov A A, Takano Y, Ohyama C, Fukuda M, Fukuda M N. 2011. Targeted drug delivery to tumor vasculature by a carbohydrate mimetic peptide. Proc Natl Acad Sci USA 108:19587-19592.
164. Zhang Y, Chen J, Zhang Y, Hu Z, Hu D, Pan Y, Ou S, Liu G, Yin X, Zhao J, Ren L, Wang J. 2007. Panning and identification of a colon tumor binding peptide from a phage display peptide library. J Biomol Screen 12:429-435.
165. Hou L, Zhu D, Liang Y, Tian X, Li L, Wang P, Zhu L, Weng X, Wang Y, Li Y, Wu T, Wang J, Meng X. 2018. Identification of a specific peptide binding to colon cancer cells from a phage-displayed peptide library. Br J Cancer 118:79-87.
166. Kelly K A, Jones D A. 2003. Isolation of a colon tumor specific binding peptide using phage display selection. Neoplasia 5:437-444.
167. Kwak M H, Yi G, Yang S M, Choe Y, Choi S, Lee H S, Kim E, Lim Y B, Na K, Choi M G, Koo H, Park J M. 2020. A Dodecapeptide Selected by Phage Display as a Potential Theranostic Probe for Colon Cancers. Transl Oncol 13:100798.
168. Rasmussen U B, Schreiber V, Schultz H, Mischler F, Schughart K. 2002. Tumor cell-targeting by phage-displayed peptides. Cancer Gene Ther 9:606-612.
169. Kubo N, Akita N, Shimizu A, Kitahara H, Parker A L, Miyagawa S. 2008. Identification of oligopeptide binding to colon cancer cells separated from patients using laser capture microdissection. J Drug Target 16:396-404.
170. Fu X Q, Liu B, Wang Y P, Li J K, Zhu P L, Li T, Tse K W, Chou J Y, Yin C L, Bai J X, Liu Y X, Chen Y J, Yu Z L. 2020. Activation of STAT3 is a key event in TLR4 signaling-mediated melanoma progression. Cell Death Dis 11:246.
171. Molteni M, Marabella D, Orlandi C, Rossetti C. 2006. Melanoma cell lines are responsive in vitro to lipopolysaccharide and express TLR-4. Cancer Lett 235:75-83.
172. Geng D, Kaczanowska S, Tsai A, Younger K, Ochoa A, Rapoport A P, Ostrand-Rosenberg S, Davila E. 2015. TLR5 Ligand-Secreting T Cells Reshape the Tumor Microenvironment and Enhance Antitumor Activity. Cancer Res 75:1959-1971.
173. Hwang H S, Cherukula K, Bang Y J, Vijayan V, Moon M J, Thiruppathi J, Puth S, Jeong Y Y, Park I K, Lee S E, Rhee J H. 2020. Combination of Photodynamic Therapy and a Flagellin-Adjuvanted Cancer Vaccine Potentiated the Anti-PD-1-Mediated Melanoma Suppression. Cells 9.
174. Zhu M, Ding X, Zhao R, Liu X, Shen H, Cai C, Ferrari M, Wang H Y, Wang R F. 2018. Co-delivery of tumor antigen and dual toll-like receptor ligands into dendritic cell by silicon microparticle enables efficient immunotherapy against melanoma. J Control Release 272:72-82.
175. Atukorale P U, Raghunathan S P, Raguveer V, Moon T J, Zheng C, Bielecki P A, Wiese M L, Goldberg A L, Covarrubias G, Hoimes C J, Karathanasis E. 2019. Nanoparticle Encapsulation of Synergistic Immune Agonists Enables Systemic Codelivery to Tumor Sites and IFNbeta-Driven Antitumor Immunity. Cancer Res 79:5394-5406.
176. Ruoslahti E. 1996. RGD and other recognition sequences for integrins. Annu Rev Cell Dev Biol 12:697-715.
177. Koivunen E, Wang B, Ruoslahti E. 1995. Phage libraries displaying cyclic peptides with different ring sizes: ligand specificities of the RGD-directed integrins. Biotechnology (N Y) 13:265-270.
178. Yacobovich S, Tuchinsky L, Kirby M, Kardash T, Agranyoni O, Nesher E, Redko B, Gellerman G, Tobi D, Gurova K, Koman I, Ashur Fabian O, Pinhasov A. 2016. Novel synthetic cyclic integrin αᵥβ₃ binding peptide ALOS4: Antitumor activity in mouse melanoma models. Oncotarget 7:63549-63560.
179. Wu X, Huang H, Wang C, Lin S, Huang Y, Wang Y, Liang G, Yan Q, Xiao J, Wu J, Yang Y, Li X. 2013. Identification of a novel peptide that blocks basic fibroblast growth factor-mediated cell proliferation. Oncotarget 4:1819-1828.
180. Fan H, Duan Y, Zhou H, Li W, Li F, Guo L, Roeske R W. 2002. Selection of peptide ligands binding to fibroblast growth factor receptor 1. IUBMB Life 54:67-72.
181. Wang W, Chen X, Li T, Li Y, Wang R, He D, Luo W, Li X, Wu X. 2013. Screening a phage display library for a novel FGF8b-binding peptide with anti-tumor effect on prostate cancer. Exp Cell Res 319:1156-1164.
182. Kim Y, Lillo A M, Steiniger S C, Liu Y, Ballatore C, Anichini A, Mortarini R, Kaufmann G F, Zhou B, Felding-Habermann B, Janda K D. 2006. Targeting heat shock proteins on cancer cells: selection, characterization, and cell-penetrating properties of a peptidic GRP78 ligand. Biochemistry 45:9434-9444.
183. Arap M A, Lahdenranta J, Mintz P J, Hajitou A, Sarkis A S, Arap W, Pasqualini R. 2004. Cell surface expression of the stress response chaperone GRP78 enables tumor targeting by circulating ligands. Cancer Cell 6:275-284.
184. Müller J, Reichel R, Vogt S, Müller S P, Sauerwein W, Brandau W, Eggert A, Schramm A. 2016. Identification and Tumour-Binding Properties of a Peptide with High Affinity to the Disialoganglioside GD2. PLoS One 11:e0163648.
185. Feng C, Yu A, Wang Z, Wang K, Chen J, Wu Y, Deng T, Chen H, Hou Y, Ma S, Dai X, Huang L. 2024. A novel PDPN antagonist peptide CY12-RP2 inhibits melanoma growth via Wnt/beta-catenin and modulates the immune cells. J Exp Clin Cancer Res 43:9.
186. Quinn T, Zhang X, Miao Y. 2010. Targeted melanoma imaging and therapy with radiolabeled alpha-melanocyte stimulating hormone peptide analogues. G Ital Dermatol Venereol 145:245-258.
187. Giblin M F, Wang N, Hoffman T J, Jurisson S S, Quinn T P. 1998. Design and characterization of alpha-melanotropin peptide analogs cyclized through rhenium and technetium metal coordination. Proc Natl Acad Sci USA 95:12814-12818.
188. Newton J R, Miao Y, Deutscher S L, Quinn T P. 2007. Melanoma imaging with pretargeted bivalent bacteriophage. J NucI Med 48:429-436.
189. Szardenings M, Muceniece R, Mutule I, Mutulis F, Wikberg J E. 2000. New highly specific agonistic peptides for human melanocortin MC(1) receptor. Peptides 21:239-243.
190. Zhou Y, Mowlazadeh Haghighi S, Zoi I, Sawyer J R, Hruby V J, Cai M. 2017. Design of MC1R Selective gamma-MSH Analogues with Canonical Amino Acids Leads to Potency and Pigmentation. J Med Chem 60:9320-9329.
191. Nosanchuk J D, Valadon P, Feldmesser M, Casadevall A. 1999. Melanization of Cryptococcus neoformans in murine infection. Mol Cell Biol 19:745-750.
192. Howell R C, Revskaya E, Pazo V, Nosanchuk J D, Casadevall A, Dadachova E. 2007. Phage display library derived peptides that bind to human tumor melanin as potential vehicles for targeted radionuclide therapy of metastatic melanoma. Bioconjug Chem 18:1739-1748.
193. Matsuo A L, Juliano M A, Figueiredo C R, Batista W L, Tanaka A S, Travassos L R. 2011. A new phage-display tumor-homing peptide fused to antiangiogenic peptide generates a novel bioactive molecule with antimelanoma activity. Mol Cancer Res 9:1471-1478.
194. Tong S, Darwish S, Ariani H H N, Lozada K A, Salehi D, Cinelli M A, Silverman R B, Kaur K, Yang S. 2022. A Small Peptide Increases Drug Delivery in Human Melanoma Cells. Pharmaceutics 14.
195. Matsuo A L, Tanaka A S, Juliano M A, Rodrigues E G, Travassos L R. 2010. A novel melanoma-targeting peptide screened by phage display exhibits antitumor activity. J Mol Med (Berl) 88:1255-1264.
196. Burg M A, Pasqualini R, Arap W, Ruoslahti E, Stallcup W B. 1999. NG2 proteoglycan-binding peptides target tumor neovasculature. Cancer Res 59:2869-2874.
197. Hardy B, Raiter A. 2005. A mimotope peptide-based anti-cancer vaccine selected by BAT monoclonal antibody. Vaccine 23:4283-4291.
198. Luo W, Ko E, Hsu J C, Wang X, Ferrone S. 2006. Targeting melanoma cells with human high molecular weight-melanoma associated antigen-specific antibodies elicited by a peptide mimotope: functional effects. J Immunol 176:6046-6054.
199. Eriksson F, Culp W D, Massey R, Egevad L, Garland D, Persson M A, Pisa P. 2007. Tumor specific phage particles promote tumor regression in a mouse melanoma model. Cancer Immunol Immunother 56:677-687.
200. Yue H, Li Y, Yang T, Wang Y, Bao Q, Xu Y, Liu X, Miao Y, Yang M, Mao C. 2024. Filamentous phages as tumour-targeting immunotherapeutic bionanofibres. Nat Nanotechnol doi:10.1038/s41565-024-01800-4.
201. Colombo N, Sessa C, du Bois A, Ledermann J, McCluggage W G, McNeish I, Morice P, Pignata S, Ray-Coquard I, Vergote I, Baert T, Belaroussi I, Dashora A, Olbrecht S, Planchamp F, Querleu D, Group E-EOCCCW. 2019. ESMO-ESGO consensus conference recommendations on ovarian cancer: pathology and molecular biology, early and advanced stages, borderline tumours and recurrent diseasedagger. Ann Oncol 30:672-705.
202. Wang L, Wang X, Zhu X, Zhong L, Jiang Q, Wang Y, Tang Q, Li Q, Zhang C, Wang H, Zou D. 2024. Drug resistance in ovarian cancer: from mechanism to clinical trial. Mol Cancer 23:66.
203. Guo Y, Chen Y, Liu X, Min J J, Tan W, Zheng J H. 2020. Targeted cancer immunotherapy with genetically engineered oncolytic Salmonella typhimurium. Cancer Lett 469:102-110.
204. Chen W, Zhu Y, Zhang Z, Sun X. 2022. Advances in Salmonella Typhimurium-based drug delivery system for cancer therapy. Adv Drug Deliv Rev 185:114295.
205. Niethammer A G, Lubenau H, Mikus G, Knebel P, Hohmann N, Leowardi C, Beckhove P, Akhisaroglu M, Ge Y, Springer M, Grenacher L, Buchler M W, Koch M, Weitz J, Haefeli W E, Schmitz-Winnenthal F H. 2012. Double-blind, placebo-controlled first in human study to investigate an oral vaccine aimed to elicit an immune reaction against the VEGF-Receptor 2 in patients with stage I V and locally advanced pancreatic cancer. BMC Cancer 12:361.
206. Wick W, Wick A, Sahm F, Riehl D, von Deimling A, Bendszus M, Mickingereder P, Beckhove P, Schmitz Winnenthal F, Jungk C, Wieckoswski S, Keller A, Podola L, Herold-Mende C, Lubenau H, Unterberg A, Platten M J. 2018. P01.031 VXM01 phase I study in patients with progressive glioblastoma—final results. Neuro Oncol 20:iii235.
207. Schmitz-Winnenthal F H, Hohmann N, Niethammer A G, Friedrich T, Lubenau H, Springer M, Breiner K M, Mikus G, Weitz J, Ulrich A, Buechler M W, Pianka F, Klaiber U, Diener M, Leowardi C, Schimmack S, Sisic L, Keller A V, Koc R, Springfeld C, Knebel P, Schmidt T, Ge Y, Bucur M, Stamova S, Podola L, Haefeli W E, Grenacher L, Beckhove P. 2015. Anti-angiogenic activity of VXM01, an oral T-cell vaccine against VEGF receptor 2, in patients with advanced pancreatic cancer: A randomized, placebo-controlled, phase 1 trial. Oncoimmunology 4:e1001217.
208. Schmitz-Winnenthal F H, Hohmann N, Schmidt T, Podola L, Friedrich T, Lubenau H, Springer M, Wieckowski S, Breiner K M, Mikus G, Buchler M W, Keller A V, Koc R, Springfeld C, Knebel P, Bucur M, Grenacher L, Haefeli W E, Beckhove P. 2018. A phase 1 trial extension to assess immunologic efficacy and safety of prime-boost vaccination with VXM01, an oral T cell vaccine against VEGFR2, in patients with advanced pancreatic cancer. Oncoimmunology 7:e1303584.
209. Nemunaitis J, Cunningham C, Senzer N, Kuhn J, Cramm J, Litz C, Cavagnolo R, Cahill A, Clairmont C, Sznol M. 2003. Pilot trial of genetically modified, attenuated Salmonella expressing the E. coli cytosine deaminase gene in refractory cancer patients. Cancer Gene Ther 10:737-744.
210. Heimann D M, Rosenberg S A. 2003. Continuous intravenous administration of live genetically modified Salmonella typhimurium in patients with metastatic melanoma. J Immunother 26:179-180.
211. Gniadek T J, Augustin L, Schottel J, Leonard A, Saltzman D, Greeno E, Batist G. 2020. A Phase I, Dose Escalation, Single Dose Trial of Oral Attenuated Salmonella typhimurium Containing Human IL-2 in Patients With Metastatic Gastrointestinal Cancers. J Immunother 43:217-221.
212. Spizzo G, Went P, Dirnhofer S, Obrist P, Moch H, Baeuerle P A, Mueller-Holzner E, Marth C, Gastl G, Zeimet A G. 2006. Overexpression of epithelial cell adhesion molecule (Ep-CAM) is an independent prognostic marker for reduced survival of patients with epithelial ovarian cancer. Gynecol Oncol 103:483-488.
213. Toffoli G, Cernigoi C, Russo A, Gallo A, Bagnoli M, Boiocchi M. 1997. Overexpression of folate binding protein in ovarian cancers. Int J Cancer 74:193-198.
214. Kalli K R, Oberg A L, Keeney G L, Christianson T J, Low P S, Knutson K L, Hartmann L C. 2008. Folate receptor alpha as a tumor target in epithelial ovarian cancer. Gynecol Oncol 108:619-626.
215. Heo Y A. 2023. Mirvetuximab Soravtansine: First Approval. Drugs 83:265-273.
216. K C T, Suga K, Isoshima T, Aigaki T, Ito Y, Shiba K, Uzawa T. 2018. Wash-free and selective imaging of epithelial cell adhesion molecule (EpCAM) expressing cells with fluorogenic peptide ligands. Biochem Biophys Res Commun 500:283-287.
217. Xing L, Xu Y, Sun K, Wang H, Zhang F, Zhou Z, Zhang J, Zhang F, Caliskan B, Qiu Z, Wang M. 2018. Identification of a peptide for folate receptor alpha by phage display and its tumor targeting activity in ovary cancer xenograft. Sci Rep 8:8426.
218. Vandepapeliere P, Horsmans Y, Moris P, Van Mechelen M, Janssens M, Koutsoukos M, Van Belle P, Clement F, Hanon E, Wettendorff M, Garcon N, Leroux-Roels G. 2008. Vaccine adjuvant systems containing monophosphoryl lipid A and QS21 induce strong and persistent humoral and T cell responses against hepatitis B surface antigen in healthy adult volunteers. Vaccine 26:1375-1386.
219. Arpaia N, Godec J, Lau L, Sivick K E, McLaughlin L M, Jones M B, Dracheva T, Peterson S N, Monack D M, Barton G M. 2011. TLR signaling is required for Salmonella typhimurium virulence. Cell 144:675-688.
220. Ma C, Yin G, Yan D, He X, Zhang L, Wei Y, Huang Z. 2013. A novel peptide specifically targeting ovarian cancer identified by in vivo phage display. J Pept Sci 19:730-736.
221. Pu X, Ma C, Yin G, You F, Wei Y. 2014. Cell adhesion and invasion inhibitory effect of an ovarian cancer targeting peptide selected via phage display in vivo. Biochem Biophys Res Commun 443:858-863.
222. Zhou C, Kang J, Wang X, Wei W, Jiang W. 2015. Phage display screening identifies a novel peptide to suppress ovarian cancer cells in vitro and in vivo in mouse models. BMC Cancer 15:889.
223. Aina O H, Marik J, Liu R, Lau D H, Lam K S. 2005. Identification of novel targeting peptides for human ovarian cancer cells using “one-bead one-compound” combinatorial libraries. Mol Cancer Ther 4:806-813.
224. Ruoslahti E, Pierschbacher M D. 1987. New perspectives in cell adhesion: RGD and integrins. Science 238:491-497.
225. Sobral D V, Fuscaldi L L, Durante A C R, Mendonca F F, de Oliveira L R, Miranda A C C, Mejia J, Montor W R, de Barboza M F, Malavolta L. 2022. Comparative Evaluation of Radiochemical and Biological Properties of (131)I- and [(99m)Tc]Tc(CO)(3)-Labeled RGD Analogues Planned to Interact with the alpha(v)beta(3) Integrin Expressed in Glioblastoma. Pharmaceuticals (Basel) 15.
226. Yokoyama Y, Ramakrishnan S. 2005. Addition of an aminopeptidase N-binding sequence to human endostatin improves inhibition of ovarian carcinoma growth. Cancer 104:321-331.
227. Hossein-Nejad-Ariani H, Althagafi E, Kaur K. 2019. Small Peptide Ligands for Targeting EGFR in Triple Negative Breast Cancer Cells. Sci Rep 9:2723.
228. Hamzeh-Mivehroud M, Mahmoudpour A, Dastmalchi S. 2012. Identification of new peptide ligands for epidermal growth factor receptor using phage display and computationally modeling their mode of binding. Chem Biol Drug Des 79:246-259.
229. Park S W, Jun S Y, Kim J S, Kim Y S. 2021. Engineering of an EpCAM-targeting cyclic peptide to improve the EpCAM-mediated cellular internalization and tumor accumulation of a peptide-fused antibody. Biochem Biophys Res Commun 573:35-41.
230. Wang Y J, Tang L, Lu X H, Liu J T, Wang Y Y, Geng H X, Li X T, An Q. 2023. Efficacy of epi-1 modified epirubicin and curcumin encapsulated liposomes targeting-EpCAM in the inhibition of epithelial ovarian cancer cells. J Liposome Res 33:197-213.
231. Ma X, Kang X, He L, Zhou J, Zhou J, Sturm M B, Beer D G, Kuick R, Nancarrow D J, Appelman H D, Pang Z, Li W, Zhang C, Zhang W, Zhang Y, Wang T D, Li M. 2019. Identification of Tumor Specific Peptide as EpCAM Ligand and Its Potential Diagnostic and Therapeutic Clinical Application. Mol Pharm 16:2199-2213.
232. Dai Y, Tang Y, Huang W, Zhao Y, Gao X, Gu Y. 2022. Multi-modal imaging probe for EpCAM overexpressed in breast cancer. Talanta 250:123715.
233. Wang W, Wang Z, Bu X, Li R, Zhou M, Hu Z. 2015. Discovering of Tumor-targeting Peptides using Bi-functional Microarray. Adv Healthc Mater 4:2802-2808.
234. Shinde S D, Rao K B, Behera S K, Arya N, Sahu B. 2022. Epithelial cell adhesion molecule (EpCAM) binding short peptides derived from antibody MOC-31; De-novo design, synthesis and their in-vitro evaluation. Biochem Biophys Res Commun 600:1-5.
235. Koolpe M, Dail M, Pasquale E B. 2002. An ephrin mimetic peptide that selectively targets the EphA2 receptor. J Biol Chem 277:46974-46979.
236. Murai K K, Nguyen L N, Koolpe M, McLennan R, Krull C E, Pasquale E B. 2003. Targeting the EphA4 receptor in the nervous system with biologically active peptides. Mol Cell Neurosci 24:1000-1011.
237. Hall J M, Chang C Y, McDonnell D P. 2000. Development of peptide antagonists that target estrogen receptor β-coactivator interactions. Mol Endocrinol 14:2010-2023.
238. Hulin-Curtis S L, Davies J A, Nestic D, Bates E A, Baker A T, Cunliffe T G, Majhen D, Chester J D, Parker A L. 2020. Identification of folate receptor alpha (FRalpha) binding oligopeptides and their evaluation for targeted virotherapy applications. Cancer Gene Ther 27:785-798.
239. Santa Coloma T A, Dattatreyamurty B, Reichert L E, Jr. 1990. A synthetic peptide corresponding to human FSH beta-subunit 33-53 binds to FSH receptor, stimulates basal estradiol biosynthesis, and is a partial antagonist of FSH. Biochemistry 29:1194-1200.
240. Santa Coloma T A, Reichert L E, Jr. 1990. Identification of a follicle-stimulating hormone receptor-binding region in hFSH-beta-(81-95) using synthetic peptides. J Biol Chem 265:5037-5042.
241. Maruta F, Parker A L, Fisher K D, Hallissey M T, Ismail T, Rowlands D C, Chandler L A, Kerr D J, Seymour L W. 2002. Identification of FGF receptor-binding peptides for cancer gene therapy. Cancer Gene Ther 9:543-552.
242. Shen Y, Ruan L, Lian C, Li R, Tu Z, Liu H. 2019. Discovery of HB-EGF binding peptides and their functional characterization in ovarian cancer cell lines. Cell Death Discov 5:82.
243. Cannazza G, Cazzato A S, Marraccini C, Pavesi G, Pirondi S, Guerrini R, Pela M, Frassineti C, Ferrari S, Marverti G, Ponterini G, Costi M P. 2014. Internalization and stability of a thymidylate synthase Peptide inhibitor in ovarian cancer cells. J Med Chem 57:10551-10556.
244. Karasseva N G, Glinsky V V, Chen N X, Komatireddy R, Quinn T P. 2002. Identification and characterization of peptides that bind human ErbB-2 selected from a bacteriophage display library. J Protein Chem 21:287-296.
245. Diderich P, Heinis C. 2014. Phage selection of bicyclic peptides binding Her2. Tetrahedron 70:7733-7739.
246. Houimel M, Schneider P, Terskikh A, Mach J P. 2001. Selection of peptides and synthesis of pentameric peptabody molecules reacting specifically with ErbB-2 receptor. Int J Cancer 92:748-755.
247. Park J, Park S, Kim S, Lee I H, Saw P E, Lee K, Kim Y C, Kim Y J, Farokhzad O C, Jeong Y Y, Jon S. 2013. HER2-specific aptide conjugated magneto-nanoclusters for potential breast cancer imaging and therapy. J Mater Chem B 1:4576-4583.
248. Pero S C, Shukla G S, Armstrong A L, Peterson D, Fuller S P, Godin K, Kingsley-Richards S L, Weaver D L, Bond J, Krag D N. 2004. Identification of a small peptide that inhibits the phosphorylation of ErbB2 and proliferation of ErbB2 overexpressing breast cancer cells. Int J Cancer 111:951-960.
249. Shadidi M, Sioud M. 2003. Identification of novel carrier peptides for the specific delivery of therapeutics into cancer cells. FASEB J 17:256-258.
250. Hu F, Huang Y, Zhang G, Zhao R, Yang H, Zhang D. 2014. Targeted bioimaging and photodynamic therapy of cancer cells with an activatable red fluorescent bioprobe. Anal Chem 86:7987-7995.
251. Lee J H, Engler J A, Collawn J F, Moore B A. 2001. Receptor mediated uptake of peptides that bind the human transferrin receptor. Eur J Biochem 268:2004-2012.
252. Kelly K A, Allport J R, Tsourkas A, Shinde-Patil V R, Josephson L, Weissleder R. 2005. Detection of vascular adhesion molecule-1 expression using a novel multimodal nanoparticle. Circ Res 96:327-336.
253. Kelly K A, Nahrendorf M, Yu A M, Reynolds F, Weissleder R. 2006. In vivo phage display selection yields atherosclerotic plaque targeted peptides for imaging. Mol Imaging Biol 8:201-207.
254. Zhao S, Li C, Gao Y, Qian L, Dong J, Zhai L, Chen B, Zhang J. 2021. Screening and Identification of a Specific Binding Peptide to Ovarian Cancer Cells from a Phage-Displayed Peptide Library. Int J Pept Res Ther 27:1741-1749.
255. Pan X, Geng Z, Li J, Li X, Zhang M, Wang X, Cong Y, Huang K, Xu J, Jia X. 2022. Peptide PDHPS1 Inhibits Ovarian Cancer Growth through Disrupting YAP Signaling. Mol Cancer Ther 21:1160-1170.
256. Shen Y A, Liu C S, Chang Y H, Chen P H, He C L, Wu H C, Chuang C M. 2015. Subtype-specific binding peptides enhance the therapeutic efficacy of nanomedicine in the treatment of ovarian cancer. Cancer Lett 360:39-47.
257. Yang C, He X, Liu X, Tang Z, Liang X. 2015. OSTP as a novel peptide specifically targeting human ovarian cancer. Oncol Rep 34:972-978.
258. Wang L, Hu Y, Li W, Wang F, Lu X, Han X, Lv J, Chen J. 2016. Identification of a peptide specifically targeting ovarian cancer by the screening of a phage display peptide library. Oncol Lett 11:4022-4026.
259. Soendergaard M, Newton-Northup J R, Deutscher S L. 2014. In vivo phage display selection of an ovarian cancer targeting peptide for SPECT/C T imaging. Am J NucI Med Mol Imaging 4:561-570.
260. Asar M, Newton-Northup J, Deutscher S, Soendergaard M. 2019. Ovarian Cancer Targeting Phage for In Vivo Near-Infrared Optical Imaging. Diagnostics (Basel) 9.
261. Wang C H, Weng C H, Che Y J, Wang K, Lee G B. 2015. Cancer cell-specific oligopeptides selected by an integrated microfluidic system from a phage display library for ovarian cancer diagnosis. Theranostics 5:431-442.
262. Zhang L, Yin G, Yan D, Wei Y, Ma C, Huang Z, Liao X, Yao Y, Chen X, Hao B. 2011. In vitro screening of ovarian tumor specific peptides from a phage display peptide library. Biotechnol Lett 33:1729-1735.

What is claimed is:

1. A genetically modified Salmonella cell (GMSC) engineered to display pattern-associated and danger-associated molecular patterns to recruit and enhance innate immunity and exhibit regulated delayed lysis in vivo, the GMSC comprising a first heterologous nucleic acid that encodes a first gene product that causes the GMSC to be selectively localized to and/or internalized by target cells in vivo and a second heterologous nucleic acid that encodes a second gene product that facilitates killing of the target cells following internalization.

2.-29. (canceled)

30. A plasmid comprising a first heterologous nucleic acid that encodes a first gene product whose expression in a GMSC causes the GMSC to be localized to and/or internalized by a target cell in vivo, wherein the first heterologous nucleic acid is operably linked to a first promoter that controls expression of the first heterologous nucleic acid in a Salmonella cell; and a second heterologous nucleic acid that encodes a second gene product whose expression facilitates killing of target cells following internalization by the target cell, wherein the second heterologous nucleic acid is operably linked to (i) a second promoter that controls expression of the second heterologous nucleic acid in the target cell and (ii) to a nucleus targeting sequence that directs the plasmid to a nucleus of target cell.

31. The plasmid of claim 30, wherein the first heterologous nucleic acid comprises a nucleic acid sequence encoding OmpA operably linked to a nucleic acid sequence encoding PLZ4.

32. The plasmid of claim 31, wherein the first heterologous nucleic acid comprises a nucleic acid sequence encoding SEQ ID NO: 2, or an amino sequence comprising at least 90% or 95% sequence identity therewith.

33. The plasmid of claim 30, wherein the second heterologous nucleic acid comprises a nucleic acid sequence encoding CXCL11 or KillerRed, or both.

34. The plasmid of claim 33, wherein the second heterologous nucleic acid comprises a nucleic acid sequence encoding KillerRed.

35. The plasmid of claim 33, wherein CXCL11 is fused to KillerRed.

36. The plasmid of claim 35, further comprising a P2A peptide is situated between CXCL11 and KillerRed.

37. The plasmid of claim 30, wherein the second heterologous nucleic acid comprises a nucleic acid sequence encoding KillerRed fused to neuromodulin N-terminal sequence, or KillerRed fused to a mitochondrial targeting sequence.

38. The plasmid of claim 37, wherein the second heterologous nucleic acid comprises a sequence encoding SEQ ID NO:18, SEQ ID NO:20, SEQ ID NO: 52 and SEQ ID NO: 53; or an amino sequence comprising at least 90% or 95% sequence identity therewith.

39. The plasmid of claim 30, wherein the first heterologous nucleic acid comprises a nucleic acid sequence encoding a LHRH peptide or HER2ScFv, or both.

40. The plasmid of claim 39, wherein the first heterologous nucleic acid comprises a nucleic acid sequence encoding at least one selected from the group consisting of SEQ ID NO: 24, SEQ ID NO:25, and SEQ ID NO: 47; or an amino sequence comprising at least 90% or 95% sequence identity therewith.

41. The plasmid of claim 30, wherein the second heterologous nucleic acid comprises a nucleic acid sequence encoding a HAC-PD1 or HaPD1-IgG, or both.

42. The plasmid of claim 41, wherein the second heterologous nucleic acid comprises a nucleic acid sequence encoding at least one selected from the group consisting of SEQ ID NO: 49, and SEQ ID NO: 51; or an amino sequence comprising at least 90% or 95% sequence identity therewith.

43. The plasmid of claim 30, wherein the second heterologous nucleic acid comprises a nucleic acid sequence encoding an HLA peptide.

44. The plasmid of claim 43, wherein the second heterologous nucleic acid comprises a nucleic acid sequence encoding SEQ ID NO: 21; or an amino sequence comprising at least 90% or 95% sequence identity therewith.

45. The plasmid of claim 30, wherein the first promoter comprises a bacterial promoter, optionally further comprising a sequence such as bla SSopt for secretion of the first gene product, and the second promoter a eukaryotic promoter for delivery to the nucleus of the target cells wherein the bacterial promoter optionally comprises P_{trc}, P_{tac}, P_{lac}, or Pₗₚₚ, wherein the sequence for secretion optionally comprises bla SSopt and wherein the eukaryotic promoter optionally comprises P_{CMV} or P_{EF1α}.

46. (canceled)

47. (canceled)

48. (canceled)

49. The GMSC of claim 1, wherein the first heterologous nucleotide encodes a peptide, wherein the peptide is selected from a peptide set forth in Tables 10, 11, or 12.

50. The GMSC of claim 1, wherein the GMSC includes one of or a combination of mutations selected from ΔpagP::Pₗₚₚ lpxE, ΔfliC, ΔarnT, ΔeptA, ΔpagL, and/or ΔlpxR,

51. The GMSC of claim 1, wherein the GMSC comprises one or more of the following mutations: ΔpurA, ΔP_{rhaBAD}::rhaRS P_{rhaBAD} purA, ΔguaBA, ΔP_{guaBA}::rhaRS P_{rhaBAD} guaBA, ΔpyrF, ΔP_{pyrF}::rhaRS P_{rhaBAD} pyrF, ΔPₜₐᵣ::P_{trcΔlacO888} tar, ΔPₜₛᵣ::P_{trcΔlacO888} tsr, or Δtrg.

52. A plasmid of claim of claim 30, wherein the plasmid is selected from a plasmid set forth in Table 6 or its derivatives.


SEQUENCE LISTING (323 sequences)

SEQ ID NO: 1 - DNA, 1053 bp, Synthetic construct
  source 1..1053: mol_type = other DNA
atgaaaaagacagctatcgcgattgcagtggcactggctggtttcgctaccgtagcgcaggccgctccgaaagataacacctggtacgctggtgctaaactgggctggtctcagtaccatgacaccggcttcattcacaatgatggcccgactcatgaaaaccaactgggcgcaggtgcttttggtggttaccaggttaacccgtatgttggctttgaaatgggctacgactggttaggccgtatgccgtacaaaggcgacaacatcaatggcgcttataaagctcagggcgttcagttgaccgctaaactgggttatccaatcactgacgatctggacgtttatacccgtctgggtggtatggtatggcgtgcagacaccaagtctaacgtccctggcggcccgtctactaaagaccacgacaccggcgtttccccggtattcgcgggcggtatcgagtatgctatcacccctgaaatcgcaacccgtctggaataccagtggactaacaacatcggtgatgccaacaccatcggcacccgtccggacaacggcctgctgagcgtaggtgtttcctaccgtttcggccagcaagaagctgctccggtagtagctccggcaccagctccggctccggaagtacagaccaagcacttcactctgaagtctgacgtactgttcaacttcaacaaatctaccctgaagccggaaggccagcaggctctggatcagctgtacagccagctgagcaacctggatccgaaagacggttccgttgtcgttctgggcttcactgaccgtatcggttctgacgcttacaaccagggtctgtccgagaaacgtgctcagtctgttgttgattacctgatctccaaaggtattccgtctgacaaaatctccgcacgtggtatgggcgaatctaacccggttaccggcaacacctgtgacaacgtgaaacctcgcgctgccctgatcgattgcctggctccggatcgtcgcgtagagatcgaagttaaaggcgttaaagacgtggtaactcagccgcaggcttaa

SEQ ID NO: 2 - protein, 350 aa, Synthetic construct
MKKTAIAIAVALAGFATVAQAAPKDNTWYAGAKLGWSQYHDTGFIHNDGPTHENQLGAGAFGGYQVNPYVGFEMGYDWLGRMPYKGDNINGAYKAQGVQLTAKLGYPITDDLDVYTRLGGMVWRADTKSNVPGGPSTKDHDTGVSPVFAGGIEYAITPEIATRLEYQWTNNIGDANTIGTRPDNGLLSVGVSYRFGQQEAAPVVAPAPAPAPEVQTKHFTLKSDVLFNFNKSTLKPEGQQALDQLYSQLSNLDPKDGSVVVLGFTDRIGSDAYNQGLSEKRAQSVVDYLISKGIPSDKISARGMGESNPVTGNTCDNVKPRAALIDCLAPDRRVEIEVKGVKDVVTQPQA

SEQ ID NO: 3 - DNA, 27 bp, Synthetic construct
  source 1..27: mol_type = other DNA
tgccaggacggtcgtatgggtttctgc

SEQ ID NO: 4 - protein, 9 aa, Synthetic construct
CQDGRMGFC

SEQ ID NO: 5-6 - skipped (2 entries)

SEQ ID NO: 7 - DNA, 1107 bp, Synthetic construct
  source 1..1107: mol_type = other DNA
ctaggccaactggcgcagcattcgacgcagcggctcggcggcgccccataacaactggtcgcctacggtaaacgccgacaagaactctggccccatgttcagcttacgcagacgaccaaccggcgtagtcaacgtgccggtcaccgccgccggggttaattcgcgcatagtgatatcacgatcgttcggcaccactttcgcccacggattatgtgccgccagcagttcttccaccgtcggaatggatacctcttttttcagcttgatggtgaacgcctggctgtgacagcgcagcgcgccgacgcgcacacacaaaccatcaaccggaatcacagaggcagtattgagaatcttgttggtttccgcctggcctttccactcttcgcggctctggccgttatcgagctgtttgtcgatccaggggatcaggcttcccgccagcggtacgccaaagttatcaaccggcagctcgccgctgcgggtcaatgccgtaactttgcgttcaatatcaagaattgcggaagacggcgtcgccagttcatcggcgacatggccatacaactgacccatctgggttaacagctcgcgcatatggcgcgcgccgccgccggaggcggcctgataggtcgcgacggatacccagtcaacgagattatgggcaaagagaccgcccagcgacatcaacatcaggctaacggtacagttaccgcccacaaaggtcttcacgccattgttcaggccgtcggtaatcacgtcctggttgaccgggtcgagaataataatggcatcatctttcatgcgcagcgtagaagccgcatcaatccagtaaccctgccatccgctttcgcgcagctttggataaatttcgttggtataatcgccgccctggcaggtcacgatgatatcgagcgcttttagcgcatccagatcaaaagcgtcctgtagcgtgccggtggaggtgtcgccgaaggtgggcgccgcctgtccaaactgggaggtagaaaagaaaacagggcgaatagcgtcgaaatcgcgctcctctaccatgcgttgcatgagaacagagccgaccattccgcgccagccgataaaaccaacatttttcat

SEQ ID NO: 8 - protein, 368 aa, Synthetic construct
ALQRLMRRLPEAAGWLLQDGVTFASLFEPGMNLKRLRGVPTTLTGTVAAPTLERMTIDRDNPVVKAWPNHAALLEEVTPISVEKKLKITFAQSHCRLAGVRVCLGDVPIVSATNLIKNTEAQGKWEERSQGNDLQKDIWPILSGALPVGFNDVPLEGSRTLATVKREIDLIASSPTALEDAVHGYLQGMQTLLERMHRAGGGSAAQYTAVSVWDVLNHAFLGGLSMLMLSVTCNGGVFTKVGNNLGDTIVDQNVPDLIIIADDKMRLTSAADIWYGQWGSERLKPYIENTYDGGQCTVIIDLAKLADLDFADQLTGTSTDGFTPAAQGFQSTSFFVPRIADFDREEVMRQMLVSGVMGRWGIFGVNKM

SEQ ID NO: 9 - DNA, 1257 bp, Synthetic construct
  source 1..1257: mol_type = other DNA
gtggataaatttcgtgttcaggggccaacgaagctccagggcgaagtcacaatttccggcgctaaaaatgctgctctgcctatcctttttgccgcactactggcggaagaaccggtagagatccagaacgtcccgaaactgaaagacgtcgatacatcaatgaagctgctaagccagctgggtgcgaaagtagaacgtaatggttctgtgcatattgatgcccgcgacgttaatgtattctgcgcaccttacgatctggttaaaaccatgcgtgcttctatctgggcgctggggccgctggtagcgcgctttggtcaggggcaagtttcactacctggcggttgtacgatcggtgcgcgtccggttgatctacacatttctggcctcgaacaattaggcgcgaccatcaaactggaagaaggttacgttaaagcttccgtcgatggtcgtttgaaaggtgcacatatcgtgatggataaagtcagcgttggcgcaacggtgaccatcatgtgtgctgcaaccctggcggaaggcaccacgattattgaaaacgcagcgcgtgaaccggaaatcgtcgataccgcgaacttcctgattacgctgggtgcgaaaattagcggtcagggcaccgatcgtatcgtcatcgaaggtgtggaacgtttaggcggcggtgtctatcgcgttctgccggatcgtatcgaaaccggtactttcctggtggcggcggcgatttctcgcggcaaaattatctgccgtaacgcgcagccagatactctcgacgccgtgctggcgaaactgcgtgacgctggagcggacatcgaagtcggcgaagactggattagcctggatatgcatggcaaacgtccgaaggctgttaacgtacgtaccgcgccgcatccggcattcccgaccgatatgcaggcccagttcacgctgttgaacctggtggcagaagggaccgggtttatcaccgaaacggtctttgaaaaccgctttatgcatgtgccagagctgagccgtatgggcgcgcacgccgaaatcgaaagcaataccgttatttgtcacggtgttgaaaaactttctggcgcacaggttatggcaaccgatctgcgtgcatcagcaagcctggtgctggctggctgtattgcggaagggacgacggtggttgatcgtatttatcacatcgatcgtggctacgaacgcattgaagacaaactgcgcgctttaggtgcaaatattgagcgtgtgaaaggcgaa

SEQ ID NO: 10 - protein, 419 aa, Synthetic construct
MDKFRVQGPTKLQGEVTISGAKNAALPILFAALLAEEPVEIQNVPKLKDVDTSMKLLSQLGAKVERNGSVHIDARDVNVFCAPYDLVKTMRASIWALGPLVARFGQGQVSLPGGCTIGARPVDLHISGLEQLGATIKLEEGYVKASVDGRLKGAHIVMDKVSVGATVTIMCAATLAEGTTIIENAAREPEIVDTANFLITLGAKISGQGTDRIVIEGVERLGGGVYRVLPDRIETGTFLVAAAISRGKIICRNAQPDTLDAVLAKLRDAGADIEVGEDWISLDMHGKRPKAVNVRTAPHPAFPTDMQAQFTLLNLVAEGTGFITETVFENRFMHVPELSRMGAHAEIESNTVICHGVEKLSGAQVMATDLRASASLVLAGCIAEGTTVVDRIYHIDRGYERIEDKLRALGANIERVKGE

SEQ ID NO: 11 - DNA, 876 bp, Synthetic construct
  source 1..876: mol_type = other DNA
tgacaacttgacggctacatcattcactttttcttcacaaccggcacgaaactcgctcgggctggccccggtgcattttttaaatactcgcgagaaatagagttgatcgtcaaaaccaacattgcgaccgacggtggcgataggcatccgggtagtgctcaaaagcagcttcgcctgactaatgcgttggtcctcgcgccagcttaagacgctaatccctaactgctggcggaaaagatgtgacagacgcgacggcgacaagcaaacatgctgtgcgacgctggcgatatcaaaattgctgtctgccaggtgatcgctgatgtactgacaagcctcgcgtacccgattatccatcggtggatggagcgactcgttaatcgcttccatgcgccgcagtaacaattgctcaagcagatttatcgccagcagctccgaatagcgcccttccccttgcccggcgttaatgatttgcccaaacaggtcgctgaaatgcggctggtgcgcttcatccgggcgaaagaaacccgtattggcaaatattgacggccagttaagccattcatgccagtaggcgcgcggacgaaagtaaacccactggtgataccattcgcgagcctccggatgacgaccgtagtgatgaatctctcctggcgggaacagcaaaatatcacccggtcggcagacaaattctcgtccctgatttttcaccaccccctgaccgcgaatggtgagattgagaatataacctttcattcccagcggtcggtcgataaaaaaatcgagataaccgttggcctcaatcggcgttaaacccgccaccagatgggcgttaaacgagtatcccggcagcaggggatcattttgcgcttcagccat

SEQ ID NO: 12 - protein, 292 aa, Synthetic construct
SLKVAVDNVKEECGARFESPSAGTCKKFVRSFYLQDDFGVNRGVTAIPMRTTSLLLKAQSIRQDERWSLVSIGLQQRFLHSLRSPSLCVHQAVSAIDFNSDALHDSIYQCAERVRNDMPPHLSENIAEMRRLLLQELLNIALLESYRGEGQGANIIQGFLDSFHPQHAEDPRFFGTNAFISPWNLWEHWYARPRFYVWQHYWERAEPHRGYHHIEGPPFLLIDGPRCVFERGQNKVVGQGRITLNLIYGKMGLPRDIFFDLYGNAEIPTLGAVLHANFSYGPLLPDNQAEAM

SEQ ID NO: 13 - DNA, 285 bp, Homo sapiens
  source 1..285: mol_type = genomic DNA
atgagtgtgaagggcatggctatagccttggctgtgatattgtgtgctacagttgttcaaggcttccccatgttcaaaagaggacgctgtctttgcataggccctggggtaaaagcagtgaaagtggcagatattgagaaagcctccataatgtacccaagtaacaactgtgacaaaatagaagtgattattaccctgaaagaaaataaaggacaacgatgcctaaatcccaaatcgaagcaagcaaggcttataatcaaaaaagttgaaagaaagaatttttaa

SEQ ID NO: 14 - protein, 94 aa, Homo sapiens
MSVKGMAIALAVILCATVVQGFPMFKRGRCLCIGPGVKAVKVADIEKASIMYPSNNCDKIEVIITLKENKGQRCLNPKSKQARLIIKKVERKNF

SEQ ID NO: 15 - DNA, 303 bp, Mus musculus
  source 1..303: mol_type = genomic DNA
atgaacaggaaggtcacagccatagccctggctgcgatcatctgggccacagctgctcaaggcttccttatgttcaaacaggggcgctgtctttgcatcggccccgggatgaaagccgtcaaaatggcagagatcgagaaagcttctgtaatttacccgagtaacggctgcgacaaagttgaagtgattgttactatgaaggctcataaacgacaaaggtgcctggaccccagatccaagcaagctcgcctcataatgcaggcaatagaaaaaaagaattttttaaggcgtcaaaacatgtaa

SEQ ID NO: 16 - protein, 100 aa, Mus musculus
MNRKVTAIALAAIIWATAAQGFLMFKQGRCLCIGPGMKAVKMAEIEKASVIYPSNGCDKVEVIVTMKAHKRQRCLDPRSKQARLIMQAIEKKNFLRRQNM

SEQ ID NO: 17 - DNA, 774 bp, Synthetic construct
  source 1..774: mol_type = other DNA
atgctgtgctgtatgagaagaaccaaacaggttgaaaagaatgatgaggaccaaaagatctccgagggcggccccgccctgttccagagcgacatgaccttcaaaatcttcatcgacggcgaggtgaacggccagaagttcaccatcgtggccgacggcagcagcaagttcccccacggcgacttcaacgtgcacgccgtgtgcgagaccggcaagctgcccatgagctggaagcccatctgccacctgatccagtacggcgagcccttcttcgcccgctaccccgacggcatcagccatttcgcccaggagtgcttccccgagggcctgagcatcgaccgcaccgtgcgcttcgagaacgacggcaccatgaccagccaccacacctacgagctggacgacacctgcgtggtgagccgcatcaccgtgaactgcgacggcttccagcccgacggccccatcatgcgcgaccagctggtggacatcctgcccaacgagacccacatgttcccccacggccccaacgccgtgcgccagctggccttcatcggcttcaccaccgccgacggcggcctgatgatgggccacttcgacagcaagatgaccttcaacggcagccgcgccatcgagatccccggcccacacttcgtgaccatcatcaccaagcagatgagggacaccagcgacaagcgcgaccacgtgtgccagcgcgaggtggcctacgcccacagcgtgccccgcatcaccagcgccatcggtagcgacgaggattaa

SEQ ID NO: 18 - protein, 257 aa, Synthetic construct
MLCCMRRTKQVEKNDEDQKISEGGPALFQSDMTFKIFIDGEVNGQKFTIVADGSSKFPHGDFNVHAVCETGKLPMSWKPICHLIQYGEPFFARYPDGISHFAQECFPEGLSIDRTVRFENDGTMTSHHTYELDDTCVVSRITVNCDGFQPDGPIMRDQLVDILPNETHMFPHGPNAVRQLAFIGFTTADGGLMMGHFDSKMTFNGSRAIEIPGPHFVTIITKQMRDTSDKRDHVCQREVAYAHSVPRITSAIGSDED

SEQ ID NO: 19 - DNA, 921 bp, Synthetic construct
  source 1..921: mol_type = other DNA
atgtccgtcctgacgccgctgctgctgcggggcttgacaggctcggcccggcggctcccagtgccgcgcgccaagatccattcgttgggggatctgtccgtcctgacgccgctgctgctgcggggcttgacaggctcggcccggcggctcccagtgccgcgcgccaagatccattcgttgggggatccaccggtcgccaccatgggttcagagggcggccccgccctgttccagagcgacatgaccttcaaaatcttcatcgacggcgaggtgaacggccagaagttcaccatcgtggccgacggcagcagcaagttcccccacggcgacttcaacgtgcacgccgtgtgcgagaccggcaagctgcccatgagctggaagcccatctgccacctgatccagtacggcgagcccttcttcgcccgctaccccgacggcatcagccatttcgcccaggagtgcttccccgagggcctgagcatcgaccgcaccgtgcgcttcgagaacgacggcaccatgaccagccaccacacctacgagctggacgacacctgcgtggtgagccgcatcaccgtgaactgcgacggcttccagcccgacggccccatcatgcgcgaccagctggtggacatcctgcccaacgagacccacatgttcccccacggccccaacgccgtgcgccagctggccttcatcggcttcaccaccgccgacggcggcctgatgatgggccacttcgacagcaagatgaccttcaacggcagccgcgccatcgagatccccggcccacacttcgtgaccatcatcaccaagcagatgagggacaccagcgacaagcgcgaccacgtgtgccagcgcgaggtggcctacgcccacagcgtgccccgcatcaccagcgccatcggtagcgacgaggattaa

SEQ ID NO: 20 - protein, 306 aa, Synthetic construct
MSVLTPLLLRGLTGSARRLPVPRAKIHSLGDLSVLTPLLLRGLTGSARRLPVPRAKIHSLGDPPVATMGSEGGPALFQSDMTFKIFIDGEVNGQKFTIVADGSSKFPHGDFNVHAVCETGKLPMSWKPICHLIQYGEPFFARYPDGISHFAQECFPEGLSIDRTVRFENDGTMTSHHTYELDDTCVVSRITVNCDGFQPDGPIMRDQLVDILPNETHMFPHGPNAVRQLAFIGFTTADGGLMMGHFDSKMTFNGSRAIEIPGPHFVTIITKQMRDTSDKRDHVCQREVAYAHSVPRITSAIGSDED

SEQ ID NO: 21 - DNA, 249 bp, Synthetic construct
  source 1..249: mol_type = other DNA
atgctggtcatggcgccccgaaccgtcctcctgctgctctcggcggccctggccctgaccgagacctgggccggctccggtaccgggcccgggcctaggggcctggctgtcctggcagttgtggtcatcggagctgtggtcgctgctgtgatgtgtaggaggaagagttcaggtggaaaaggagggagctactctcaggctgcgtgcagcgacagtgcccagggctctgatgtgtctctcacagcttga

SEQ ID NO: 22 - protein, 75 aa, Synthetic construct
MLVMAPRTVLLLLSAALALTETWAGSGLAVLAVVVIGAVVAAVMCRRKSSGGKGGSYSQAACSDSAQGSDVSLTA

SEQ ID NO: 23 - DNA, 36 bp, Synthetic construct
  source 1..36: mol_type = other DNA
tgcgaacattggagctatggcctgcgtccgggctgc

SEQ ID NO: 24 - protein, 12 aa, Synthetic construct
CEHWSYGLRPGC

SEQ ID NO: 25 - protein, 10 aa, Synthetic construct
EHWSYGLRPG

SEQ ID NO: 26 - DNA, 60 bp, Synthetic construct
  source 1..60: mol_type = other DNA
atgctgtgctgtatgagaagaaccaaacaggttgaaaagaatgatgaggaccaaaagatc

SEQ ID NO: 27 - protein, 20 aa, Synthetic construct
MLCCMRRTKQVEKNDEDQKI

SEQ ID NO: 28 - DNA, 711 bp, Synthetic construct
  source 1..711: mol_type = other DNA
tccgagggcggccccgccctgttccagagcgacatgaccttcaaaatcttcatcgacggcgaggtgaacggccagaagttcaccatcgtggccgacggcagcagcaagttcccccacggcgacttcaacgtgcacgccgtgtgcgagaccggcaagctgcccatgagctggaagcccatctgccacctgatccagtacggcgagcccttcttcgcccgctaccccgacggcatcagccatttcgcccaggagtgcttccccgagggcctgagcatcgaccgcaccgtgcgcttcgagaacgacggcaccatgaccagccaccacacctacgagctggacgacacctgcgtggtgagccgcatcaccgtgaactgcgacggcttccagcccgacggccccatcatgcgcgaccagctggtggacatcctgcccaacgagacccacatgttcccccacggccccaacgccgtgcgccagctggccttcatcggcttcaccaccgccgacggcggcctgatgatgggccacttcgacagcaagatgaccttcaacggcagccgcgccatcgagatccccggcccacacttcgtgaccatcatcaccaagcagatgagggacaccagcgacaagcgcgaccacgtgtgccagcgcgaggtggcctacgcccacagcgtgccccgcatcaccagcgccatcggtagcgacgaggat

SEQ ID NO: 29 - protein, 237 aa, Synthetic construct
SEGGPALFQSDMTFKIFIDGEVNGQKFTIVADGSSKFPHGDFNVHAVCETGKLPMSWKPICHLIQYGEPFFARYPDGISHFAQECFPEGLSIDRTVRFENDGTMTSHHTYELDDTCVVSRITVNCDGFQPDGPIMRDQLVDILPNETHMFPHGPNAVRQLAFIGFTTADGGLMMGHFDSKMTFNGSRAIEIPGPHFVTIITKQMRDTSDKRDHVCQREVAYAHSVPRITSAIGSDED

SEQ ID NO: 30 - DNA, 66 bp, Synthetic construct
  source 1..66: mol_type = other DNA
ggaagcggagctactaacttcagcctgctgaagcaggctggcgacgtggaggagaaccctggacct

SEQ ID NO: 31 - protein, 22 aa, Synthetic construct
GSGATNFSLLKQAGDVEENPGP

SEQ ID NO: 32 - DNA, 282 bp, Homo sapiens
  source 1..282: mol_type = genomic DNA
atgagtgtgaagggcatggctatagccttggctgtgatattgtgtgctacagttgttcaaggcttccccatgttcaaaagaggacgctgtctttgcataggccctggggtaaaagcagtgaaagtggcagatattgagaaagcctccataatgtacccaagtaacaactgtgacaaaatagaagtgattattaccctgaaagaaaataaaggacaacgatgcctaaatcccaaatcgaagcaagcaaggcttataatcaaaaaagttgaaagaaagaatttt

SEQ ID NO: 33 - protein, 94 aa, Homo sapiens; identical to SEQ ID NO: 14

SEQ ID NO: 34 - DNA, 60 bp, Synthetic construct; identical to SEQ ID NO: 26
  source 1..60: mol_type = other DNA

SEQ ID NO: 35 - protein, 20 aa, Synthetic construct
MLCCMRRTKQVEKNDEDQKI

SEQ ID NO: 36 - DNA, 711 bp, Synthetic construct; identical to SEQ ID NO: 28
  source 1..711: mol_type = other DNA

SEQ ID NO: 37 - protein, 237 aa, Synthetic construct; identical to SEQ ID NO: 29

SEQ ID NO: 38 - DNA, 66 bp, Synthetic construct; identical to SEQ ID NO: 30
  source 1..66: mol_type = other DNA

SEQ ID NO: 39 - protein, 22 aa, Synthetic construct
GSGATNFSLLKQAGDVEENPGP

SEQ ID NO: 40 - DNA, 300 bp, Mus musculus
  source 1..300: mol_type = genomic DNA
atgaacaggaaggtcacagccatagccctggctgcgatcatctgggccacagctgctcaaggcttccttatgttcaaacaggggcgctgtctttgcatcggccccgggatgaaagccgtcaaaatggcagagatcgagaaagcttctgtaatttacccgagtaacggctgcgacaaagttgaagtgattgttactatgaaggctcataaacgacaaaggtgcctggaccccagatccaagcaagctcgcctcataatgcaggcaatagaaaaaaagaattttttaaggcgtcaaaacatg

SEQ ID NO: 41 - protein, 100 aa, Mus musculus; identical to SEQ ID NO: 16

SEQ ID NO: 42 - DNA, 66 bp, Synthetic construct; identical to SEQ ID NO: 30
  source 1..66: mol_type = other DNA

SEQ ID NO: 43 - protein, 22 aa, Synthetic construct
GSGATNFSLLKQAGDVEENPGP

SEQ ID NO: 44 - DNA, 1095 bp, Synthetic construct
  source 1..1095: mol_type = other DNA
atgaaaaagacagctatcgcgattgcagtggcactggctggtttcgctaccgtagcgcaggccgctccgaaagataacacctggtacgctggtgctaaactgggctggtctcagtaccatgacaccggcttcattcacaatgatggcccgactcatgaaaaccaactgggcgcaggtgcttttggtggttaccaggttaacccgtatgttggctttgaaatgggctacgactggttaggccgtatgccgtacaaaggcgacaacatcaatggcgcttataaagctcagggcgttcagttgaccgctaaactgggttatccaatcactgacgatctggacgtttatacccgtctgggtggtatggtatggcgtgcagacaccaagtctaacgtccctggcgcggccgccggcggaggaggatctcatcatcaccatcaccatggcccgtctactaaagaccacgacaccggcgtttccccggtattcgcgggcggtatcgagtatgctatcacccctgaaatcgcaacccgtctggaataccagtggactaacaacatcggtgatgccaacaccatcggcacccgtccggacaacggcctgctgagcgtaggtgtttcctaccgtttcggccagcaagaagctgctccggtagtagctccggcaccagctccggctccggaagtacagaccaagcacttcactctgaagtctgacgtactgttcaacttcaacaaatctaccctgaagccggaaggccagcaggctctggatcagctgtacagccagctgagcaacctggatccgaaagacggttccgttgtcgttctgggcttcactgaccgtatcggttctgacgcttacaaccagggtctgtccgagaaacgtgctcagtctgttgttgattacctgatctccaaaggtattccgtctgacaaaatctccgcacgtggtatgggcgaatctaacccggttaccggcaacacctgtgacaacgtgaaacctcgcgctgccctgatcgattgcctggctccggatcgtcgcgtagagatcgaagttaaaggcgttaaagacgtggtaactcagccgcaggcttaa

SEQ ID NO: 45 - protein, 364 aa, Synthetic construct
MKKTAIAIAVALAGFATVAQAAPKDNTWYAGAKLGWSQYHDTGFIHNDGPTHENQLGAGAFGGYQVNPYVGFEMGYDWLGRMPYKGDNINGAYKAQGVQLTAKLGYPITDDLDVYTRLGGMVWRADTKSNVPGAAAGGGGSHHHHHHGPSTKDHDTGVSPVFAGGIEYAITPEIATRLEYQWTNNIGDANTIGTRPDNGLLSVGVSYRFGQQEAAPVVAPAPAPAPEVQTKHFTLKSDVLFNFNKSTLKPEGQQALDQLYSQLSNLDPKDGSVVVLGFTDRIGSDAYNQGLSEKRAQSVVDYLISKGIPSDKISARGMGESNPVTGNTCDNVKPRAALIDCLAPDRRVEIEVKGVKDVVTQPQA

SEQ ID NO: 46 - DNA, 750 bp, Synthetic construct
  source 1..750: mol_type = other DNA
cagtctggggcagaggtgaaaaagcccggggagtctctgaagatctcctgtaagggttctggatacagctttaccagctactggatcgcctgggtgcgccagatgcccgggaaaggcctggagtacatggggctcatctatcctggtgactctgacaccaaatacagcccgtccttccaaggccaggtcaccatctcagtcgacaagtccgtcagcactgcctacttgcaatggagcagtctgaagccctcggacagcgccgtgtatttttgtgcgagacatgacgtgggatattgcagtagttccaactgcgcaaagtggcctgaatacttccagcattggggccagggcaccctggtcaccgtctcctcaggtggaggcggttcaggcggaggtggctctggcggtggcggatcgcagtctgtgttgacgcagccgccctcagtgtctgcggccccaggacagaaggtcaccatctcctgctctggaagcagctccaacattgggaataattatgtatcctggtaccagcagctcccaggaacagcccccaaactcctcatctatgatcacaccaatcggcccgcaggggtccctgaccgattctctggctccaagtctggcacctcagcctccctggccatcagtgggttccggtccgaggatgaggctgattattactgtgcctcctgggactacaccctctcgggctgggtgttcggcggaggaaccaagctgaccgtcctaggt

SEQ ID NO: 47 - protein, 250 aa, Synthetic construct
QSGAEVKKPGESLKISCKGSGYSFTSYWIAWVRQMPGKGLEYMGLIYPGDSDTKYSPSFQGQVTISVDKSVSTAYLQWSSLKPSDSAVYFCARHDVGYCSSSNCAKWPEYFQHWGQGTLVTVSSGGGGSGGGGSGGGGSQSVLTQPPSVSAAPGQKVTISCSGSSSNIGNNYVSWYQQLPGTAPKLLIYDHTNRPAGVPDRFSGSKSGTSASLAISGFRSEDEADYYCASWDYTLSGWVFGGGTKLTVLG

SEQ ID NO: 48 - DNA, 369 bp, Synthetic construct
  source 1..369: mol_type = other DNA
atggattccccagatagaccatggaacccaccaactttctccccagctttgttggtcgtcactgaaggtgataacgctactttcacttgttccttctccaacacttccgaatccttccatgttgtttggcatcgtgaatccccatccggtcaaactgatacattggctgctttcccagaagatagatcccaaccaggtcaagatgctagattcagagttactcaattgccaaacggtagagatttccacatgtccgtcgtcagagctagaagaaacgattccggtacttatgtttgtggtgttatttcccttgctccaaagattcaaattaaggaatccttgagagctgaattgagagtcactgaaaga

SEQ ID NO: 49 - protein, 123 aa, Synthetic construct
MDSPDRPWNPPTFSPALLVVTEGDNATFTCSFSNTSESFHVVWHRESPSGQTDTLAAFPEDRSQPGQDARFRVTQLPNGRDFHMSVVRARRNDSGTYVCGVISLAPKIQIKESLRAELRVTER

SEQ ID NO: 50 - DNA, 1197 bp, Synthetic construct
  source 1..1197: mol_type = other DNA
atgggctggtcctgtatcatcctgttcctggtggctacagccacaggagtgcatagtttcttagactccccagacaggccctggaacccccccaccttctccccagccctgctcgtggtgaccgaaggggacaacgccaccttcacctgcagcttctccaacacatcggagagcttccatgtaatctggcaccgcgagagccccagcggccagacggacacgctggccgccttccccgaggaccgcagccagcccggccaggactgccgcttccgtgtcacacaactgcccaacgggcgtgacttccacatgagcgtggtcagggcccggcgcaatgacagcggcacctacgtctgtggggtcatctccctggcccccaagatccagatcaaagagagcctgcgggcagagctcagggtgacagagagaagggcagaagtgcccacagcccaccccagcccctcacccaggccagccggccagttccaaaccctggagcccagagggcccacaatcaagccctgtcctccatgcaaatgcccagcacctaacctcttgggtggaccatccgtcttcatcttccctccaaagatcaaggatgtactcatgatctccctgagccccatagtcacatgtgtggtggtggatgtgagcgaggatgacccagatgtccagatcagctggtttgtgaacaacgtggaagtacacacagctcagacacaaacccatagagaggattacaacagtactctccgggtggtcagtgccctccccatccagcaccaggactggatgagtggcaaggagttcaaatgcaaggtcaacaacaaagacctcccagcgcccatcgagagaaccatctcaaaacccaaagggtcagtaagagctccacaggtatatgtcttgcctccaccagaagaagagatgactaagaaacaggtcactctgacctgcatggtcacagacttcatgcctgaagacatttacgtggagtggaccaacaacgggaaaacagagctaaactacaagaacactgaaccagtcctggactctgatggttcttacttcatgtacagcaagctgagagtggaaaagaagaactgggtggaaagaaatagctactcctgttcagtggtccacgagggtctgcacaatcaccacacgactaagagcttctcccggactccaggtaaataa

SEQ ID NO: 51 - protein, 398 aa, Synthetic construct
MGWSCIILFLVATATGVHSFLDSPDRPWNPPTFSPALLVVTEGDNATFTCSFSNTSESFHVIWHRESPSGQTDTLAAFPEDRSQPGQDCRFRVTQLPNGRDFHMSVVRARRNDSGTYVCGVISLAPKIQIKESLRAELRVTERRAEVPTAHPSPSPRPAGQFQTLEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 52 - protein, 373 aa, Synthetic construct
MLCCMRRTKQVEKNDEDQKISEGGPALFQSDMTFKIFIDGEVNGQKFTIVADGSSKFPHGDFNVHAVCETGKLPMSWKPICHLIQYGEPFFARYPDGISHFAQECFPEGLSIDRTVRFENDGTMTSHHTYELDDTCVVSRITVNCDGFQPDGPIMRDQLVDILPNETHMFPHGPNAVRQLAFIGFTTADGGLMMGHFDSKMTFNGSRAIEIPGPHFVTIITKQMRDTSDKRDHVCQREVAYAHSVPRITSAIGSDEDGSGATNFSLLKQAGDVEENPGPMSVKGMAIALAVILCATVVQGFPMFKRGRCLCIGPGVKAVKVADIEKASIMYPSNNCDKIEVIITLKENKGQRCLNPKSKQARLIIKKVERKNF

SEQ ID NO: 53 - protein, 379 aa, Synthetic construct
MLCCMRRTKQVEKNDEDQKISEGGPALFQSDMTFKIFIDGEVNGQKFTIVADGSSKFPHGDFNVHAVCETGKLPMSWKPICHLIQYGEPFFARYPDGISHFAQECFPEGLSIDRTVRFENDGTMTSHHTYELDDTCVVSRITVNCDGFQPDGPIMRDQLVDILPNETHMFPHGPNAVRQLAFIGFTTADGGLMMGHFDSKMTFNGSRAIEIPGPHFVTIITKQMRDTSDKRDHVCQREVAYAHSVPRITSAIGSDEDGSGATNFSLLKQAGDVEENPGPMNRKVTAIALAAIIWATAAQGFLMFKQGRCLCIGPGMKAVKMAEIEKASVIYPSNGCDKVEVIVTMKAHKRQRCLDPRSKQARLIMQAIEKKNFLRRQNM

SEQ ID NO: 54 - DNA, 48 bp, Synthetic construct
  source 1..48: mol_type = other DNA
gataacaatttcacacaggaaacagaccatgaaaaagacagctatcgc

SEQ ID NO: 55 - DNA, 46 bp, Synthetic construct
  source 1..46: mol_type = other DNA
gaaacccatacgaccgtcctggcacgcgccagggacgttagacttg

SEQ ID NO: 56 - DNA, 49 bp, Synthetic construct
  source 1..49: mol_type = other DNA
ccaggacggtcgtatgggtttctgcggtggcccgtctactaaagaccac

SEQ ID NO: 57 - DNA, 46 bp, Synthetic construct
  source 1..46: mol_type = other DNA
gccaaaacagccaagcttgagctcattaagcctgcggctgagttac

SEQ ID NO: 58 - DNA, 34 bp, Synthetic construct
  source 1..34: mol_type = other DNA
cccagcagtctagaatgaaaaagacagctatcgc

SEQ ID NO: 59 - DNA, 40 bp, Synthetic construct
  source 1..40: mol_type = other DNA
ctgcaaagagatgtgcggatctctagattatgcgaaaggc

SEQ ID NO: 60 - DNA, 45 bp, Synthetic construct
  source 1..45: mol_type = other DNA
caacagctcatttcagaatggaagaaaaaaaagcccgctcattag

SEQ ID NO: 61 - DNA, 45 bp, Synthetic construct
  source 1..45: mol_type = other DNA
ctaatgagcgggcttttttttcttccattctgaaatgagctgttg

SEQ ID NO: 62 - DNA, 40 bp, Synthetic construct
  source 1..40: mol_type = other DNA
gcctttcgcataatctagagatccgcacatctctttgcag

SEQ ID NO: 63 - DNA, 58 bp, Synthetic construct
  source 1..58: mol_type = other DNA
gtaactcagccgcaggcttaatgagcttcgaaacagattaaatcagaacgcagaagcg

SEQ ID NO: 64 - DNA, 60 bp, Synthetic construct
  source 1..60: mol_type = other DNA
aaaaaaaaccccgccctgtcaggggcggggtttttttttcctacgctcacccatcaattg

SEQ ID NO: 65 - DNA, 59 bp, Synthetic construct
  source 1..59: mol_type = other DNA
gattaattgtcaacagctcatttcagaatgatcaaaaaaaaccccgccctgtcaggggc

SEQ ID NO: 66 - DNA, 52 bp, Synthetic construct
  source 1..52: mol_type = other DNA
cgcccctgacagggcggggttttttttgatcattctgaaatgagctgttgac

SEQ ID NO: 67 - DNA, 52 bp, Synthetic construct
  source 1..52: mol_type = other DNA
cttctgcgttctgatttaatctgtttcgaagctcattaagcctgcggctgag

SEQ ID NO: 68 - DNA, 46 bp, Synthetic construct
  source 1..46: mol_type = other DNA
cgtttaaacttaagcttggtaccgccatgagtgtgaagggcatggc

SEQ ID NO: 69 - DNA, 50 bp, Synthetic construct
  source 1..50: mol_type = other DNA
gtctgctcgaagcattctcgagcggccgcttaaaaattctttctttcaac

SEQ ID NO: 70 - DNA, 47 bp, Synthetic construct
  source 1..47: mol_type = other DNA
gcgtttaaacttaagcttggtaccgccatgaacaggaaggtcacagc

SEQ ID NO: 71 - DNA, 52 bp, Synthetic construct
  source 1..52: mol_type = other DNA
ctcgaagcattctcgagcggccgcttacatgttttgacgccttaaaaaattc

SEQ ID NO: 72 - DNA, 58 bp, Synthetic construct
  source 1..58: mol_type = other DNA
gcgtttaaacttaagcttggtaccgccaccatgctgtgctgtatgagaagaaccaaac

SEQ ID NO: 73 - DNA, 45 bp, Synthetic construct
  source 1..45: mol_type = other DNA
gctcgaagcattctcgagcggccgctttaatcctcgtcgctaccg

SEQ ID NO: 74 - DNA, 52 bp, Synthetic construct
  source 1..52: mol_type = other DNA
gcgtttaaacttaagcttggtaccgccaccatgtccgtcctgacgccgctgc

SEQ ID NO: 75 - DNA, 49 bp, Synthetic construct
  source 1..49: mol_type = other DNA
cgcaggcttaatgagctcaaggaacagtcaatgagtaaaggagaagaac

SEQ ID NO: 76 - DNA, 47 bp, Synthetic construct
  source 1..47: mol_type = other DNA
catccgccaaaacagccaagcttattatttgtatagttcatccatgc

SEQ ID NO: 77 - DNA, 46 bp, Synthetic construct
  source 1..46: mol_type = other DNA
gtttaaacttaagcttggtaccaccaaaatggtgagcaagggcgag

SEQ ID NO: 78 - DNA, 42 bp, Synthetic construct
  source 1..42: mol_type = other DNA
ctgctcgaagcattctcgagttacttgtacagctcgtccatg

SEQ ID NO: 79 - DNA, 58 bp, Synthetic construct
  source 1..58: mol_type = other DNA
gcctgcttcagcaggctgaagttagtagctccgcttccatcctcgtcgctaccgatgg

SEQ ID NO: 80 - DNA, 49 bp, Synthetic construct
  source 1..49: mol_type = other DNA
aggtccagggttctcctccacgtcgccagcctgcttcagcaggctgaag

SEQ ID NO: 81 - DNA, 45 bp, Synthetic construct
  source 1..45: mol_type = other DNA
cgacgtggaggagaaccctggacctatgaacaggaaggtcacagc

SEQ ID NO: 82 - DNA, 45 bp, Synthetic construct
  source 1..45: mol_type = other DNA
cgacgtggaggagaaccctggacctatgagtgtgaagggcatggc

SEQ ID NO: 83 - DNA, 43 bp, Synthetic construct
  source 1..43: mol_type = other DNA
cttaagcttggtacgccgccaccatgctggtcatggcgccccg

SEQ ID NO: 84 - DNA, 41 bp, Synthetic construct
  source 1..41: mol_type = other DNA
cctaggcccgggcccggtaccggagccggcccaggtctcgg

SEQ ID NO: 85 - DNA, 40 bp, Synthetic construct
  source 1..40: mol_type = other DNA
ggtaccgggcccgggcctaggggcctggctgtcctggcag

SEQ ID NO: 86 - DNA, 41 bp, Synthetic construct
  source 1..41: mol_type = other DNA
ctgctcgaagcattctcgagtcaagctgtgagagacacatc

SEQ ID NO: 87 - DNA, 40 bp, Synthetic construct
  source 1..40: mol_type = other DNA
ctgggccggctccggtaccatggtgagcaagggcgaggag

SEQ ID NO: 88 - DNA, 44 bp, Synthetic construct
  source 1..44: mol_type = other DNA
ctaggacagccaggcccctaggcttgtacagctcgtccatgccg

SEQ ID NO: 89 - DNA, 60 bp, Synthetic construct
  source 1..60: mol_type = other DNA
ggctagcgtttaaacttaagcttggtacaatttgtaatacgactcactatagggcggccg

SEQ ID NO: 90 - DNA, 43 bp, Synthetic construct
  source 1..43: mol_type = other DNA
ctgctcgaagcattctcgaggtacgactatggaaccgcggccg

SEQ ID NO: 91 - DNA, 40 bp, Synthetic construct; identical to SEQ ID NO: 87
  source 1..40: mol_type = other DNA

SEQ ID NO: 92 - DNA, 44 bp, Synthetic construct; identical to SEQ ID NO: 88
  source 1..44: mol_type = other DNA

SEQ ID NO: 93 - DNA, 42 bp, Synthetic construct
  source 1..42: mol_type = other DNA
gaggtacctgcaggcccggggcggccgctcgagaatgcttcg

SEQ ID NO: 94 - DNA, 38 bp, Synthetic construct
  source 1..38: mol_type = other DNA
cgggcaccggagcggaaagtccccggaaagtccccgcc

SEQ ID NO: 95 - DNA, 37 bp, Synthetic construct
  source 1..37: mol_type = other DNA
ctttccggggactttccgctccggtgcccgtcagtgg

SEQ ID NO: 96 - DNA, 39 bp, Synthetic construct
  source 1..39: mol_type = other DNA
cccgggcctgcaggtacctcacgacacctgaaatggaag

SEQ ID NO: 97 - DNA, 50 bp, Synthetic construct
  source 1..50: mol_type = other DNA
gcgtttaaacttaagcttggtaccgccatggattccccagatagaccatg

SEQ ID NO: 98 - DNA, 76 bp, Synthetic construct
  source 1..76: mol_type = other DNA
cctcgcttcctccgccttcacttccaccgccctcactgccgccgccggagccgcctctttcagtgactctcaattc

SEQ ID NO: 99 - DNA, 74 bp, Synthetic construct
  source 1..74: mol_type = other DNA
gcttccgccgccgctgcctccaccctcagacccgcctccttcggagcctcctccctcgcttcctccgccttcac

SEQ ID NO: 100 - DNA, 36 bp, Synthetic construct
  source 1..36: mol_type = other DNA
cagcggcggcggaagcatgaacaggaaggtcacagc

SEQ ID NO: 101 - DNA, 36 bp, Synthetic construct
  source 1..36: mol_type = other DNA
cagcggcggcggaagcatgagtgtgaagggcatggc

SEQ ID NO: 102 - DNA, 73 bp, Synthetic construct
  source 1..73: mol_type = other DNA
gtttaaacttaagcttggtacgccaccatgtacaggatgcaactcctgtcttgcattgcactaagtcttgcac

SEQ ID NO: 103 - DNA, 73 bp, Synthetic construct
  source 1..73: mol_type = other DNA
gactagtggatccgagctcggtacctgcactgtttgtgacaagtgcaagacttagtgcaatgcaagacaggag

SEQ ID NO: 104 - DNA, 48 bp, Synthetic construct
  source 1..48: mol_type = other DNA
ctcgaagcattctcgagcggccgcttatctttcagtgactctcaattc

SEQ ID NO: 105 - DNA, 36 bp, Synthetic construct
  source 1..36: mol_type = other DNA
ggtggcgaccggtggtctttcagtgactctcaattc

SEQ ID NO: 106 - DNA, 36 bp, Synthetic construct
  source 1..36: mol_type = other DNA
ccaccggtcgccaccatggtgagcaagggcgaggag

SEQ ID NO: 107 - DNA, 48 bp, Synthetic construct
  source 1..48: mol_type = other DNA
ctcgaagcattctcgagcggccgcttacttgtacagctcgtccatgcc

SEQ ID NO: 108 - DNA, 40 bp, Synthetic construct
  source 1..40: mol_type = other DNA
ggtggcgaccggtggaaaattctttctttcaacttttttg

SEQ ID NO: 109 - DNA, 40 bp, Synthetic construct
  source 1..40: mol_type = other DNA
ggtggcgaccggtggcatgttttgacgccttaaaaaattc

SEQ ID NO: 110 - DNA, 43 bp, Synthetic construct
  source 1..43: mol_type = other DNA
cgcaggccatagctccaatgttcgcacgcgccagggacgttag

SEQ ID NO: 111 - DNA, 44 bp, Synthetic construct
  source 1..44: mol_type = other DNA
gagctatggcctgcgtccgggctgcggtggcccgtctactaaag

SEQ ID NO: 112 - DNA, 40 bp, Synthetic construct
  source 1..40: mol_type = other DNA
ctcatggtgacgagccttcgaatcattaagcctgcggctg

SEQ ID NO: 113 - DNA, 35 bp, Synthetic construct
  source 1..35: mol_type = other DNA
ggctcgtcaccatgagtaaaggagaagaacttttc

SEQ ID NO: 114 - DNA, 42 bp, Synthetic construct
  source 1..42: mol_type = other DNA
cgttctgatttaatctgtttcgaattatttgtatagttcatc

SEQ ID NO: 115 - DNA, 47 bp, Synthetic construct
  source 1..47: mol_type = other DNA
cgtttaaacttaagcttggtaccgccatgggctggtcctgtatcatc

SEQ ID NO: 116 - DNA, 48 bp, Synthetic construct
  source 1..48: mol_type = other DNA
gtctgctcgaagcattctcgagcggccgcttatttacctggagtccgg

SEQ ID NO: 117 - DNA, 45 bp, Synthetic construct
  source 1..45: mol_type = other DNA
cgacgtggaggagaaccctggacctatgctgtgctgtatgagaag

SEQ ID NO: 118 - DNA, 48 bp, Synthetic construct
  source 1..48: mol_type = other DNA
ctaacttcagcctgctgaagcaggctggcgacgtggaggagaaccctg

SEQ ID NO: 119 - DNA, 40 bp, Synthetic construct
  source 1..40: mol_type = other DNA
ggagaaccctggacctatgggctggtcctgtatcatcgtg

SEQ ID NO: 120 - DNA, 40 bp, Synthetic construct
  source 1..40: mol_type = other DNA
cctctgccccagactggccagggacgttagacttggtgtc

SEQ ID NO: 121 - DNA, 36 bp, Synthetic construct
  source 1..36: mol_type = other DNA
catcaccatcaccatggcccgtctactaaagaccac

SEQ ID NO: 122 - DNA, 24 bp, Synthetic construct
  source 1..24: mol_type = other DNA
cagtctggggcagaggtgaaaaag

SEQ ID NO: 123 - DNA, 24 bp, Synthetic construct
  source 1..24: mol_type = other DNA
atggtgatggtgatgatgagatcc

SEQ ID NO: 124 - DNA, 50 bp, Synthetic construct
  source 1..50: mol_type = other DNA
gcaggctgaagttagtagctccgcttcctttacctggagtccgggagaag

SEQ ID NO: 125 - DNA, 76 bp, Synthetic construct
  source 1..76: mol_type = other DNA
ctgatttaatctgtttcgaacctgcaggggcccgggccgcggagtactcctagggtctgtttcctgtgtgaaattg

SEQ ID NO: 126 - DNA, 75 bp, Synthetic construct
  source 1..75: mol_type = other DNA
ctgatttaatctgtttcgaacctgcaggggcccgggccgcggagtactcctaggttcagcatcttttactttcac

SEQ ID NO: 127 - DNA, 105 bp, Synthetic construct
  source 1..105: mol_type = other DNA
atgaaaaaacaacatttccgtgtcgcccttattcccttttttgcggcattttgccttcctgtttttgctcacccagaaacgctggtgaaagtaaaagatgctgaa

SEQ ID NO: 128 - protein, 35 aa, Synthetic construct
MKKQHFRVALIPFFAAFCLPVFAHPETLVKVKDAE

SEQ ID NO: 129 - protein, 21 aa, unidentified
  source 1..21: note = Thosea asigna virus 2A
GSGEGRGSLLTCGDVEENPGP

SEQ ID NO: 130 - protein, 23 aa, Equine rhinitis A virus
GSGQCTNYALLKLAGDVESNPGP

SEQ ID NO: 131 - protein, 25 aa, Foot-and-mouth disease virus
GSGVKQTLNFDLLKLAGDVESNPGP

SEQ ID NO: 132 - DNA, 108 bp, Synthetic construct
  source 1..108: mol_type = other DNA
ggcggctccggcggcggcagtgagggcggtggaagtgaaggcggaggaagcgagggaggaggctccgaaggaggcgggtctgagggtggaggcagcggcggcggaagc

SEQ ID NO: 133 - protein, 36 aa, Synthetic construct
GGSGGGSEGGGSEGGGSEGGGSEGGGSEGGGSGGGS

SEQ ID NO: 134 - DNA, 4056 bp, Synthetic construct
  source 1..4056: mol_type = other DNA
ggatcttccggaagaccttccattctgaaatgagctgttgacaattaatcatccggctcgtataatgtgtggaattgtgagcggataacaatttcacacaggaaacagaccatgaaaaagacagctatcgcgattgcagtggcactggctggtttcgctaccgtagcgcaggccgctccgaaagataacacctggtacgctggtgctaaactgggctggtctcagtaccatgacaccggcttcattcacaatgatggcccgactcatgaaaaccaactgggcgcaggtgcttttggtggttaccaggttaacccgtatgttggctttgaaatgggctacgactggttaggccgtatgccgtacaaaggcgacaacatcaatggcgcttataaagctcagggcgttcagttgaccgctaaactgggttatccaatcactgacgatctggacgtttatacccgtctgggtggtatggtatggcgtgcagacaccaagtctaacgtccctggcgcgtgccaggacggtcgtatgggtttctgcggtggcccgtctactaaagaccacgacaccggcgtttccccggtattcgcgggcggtatcgagtatgctatcacccctgaaatcgcaacccgtctggaataccagtggactaacaacatcggtgatgccaacaccatcggcacccgtccggacaacggcctgctgagcgtaggtgtttcctaccgtttcggccagcaagaagctgctccggtagtagctccggcaccagctccggctccggaagtacagaccaagcacttcactctgaagtctgacgtactgttcaacttcaacaaatctaccctgaagccggaaggccagcaggctctggatcagctgtacagccagctgagcaacctggatccgaaagacggttccgttgtcgttctgggcttcactgaccgtatcggttctgacgcttacaaccagggtctgtccgagaaacgtgctcagtctgttgttgattacctgatctccaaaggtattccgtctgacaaaatctccgcacgtggtatgggcgaatctaacccggttaccggcaacacctgtgacaacgtgaaacctcgcgctgccctgatcgattgcctggctccggatcgtcgcgtagagatcgaagttaaaggcgttaaagacgtggtaactcagccgcaggcttaatgagctcaagcttggctgttttggcggatgagagaagattttcagcctgatacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttttgtttatttttctaaatacattcaaatatgtatccgctcatgagacaataaccctgataaatgcttcaataatggaagatcttccaacatcacaggtaaacagaaacgtcgggtcgatcgggaaattctttcccggacggcgcggggttgggcaagccgcaggcgcgtcagtgcttttagcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagtctagactaggccaactggcgcagcattcgacgcagcggctcggcggcgccccataacaactggtcgcctacggtaaacgccgacaagaactctggccccatgttcagcttacgcagacgaccaaccggcgtagtcaacgtgccggtcaccgccgccggggttaattcgcgcatagtgatatcacgatcgttcggcaccactttcgcccacggattatgtgccgccagcagttcttccaccgtcggaatggatacctcttttttcagcttgatggtgaacgcctggctgtgacagcgcagcgcgccgacgcgcacacacaaaccatcaaccggaatcacagaggcagtattgagaatcttgttggtttccgcctggcctttccactcttcgcggctctggccgttatcgagctgtttgtcgatccaggggatcaggcttcccgccagcggtacgccaaagttatcaaccggcagctcgccgctgcgggtcaatgccgtaactttgcgttcaatatcaagaattgcggaagacggcgtcgccagttcatcggcgacatggccatacaactgacccatctgggttaacagctcgcgcatatggcgcgcgccgccgccggaggcggcctgataggtcgcgacggatacccagtcaacgagattatgggcaaagagaccgcccagcgacatcaacatcaggctaacggtacagttaccgcccacaaaggtcttcacgccattgttcaggccgtcggtaatcacgtcctggttgaccgggtcgagaataataatggcatcatctttcatgcgcagcgtagaagccgcatcaatccagtaaccctgccatccgctttcgcgcagctttggataaatttcgttggtataatcgccgccctggcaggtcacgatgatatcgagcgcttttagcgcatccagatcaaaagcgtcctgtagcgtgccggtggaggtgtcgccgaaggtgggcgccgcctgtccaaactgggaggtagaaaagaaaacagggcgaatagcgtcgaaatcgcgctcctctaccatgcgttgcatgagaacagagccgaccattccgcgccagccgataaaaccaacatttttcatagcgtttttttcctgcaaagagatgtgc

SEQ ID NO: 135 - DNA, 8160 bp, Synthetic construct
  source 1..8160: mol_type = other DNA
gactcttcgcgatgtacgggccagatatacgcgttaactgcagtctagattatgcgaaaggccatcctgacggatggcctttttgtttaaacggatccgcgacattgattattgactagttattaatagtaatcaattacggggtcattaggggactttccggggactttcctccccacgcgggggactttccgccacgggcggggactttccggggactttccgttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttggtaccgagctcggatccactagtccagtgtggtggaattctgcagatatccagcacagtggcggccgctcgagaatgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatccgataaggatcgatccggggcatgcaaccagctgtggaatgtgtgtcagttagggtgtggaaagtccccaggctccccagcaggcagaagtatgcaaagcatgtggggatgcggtgggctctatggcttctactgggcggttttatggacagcaagcgaaccggaattgccagctggggcgccctctggtaaggttgggaagccctgcaaagtaaactggatggctttctcgccgccaaggatctgtcgacccctagatttcagtgcaatttatctcttcaaatgtagcacctgaagtcagccccatacgatataagttgttggaagatctagcccgcctaatgagcgggcttttttttaattcgcaattccccgatgcataatgtgcctgtcaaatggacgaagcagggattctgcaaaccctatgctactccgtcaagccgtcaattgtctgattcgttaccaattatgacaacttgacggctacatcattcactttttcttcacaaccggcacgaaactcgctcgggctggccccggtgcattttttaaatactcgcgagaaatagagttgatcgtcaaaaccaacattgcgaccgacggtggcgataggcatccgggtagtgctcaaaagcagcttcgcctgactaatgcgttggtcctcgcgccagcttaagacgctaatccctaactgctggcggaaaagatgtgacagacgcgacggcgacaagcaaacatgctgtgcgacgctggcgatatcaaaattgctgtctgccaggtgatcgctgatgtactgacaagcctcgcgtacccgattatccatcggtggatggagcgactcgttaatcgcttccatgcgccgcagtaacaattgctcaagcagatttatcgccagcagctccgaatagcgcccttccccttgcccggcgttaatgatttgcccaaacaggtcgctgaaatgcggctggtgcgcttcatccgggcgaaagaaacccgtattggcaaatattgacggccagttaagccattcatgccagtaggcgcgcggacgaaagtaaacccactggtgataccattcgcgagcctccggatgacgaccgtagtgatgaatctctcctggcgggaacagcaaaatatcacccggtcggcagacaaattctcgtccctgatttttcaccaccccctgaccgcgaatggtgagattgagaatataacctttcattcccagcggtcggtcgataaaaaaatcgagataaccgttggcctcaatcggcgttaaacccgccaccagatgggcgttaaacgagtatcccggcagcaggggatcattttgcgcttcagccatacttttcatactcccaccattcagagaagaaaccaattgtccatattgcatcagacattgccgtcactgcgtcttttactggctcttctcgctaacccaaccggtaaccccgcttattaaaagcattctgtaacaaagcgggaccaaagccatgacaaaaacgcgtaacaaaagtgtctataatcacggcagaaaagtccacattgattatttgcacggcgtcacactttgctatgccatagcatttttatccataagattagcggatcctacctgacgctttttatcgcaactctctactgtttctccatacccgtttttttgggctagcgaattctgagaacaaactaagtggataaatttcgtgttcaggggccaacgaagctccagggcgaagtcacaatttccggcgctaaaaatgctgctctgcctatcctttttgccgcactactggcggaagaaccggtagagatccagaacgtcccgaaactgaaagacgtcgatacatcaatgaagctgctaagccagctgggtgcgaaagtagaacgtaatggttctgtgcatattgatgcccgcgacgttaatgtattctgcgcaccttacgatctggttaaaaccatgcgtgcttctatctgggcgctggggccgctggtagcgcgctttggtcaggggcaagtttcactacctggcggttgtacgatcggtgcgcgtccggttgatctacacatttctggcctcgaacaattaggcgcgaccatcaaactggaagaaggttacgttaaagcttccgtcgatggtcgtttgaaaggtgcacatatcgtgatggataaagtcagcgttggcgcaacggtgaccatcatgtgtgctgcaaccctggcggaaggcaccacgattattgaaaacgcagcgcgtgaaccggaaatcgtcgataccgcgaacttcctgattacgctgggtgcgaaaattagcggtcagggcaccgatcgtatcgtcatcgaaggtgtggaacgtttaggcggcggtgtctatcgcgttctgccggatcgtatcgaaaccggtactttcctggtggcggcggcgatttctcgcggcaaaattatctgccgtaacgcgcagccagatactctcgacgccgtgctggcgaaactgcgtgacgctggagcggacatcgaagtcggcgaagactggattagcctggatatgcatggcaaacgtccgaaggctgttaacgtacgtaccgcgccgcatccggcattcccgaccgatatgcaggcccagttcacgctgttgaacctggtggcagaagggaccgggtttatcaccgaaacggtctttgaaaaccgctttatgcatgtgccagagctgagccgtatgggcgcgcacgccgaaatcgaaagcaataccgttatttgtcacggtgttgaaaaactttctggcgcacaggttatggcaaccgatctgcgtgcatcagcaagcctggtgctggctggctgtattgcggaagggacgacggtggttgatcgtatttatcacatcgatcgtggctacgaacgcattgaagacaaactgcgcgctttaggtgcaaatattgagcgtgtgaaaggcgaataagaattcaggaaaaaaacgctgtgaaaaatgttggttttatcggctggcgcggaatggtcggctctgttctcatgcaacgcatggtagaggagcgcgatttcgacgctattcgccctgttttcttttctacctcccagtttggacaggcggcgcccaccttcggcgacacctccaccggcacgctacaggacgcttttgatctggatgcgctaaaagcgctcgatatcatcgtgacctgccagggcggcgattataccaacgaaatttatccaaagctgcgcgaaagcggatggcagggttactggattgatgcggcttctacgctgcgcatgaaagatgatgccattattattctcgacccggtcaaccaggacgtgattaccgacggcctgaacaatggcgtgaagacctttgtgggcggtaactgtaccgttagcctgatgttgatgtcgctgggcggtctctttgcccataatctcgttgactgggtatccgtcgcgacctatcaggccgcctccggcggcggcgcgcgccatatgcgcgagctgttaacccagatgggtcagttgtatggccatgtcgccgatgaactggcgacgccgtcttccgcaattcttgatattgaacgcaaagttacggcattgacccgcagcggcgagctgccggttgataactttggcgtaccgctggcgggaagcctgatcccctggatcgacaaacagctcgataacggccagagccgcgaagagtggaaaggccaggcggaaaccaacaagattctcaatactgcctctgtgattccggttgatggtttgtgtgtgcgcgtcggcgcgctgcgctgtcacagccaggcgttcaccatcaagctgaaaaaagaggtatccattccgacggtggaagaactgctggcggcacataatccgtgggcgaaagtggtgccgaacgatcgtgatatcactatgcgcgaattaaccccggcggcggtgaccggcacgttgactacgccggttggtcgtctgcgtaagctgaacatggggccagagttcttgtcggcgtttaccgtaggcgaccagttgttatggggcgccgccgagccgctgcgtcgaatgctgcgccagttggcgtagtctagctgcacgataccgtcgacttgtacatagactcgctccgaaattaaagaacacttaaattatctactaaaggaatctttagtcaagtttatttaagatgacttaactatgaatacacaattgatgggtgagcgtaggaaaaaaaaaccccgcccctgacagggcggggttttttttgatcattctgaaatgagctgttgacaattaatcatccggctcgtataatgtgtggaattgtgagcggataacaatttcacacaggaaacagaccatgaaaaagacagctatcgcgattgcagtggcactggctggtttcgctaccgtagcgcaggccgctccgaaagataacacctggtacgctggtgctaaactgggctggtctcagtaccatgacaccggcttcattcacaatgatggcccgactcatgaaaaccaactgggcgcaggtgcttttggtggttaccaggttaacccgtatgttggctttgaaatgggctacgactggttaggccgtatgccgtacaaaggcgacaacatcaatggcgcttataaagctcagggcgttcagttgaccgctaaactgggttatccaatcactgacgatctggacgtttatacccgtctgggtggtatggtatggcgtgcagacaccaagtctaacgtccctggcgcgtgccaggacggtcgtatgggtttctgcggtggcccgtctactaaagaccacgacaccggcgtttccccggtattcgcgggcggtatcgagtatgctatcacccctgaaatcgcaacccgtctggaataccagtggactaacaacatcggtgatgccaacaccatcggcacccgtccggacaacggcctgctgagcgtaggtgtttcctaccgtttcggccagcaagaagctgctccggtagtagctccggcaccagctccggctccggaagtacagaccaagcacttcactctgaagtctgacgtactgttcaacttcaacaaatctaccctgaagccggaaggccagcaggctctggatcagctgtacagccagctgagcaacctggatccgaaagacggttccgttgtcgttctgggcttcactgaccgtatcggttctgacgcttacaaccagggtctgtccgagaaacgtgctcagtctgttgttgattacctgatctccaaaggtattccgtctgacaaaatctccgcacgtggtatgggcgaatctaacccggttaccggcaacacctgtgacaacgtgaaacctcgcgctgccctgatcgattgcctggctccggatcgtcgcgtagagatcgaagttaaaggcgttaaagacgtggtaactcagccgcaggcttaatgagcttcgaaacagattaaatcagaacgcagaagcggtctgataaaacagtttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactctttttgtttatttttctaaatacattcaaatatgtatccgctcatgagacaataaccctgataaatgcttcaataatggaagatcttccaacatcacaggtaaacagaaacgtcgggtcgatcgggaaattctttcccggacggcgcggggttgggcaagccgcaggcgcgtcagtgcttttagcgggtgtcggggcagccctgaaccagtcacgggatcgatctgtgcggtatttcacaccgcatacaggtggcacttttcggggaaatgtgcgcggaacccctatttgtttatttttctaaatacattcaaatatgtatccgctcatgagacaataaccctgataaatgcttcaataatagcacgtgctaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctgggcttttgctggccttttgctcacatgttct

SEQ ID NO: 136 - protein, 364 aa, Synthetic construct
MKKTAIAIAVALAGFATVAQAAPKDNTWYAGAKLGWSQYHDTGFIHNDGPTHENQLGAGAFGGYQVNPYVGFEMGYDWLGRMPYKGDNINGAYKAQGVQLTAKLGYPITDDLDVYTRLGGMVWRADTKSNVPGACEHWSYGLRPGCGGPSTKDHDTGVSPVFAGGIEYAITPEIATRLEYQWTNNIGDANTIGTRPDNGLLSVGVSYRFGQQEAAPVVAPAPAPAPEVQTKHFTLKSDVLFNFNKSTLKPEGQQALDQLYSQLSNLDPKDGSVVVLGFTDRIGSDAYNQGLSEKRAQSVVDYLISKGIPSDKISARGMGESNPVTGNTCDNVKPRAALIDCLAPDRRVEIEVKGVKDVVTQPQA

SEQ ID NO: 137 - protein, 614 aa, Synthetic construct
MKKTAIAIAVALAGFATVAQAAPKDNTWYAGAKLGWSQYHDTGFIHNDGPTHENQLGAGAFGGYQVNPYVGFEMGYDWLGRMPYKGDNINGAYKAQGVQLTAKLGYPITDDLDVYTRLGGMVWRADTKSNVPGQSGAEVKKPGESLKISCKGSGYSFTSYWIAWVRQMPGKGLEYMGLIYPGDSDTKYSPSFQGQVTISVDKSVSTAYLQWSSLKPSDSAVYFCARHDVGYCSSSNCAKWPEYFQHWGQGTLVTVSSGGGGSGGGGSGGGGSQSVLTQPPSVSAAPGQKVTISCSGSSSNIGNNYVSWYQQLPGTAPKLLIYDHTNRPAGVPDRFSGSKSGTSASLAISGFRSEDEADYYCASWDYTLSGWVFGGGTKLTVLGAAAGGGGSHHHHHHGPSTKDHDTGVSPVFAGGIEYAITPEIATRLEYQWTNNIGDANTIGTRPDNGLLSVGVSYRFGQQEAAPVVAPAPAPAPEVQTKHFTLKSDVLFNFNKSTLKPEGQQALDQLYSQLSNLDPKDGSVVVLGFTDRIGSDAYNQGLSEKRAQSVVDYLISKGIPSDKISARGMGESNPVTGNTCDNVKPRAALIDCLAPDRRVEIEVKGVKDVVTQPQA

SEQ ID NO: 138 - DNA, 7104 bp, Synthetic construct
  source 1..7104: mol_type = other DNA
gactcttcgcgatgtacgggccagatatacgcgttaactgcagtctagattatgcgaaaggccatcctgacggatggcctttttgtttaaacggatccgcgacattgattattgactagttattaatagtaatcaattacggggtcattaggggactttccggggactttcctccccacgcgggggactttccgccacgggcggggactttccggggactttccgttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttggtaccgagctcggatccactagtccagtgtggtggaattctgcagatatccagcacagtggcggccgctcgagaatgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatccgataaggatcgatccggggcatgcaaccagctgtggaatgtgtgtcagttagggtgtggaaagtccccaggctccccagcaggcagaagtatgcaaagcatgtggggatgcggtgggctctatggcttctactgggcggttttatggacagcaagcgaaccggaattgccagctggggcgccctctggtaaggttgggaagccctgcaaagtaaactggatggctttctcgccgccaaggatctgtcgacccctagatttcagtgcaatttatctcttcaaatgtagcacctgaagtcagccccatacgatataagttgttggaagatctagcccgcctaatgagcgggcttttttttaattcgcaattccccgatgcataatgtgcctgtcaaatggacgaagcagggattctgcaaaccctatgctactccgtcaagccgtcaattgtctgattcgttaccaattatgacaacttgacggctacatcattcactttttcttcacaaccggcacgaaactcgctcgggctggccccggtgcattttttaaatactcgcgagaaatagagttgatcgtcaaaaccaacattgcgaccgacggtggcgataggcatccgggtagtgctcaaaagcagcttcgcctgactaatgcgttggtcctcgcgccagcttaagacgctaatccctaactgctggcggaaaagatgtgacagacgcgacggcgacaagcaaacatgctgtgcgacgctggcgatatcaaaattgctgtctgccaggtgatcgctgatgtactgacaagcctcgcgtacccgattatccatcggtggatggagcgactcgttaatcgcttccatgcgccgcagtaacaattgctcaagcagatttatcgccagcagctccgaatagcgcccttccccttgcccggcgttaatgatttgcccaaacaggtcgctgaaatgcggctggtgcgcttcatccgggcgaaagaaacccgtattggcaaatattgacggccagttaagccattcatgccagtaggcgcgcggacgaaagtaaacccactggtgataccattcgcgagcctccggatgacgaccgtagtgatgaatctctcctggcgggaacagcaaaatatcacccggtcggcagacaaattctcgtccctgatttttcaccaccccctgaccgcgaatggtgagattgagaatataacctttcattcccagcggtcggtcgataaaaaaatcgagataaccgttggcctcaatcggcgttaaacccgccaccagatgggcgttaaacgagtatcccggcagcaggggatcattttgcgcttcagccatacttttcatactcccaccattcagagaagaaaccaattgtccatattgcatcagacattgccgtcactgcgtcttttactggctcttctcgctaacccaaccggtaaccccgcttattaaaagcattctgtaacaaagcgggaccaaagccatgacaaaaacgcgtaacaaaagtgtctataatcacggcagaaaagtccacattgattatttgcacggcgtcacactttgctatgccatagcatttttatccataagattagcggatcctacctgacgctttttatcgcaactctctactgtttctccatacccgtttttttgggctagcgaattctgagaacaaactaagtggataaatttcgtgttcaggggccaacgaagctccagggcgaagtcacaatttccggcgctaaaaatgctgctctgcctatcctttttgccgcactactggcggaagaaccggtagagatccagaacgtcccgaaactgaaagacgtcgatacatcaatgaagctgctaagccagctgggtgcgaaagtagaacgtaatggttctgtgcatattgatgcccgcgacgttaatgtattctgcgcaccttacgatctggttaaaaccatgcgtgcttctatctgggcgctggggccgctggtagcgcgctttggtcaggggcaagtttcactacctggcggttgtacgatcggtgcgcgtccggttgatctacacatttctggcctcgaacaattaggcgcgaccatcaaactggaagaaggttacgttaaagcttccgtcgatggtcgtttgaaaggtgcacatatcgtgatggataaagtcagcgttggcgcaacggtgaccatcatgtgtgctgcaaccctggcggaaggcaccacgattattgaaaacgcagcgcgtgaaccggaaatcgtcgataccgcgaacttcctgattacgctgggtgcgaaaattagcggtcagggcaccgatcgtatcgtcatcgaaggtgtggaacgtttaggcggcggtgtctatcgcgttctgccggatcgtatcgaaaccggtactttcctggtggcggcggcgatttctcgcggcaaaattatctgccgtaacgcgcagccagatactctcgacgccgtgctggcgaaactgcgtgacgctggagcggacatcgaagtcggcgaagactggattagcctggatatgcatggcaaacgtccgaaggctgttaacgtacgtaccgcgccgcatccggcattcccgaccgatatgcaggcccagttcacgctgttgaacctggtggcagaagggaccgggtttatcaccgaaacggtctttgaaaaccgctttatgcatgtgccagagctgagccgtatgggcgcgcacgccgaaatcgaaagcaataccgttatttgtcacggtgttgaaaaactttctggcgcacaggttatggcaaccgatctgcgtgcatcagcaagcctggtgctggctggctgtattgcggaagggacgacggtggttgatcgtatttatcacatcgatcgtggctacgaacgcattgaagacaaactgcgcgctttaggtgcaaatattgagcgtgtgaaaggcgaataagaattcaggaaaaaaacgctgtgaaaaatgttggttttatcggctggcgcggaatggtcggctctgttctcatgcaacgcatggtagaggagcgcgatttcgacgctattcgccctgttttcttttctacctcccagtttggacaggcggcgcccaccttcggcgacacctccaccggcacgctacaggacgcttttgatctggatgcgctaaaagcgctcgatatcatcgtgacctgccagggcggcgattataccaacgaaatttatccaaagctgcgcgaaagcggatggcagggttactggattgatgcggcttctacgctgcgcatgaaagatgatgccattattattctcgacccggtcaaccaggacgtgattaccgacggcctgaacaatggcgtgaagacctttgtgggcggtaactgtaccgttagcctgatgttgatgtcgctgggcggtctctttgcccataatctcgttgactgggtatccgtcgcgacctatcaggccgcctccggcggcggcgcgcgccatatgcgcgagctgttaacccagatgggtcagttgtatggccatgtcgccgatgaactggcgacgccgtcttccgcaattcttgatattgaacgcaaagttacggcattgacccgcagcggcgagctgccggttgataactttggcgtaccgctggcgggaagcctgatcccctggatcgacaaacagctcgataacggccagagccgcgaagagtggaaaggccaggcggaaaccaacaagattctcaatactgcctctgtgattccggttgatggtttgtgtgtgcgcgtcggcgcgctgcgctgtcacagccaggcgttcaccatcaagctgaaaaaagaggtatccattccgacggtggaagaactgctggcggcacataatccgtgggcgaaagtggtgccgaacgatcgtgatatcactatgcgcgaattaaccccggcggcggtgaccggcacgttgactacgccggttggtcgtctgcgtaagctgaacatggggccagagttcttgtcggcgtttaccgtaggcgaccagttgttatggggcgccgccgagccgctgcgtcgaatgctgcgccagttggcgtagtctagctgcacgataccgtcgacttgtacatagactcgctccgaaattaaagaacacttaaattatctactaaaggaatctttagtcaagtttatttaagatgacttaactatgaatacacaattgatgggtgagcgtaggaaaaaaaaaccccgcccctgacagggcggggttttttttgatcattctgaaatgagctgttgacaattaatcatccggctcgtataatgtgtggaattgtgagcggataacaatttcacacaggaaacagaccctaggagtactccgcggcccgggcccctgcaggttcgaaacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactctttttgtttatttttctaaatacattcaaatatgtatccgctcatgagacaataaccctgataaatgcttcaataatggaagatcttccaacatcacaggtaaacagaaacgtcgggtcgatcgggaaattctttcccggacggcgcggggttgggcaagccgcaggcgcgtcagtgcttttagcgggtgtcggggcagccctgaaccagtcacgggatcgatctgtgcggtatttcacaccgcatacaggtggcacttttcggggaaatgtgcgcggaacccctatttgtttatttttctaaatacattcaaatatgtatccgctcatgagacaataaccctgataaatgcttcaataatagcacgtgctaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctgggcttttgctggccttttgctcacatgttct

SEQ ID NO: 139 - DNA, 7210 bp, Synthetic construct
  source 1..7210: mol_type = other DNA
gactcttcgcgatgtacgggccagatatacgcgttaactgcagtctagattatgcgaaaggccatcctgacggatggcctttttgtttaaacggatccgcgacattgattattgactagttattaatagtaatcaattacggggtcattaggggactttccggggactttcctccccacgcgggggactttccgccacgggcggggactttccggggactttccgttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagctggctagcgtttaaacttaagcttggtaccgagctcggatccactagtccagtgtggtggaattctgcagatatccagcacagtggcggccgctcgagaatgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatccgataaggatcgatccggggcatgcaaccagctgtggaatgtgtgtcagttagggtgtggaaagtccccaggctccccagcaggcagaagtatgcaaagcatgtggggatgcggtgggctctatggcttctactgggcggttttatggacagcaagcgaaccggaattgccagctggggcgccctctggtaaggttgggaagccctgcaaagtaaactggatggctttctcgccgccaaggatctgtcgacccctagatttcagtgcaatttatctcttcaaatgtagcacctgaagtcagccccatacgatataagttgttggaagatctagcccgcctaatgagcgggcttttttttaattcgcaattccccgatgcataatgtgcctgtcaaatggacgaagcagggattctgcaaaccctatgctactccgtcaagccgtcaattgtctgattcgttaccaattatgacaacttgacggctacatcattcactttttcttcacaaccggcacgaaactcgctcgggctggccccggtgcattttttaaatactcgcgagaaatagagttgatcgtcaaaaccaacattgcgaccgacggtggcgataggcatccgggtagtgctcaaaagcagcttcgcctgactaatgcgttggtcctcgcgccagcttaagacgctaatccctaactgctggcggaaaagatgtgacagacgcgacggcgacaagcaaacatgctgtgcgacgctggcgatatcaaaattgctgtctgccaggtgatcgctgatgtactgacaagcctcgcgtacccgattatccatcggtggatggagcgactcgttaatcgcttccatgcgccgcagtaacaattgctcaagcagatttatcgccagcagctccgaatagcgcccttccccttgcccggcgttaatgatttgcccaaacaggtcgctgaaatgcggctggtgcgcttcatccgggcgaaagaaacccgtattggcaaatattgacggccagttaagccattcatgccagtaggcgcgcggacgaaagtaaacccactggtgataccattcgcgagcctccggatgacgaccgtagtgatgaatctctcctggcgggaacagcaaaatatcacccggtcggcagacaaattctcgtccctgatttttcaccaccccctgaccgcgaatggtgagattgagaatataacctttcattcccagcggtcggtcgataaaaaaatcgagataaccgttggcctcaatcggcgttaaacccgccaccagatgggcgttaaacgagtatcccggcagcaggggatcattttgcgcttcagccatacttttcatactcccaccattcagagaagaaaccaattgtccatattgcatcagacattgccgtcactgcgtcttttactggctcttctcgctaacccaaccggtaaccccgcttattaaaagcattctgtaacaaagcgggaccaaagccatgacaaaaacgcgtaacaaaagtgtctataatcacggcagaaaagtccacattgattatttgcacggcgtcacactttgctatgccatagcatttttatccataagattagcggatcctacctgacgctttttatcgcaactctctactgtttctccatacccgtttttttgggctagcgaattctgagaacaaactaagtggataaatttcgtgttcaggggccaacgaagctccagggcgaagtcacaatttccggcgctaaaaatgctgctctgcctatcctttttgccgcactactggcggaagaaccggtagagatccagaacgtcccgaaactgaaagacgtcgatacatcaatgaagctgctaagccagctgggtgcgaaagtagaacgtaatggttctgtgcatattgatgcccgcgacgttaatgtattctgcgcaccttacgatctggttaaaaccatgcgtgcttctatctgggcgctggggccgctggtagcgcgctttggtcaggggcaagtttcactacctggcggttgtacgatcggtgcgcgtccggttgatctacacatttctggcctcgaacaattaggcgcgaccatcaaactggaagaaggttacgttaaagcttccgtcgatggtcgtttgaaaggtgcacatatcgtgatggataaagtcagcgttggcgcaacggtgaccatcatgtgtgctgcaaccctggcggaaggcaccacgattattgaaaacgcagcgcgtgaaccggaaatcgtcgataccgcgaacttcctgattacgctgggtgcgaaaattagcggtcagggcaccgatcgtatcgtcatcgaaggtgtggaacgtttaggcggcggtgtctatcgcgttctgccggatcgtatcgaaaccggtactttcctggtggcggcggcgatttctcgcggcaaaattatctgccgtaacgcgcagccagatactctcgacgccgtgctggcgaaactgcgtgacgctggagcggacatcgaagtcggcgaagactggattagcctggatatgcatggcaaacgtccgaaggctgttaacgtacgtaccgcgccgcatccggcattcccgaccgatatgcaggcccagttcacgctgttgaacctggtggcagaagggaccgggtttatcaccgaaacggtctttgaaaaccgctttatgcatgtgccagagctgagccgtatgggcgcgcacgccgaaatcgaaagcaataccgttatttgtcacggtgttgaaaaactttctggcgcacaggttatggcaaccgatctgcgtgcatcagcaagcctggtgctggctggctgtattgcggaagggacgacggtggttgatcgtatttatcacatcgatcgtggctacgaacgcattgaagacaaactgcgcgctttaggtgcaaatattgagcgtgtgaaaggcgaataagaattcaggaaaaaaacgctgtgaaaaatgttggttttatcggctggcgcggaatggtcggctctgttctcatgcaacgcatggtagaggagcgcgatttcgacgctattcgccctgttttcttttctacctcccagtttggacaggcggcgcccaccttcggcgacacctccaccggcacgctacaggacgcttttgatctggatgcgctaaaagcgctcgatatcatcgtgacctgccagggcggcgattataccaacgaaatttatccaaagctgcgcgaaagcggatggcagggttactggattgatgcggcttctacgctgcgcatgaaagatgatgccattattattctcgacccggtcaaccaggacgtgattaccgacggcctgaacaatggcgtgaagacctttgtgggcggtaactgtaccgttagcctgatgttgatgtcgctgggcggtctctttgcccataatctcgttgactgggtatccgtcgcgacctatcaggccgcctccggcggcggcgcgcgccatatgcgcgagctgttaacccagatgggtcagttgtatggccatgtcgccgatgaactggcgacgccgtcttccgcaattcttgatattgaacgcaaagttacggcattgacccgcagcggcgagctgccggttgataactttggcgtaccgctggcgggaagcctgatcccctggatcgacaaacagctcgataacggccagagccgcgaagagtggaaaggccaggcggaaaccaacaagattctcaatactgcctctgtgattccggttgatggtttgtgtgtgcgcgtcggcgcgctgcgctgtcacagccaggcgttcaccatcaagctgaaaaaagaggtatccattccgacggtggaagaactgctggcggcacataatccgtgggcgaaagtggtgccgaacgatcgtgatatcactatgcgcgaattaaccccggcggcggtgaccggcacgttgactacgccggttggtcgtctgcgtaagctgaacatggggccagagttcttgtcggcgtttaccgtaggcgaccagttgttatggggcgccgccgagccgctgcgtcgaatgctgcgccagttggcgtagtctagctgcacgataccgtcgacttgtacatagactcgctccgaaattaaagaacacttaaattatctactaaaggaatctttagtcaagtttatttaagatgacttaactatgaatacacaattgatgggtgagcgtaggaaaaaaaaaccccgcccctgacagggcggggttttttttgatcattctgaaatgagctgttgacaattaatcatccggctcgtataatgtgtggaattgtgagcggataacaatttcacacaggaaacagaccatgaaaaaacaacatttccgtgtcgcccttattcccttttttgcggcattttgccttcctgtttttgctcacccagaaacgctggtgaaagtaaaagatgctgaacctaggagtactccgcggcccgggcccctgcaggttcgaaacagattaaatcagaacgcagaagcggtctgataaaacagaatttgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactctttttgtttatttttctaaatacattcaaatatgtatccgctcatgagacaataaccctgataaatgcttcaataatggaagatcttccaacatcacaggtaaacagaaacgtcgggtcgatcgggaaattctttcccggacggcgcggggttgggcaagccgcaggcgcgtcagtgcttttagcgggtgtcggggcagccctgaaccagtcacgggatcgatctgtgcggtatttcacaccgcatacaggtggcacttttcggggaaatgtgcgcggaacccctatttgtttatttttctaaatacattcaaatatgtatccgctcatgagacaataaccctgataaatgcttcaataatagcacgtgctaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctgggcttttgctggccttttgctcacatgttct

SEQ ID NO: 140 - DNA, 39 bp, synthetic construct
  source 1..39: mol_type = other DNA
ccgttgttgcttttcgggcacgcgccagggacgttagac

SEQ ID NO: 141 - DNA, 41 bp, synthetic construct
  source 1..41: mol_type = other DNA
gaaaagcaacaacggcgtgtgcggtggcccgtctactaaag

SEQ ID NO: 142 - DNA, 46 bp, synthetic construct
  source 1..46: mol_type = other DNA
cgctgaaaagcaattttataccaggtgcacgcgccagggacgttag

SEQ ID NO: 143 - DNA, 48 bp, synthetic construct
  source 1..48: mol_type = other DNA
aaattgcttttcagcgtaaccgtaaatgcggtggcccgtctactaaag

SEQ ID NO: 144 - DNA, 51 bp, synthetic construct
  source 1..51: mol_type = other DNA
attcatgtggtgcaactggacgagtttccatgcacgcgccagggacgttag

SEQ ID NO: 145 - DNA, 43 bp, synthetic construct
  source 1..43: mol_type = other DNA
gttgcaccacatgaatttcgttgcggtggcccgtctactaaag

SEQ ID NO: 146 - DNA, 45 bp, synthetic construct
  source 1..45: mol_type = other DNA
cgcaatgacgcggataaattgcatggcacgcgccagggacgttag

SEQ ID NO: 147 - DNA, 37 bp, synthetic construct
  source 1..37: mol_type = other DNA
atttatccgcgtcattgcggtggcccgtctactaaag

SEQ ID NO: 148 - DNA, 51 bp, synthetic construct
  source 1..51: mol_type = other DNA
aaactggagaccacatcggtggacgatgagtgcacgcgccagggacgttag

SEQ ID NO: 149 - DNA, 44 bp, synthetic construct
  source 1..44: mol_type = other DNA
gatgtggtctccagtttggccatgcggtggcccgtctactaaag

SEQ ID NO: 150 - DNA, 56 bp, synthetic construct
  source 1..56: mol_type = other DNA
aataacattttgcggagtataaccataccaatgatagcacgcgccagggacgttag

SEQ ID NO: 151 - DNA, 39 bp, synthetic construct
  source 1..39: mol_type = other DNA
ctccgcaaaatgttatttgcggtggcccgtctactaaag

SEQ ID NO: 152 - DNA, 38 bp, synthetic construct
  source 1..38: mol_type = other DNA
gtttggaccccagccatcgcacgcgccagggacgttag

SEQ ID NO: 153 - DNA, 37 bp, synthetic construct
  source 1..37: mol_type = other DNA
ggctggggtccaaactgcggtggcccgtctactaaag

SEQ ID NO: 154 - DNA, 69 bp, synthetic construct
  source 1..69: mol_type = other DNA
tttgaatttttggacgagctggatctttataaaccagatcacgagtatagcacgcgccagggacgttag

SEQ ID NO: 155 - DNA, 52 bp, synthetic construct
  source 1..52: mol_type = other DNA
cgtccaaaaattcaaaaaacttgtactttttgcggtggcccgtctactaaag

SEQ ID NO: 156 - DNA, 59 bp, synthetic construct
  source 1..59: mol_type = other DNA
atcagtagaatcagaatcacatttaccacaatgacactggcacgcgccagggacgttag

SEQ ID NO: 157 - DNA, 46 bp, synthetic construct
  source 1..46: mol_type = other DNA
gattctgattctactgattgtacttgcggtggcccgtctactaaag

SEQ ID NO: 158 - DNA, 59 bp, synthetic construct
  source 1..59: mol_type = other DNA
attcagcagatgcagcagcagagaaacaccgccaccagtgcacgcgccagggacgttag

SEQ ID NO: 159 - DNA, 56 bp, synthetic construct
  source 1..56: mol_type = other DNA
gctgcatctgctgaatactgaacaaggtgaatcttgcggtggcccgtctactaaag

SEQ ID NO: 160 - DNA, 51 bp, synthetic construct
  source 1..51: mol_type = other DNA
ccgcaatgatgcatagtacgagaagagcacgcgccagggacgttagacttg

SEQ ID NO: 161 - DNA, 33 bp, synthetic construct
  source 1..33: mol_type = other DNA
ctatgcatcattgcggtggcccgtctactaaag

SEQ ID NO: 162 - DNA, 59 bp, synthetic construct
  source 1..59: mol_type = other DNA
gcaatgatgagtataatcacaagcagtacacagataagtgcacgcgccagggacgttag

SEQ ID NO: 163 - DNA, 37 bp, synthetic construct
  source 1..37: mol_type = other DNA
gattatactcatcattgcggtggcccgtctactaaag

SEQ ID NO: 164 - DNA, 59 bp, synthetic construct
  source 1..59: mol_type = other DNA
aactggtttaccccaacgaaaatgttccatagaataagagcacgcgccagggacgttag

SEQ ID NO: 165 - DNA, 39 bp, synthetic construct
  source 1..39: mol_type = other DNA
gttggggtaaaccagtttgcggtggcccgtctactaaag

SEQ ID NO: 166 - DNA, 48 bp, synthetic construct
  source 1..48: mol_type = other DNA
caccgcaagcaaatgctggatgattaacgcacgcgccagggacgttag

SEQ ID NO: 167 - DNA, 31 bp, synthetic construct
  source 1..31: mol_type = other DNA
gcatttgcttgcggtggcccgtctactaaag

SEQ ID NO: 168 - protein, 9 aa, synthetic construct
CQDGRMGFC

SEQ ID NO: 169 - protein, 36 aa, synthetic construct; identical to SEQ ID NO: 133

SEQ ID NO: 170 - protein, 4 aa, synthetic construct
GGGS

SEQ ID NO: 171 - protein, 5 aa, synthetic construct
GGGGS

SEQ ID NO: 172 - protein, 5 aa, synthetic construct
EAAAK

SEQ ID NO: 173 - protein, 21 aa, synthetic construct
GCTKESGSVSSEQLAQFRSLD

SEQ ID NO: 174 - protein, 14 aa, synthetic construct
EGKSSGSGSESKST

SEQ ID NO: 175 - protein, 12 aa, synthetic construct
GSAGSAAGSGEF

SEQ ID NO: 176 - protein, 378 aa, unidentified
  source 1..378: note = Salmonella enterica subsp. enterica serovar Typhimurium str. UK-1
MKVKVLSLLVPALLVAGAANAAEIYNKDGNKLDLFGKVDGLHYFSDDKGSDGDQTYMRIGFKGETQVNDQLTGYGQWEYQIQGNQTEGSNDSWTRVAFAGLKFADAGSFDYGRNYGVTYDVTSWTDVLPEFGGDTYGADNFMQQRGNGYATYRNTDFFGLVDGLDFALQYQGKNGSVSGENTNGRSLLNQNGDGYGGSLTYAIGEGFSVGGAITTSKRTADQNNTANARLYGNGDRATVYTGGLKYDANNIYLAAQYSQTYNATRFGTSNGSNPSTSYGFANKAQNFEVVAQYQFDFGLRPSVAYLQSKGKDISNGYGASYGDQDIVKYVDVGATYYFNKNMSTYVDYKINLLDKNDFTRDAGINTDDIVALGLVYQF

SEQ ID NO: 177 - protein, 362 aa, unidentified
  source 1..362: note = Salmonella enterica subsp. enterica serovar Typhimurium str. UK-1
MKLKLVAVAVTSLLAAGVVNAAEVYNKDGNKLDLYGKVHAQHYFSDDNGSDGDKTYARLGFKGETQINDQLTGFGQWEYEFKGNRTESQGADKDKTRLAFAGLKFADYGSFDYGRNYGVAYDIGAWTDVLPEFGGDTWTQTDVFMTGRTTGVATYRNTDFFGLVEGLNFAAQYQGKNDRDGAYESNGDGFGLSATYEYEGFGVGAAYAKSDRTNNQVKAASNLNAAGKNAEVWAAGLKYDANNIYLATTYSETLNMTTFGEDAAGDAFIANKTQNFEAVAQYQFDFGLRPSIAYLKSKGKNLGTYGDQDLVEYIDVGATYYFNKNMSTFVDYKINLLDDSDFTKAAKVSTDNIVAVGLNYQF

SEQ ID NO: 178 - protein, 363 aa, unidentified
  source 1..363: note = Salmonella enterica subsp. enterica serovar Typhimurium str. UK-1
MMKRKILAAVIPALLAAATANAAEIYNKDGNKLDLYGKAVGRHVWTTTGDSKNADQTYAQIGFKGETQINTDLTGFGQWEYRTKADRAEGEQQNSNLVRLAFAGLKYAEVGSIDYGRNYGIVYDVESYTDMAPYFSGETWGGAYTDNYMTSRAGGLLTYRNSDFFGLVDGLSFGIQYQGKNQDNHSINSQNGDGVGYTMAYEFDGFGVTAAYSNSKRTNDQQDRDGNGDRAESWAVGAKYDANNVYLAAVYAETRNMSIVENTVTDTVEMANKTQNLEVVAQYQFDFGLRPAISYVQSKGKQLNGAGGSADLAKYIQAGATYYFNKNMNVWVDYRFNLLDENDYSSSYVGTDDQAAVGITYQF

SEQ ID NO: 179 - protein, 377 aa, unidentified
  source 1..377: note = Salmonella enterica subsp. enterica serovar Typhimurium str. UK-1
MKRKVLALVIPALLAAGAAHAAEIYNKDGNKLDLYGKVDGLHYFSDDSSKDGDQTYMRVGFKGETQINDQLTGYGQWEYNVQANTTEGEGANSWTRLAFAGLKFGDYGSFDYGRNYGVLYDVEGWTDMLPEFGGDSYTYADNFMTGRANGVATYRNTDFFGLVDGLNFALQYQGANENQVSHEQEGTNNGGDRNVKNANGDGFGISSTYDLGMGVSFGTAYTTSDRTNGQVNYSTAGGDKADAWTVGAKYDANNIYLATMYSETRNMTPYGNDDCRSCVANKTQNFEVTAQYQFDFGLRPAVSFLMSKGKDLTYNNVNGDDKDLVKYADVGATYYFNKNFSTYVDYKINLLDDDDQFYKDAGISTDDIVALGMVYQF

SEQ ID NO: 180 - protein, 212 aa, unidentified
  source 1..212: note = Salmonella enterica subsp. enterica serovar Typhimurium str. UK-1
MKKFTVAALALTTLLSGSAFAHEAGEFFMRAGPATVRPTEGAGGTLGHLNGFDVSNNTQLGLTFTYMATDNIGVELLAATPFRHKVGTGATGDIATVHLLPPTLMAQWYFGDSSSKVRPYVGVGVNYTTFFDNDFNDNGKNAGLSDLSFKDSWGAAGQVGVDYLINRDWLIGASVWYMDIDTTANYKMGGVQHHDSVRLDPWVFMFSAGYRF

SEQ ID NO: 181 - protein, 171 aa, unidentified
  source 1..171: note = Salmonella enterica subsp. enterica serovar Typhimurium str. UK-1
MKKIACLSALAAVLAFSAGTAVAATSTVTGGYAQSDAQGVANKMSGFNLKYRYEQDDNPLGVIGSFTYTEKDRTNGAGDYNKGQYYGITAGPAYRLNDWASIYGVVGVGYGKFQTTDYPTYKHDTSDYGFSYGAGLQFNPMENVALDFSYEQSRIRSVDVGTWIAGVGYRF

SEQ ID NO: 182 - protein, 656 aa, unidentified
  source 1..656: note = Salmonella enterica subsp. enterica serovar Typhimurium str. UK-1
MTQKRTLLKYGILSLALAAPLSACAFDSLTVIGDSLSDTGNNGRWTWDSGQNKLYDEQLAERYGLELSPSSNGGSNYAAGGATATPELNPQDNTADQVRQWLAKTGGKADHNGLYIHWVGGNDLAAAIAQPTMAQQIAGNSATSAAAQVGLLLDAGAGLVVVPNVPDISATPMILEAVITAGLGAAAPPALKAALDALAEGATPDFASRQQAIRKALLAAAATVSSNPFIQQLLVEQLLAGYEAAAGQASALTDYYNQMEEKGLEQHGGNIARADINGLFKEILANPQAFGLTNTVGMACPPGVSASACSSAMPGFNASQDYLFADHLHPGPQVHTIIAQYIQSIIAAPVQATYLNQSVQSMAQGSRTTLDSRYQQLRQGENPVGSLGMFGGYSGGYQRYDNNEADGNGNHNNLTVGVDYQLNEQVLLGGLIAGSLDKQHPDDNYRYDARGFQAAVFSHLRAGQAWLDSDLHFLSAKFSNIQRSITLGALRRVEEGETNGRLWGARLTSGYDFVMVPWLTTGPMLQYAWDYSHVNGYSEKLNTSTSMRFGDQNAHSQVGSAGWRLDLRHSIIHSWAQINYRRQFGDDTYVANGGLKSTALTFSRDGKTQDKNWVDIAIGADFPLSATVSAFAGLSQTAGLSDGNQTRYNVGFSARF

SEQ ID NO: 183 - protein, 614 aa, unidentified
  source 1..614: note = Salmonella enterica subsp. enterica serovar Typhimurium str. UK-1
MIKKATLLTAFSVTAFSAWAQDTSPDTLVVTANRFQQPRSAVLAPVTIVTRQDIERWQSTSVNDVLRRLPGVDIAQSGGAGQNSSIFIRGTNSSHVLVLIDGVRLNLAGVSGSADLSQFPVSLVQRIEYIRGPRSAIYGSDAIGGVVNIITTRDNPGTELTAGWGSNSYQNYDISTQQQLGENTRATLIGDYEYTKGFDVVAKGGTGMQAQPDRDGFLSKTLYGALEHTFSDRWSGFVRGYGYDNRTDYDAYYSPGSPLIDTRKLYSQSWDAGLHFNGERIQSQLVSSYSHSKDYNYDPHYGRYDTSATLDEMKQYNVQWTNSVVVGHGNVGAGVDWQKQTTTPGTGYVPEGYDQRNTGVYLTGLQQLGDFTLEAAARSDDNSQFGRHGTWQTSAGWEFIEGYRFIASYGTSYKAPNLGQLYGYYGNPNLNPEKSKQWEGAFEGLTAGVSWRISGYRNDINDMIDYDDHLQKYYNEGKARIKGIEATANFDTGPLTHTVSYDYVDARNAITDTPLPRRSKQMAKYQLDWDVYDFDWGMTYQYLGSRYDSDYSAYPYRTVKMGGVSLWDLTVAYPVTSHLTVRGKIANLFDKDYETVYGYQTAGREYTLSGSYTF

SEQ ID NO: 184 - protein, 663 aa, unidentified
  source 1..663: note = Salmonella enterica subsp. enterica serovar Typhimurium str. UK-1
MFRFNPFVRVGLCMSAVTLAWPVAAATDDGETMVVTASAIEQNLKDAPASISVITQQDLQRRPVQNLKDVLKEVPGVQLTNEGDNRKGVSIRGLDSSYTLILIDGKRVNSRNAVFRHNDFDLNWIPVDAIERIEVVRGPMSSLYGSDALGGVVNIITKKIGQKWHGSVTVDSTIQEHRDRGDTYNGQFFTSGPLIDGVLGMKAYGSLAKREKDEQQSSATTATGETPRIEGFTSRDGNVEFAWTPNENHDVTAGYGFDRQDRDSDSLDKNRLERQNYALSHNGRWDLGNSELKFYGEKVENKNPGNSSPITSESNSIDGKYVLPLASVNQFLTFGGEWRHDKLSDAVNLTGGSSTKTSASQYALFLEDEWRIFEPLALTTGIRMDDHETYGDHWSPRAYLVYNATDTLTVKGGWATAFKAPSLLQLSPDWATNSCRGGCRIVGSPDLKPETSESWELGLYYRGEEGILEGVEASVTTFRNDVDNRISISRTPDVNAAPGYSNFVGFETNSRGQRVPVFRYYNVNKARIQGVETELKVPFNEAWKLSLNYTYNDGRDVSNGGNKPLSDLPFHTANGTLDWKPVQLEDWSFYVSGNYTGRKRADSATAKTPGGYVVWDTGAAWQATKNVKLRAGVLNVGDKDLKRDDYGYTEDGRRYFMAVDYRF

SEQ ID NO: 185 - protein, 751 aa, unidentified
  source 1..751: note = Salmonella enterica subsp. enterica serovar Typhimurium str. UK-1
MNKKIHSLTLLVNLGIYGAALPVMAEDKTDSAALTNEDTIVVTAAQQNLQAPGVSTITADEIRKNPPARDVSEIIRTMPGVNLTGNSTSGQRGNNRQIDIRGMGPENTLILIDGKPVTSRNSVRLGWRGERDTRGDTAWVPPEMIERIEVLRGPAAARYGNGAAGGVVNIITKKGGSEWHGSWNTYFNAPEHKDEGATKRTNFSLNGPLGGDFSFRLYGNLDKTQADARNINQGHQSERTGSYADTLPAGREGVINKDINGVVRWDFAPLQSLELEAGYSRQGNLYAGDTQNTNTNQLVKDNYGKETNRLYRQNYSLTWNGGWNNGVTTSNWVQYEHTRNSRMPEGLAGGTEGIFDPKASQKYADADLNDVTLHSEVSLPFDLLVNQNLTLGTEWAQQRMKDQLSNSQTFMGGNIPGYSSTNRSPYSKAEIFSLFAENNMELTDSTMLTPGLRFDHHSIVGDNWSPSLNLSQGLGDDFTLKMGIARAYKAPSLYQTNPNYILYSKGQGCYATGAGTGIGCYMMGNDDLKAETSINKEIGLEFKRDGWLAGVTWFRNDYRNKIEAGTVPLQRINNGKTDVYQWENVPKAVVEGLEGTLNVPVSDTVNWTNNVTYMLQSKNKETGERLSIIPQYTLNSTLSWQVRQDVSLQSTFTWYGKQEPKKYDYQGNPVTGTDKQAVSPYSIVGLSATWDVTKNVSLTGGVDNLFDKRLWREGNAQTVRDTQTGAYMAGAGAYTYNEPGRTWYMSINTHF

SEQ ID NO: 186 - protein, 729 aa, unidentified
  source 1..729: note = Salmonella enterica subsp. enterica serovar Typhimurium str. UK-1
MARLKTAQPNSSLRKIAVVVATAVSGMSVYAQAAVQPKEETITVTAAPAAQESAWGPAPTIAAKRSATTTKTDTPIEKTPQSVSVVTNEEMQMHQFQSVKEALGYTPGVTVSSRGASNTYDFVIIRGFSSVGLSQNNYLDGLKLQGNFYNDAVIDPYMLERVELMRGPTSVLYGKSNPGGIISMVSKRPTTEPLKEIQFKMGTDNLFQTGFDFSDSLDDNGEFSYRLTGLARSTNEQQKSSESQRYAIAPSFTWRPDEKTNFTFLSYFQNEPETGYYGWLPKEGTVEPLPNGKRLPTDFNEGASNNTYSRNEKMVGYSFEHGFNDTFTVRQNLRFVEMKTAQKSVYGTGIAADGHTLNRGTIVDNERLQNFSVDTQLESKFATGDIDHTLLTGVDFMRMRNDINATFGSAPSIDLYNNYHPEYFAFGGAEPYQMNESKQTGLYVQDQAEWNKWVFTLGGRYDWSKQATTVRQNSTTPTEGYIERNDHQFTWRGGVNYVFDNGISPYFSYSQSFEPSAFDLWSTPRVSYKPSKGEQYEAGVKYVPKDMPVVVTGAVYQLTKTNNLTADPTNPLAQVPAGEIRARGVELEAKAALTANINMTASYTYTDAEYTKDTNLKGNTPEQVPEHMASLWGDYTFNEGPLSGLTLGTGGRFIGSSYGDPANSFKVGSAAVMDAVVKYDLARFGMAGSSIAVNVNNLLDREYVASCFQTYGCFWGAERQVVATATFRF

SEQ ID NO: 187 - protein, 726 aa, unidentified
  source 1..726: note = Salmonella enterica subsp. enterica serovar Typhimurium str. UK-1
MGMRVKKFIWLITVVSTGVNSPLSAAESTDDNGETMVVESTAEQVLKQQPGVSIITRDDIQKNPPVNDLADIIRKMPGVNLTGNSASGTRGNNRQIDIRGLGPENTLVLIDGVPVTSRNSVRYSWRGERDTRGDTNWVPPEMVERIEVIRGPAAARYGSGAAGGVVNIITKRPTNDWHGSLSLYTNQPESSKEGDTRRGNFSLSGPLAGDTLTMRLYGNLNRTDADSWDINSSAGTKNAAGREGVTNKDINSVFSWKMTPQQILDFEAGYSRQGNIYAGDTQNSTSNAVTKSLAQSGRETNRLYRQNYGLTHNGIWDWGQSRLGFYYEKTDNTRMNEGLSGGGEGRITNDQTFTTNRLTSYRTSGEVNVPVIWLFEQTLTVGAEWNRDELNDPSSTSLTVKDSNIAGIPGSAANRSSKNKSEISALYVEDNIEPMAGTNIIPGLRFDYLSESGSNFSPSLNLSQELGEYVKVKAGIARAFKAPNLYQTSEGYLLYSKGNGCPKDITSGGCYLVGNKNLDPEISINKEIGLEFTVDDYHASVTYFRNDYQNKIVAGDQIIGRSASGAYVLQWQNGGKALIEGIEASMAVPLMPDRLNWNTNATYMIASEQKDTGNPLSIIPKYTVNTFLDWTITSALSANVNWTLYGKQKPRTHAESRSEETKGLSGKALGAYSLVGANVNYDINKNLRLNVGISNIFDKQIYRSAEGANTYNEPGRAYYAGVTASF

SEQ ID NO: 188 - protein, 185 aa, unidentified
  source 1..185: note = Salmonella enterica subsp. enterica serovar Typhimurium str. UK-1
MKNIILSTLVITTSVLVVNVAQADTNAFSVGYAQSKVQDFKNIRGVNVKYRYEDDSPVSFISSLSYLYGDRQASGSVEPEGIHYHDKFEVKYGSLMVGPAYRLSDNFSLYALAGVGTVKATFKEHSTQDGDSFSNKISSRKTGFAWGAGVQMNPLENIVVDVGYEGSNISSTKINGFNVGVGYRF

SEQ ID NO: 189 - protein, 185 aa, unidentified
  source 1..185: note = Salmonella enterica subsp. enterica serovar Typhimurium str. UK-1
MKKIVLSSLLLSAAGLAAVPVAQADTHSVSVGYAQSRIEHFKDIRGVNLKYRYEAQTPLGLMASFSWQSGKRGESGGIPGGMSWRDDVKATYWSLMAGPAVRVNELVSLYALAGAGTGRAEVKERISMPGYNGRFTGSERRTGFAWGAGVQFNPVENVVIDLGYEGSKVGAAKLNGVNVGVGYRF

SEQ ID NO: 190 - protein, 12 aa, synthetic construct
TWYKIAFQRNRK

SEQ ID NO: 191 - protein, 9 aa, synthetic construct
CRWYDENAC

SEQ ID NO: 192 - protein, 9 aa, synthetic construct
CRWYDANAC

SEQ ID NO: 193 - protein, 7 aa, synthetic construct
KSPNPRF

SEQ ID NO: 194 - protein, 7 aa, synthetic construct
QRHKPRE

SEQ ID NO: 195 - protein, 12 aa, synthetic construct
YHWYGYTPQNVI

SEQ ID NO: 196 - protein, 6 aa, synthetic construct
LARLLT

SEQ ID NO: 197 - protein, 12 aa, synthetic construct
CMYIEALDKYAC

SEQ ID NO: 198 - protein, 12 aa, synthetic construct
FPMFNHWEQWPP

SEQ ID NO: 199 - protein, 16 aa, synthetic construct
RCSHGYTGIRCQAVVL

SEQ ID NO: 200 - protein, 6 aa, synthetic construct
FALGEA

SEQ ID NO: 201 - protein, 15 aa, synthetic construct
HFLIIGFMRRALCGA

SEQ ID NO: 202 - protein, 15 aa, synthetic construct
HFLIIGFMRRAACGA

SEQ ID NO: 203 - protein, 16 aa, synthetic construct
KYFPPLALYNPTEYFY

SEQ ID NO: 204 - protein, 12 aa, synthetic construct
VLGREEWSTSYW

SEQ ID NO: 205 - protein, 14 aa, synthetic construct
WSGENGPGFYDYEA

SEQ ID NO: 206 - protein, 5 aa, synthetic construct
AEYLR

SEQ ID NO: 207 - protein, 9 aa, synthetic construct
EEEEYFELV

SEQ ID NO: 208 - protein, 9 aa, synthetic construct
DEDEYFELV

SEQ ID NO: 209 - protein, 11 aa, synthetic construct
PLLQATLGGGS

SEQ ID NO: 210 - protein, 8 aa, synthetic construct
QWAVGHLM

SEQ ID NO: 211 - protein, 6 aa, synthetic construct
GNQWFI

SEQ ID NO: 212 - protein, 7 aa, synthetic construct
VATANST

SEQ ID NO: 213 - protein, 7 aa, synthetic construct
HAMRAQP

SEQ ID NO: 214 - protein, 9 aa, synthetic construct
CPKSNNGVC

SEQ ID NO: 215 - protein, 9 aa, synthetic construct
CGNKRTRGC

SEQ ID NO: 216 - protein, 9 aa, synthetic construct
CNKRTRGGC

SEQ ID NO: 217 - protein, 9 aa, synthetic construct
CTPSPFSHC

SEQ ID NO: 218 - protein, 7 aa, synthetic construct
IFLLWQR

SEQ ID NO: 219 - protein, 7 aa, synthetic construct
VHLGYAT

SEQ ID NO: 220 - protein, 12 aa, synthetic construct
DWSSWVYRDPQT

SEQ ID NO: 221 - protein, 9 aa, synthetic construct
CPIEDRPMC

SEQ ID NO: 222 - protein, 12 aa, synthetic construct
ANLNLWTDYIRW

SEQ ID NO: 223 - protein, 12 aa, synthetic construct
HEWSYLAPYPWF

SEQ ID NO: 224 - protein, 7 aa, synthetic construct
SPTKSNS

SEQ ID NO: 225 - protein, 9 aa, synthetic construct
CDCRGDCFC

SEQ ID NO: 226 - protein, 9 aa, synthetic construct
CSSAGSLFC

SEQ ID NO: 227 - protein, 12 aa, synthetic construct
NPWHSIYITRFG

SEQ ID NO: 228 - protein, 7 aa, synthetic construct
PLLQATL

SEQ ID NO: 229 - protein, 4 aa, synthetic construct
GGGS

SEQ ID NO: 230 - protein, 7 aa, synthetic construct
LSPPRYP

SEQ ID NO: 231 - protein, 6 aa, synthetic construct
VYMSPF

SEQ ID NO: 232 - protein, 10 aa, synthetic construct
CAVYMSPFAC

SEQ ID NO: 233 - protein, 7 aa, synthetic construct
HSQAAVP

SEQ ID NO: 234 - protein, 13 aa, synthetic construct
CTVALPGGYVRVC

SEQ ID NO: 235 - protein, 8 aa, synthetic construct
WIFPWIQL

SEQ ID NO: 236 - protein, 12 aa, synthetic construct
WDLAWMFRLPVG

SEQ ID NO: 237 - protein, 7 aa, synthetic construct
WHWRLPS

SEQ ID NO: 238 - protein, 12 aa, synthetic construct
TYLCTACDYTHH

SEQ ID NO: 239 - protein, 13 aa, synthetic construct
SYSMEHFRWGKPV

SEQ ID NO: 240 - protein, 10 aa, synthetic construct
CEHFRWCKPV

SEQ ID NO: 241 - protein, 11 aa, synthetic construct
CCEHFRWCKPV

SEQ ID NO: 242 - protein, 24 aa, synthetic construct
AMEHFRWGRPVGSGSGSGSVWYAG

SEQ ID NO: 243 - protein, 13 aa, synthetic construct
SSIISHFRWGKPV

SEQ ID NO: 244 - protein, 12 aa, synthetic construct
YVLGHFLFDRFG

SEQ ID NO: 245 - protein, 10 aa, synthetic construct
YERKFWHGRH

SEQ ID NO: 246 - protein, 7 aa, synthetic construct
NPNWGPR

SEQ ID NO: 247 - protein, 7 aa, synthetic construct
TTHQFPF

SEQ ID NO: 248 - protein, 9 aa, synthetic construct
CVNHPAFAC

SEQ ID NO: 249 - protein, 12 aa, synthetic construct
VPWXEPAYQRFL

SEQ ID NO: 250 - protein, 9 aa, synthetic construct
CSSRTMHHC

SEQ ID NO: 251 - protein, 10 aa, synthetic construct
TAASGVRSMH

SEQ ID NO: 252 - protein, 10 aa, synthetic construct
LTLRWVGLMS

SEQ ID NO: 253 - protein, 12 aa, synthetic construct
PRRIKPRKIMLQ

SEQ ID NO: 254 - protein, 12 aa, synthetic construct
QRILQQINLPRI

SEQ ID NO: 255 - protein, 12 aa, synthetic construct
QCTGPNVATNCR

SEQ ID NO: 256 - protein, 12 aa, synthetic construct
TRTKLPRLHLQS

SEQ ID NO: 257 - protein, 12 aa, synthetic construct
INQDARTMVMVP

SEQ ID NO: 258 - protein, 6 aa, synthetic construct
DGLGDD

SEQ ID NO: 259 - protein, 6 aa, synthetic construct
DGWGPN

SEQ ID NO: 260 - protein, 6 aa, synthetic construct
DGLGDA

SEQ ID NO: 261 - protein, 6 aa, synthetic construct
DGWGPA

SEQ ID NO: 262 - protein, 6 aa, synthetic construct
LDWDAI

SEQ ID NO: 263 - protein, 12 aa, synthetic construct
WSGPGVWGASVK

SEQ ID NO: 264 - protein, 6 aa, synthetic construct
GRGDYV

SEQ ID NO: 265 - protein, 13 aa, synthetic construct
CNGRCVSGCAGRC

SEQ ID NO: 266 - protein, 5 aa, synthetic construct
CNGRC

SEQ ID NO: 267 - protein, 12 aa, synthetic construct
YHWYGYTPENVI

SEQ ID NO: 268 - protein, 7 aa, synthetic construct
SYPIPDT

SEQ ID NO: 269 - protein, 7 aa, synthetic construct
HTSDQTN

SEQ ID NO: 270 - protein, 12 aa, synthetic construct
KHLQCVRNICWS

SEQ ID NO: 271 - protein, 12 aa, synthetic construct
EHLHCLGSLCWP

SEQ ID NO: 272 - protein, 12 aa, synthetic construct
NNLRCIGNICWS

SEQ ID NO: 273 - protein, 14 aa, synthetic construct
WRPTRYRLLPWWIC

SEQ ID NO: 274 - protein, 12 aa, synthetic construct
WSRIALRLGLIC

SEQ ID NO: 275 - protein, 7 aa, synthetic construct
SNFYMPL

SEQ ID NO: 276 - protein, 8 aa, synthetic construct
SNFYMPLK

SEQ ID NO: 277 - protein, 10 aa, synthetic construct
YEVHTYYLDC

SEQ ID NO: 278 - protein, 7 aa, synthetic construct
FALKGDY

SEQ ID NO: 279 - protein, 7 aa, synthetic construct
FAAKGDY

SEQ ID NO: 280 - protein, 12 aa, synthetic construct
YSAYPDSVPMMS

SEQ ID NO: 281 - protein, 12 aa, synthetic construct
SWLAYPGAVSYR

SEQ ID NO: 282 - protein, 12 aa, synthetic construct
APYCVYRGSWSC

SEQ ID NO: 283 - protein, 12 aa, synthetic construct
KYLPYWPVLSSL

SEQ ID NO: 284 - protein, 12 aa, synthetic construct
VTMEAINLAFPG

SEQ ID NO: 285 - protein, 12 aa, synthetic construct
NHWLDTLFPMHM

SEQ ID NO: 286 - protein, 19 aa, synthetic construct
TGGGVSLLLHLLNTEQGES

SEQ ID NO: 287 - protein, 12 aa, synthetic construct
MHTAPGWGYRLS

SEQ ID NO: 288 - protein, 9 aa, synthetic construct
CTVRTSAEC

SEQ ID NO: 289 - protein, 9 aa, synthetic construct
CIGNSNTLC

SEQ ID NO: 290 - protein, 21 aa, synthetic construct
YTRDLVYKDPARPKIQKTCTF

SEQ ID NO: 291 - protein, 15 aa, synthetic construct
QCHCGKCDSDSTDCT

SEQ ID NO: 292 - protein, 7 aa, synthetic construct
MQLPLAT

SEQ ID NO: 293 - protein, 12 aa, synthetic construct
DRWVARDPASIF

SEQ ID NO: 294 - protein, 12 aa, synthetic construct
TVGLPMTYYMHT

SEQ ID NO: 295 - protein, 8 aa, synthetic construct
LSCQLYQR

SEQ ID NO: 296 - protein, 6 aa, synthetic construct
KCCYSL

SEQ ID NO: 297 - protein, 17 aa, synthetic construct
ACSLQDPNCDWWGHYCG

SEQ ID NO: 298 - protein, 17 aa, synthetic construct
ACYLQDPNCDWWGPYCG

SEQ ID NO: 299 - protein, 17 aa, synthetic construct
ACGLQGYGCWGMYGKCG

SEQ ID NO: 300 - protein, 6 aa, synthetic construct
MARSGL

SEQ ID NO: 301 - protein, 6 aa, synthetic construct
MARAKE

SEQ ID NO: 302 - protein, 6 aa, synthetic construct
MSRTMS

SEQ ID NO: 303 - protein, 26 aa, synthetic construct
PFTPLAGSWTWENGKWTWKGHYRGKT

SEQ ID NO: 304 - protein, 20 aa, synthetic construct
WTGWCLNPEESTWGFCTGSF

SEQ ID NO: 305 - protein, 7 aa, synthetic construct
LTVSPWY

SEQ ID NO: 306 - protein, 10 aa, synthetic construct
IHGHHIISVG

SEQ ID NO: 307 - protein, 10 aa, synthetic construct
EHWSYGLRPG

SEQ ID NO: 308 - protein, 7 aa, synthetic construct
HAIYPRH

SEQ ID NO: 309 - protein, 12 aa, synthetic construct
THRPPMWSPVWP

SEQ ID NO: 310 - protein, 9 aa, synthetic construct
CVHSPNKKC

SEQ ID NO: 311 - protein, 7 aa, synthetic construct
VHPKQHR

SEQ ID NO: 312 - protein, 7 aa, synthetic construct
SWQIGGN

SEQ ID NO: 313 - protein, 12 aa, synthetic construct
METRPVAPHEFR

SEQ ID NO: 314 - protein, 21 aa, synthetic construct
IATTTASAATAAAIGATPRAK

SEQ ID NO: 315 - protein, 12 aa, synthetic construct
LTPPGRLSSWPL

SEQ ID NO: 316 - protein, 12 aa, synthetic construct
SVTLSLRLPFPS

SEQ ID NO: 317 - protein, 7 aa, synthetic construct
PHLTALF

SEQ ID NO: 318 - protein, 7 aa, synthetic construct
NPMIRRQ

SEQ ID NO: 319 - protein, 15 aa, synthetic construct
RSLWSDFYASASRGP

SEQ ID NO: 320 - protein, 15 aa, synthetic construct
RRLPHLMPFEGSVFL

SEQ ID NO: 321 - protein, 12 aa, synthetic construct
MPHPTKNFDLYV

SEQ ID NO: 322 - protein, 12 aa, synthetic construct
SVSVGMKPSPRP

SEQ ID NO: 323 - protein, 5 aa, synthetic construct
PPVAT